SEQUENCE LISTING (64 sequences)

SEQ ID NO: 1 - protein, 16 aa, Artificial Sequence
  Synthetic Peptide
RSSQSIVHSVGNTFLE

SEQ ID NO: 2 - protein, 7 aa, Artificial Sequence
  Synthetic Peptide
KVSNRFS

SEQ ID NO: 3 - protein, 9 aa, Artificial Sequence
  Synthetic Peptide
FQGSQFPYT

SEQ ID NO: 4 - protein, 10 aa, Artificial Sequence
  Synthetic Peptide
GYEFSRSWMN

SEQ ID NO: 5 - protein, 18 aa, Artificial Sequence
  Synthetic Peptide
GRIYPGDGDTNYSGKFKG

SEQ ID NO: 6 - protein, 11 aa, Artificial Sequence
  Synthetic Peptide
DGSSWDWYFDV

SEQ ID NO: 7 - protein, 107 aa, Artificial Sequence
  Synthetic Polypeptide
DIQMTQSPSSLSASVGDRVTITCSASQGISNYLNWYQQKPGKTVKLLIYYTSNLHSGVPSRFSGSGSGTDYTLTISSLQPEDFATYYCQQYSELPWTFGQGTKVEIK

SEQ ID NO: 8 - protein, 120 aa, Artificial Sequence
  Synthetic Polypeptide
EVQLVESGPALVKPTQTLTLTCTVSGFSLTGYSVNWIRQPPGKALEWLGMIWGDGSTDYNSALKSRLTISKDTSKNQVVLTMTNMDPVDTATYYCARDYYFNYASWFAYWGQGTLVTVSS

SEQ ID NO: 9 - protein, 11 aa, Artificial Sequence
  Synthetic Peptide
SASQGISNYLN

SEQ ID NO: 10 - protein, 7 aa, Artificial Sequence
  Synthetic Peptide
YTSNLHS

SEQ ID NO: 11 - protein, 9 aa, Artificial Sequence
  Synthetic Peptide
QQYSELPWT

SEQ ID NO: 12 - protein, 10 aa, Artificial Sequence
  Synthetic Peptide
GFSLTGYSVN

SEQ ID NO: 13 - protein, 16 aa, Artificial Sequence
  Synthetic Peptide
MIWGDGSTDYNSALKS

SEQ ID NO: 14 - protein, 12 aa, Artificial Sequence
  Synthetic Peptide
DYYVNYASWFAY

SEQ ID NO: 15 - protein, 214 aa, Artificial Sequence
  Synthetic Polypeptide
DIQMTQSPSSLSASVGDRVTITCSASQGISNYLNWYQQKPGKTVKLLIYYTSNLHSGVPSRFSGSGSGTDYTLTISSLQPEDFATYYCQQYSELPWTFGQGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 16 - protein, 450 aa, Artificial Sequence
  Synthetic Polypeptide
EVQLVESGPALVKPTQTLTLTCTVSGFSLTGYSVNWIRQPPGKALEWLGMIWGDGSTDYNSALKSRLTISKDTSKNQVVLTMTNMDPVDTATYYCARDYYFNYASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 112 aa, Artificial Sequence
  Synthetic Polypeptide
DIVMTQSPDSLAVSLGERATINCRASQSVSGSRFTYMHWYQQKPGQPPKLLIKYASILESGVPDRFSGSGSGTDFTLTISSLQAEDVAVYYCQHSWEIPPWTFGQGTKVEIK

SEQ ID NO: 18 - protein, 118 aa, Artificial Sequence
  Synthetic Polypeptide
EVQLVQSGAEVKKPGASVKVSCKASGYSFTGYWMNWVRQAPGQGLEWIGMIHPLDAEIRANQKFRDRVTITVDTSTSTAYLELSSLRSEDTAVYYCARGTYDGGFEYWGQGTLVTVSS

SEQ ID NO: 19 - protein, 15 aa, Artificial Sequence
  Synthetic Peptide
RASQSVSGSRFTYMH

SEQ ID NO: 20 - protein, 7 aa, Artificial Sequence
  Synthetic Peptide
YASILES

SEQ ID NO: 21 - protein, 10 aa, Artificial Sequence
  Synthetic Peptide
QHSWEIPPWT

SEQ ID NO: 22 - protein, 5 aa, Artificial Sequence
  Synthetic Peptide
GYWMN

SEQ ID NO: 23 - protein, 17 aa, Artificial Sequence
  Synthetic Peptide
MIHPLDAEIRANQKFRD

SEQ ID NO: 24 - protein, 9 aa, Artificial Sequence
  Synthetic Peptide
GTYDGGFEY

SEQ ID NO: 25 - protein, 219 aa, Artificial Sequence
  Synthetic Polypeptide
DIVMTQSPDSLAVSLGERATINCRASQSVSGSRFTYMHWYQQKPGQPPKLLIKYASILESGVPDRFSGSGSGTDFTLTISSLQAEDVAVYYCQHSWEIPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 448 aa, Artificial Sequence
  Synthetic Polypeptide
EVQLVQSGAEVKKPGASVKVSCKASGYSFTGYWMNWVRQAPGQGLEWIGMIHPLDAEIRANQKFRDRVTITVDTSTSTAYLELSSLRSEDTAVYYCARGTYDGGFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 17 aa, Artificial Sequence
  Synthetic Peptide
MIHPMDSEIRANQKFRD

SEQ ID NO: 28 - protein, 17 aa, Artificial Sequence
  Synthetic Peptide
MIHPLDSEIRANQKFRD

SEQ ID NO: 29 - protein, 9 aa, Artificial Sequence
  Synthetic Peptide
GTYDGGFKY

SEQ ID NO: 30 - protein, 219 aa, Artificial Sequence
  Synthetic Polypeptide
DIVMTQSPDSLAVSLGERATINCRASQSVSGSRFTYMHWYQQKPGQPPKLLIKYASILESGVPDRFSGSGSGTDFTLTISSLQAEDVAVYYCQHSWEIPPWTFGQGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 31 - protein, 448 aa, Artificial Sequence
  Synthetic Polypeptide
EVQLVQSGAEVKKPGASVKVSCKASGYSFTGYWMNWVRQAPGQGLEWIGMIHPLDAEIRANQKFRDRVTITVDTSTSTAYLELSSLRSEDTAVYYCARGTYDGGFEYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 11 aa, Artificial Sequence
  Synthetic Peptide
KASDLIHNWLA

SEQ ID NO: 33 - protein, 8 aa, Artificial Sequence
  Synthetic Peptide
YGATSLET

SEQ ID NO: 34 - protein, 9 aa, Artificial Sequence
  Synthetic Peptide
QQYWTTPFT

SEQ ID NO: 35 - protein, 11 aa, Artificial Sequence
  Synthetic Peptide
GYSITNDYAWN

SEQ ID NO: 36 - protein, 17 aa, Artificial Sequence
  Synthetic Peptide
GYISYSGYTTYNPSLKS

SEQ ID NO: 37 - protein, 9 aa, Artificial Sequence
  Synthetic Peptide
ARWASGLDY

SEQ ID NO: 38 - protein, 108 aa, Artificial Sequence
  Synthetic Polypeptide
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIKR

SEQ ID NO: 39 - protein, 116 aa, Artificial Sequence
  Synthetic Polypeptide
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWASGLDYWGQGTLVTVSS

SEQ ID NO: 40 - protein, 11 aa, Artificial Sequence
  Synthetic Peptide
GYSITSDYAWN

SEQ ID NO: 41 - protein, 17 aa, Artificial Sequence
  Synthetic Peptide
GYISNSGSTSYNPSLKS

SEQ ID NO: 42 - protein, 15 aa, Artificial Sequence
  Synthetic Peptide
ERNYDYDDYYYAMDY

SEQ ID NO: 43 - protein, 17 aa, Artificial Sequence
  Synthetic Peptide
KSSQSLLYRSNQKNYLA

SEQ ID NO: 44 - protein, 7 aa, Artificial Sequence
  Synthetic Peptide
WASTRES

SEQ ID NO: 45 - protein, 9 aa, Artificial Sequence
  Synthetic Peptide
QQYYNYPRT

SEQ ID NO: 46 - protein, 124 aa, Artificial Sequence
  Synthetic Polypeptide
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSS

SEQ ID NO: 47 - protein, 113 aa, Artificial Sequence
  Synthetic Polypeptide
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIK

SEQ ID NO: 48 - protein, 10 aa, Artificial Sequence
  Synthetic Peptide
GFSFSDFAMS

SEQ ID NO: 49 - protein, 18 aa, Artificial Sequence
  Synthetic Peptide
ATIGRVAFHTYYPDSMKG

SEQ ID NO: 50 - protein, 13 aa, Artificial Sequence
  Synthetic Peptide
ARHRGFDVGHFDF

SEQ ID NO: 51 - protein, 16 aa, Artificial Sequence
  Synthetic Peptide
RSSETLVHSSGNTYLE

SEQ ID NO: 52 - protein, 7 aa, Artificial Sequence
  Synthetic Peptide
RVSNRFS

SEQ ID NO: 53 - protein, 9 aa, Artificial Sequence
  Synthetic Peptide
FQGSFNPLT

SEQ ID NO: 54 - protein, 120 aa, Artificial Sequence
  Synthetic Polypeptide
EVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSS

SEQ ID NO: 55 - protein, 113 aa, Artificial Sequence
  Synthetic Polypeptide
DIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIKR

SEQ ID NO: 56 - protein, 117 aa, Artificial Sequence [sequence omitted]

SEQ ID NO: 57 - protein, 112 aa, Artificial Sequence
  Synthetic Polypeptide
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKR

SEQ ID NO: 58 - protein, 10 aa, Artificial Sequence
  Synthetic Peptide
GYTFSSYWIE

SEQ ID NO: 59 - protein, 18 aa, Artificial Sequence
  Synthetic Peptide
GEILPGGGDTNYNEIFKG

SEQ ID NO: 60 - protein, 10 aa, Artificial Sequence
  Synthetic Peptide
TRRVPIRLDY

SEQ ID NO: 61 - protein, 15 aa, Artificial Sequence
  Synthetic Peptide
KASQSVDYEGDSFLN

SEQ ID NO: 62 - protein, 7 aa, Artificial Sequence
  Synthetic Peptide
AASNLES

SEQ ID NO: 63 - protein, 9 aa, Artificial Sequence
  Synthetic Peptide
QQSNEDPLT

SEQ ID NO: 64 - protein, 1255 aa, Homo sapiens [sequence omitted]

(19) United States
(12) Patent Application Publication
(10) Pub. No.: US 2022/0098191 A1
(43) Pub. Date: Mar. 31, 2022

(54) G-A CROSSLINKING CYTOTOXIC AGENTS

(71) Applicant: FEMOGENIX LIMITED, Harpenten Hertfordshire (GB)

(72) Inventors: Paul JACKSON, Harpenden Hertfordshire (GB); David THURSTON, Harpenten Hertfordshire (GB); George PROCOPIOU, Harpenten Hertfordshire (GB)

(21) Appl. No.: 17/426,492

(22) PCT Filed: Jan. 28, 2020
(86) PCT No.: PCT/GB2020/050200

§ 371 Date: Jul. 28, 2021

(30) Foreign Application Priority Data
Jan. 29, 2019 (GB) 1901197.2

(51) Int. Cl. C07D 471/04; A61P 35/00; A61K 47/54

(52) U.S. Cl. CPC C07D 471/04; A61K 47/545; A61P 35/00

(57) ABSTRACT

The invention relates to a compound of formula (I): or salts, solvates, isomers or tautomers thereof, wherein; A is a group selected from: R₁ is selected from H and halogen; either R₂ is selected from —CH₂-halogen, C₁₋₆ alkyl and H, and R₃ is H or is absent; or R₂ and R₃ together with the carbon atoms to which they are attached form a cyclopropyl ring; SP is a spacer group; B is a polycyclic group: R₁₁, R₁₂, R₁₃ and R₁₄ are selected such that either: (aa) one of R₁₁, R₁₂, R₁₃ and R₁₄ is E and another of R₁₁, R₁₂, R₁₃ and R₁₄ is an optionally substituted Ar¹ group; or (ab) one of R₁₁, R₁₂, R₁₃ and R₁₄ is Rx, wherein R^{x} is Ar¹—Z¹-E or -E¹-Z¹—Ar¹; and Ar¹ is an optionally substituted C₅₋₂₀ aryl or C₅₋₁₀ heteroaryl group each E is independently selected from (CH₂)ⱼ—S(0)₂—NR₂₅R₂₆, (CH₂)ⱼ—S(0)₂-0H, CH₂CH₂[0CH₂CH₂]_{W}R₂₅ and E¹; and Z₁ is NR₂₆, C(=0)-0, O or is absent and these compounds are useful as medicaments, in particular as anti-proliferative agents.

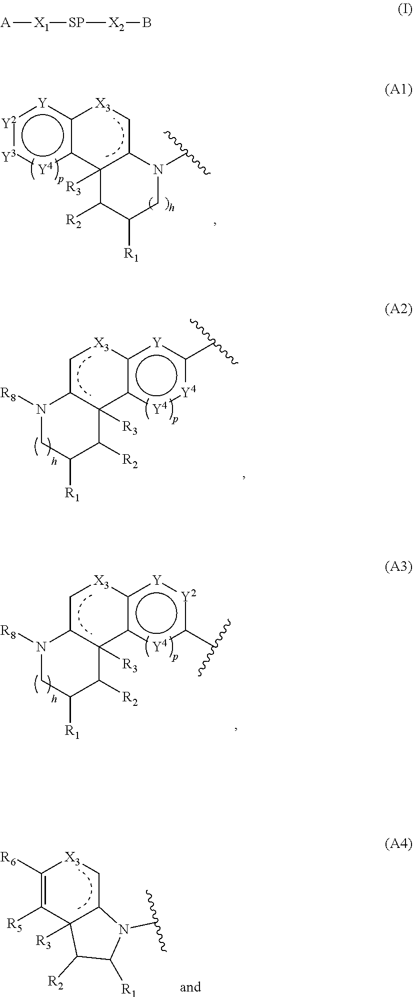

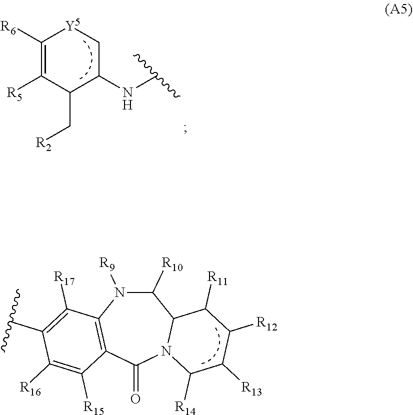

### FIELD OF THE INVENTION

The invention relates to DNA-alkylating units comprising fused rings. In particular it relates to compounds comprising a substituted C-ring of a G-alkylator linked via the A-ring to other aromatic A-alkylating units (e.g., CBI, CPI, CTI, etc.), or salts, solvates, isomers or tautomers thereof, which are useful as medicaments, in particular as anti-proliferative agents.

### BACKGROUND TO THE INVENTION

The pyrrolobenzodiazepines (PBDs) are a group of compounds some of which have been shown to be sequence-selective DNA minor-groove binding agents. The PBDs were originally discovered in Streptomyces species^{[1b, 2]} They are tricyclic in nature, and are comprised of of fused 6-7-5-membered rings and can be identified as an anthranilate (A ring), a diazepine (B ring) and a pyrrolidine (C ring)^{[2b]} They are characterized by an electrophilic N10-C11 imine group (as shown below) or the hydrated equivalent, a carbinolamine (i.e., hemiaminal) [NH—CH(OH)], or a carbinolamine alkyl ether ([NH—CH(OR, where R=alkyl)] which can form a covalent bond to a C2-amino group of guanine in DNA to form a DNA adduct^{[3]}. The natural products interact in the minor groove of the DNA helix with excellent fit (i.e., good “isohelicity”) due to a right-handed longitudinal twist induced by a stereogenic centre at the C11a-position which has the (S)-configuration^{[4]}.

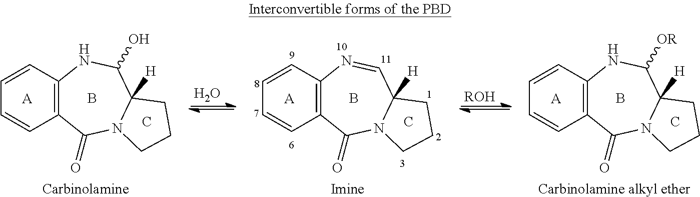

The DNA adduct has been reported to inhibit a number of biological processes including the binding of transcription factors^{[5] [6]} and the function of enzymes such as endonucleases^{[7]} and RNA polymerase^{[8]}. PBD monomers (e.g., anthramycin) have been shown by footprinting^{[4]}, NMR^{[9]}, molecular modeling^{[10]} and X-ray crystallography^{[11]} to span three base pairs and to have a thermodynamic preference for the sequence 5′-Pu-G-Pu-3′ (where Pu=purine, and G is the reacting guanine)^{[12]} and a kinetic preference for the sequence 5′-Py-G-Py-3′ (where Py=pyridine).

PBDs are thought to interact with DNA by first locating at a low-energy binding sequence (i.e., a 5′-Pu-G-Pu-3′ triplet) through Van der Waals, hydrogen bonding and electrostatic interactions^{[5]}. Then, once in place, a nucleophilic attack by the exocyclic C2-amino group of the central guanine occurs to form the covalent adduct^{[5]}. Once bound, the PBD remains anchored in the DNA minor groove, avoiding DNA repair by causing negligible distortion of the DNA helix^{[11]}. The ability of PBDs to form an adduct in the minor groove and either monoalkylated or crosslink DNA depending on their structure enables them to interfere with DNA processing and, hence, their potential for use as antiproliferative agents.

Bizelesin and related dimeric cyclopropylpyrroloindole (CPI) molecules have been investigated as stand-alone anticancer agents but they were abandoned as potential clinical agents due to significant liver toxicity. Such dimeric CPI molecules are capable of binding to adenine bases (A) and so forming sequence selective A-A cross-links in the DNA minor groove.

More recently PBD and CPI/CBI units have been joined together to create asymmetric molecules capable of forming cross-links to both G and A bases, the first example was UTA-6026^{[13]}.

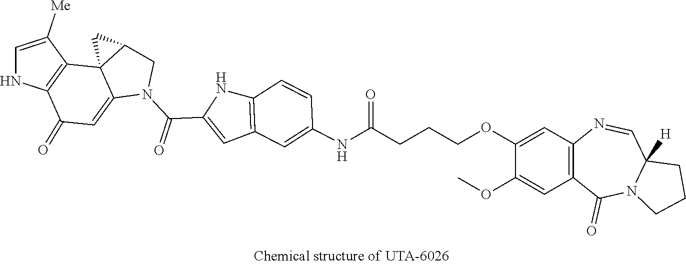 [SMILES: COc1cc2c(cc1OCCCC(=O)Nc1ccc3[nH]c(C(=O)N4C[C@H]5C[C@@]56C4=CC(=O)c4[nH]cc(C)c46)cc3c1)N=C[C@@H]1CCCN1C2=O]

The most persuasive evidence for significant interstrand cross-linking ability and cytotoxicity of asymmetric molecules of this type relate to 27eS^{14]} which was significantly more cytotoxic than a number of PBD dimers.

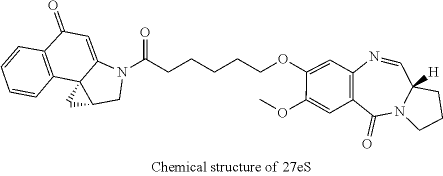 [SMILES: COc1cc2c(cc1OCCCCCC(=O)N1C[C@@H]3C[C@]34C1=CC(=O)c1ccccc14)N=C[C@@H]1CCCN1C2=O]

WO2015023355 discloses drug moieties comprising 1,2,9,9a-tetrahydrocyclopropa-[1,2-c]benz[1,2-e]indol-4-one (CBI) dimers and also drug moieties comprising a CBI linked to an unsubstituted PBD. WO2015023355 also discloses antibody-drug conjugates comprising such drug moieties; furthermore, immunoconjugates comprising such drug moieties linked to antibodies that bind HER2 are disclosed in WO2016040723. WO 2004/087711, WO 2011/117882 and WO 2013/164593 disclose PBD (6-7-5) dimers linked via their A-rings, and more recently WO2012128868 and WO2016115191 disclose G-alkylating agents containing a D-ring (i.e., 6-7-5-6 and 6-7-6-6 respectively), all of which have been shown to act as cytotoxic agents in vitro and as anti-tumour agents in vivo in animal tumour models. Further PBD and PDD compounds have been disclosed^{[15]} in WO2013055990, WO2024140862, WO2015028850, WO2016198869 and WO2016115201. Finally WO2017194960 discloses PDD-CBI dimers (G-A cross-linkers).

No agents that act through cross-linking A to G base pairs have been developed for clinical use.

The present application reports asymmetric conjugate compounds comprising a G-alkylating unit and an A-alkylating unit. The inventors have discovered that unsymmetrical dimers based on these constructs provide properties such as reduced hydrophobicity (compared to known cytotoxic agents), potent cytotoxicity and sequence-selective DNA binding ability, all of which result in effective compounds. Reduced hydrophobicity, in particular, promotes efficient conjugation, which is a significant issue in the development of antibody-drug conjugates.

The present invention seeks to overcome problem(s) associated with the prior art.

### SUMMARY

In a first aspect, there is provided a compound of formula (I):

A-X₁—SP—X₂—B (I)

or salts, solvates, isomers or tautomers thereof,
wherein;
A is a group selected from:

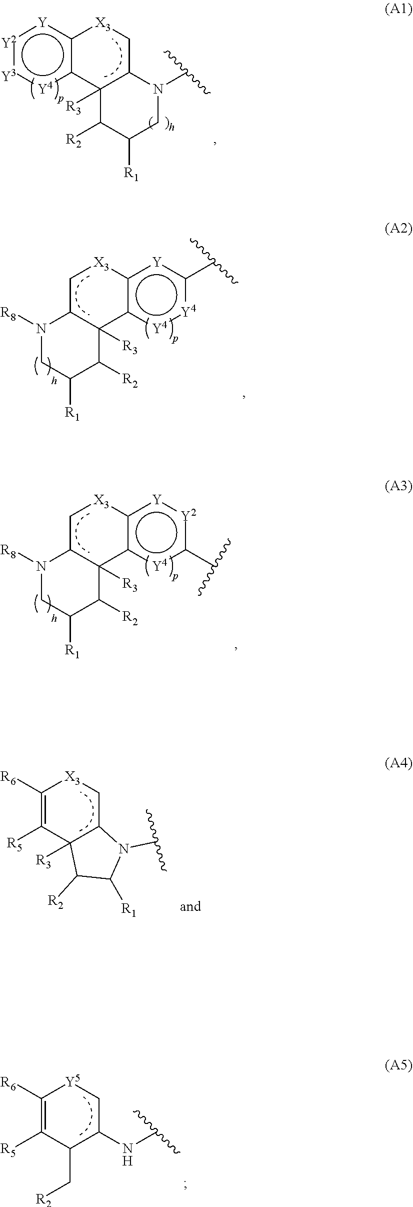

h is 0 or 1;
R₁ is selected from H and halogen;
either R₂ is selected from —CH₂-halogen, C₁₋₆ alkyl and H, and R₃ is H or is absent;
   or R₂ and R₃ together with the carbon atoms to which they are attached form a cyclopropyl ring;
p is 0 or 1; and when p is 1 then Y is C—R₇, Y² is C—R₆, Y³ is C—R₅ and Y⁴ is C—R₄; and for (A1) and (A2) when p is 0 either (a) Y is selected from N—R₁₉, O and S; Y² is selected from C—R₆ and N; and Y³ is C—R₅; or (b) Y³ is selected from N—R₁₉, O and S; Y² is selected from C—R₆ and N; and Y is C—R₇; and for (A3) when p is 0, Y is selected from N—R₁₉, O and S; and Y² is selected from C—R₆ and N;
R₄, R₅, R₆ and R₇ are each independently selected from H, C₁₋₆ alkyl, OC₁₋₆ alkyl, CO₂H, CO₂C₁₋₆ alkyl, OCH₂Ph and R₂₀;
   or one of R₄ and R₅, or R₅ and R₆, or R₆ and R₇ together with the carbon atoms to which they are attached form a 6-membered aryl, or a 5- or 6-membered cyclic, heterocyclic, or heteroaryl ring optionally substituted with 1, 2 or 3 optional groups independently selected from C₁₋₆ alkyl, OC₁₋₆ alkyl, OCH₂Ph and R₂₀;
R₈ is selected from selected from H, C₁₋₆ alkyl, OC₁₋₆ alkyl, OCH₂Ph, nitrogen
protecting groups and R₂₀;
X₃ is selected from C═O, C—OH and C—R₁₈; or Y⁵ is selected from C═O, C—OH, C—NH₂ and C—R₁₈; with the carbon forming part of the ring; and
   when X₃ or Y⁵ is C═O then [symbol] represents an α,β-unsaturated double bond conjugated with the C═O; and when X₃ is C—OH or C—R₁₈ or Y⁵ is C—OH, C—NH₂ or C—R₁₈ then [symbol] represents the double bonds of an aromatic 6-membered ring and R₃ is absent;
wherein R¹⁸ is a prodrug moiety comprising carbonyl, carbamoyl, glycosyl, O-amino, O-acylamino, para-aminobenzyl ether, peptidyl or phosphate groups;
   X₁ is selected from O, S, NR₂₁, CR₂₁R₂₂, CR₂₁R₂₂O, C(O), C(O)NR₂₁, NR₂₁C(O), O—C(O), C(O)—O or is absent;
   SP is selected from an amino acid, a peptide chain having from 2 to 12 amino acids, a paraformaldehyde chain —(CH₂O)₁₋₂₄—, a polyethylene glycol chain —(CH₂CH₂O)₁₋₁₂— and —(CH₂)ₘ—Y⁶—(CH₂)ₙ— wherein
m is 0, 1, 2, 3, 4, 5, 6, 7, 8, 9, 10, 11 or 12;
n is 0, 1, 2, 3, 4, 5, 6, 7, 8, 9, 10, 11 or 12; and
Y⁶ is selected from —(CH₂)ᵣ—, arylene, heteroarylene, cycloalkylene, cycloalkenylene and heterocyclylene and the Y⁶ group is optionally substituted with 1, 2 or 3 independently selected optional C₁₋₆ alkyl, OC₁₋₆ alkyl, OCH₂Ph and
R₂₀ groups;
z is 1, 2, 3, 4 or 5;
   X₂ is selected from O, S, NR₂₃, CR₂₃R₂₄, CR₂₃R₂₄O, C(O), C(O)NR₂₃, NR₂₄C(O), O—C(O), C(O)—O or is absent;
   B is a polycyclic group:

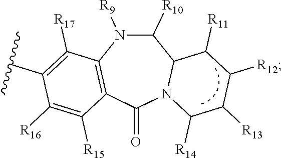

wherein the dotted lines indicate the optional presence of one or more double bonds;
and R₉ and R₁₀ are selected such that either:
   (i) R₉ and R₁₀ together form a double bond;
   (ii) R₉ is H and R₁₀ is OH;
   (iii) R₉ is H and R₁₀ is OC₁₋₆ alkyl;
   (iv) R₉ is SO₃H, a nitrogen protecting group or R₂₀; and R₁₀ is H; or
   (v) R₉ is H or C₁₋₆ alkyl, and R₁₀ is oxo or H;
R₁₁, R₁₂, R₁₃ and R₁₄ are selected such that either:
(aa) one of R₁₁, R₁₂, R₁₃ and R₁₄ is E and another of R₁₁, R₁₂, R₁₃ and R₁₄ is an Ar¹ group optionally substituted with 1, 2 or 3 optional groups independently selected from C₁₋₆ alkyl, OC₁₋₆ alkyl, OCH₂Ph, E, R₂₀ and R₂₅; or
(ab) one of R₁₁, R₁₂, R₁₃ and R₁₄ is R^{X}, wherein R^{X} is —Ar¹—Z¹-E or -E¹-Z¹—Ar¹ and the Ar¹ is optionally further substituted with 1 or 2 optional groups independently selected from C₁₋₆ alkyl, OC₁₋₆ alkyl, OCH₂Ph, E, R₂₀ and R₂₅; or
(ac) one of R₁₁, R₁₂, R₁₃ and R₁₄ is R^{Y}, wherein R^{Y} is a 5- or 6-membered cyclic, heterocyclic, or heteroaryl ring optionally substituted with up to two groups independently selected from C₁₋₆ alkyl, OC₁₋₆ alkyl, OCH₂Ph, E, R₂₀ and R₂₅;
and for any of (aa) or (ab) or (ac) the remaining of R₁₁, R₁₂, R₁₃ and R₁₄ are independently selected from H, C₁₋₆ alkyl, OC₁₋₆ alkyl, OCH₂Ph, (CH₂)ⱼ—S(O)₂—NR₂₆R₂₇, R₂₀, R₂₅, ═CH₂, ═CH—(CH₂)ₛ—CH₃, ═CH—(CH₂)ₛ—R₂₅, ═O, (CH₂)ₛ—OR₂₅, (CH₂)ₛ—CO₂R₂₅, (CH₂)ₛ—NR₂₅R₂₆, O—(CH₂)ₜ—NR₂₅R₂₆, NH—C(O)—R₂₅, O—(CH₂)ₜ—NH—C(O)—R₂₅, O—(CH₂)ₜ—C(O)—NH—R₂₅, (CH₂)ₛ—SO₂R₂₅, O—SO₂R₂₅, (CH₂)ₛ—C(O)R₂₅ and (CH₂)ₛ—C(O)NR₂₅R₂₆;
wherein Ar¹ is an optionally substituted C₅₋₂₀ aryl or C₅₋₁₀ heteroaryl group; and each E is independently selected from (CH₂)ⱼ—S(O)₂—NR₂₅R₂₆, (CH₂)ⱼ—S(O)₂—OH, CH₂CH₂[OCH₂CH₂]_{w}R₂₅ and E¹;
   each E¹ is independently selected from pentose; hexose; C₅₋₆ heterocyclyl; C₅₋₁₀ heteroaryl group; C₅₋₁₀ heteroaryl group substituted with a C₅₋₆ heteroaryl or a C₅₋₆ heterocyclyl group; and phenyl substituted with a C₅₋₆ heteroaryl or a C₅₋₆ heterocyclyl group; wherein each E¹ group may be independently optionally substituted with 1, 2 or 3 optional groups independently selected from OC₁₋₆ alkyl, OCH₂Ph, R₂₀ and R₂₇; with the proviso that the E¹ group comprises at least two heteroatoms;
Z¹ is NR₂₆, C(═O)—O, O or is absent;
   each w is independently 1, 2, 3, 4, 5, 6, 7, 8, 9 or 10;
each s is independently 0, 1, 2, 3, 4, 5 or 6;
each t is independently 1, 2, 3, 4, 5 or 6;
R₁₅, R₁₆ and R₁₇ are independently selected from H, C₁₋₆ alkyl, OC₁₋₆ alkyl, OCH₂Ph, R₂₀ and R₂₅;
   each R₂₀ is independently selected from (CH₂)ⱼ—OH, (CH₂)ⱼ—CO₂R₂₇, C(O)R₂₇, O—(CH₂)ₖ—NR₂₇R₂₈, (CH₂)ⱼ—NR₂₇R₂₈, K₁—R*, NR₂₇NR₂₇R₂₈, C(O)—O—(CH₂)ₖ—NR₂₇R₂₈, C(O)NR₂₇R₂₈, NR₂₈—CO₂R₂₇, C(O)—NH—(CH₂)ₖ—NR₂₇R₂₈, C(O)—NH—C₆H₄—(CH₂)ⱼ—R₂₇ and C(O)—NH—(CH₂)ₖ—C(═NH)NR₂₇R₂₈;
each j is independently 0, 1, 2, 3, 4, 5 or 6;
each k is independently 1, 2, 3, 4, 5 or 6;
   each R₁₉, R₂₁, R₂₂, R₂₃, R₂₄, R₂₆ and R₂₈ is independently selected from H and C₁₋₆ alkyl;
   each R₂₅ is independently selected from H, C₁₋₁₂ alkyl, C₅₋₉ heteroaryl, C₆₋₁₆ heteroarylalkyl, phenyl and C₆₋₂₆ aralkyl groups; wherein the heteroaryl, heteroarylalkyl, phenyl and aralkyl groups are optionally substituted with 1, 2 or 3 independently selected optional C₁₋₆ alkyl, OC₁₋₆ alkyl and R₂₀ groups;
   each R₂₇ is independently selected from H, C₁₋₆ alkyl, C₅₋₂₀ aryl and C₆₋₂₆ aralkyl; and
K₁ is a bond or a linker moiety having 1-200 non-hydrogen atoms selected from C, N, O, S or halogen, and optionally incorporates alkyl, ether, oxo, carboxyl, carboxamide, carboxamidyl, ester, urethanyl, branched, cyclic, unsaturated, heterocyclyl, aryl or heteroaryl moieties; and
R* is an azide, alkyne, bisulfone, carbohydrazide, hydrazine, hydroxylamine, iodoacetamide, isothiocyanate, maleimide, phosphine, pyrridopyridazine, semihydrazide, succinimidyl ester, sulfodichlorophenol ester, sulfonyl halide, sulfosuccinimidyl ester, 4-sulfotetrafluorophenyl ester, tetrafluorophenyl ester, thiazole, R₂₀, O—(CH₂)ₖ—NR₂₆R₂₆, NHNH₂, or is a targeting agent wherein the targeting agent is selected from a protein, a portion of a protein, a polypeptide, a nucleic acid, a hormone, an antibody or an antibody fragment;
   with the proviso that when R₂ is C₁₋₆ alkyl or H, that R₉ and R₁₀ are selected from options (i), (ii), (iii) or (iv); and
   with the proviso that when (v) R₉ is H or C₁₋₆ alkyl, and R₁₀ is oxo or H; then either R₂ is —CH₂-halogen and R₃ is H;
   or R₂ and R₃ together with the carbon atoms to which they are attached form a cyclopropyl ring.

In a further aspect, there is described a pharmaceutical composition comprising a compound of formula (I) or salts, solvates, tautomers, isomers or mixtures thereof, and a pharmaceutically acceptable carrier, diluent, or excipient. The pharmaceutical composition of the present invention may further comprise one or more (e.g. two, three or four) further active agents.

In a further aspect, there is described a compound of formula (I) or salts, solvates, tautomers, isomers or mixtures thereof, or a pharmaceutical composition as described herein, for use as a medicament.

In a further aspect, there is described a compound of formula (I) or salts, solvates, tautomers, isomers or mixtures thereof, for use as a drug in an antibody-drug conjugate.

In a further aspect, there is described a compound of formula (I) or salts, solvates, tautomers, isomers or mixtures thereof, or a pharmaceutical composition, as described herein, for use in the treatment of a proliferative disease, a bacterial infection, a malarial infection and inflammation.

In a further aspect, there is provided a compound of formula (I) or salts, solvates, tautomers, isomers or mixtures thereof as described herein, linked, either directly or indirectly, to a targeting agent to provide a targeting conjugate.

In a further aspect, there is provided a compound of formula (I) or salts, solvates, tautomers, isomers or mixtures thereof as described herein, linked to a linking group.

In a further aspect, there is provided a compound of formula (I) or salts, solvates, tautomers, isomers or mixtures thereof, or a pharmaceutical composition as described herein, for use in a method of therapy.

In certain aspects, the compound of formula (I) or salts, solvates, tautomers, isomers or mixtures thereof, may be used as a payload on a tumour-targeting agent (e.g., a protein, a portion of a protein, a polypeptide, a nucleic acid, a hormone, an antibody, an antibody fragment, etc.).

In a further aspect, the compound of formula (I) or salts, solvates, tautomers, isomers or mixtures thereof, may be linked, either directly or indirectly, to a targeting agent to provide a targeted conjugate. In a further aspect, the compound of formula (I) or salts, solvates, tautomers, isomers or mixtures thereof, may contain a linker group, wherein the targeting agent is attached to the compound of formula (I) or salts, solvates, tautomers, isomers or mixtures thereof, through the linker group. The target conjugates of the present disclosure may contain one or multiple compounds of formula (I) or salts, solvates, tautomers, isomers or mixtures thereof. A variety of target conjugates are known in the art and maybe used with a compound of formula (I) and salts or solvates thereof. For example, in a particular aspect the target conjugate is an antibody-drug conjugate, wherein one or more compounds of formula (I) are linked, directly or indirectly, to the antibody. Therefore, the compound of formula (I) and salts or solvates thereof, may be used as a payload on a targeted conjugate.

In a further aspect, the present invention provides a method of treatment of a patient suffering from a proliferative disease, comprising administering to said patient a therapeutically effective amount of a compound of formula (I) or salts, solvates, tautomers, isomers or mixtures thereof, or a pharmaceutical composition comprising a compound of formula (I).

In a further aspect, the compound of formula (I) or salts, solvates, tautomers, isomers or mixtures thereof, may be administered alone or in combination with other treatments, either simultaneously or sequentially depending upon the condition to be treated.

Further particular and preferred aspects are set out in the accompanying independent and dependent claims. Features of the dependent claims may be combined with features of the independent claims as appropriate, and in combinations other than those explicitly set out in the claims.

### Definitions

The following abbreviations are used throughout the specification: Ac acetyl; Alloc allyloxycarbonyl; Bn benzyl; Boc tert-butoxycarbonyl; CBI 1,2,9,9a-tetrahydrocyclopropa[1,2-c]benz[1,2-e]indol-4-one; CPI cyclopropylpyrroloindole; CTI cyclopropylthiophene[e]indoline; DCM dichloromethane; DIPEA N,N-diisopropylethylamine; DMA dimethylacetamide; DMAP 4-dimethylaminopyridine; DMF dimethylformamide; DMSO dimethylsulfoxide; DPPA diphenyl-phosphoryl azide; EDCI 1-Ethyl-3-(3-dimethylaminopropyl)carbodiimide; Et ethyl; Et₂O diethyl ether; EtOAc ethyl acetate; EtOH ethanol; HATU (1-[Bis(dimethylamino)methylene]-1H-1,2,3-triazolo[4,5-b]-pyridinium 3-oxid hexafluorophosphate); Me methyl; MeOH methanol; NaHMDS sodium hexamethyldisilazide or sodium bis(trimethylsilyl)amide; PBDs pyrrolo[2,1-c][1,4]benzo-diazepines; PDDs pyrridinobenzodiazepines; Pd(dppf)Cl2 [1,1′-Bis(diphenylphosphino)ferrocene]-dichloropalladium(II); Ph phenyl; PIDA (diacetoxyiodo)benzene; Pyr pyridine; TBAF tetrabutylammonium fluoride; TEMPO (2,2,6,6-tetramethyl-piperidin-1-yl)oxyl; TBS-Cl/TBDMSCl tert-butyldimethylsilyl chloride; TFAA trifluoroacetic anhydride; THF tetrahydrofuran; and TMS-NCS trimethylsilyl isothiocyanate.

“Substituted”, when used in connection with a chemical substituent or moiety (e.g., an alkyl group), means that one or more hydrogen atoms of the substituent or moiety have been replaced with one or more non-hydrogen atoms or groups, provided that valence requirements are met and that a chemically stable compound results from the substitution.

“Optionally substituted” refers to a parent group which may be unsubstituted or which may be substituted with one or more substituents. Suitably, unless otherwise specified, when optional substituents are present the optional substituted parent group comprises from one to three optional substituents. Where a group may be “optionally substituted with 1, 2 or 3 groups”, this means that the group may be substituted with 0, 1, 2 or 3 of the optional substituents. Suitably, the group is substituted with 1, 2 or 3 of the optional substituents. Where a group is “optionally substituted with one or two optional substituents”, this means that the group may be substituted with 0, 1 or 2 of the optional substituents. Suitably, the group may be optionally substituted with 0 or 1 optional substituents. In some aspects, suitably the group is not optionally substituted. In other aspects, suitably the group is substituted with 1 of the optional substituents.

Optional substituents may be selected from C₁₋₁₂ alkyl, C₂₋₇ alkenyl, C₂₋₇ alkynyl, C₁₋₁₂ alkoxy, C₅₋₂₀ aryl, C₃₋₁₀ cycloalkyl, C₃₋₁₀ cycloalkenyl, C₃₋₁₀ cycloalkynyl, C₃₋₂₀ heterocyclyl, C₃₋₂₀ heteroaryl, acetal, acyl, acylamido, acyloxy, amidino, amido, amino, aminocarbonyloxy, azido, carboxy, cyano, ether, formyl, guanidino, halo, hemiacetal, hemiketal, hydroxamic acid, hydroxyl, imidic acid, imino, ketal, nitro, nitroso, oxo, oxycarbonyl, oxycarboyloxy, sulfamino, sulfamyl, sulfate, sulfhydryl, sulfinamino, sulfinate, sulfino, sulfinyl, sulfinyloxy, sulfo, sulfonamido, sulfonamino, sulfonate, sulfonyl, sulfonyloxy, uredio groups. In some aspects, the optional substituents are 1, 2 or 3 optional substituents independently selected from OH, C₁₋₁₂ alkyl, OC₁₋₁₂ alkyl, R_{A} and halogen. More suitably, the optional substituents are selected from OH, C₁₋₁₂ alkyl and OC₁₋₁₂ alkyl; more suitably, the optional substituents are selected from C₁₋₁₂ alkyl and OC₁₋₁₂ alkyl.

“Independently selected” is used in the context of statement that, for example, “each R₂₃ and R₂₄ are independently selected from H and C₁₋₆ alkyl, . . . ” and means that each instance of the functional group, e.g. R₂₃, is selected from the listed options independently of any other instance of R₂₃ or R₂₄ in the compound. Hence, for example, H may be selected for the first instance of R₂₃ in the compound; methyl may be selected for the next instance of R₂₃ in the compound; and ethyl may be selected for the first instance of R₂₄ in the compound.

C₁₋₁₂ alkyl: refers to straight chain and branched saturated hydrocarbon groups, generally having from 1 to 12 carbon atoms; suitably a C₁₋₁₁ alkyl; suitably a C₁₋₁₀ alkyl; suitably a C₁₋₉ alkyl; suitably a C₁₋₈ alkyl; more suitably a C₁₋₇ alkyl; more suitably a C₁₋₆ alkyl; more suitably a C₁₋₅ alkyl; more suitably a C₁₋₄ alkyl; more suitably a C₁₋₃ alkyl.

Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, n-heptyl, and the like.

“Alkylene” refers to a divalent radical derived from an alkane which may be a straight chain or branched, as exemplified by —CH₂CH₂CH₂CH₂—.

The term “amino acid” refers to both the twenty “canonical” or “natural” amino acids, as well “non-canonical” amino acids, also referred to as “unnatural” amino acids, such as modified or synthetic amino acids, as well as amino acid analogs and amino acid mimetics that function similarly to naturally occurring amino acids. Naturally occurring amino acids are those encoded by the genetic code, i.e. they are amino acids selected from alanine, argenine, asparagine, aspartic acid, cysteine, glutamine, glutamic acid, glycine, histidine, isoleucine, leucine, lysine, methionine, phenylalanine, proline, serine, threonine, tryptophan, tyrosine and valine. Modified amino acids include, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

“C₆₋₂₆ aralkyl” refers to an arylalkyl group having 6 to 26 carbon atoms and comprising an alkyl group substituted with an aryl group. Suitably the alkyl group is a C₁₋₆ alkyl group. Suitably, the aryl group is phenyl. Examples of C₆₋₂₆ aralkyl include benzyl and phenethyl. In some cases the C₆₋₂₆ aralkyl group may be optionally substituted and an example of an optionally substituted C₆₋₂₆ aralkyl group is 4-methoxylbenzyl.

“C₅₋₂₀ Aryl”: refers to fully unsaturated monocyclic, bicyclic and polycyclic aromatic hydrocarbons having at least one aromatic ring and having a specified number of carbon atoms that comprise their ring members (e.g., C₅₋₂₀ aryl refers to an aryl group having from 5 to 20 carbon atoms as ring members). The aryl group may be attached to a parent group or to a substrate at any ring atom and may include one or more non-hydrogen substituents unless such attachment or substitution would violate valence requirements. Suitably, a C₆₋₁₄ aryl is selected from a C₆₋₁₂ aryl, more suitably, a C₆₋₁₀ aryl. Examples of aryl groups include phenyl, biphenyl, indenyl and naphthalenyl.

“Arylene” refers to a divalent radical derived from an aryl group, e.g. —C₆H₄— which is the arylene derived from phenyl.

“C₃₋₈ cycloalkyl” or “3- to 8-membered cycloalkyl” means a closed ring of carbon atoms having 3 to 8 carbon atoms, preferably 3 to 7 carbon atoms, more preferably 3 to 6 carbon atoms and encompasses, for example, cyclopropyl, cyclobutyl, cyclopentyl, cyclohexyl, cycloheptyl and cyclooctyl.

“C₃₋₈ cycloalkylene” or “3- to 8-membered cycloalkylene” refers to a divalent radical derived from a cycloalkyl group, e.g. —C₆H₁₂—.

Halogen or halo: refers to a group selected from F, Cl, Br, and I. Each halogen in a compound of formula (I) is independently selected. Suitably, the halogen or halo is F or Cl. More suitably, the halogen is Cl.

“C₅₋₁₀ heteroaryl” or “5- to 10-membered heteroaryl”: refers to unsaturated monocyclic or bicyclic aromatic groups comprising from 5 to 10 ring atoms, whether carbon or heteroatoms, of which from 1 to 5 are ring heteroatoms. Suitably. the heteroaryl may be a C₅₋₉ heteroaryl. Suitably, any monocyclic heteroaryl ring has from 5 to 6 ring atoms (i.e. is a C₅₋₆ heteroaryl) and from 1 to 3 ring heteroatoms. Suitably each ring heteroatom is independently selected from nitrogen, oxygen, and sulfur. The bicyclic rings include fused ring systems and, in particular, include bicyclic groups in which a monocyclic heterocycle comprising 5 ring atoms is fused to a benzene ring. The heteroaryl group may be attached to a parent group or to a substrate at any ring atom and may include one or more non-hydrogen substituents unless such attachment or substitution would violate valence requirements or result in a chemically unstable compound.

Examples of monocyclic heteroaryl groups include, but are not limited to, those derived from:

N₁: pyrrole, pyridine;
O₁: furan;
S₁: thiophene;
N₁O₁: oxazole, isoxazole, isoxazine;
N₂O₁: oxadiazole (e.g. 1-oxa-2,3-diazolyl, 1-oxa-2,4-diazolyl, 1-oxa-2,5-diazolyl, 1-oxa-3,4-diazolyl);
N₃O₁: oxatriazole;
N₁S₁: thiazole, isothiazole;
N₂: imidazole, pyrazole, pyridazine, pyrimidine, pyrazine;
N₃: triazole, triazine; and,
N₄: tetrazole.

Examples of heteroaryl which comprise fused rings, include, but are not limited to, those derived from:

O₁: benzofuran, isobenzofuran;
N₁: indole, isoindole, indolizine, isoindoline;
S₁: benzothiofuran;
N₁O₁: benzoxazole, benzisoxazole;
N₁S₁: benzothiazole;
N₂: benzimidazole, indazole;
O₂: benzodioxole;
N₂O₁: benzofurazan;
N₂S₁: benzothiadiazole;
N₃: benzotriazole; and
N₄: purine (e.g., adenine, guanine), pteridine;

“heteroarylene” refers to a divalent radical derived from a heteroaryl group (such as those described above) as exemplified by pyridinyl —[C₅H₃N]—. Heteroarylenes may be monocyclic, bicyclic, or tricyclic ring systems. Representative heteroarylenes, are not limited to, but may be selected from triazolylene, tetrazolylene, oxadiazolylene, pyridylene, furylene, benzofuranylene, thiophenylene, benzothiophenylene, quinolinylene, pyrrolylene, indolylene, oxazolylene, benzoxazolylene, imidazolylene, benzimidazolylene, thiazolylene, benzothiazolylene, isoxazolylene, pyrazolylene, isothiazolylene, pyridazinylene, pyrimidinylene, pyrazinylene, triazinylene, cinnolinylene, phthalazinylene, quinazolinylene, pyrimidylene, azepinylene, oxepinylene, and quinoxalinylene. Heteroarylenes are optionally substituted.

“C₆₋₁₆ heteroarylalkyl” refers to an alkyl group substituted with a heteroaryl group.

Suitably the alkyl is a C₁₋₆ alkyl group and the heteroaryl group is C₅₋₁₀ heteroaryl as defined above. Examples of C₆₋₁₆ heteroarylalkyl groups include pyrrol-2-ylmethyl, pyrrol-3-ylmethyl, pyrrol-4-ylmethyl, pyrrol-3-ylethyl, pyrrol-4-ylethyl, imidazol-2-ylmethyl, imidazol-4-ylmethyl, imidazol-4-ylethyl, thiophen-3-ylmethyl, furan-3-ylmethyl, pyridin-2-ylmethyl, pyridin-2-ylethyl, thiazol-2-ylmethyl, thiazol-4-ylmethyl, thiazol-2-ylethyl, pyrimidin-2-ylpropyl, and the like.

“C₃₋₂₀ heterocyclyl”: refers to saturated or partially unsaturated monocyclic, bicyclic or polycyclic groups having ring atoms composed of 3 to 20 ring atoms, suitably 5 or 6 ring atoms, whether carbon atoms or heteroatoms, of which from 1 to 10 are ring heteroatoms. Suitably, each ring has from 3 to 7 ring atoms and from 1 to 4 ring heteroatoms (e.g., suitably C₃₋₅ heterocyclyl refers to a heterocyclyl group having 3 to 5 ring atoms and 1 to 4 heteroatoms as ring members). The ring heteroatoms are independently selected from nitrogen, oxygen, and sulphur.

As with bicyclic cycloalkyl groups, bicyclic heterocyclyl groups may include isolated rings, spiro rings, fused rings, and bridged rings. The heterocyclyl group may be attached to a parent group or to a substrate at any ring atom and may include one or more non-hydrogen substituents unless such attachment or substitution would violate valence requirements or result in a chemically unstable compound.

Examples of monocyclic heterocyclyl groups include, but are not limited to, those derived from:

N₁: aziridine, azetidine, pyrrolidine, pyrroline, 2H-pyrrole or 3H-pyrrole, piperidine, dihydropyridine, tetrahydropyridine, azepine;
O₁: oxirane, oxetane, tetrahydrofuran, dihydrofuran, tetrahydropyran, dihydropyran, pyran, oxepin;
S₁: thiirane, thietane, tetrahydrothiophene, tetrahydrothiopyran, thiepane;
O₂: dioxolane, dioxane, and dioxepane;
O₃: trioxane;
N₂: imidazoiidine, pyrazolidine, imidazoline, pyrazoline, piperazine:
N₁O₁: tetrahydrooxazole, dihydrooxazole, tetrahydroisoxazole, dihydroisoxazole, morpholine, tetrahydrooxazine, dihydrooxazine, oxazine;
N₁S₁: thiazoline, thiazolidine, thiomorpholine;
N₂O₁: oxadiazine;
O₁S₁: oxathiole and oxathiane (thioxane); and
N₁O₁S₁: oxathiazine.

Examples of substituted monocyclic heterocyclyl groups include those derived from saccharides, in cyclic form, for example, furanoses, such as arabinofuranose, lyxofuranose, ribofuranose, and xylofuranse, and pyranoses, such as aliopyranose, altropyranose, glucopyranose, mannopyranose, gulopyranose, idopyranose, galactopyranose, and talopyranose.

“3- to 8-membered heterocycloalkyl,” refers to a closed ring of comprising carbon atoms and heteroatoms. The heterocyclosalkyl may comprise one, or three heteroatoms. Suitably the heteroatoms are selected from the group consisting of O, N and S, and wherein the nitrogen and sulfur atoms may optionally be oxidized and the nitrogen heteroatom may optionally be quaternized. Heterocycloalkyl groups typically comprise from 3 to 8 ring member atoms, preferably from 3 to 7 ring member atoms, more preferably from 3 to 6 ring member atoms, and most preferably from 3 to 5 ring member atoms. Heteroalkyl groups may be optionally substituted.

“heterocycloalkylene” refers to a divalent group derived from heteroalkyl (as discussed above). For heterocycloalkylene groups, heteroatoms can also occupy either or both of the positions where the heterocycloalkylene group is attached to the rest of the compound. Heteroalkylene groups may be optionally substituted.

“Nucleic acid”, refers to a linear polymer of nucleosides (including deoxyribo-nucleosides, ribonucleosides, or analogs thereof) joined by inter-nucleosidic linkages. Nucleic acid may encompass the term “polynucleotide” as well as “oligonucleotide”. The linear polymer may be represented by a sequence of letters, such as “ATGCCTG,” where it will be understood that the nucleotides are in 5′ to 3′ order from left to right and that “A” denotes deoxyadenosine, “C” denotes deoxycytidine, “G” denotes deoxyguanosine, and “T” denotes deoxythymidine, unless otherwise noted. Another natural nucleotide is “U”, denoting uridine. The letters A, C, G, T and U can be used to refer to the bases themselves, to nucleosides, or to nucleotides comprising the bases, as is standard in the art. In naturally occurring nucleic acids, the inter-nucleoside linkage is typically a phosphodiester bond, and the subunits are referred to as “nucleotides.” Nucleic acids may also include other inter-nucleoside linkages, such as phosphoro-thioate linkages, and the like. Such analogs of nucleotides that do not include a phosphate group are considered to fall within the scope of the term “nucleotid”” as used herein, and nucleic acids comprising one or more inter-nucleoside linkages that are not phosphodiester linkages are still referred to as “polynucleotides”, “oligonucleotides”, etc.

### Nitrogen Protecting Groups

Nitrogen protecting groups are well known in the art and are groups that block or protect the nitrogen groups from further reaction. Nitrogen protecting groups are exemplified by carbamates, such as methyl or ethyl carbamate, 9-fluorenylmethyloxy-carbonyl (Fmoc), substituted ethyl carbamates, carbamates cleaved by 1,6-beta-elimination, ureas, amides, peptides, alkyl and aryl derivatives. Carbamate protecting groups have the general formula:

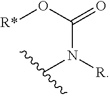

In this specification a zig-zag line (or wavy line [symbol] ) indicates the point of attachment of the shown group (e.g. the protecting group above) to the rest of the compound of formula (I). Suitable nitrogen protecting groups may be selected from acetyl, trifluoroacetyl, t-butyloxy-carbonyl (BOC), benzyloxycarbonyl (Cbz) and 9-fluorenylmethyloxy-carbonyl (Fmoc).

A large number of possible carbamate nitrogen protecting groups are listed on pages 706 to 771 of Wuts, P. G. M. and Greene, T. W., Protective Groups in Organic Synthesis, 4^{th} Edition, Wiley-Interscience, 2007, and in P. Kocienski, Protective Groups, 3rd Edition (2005) which are incorporated herein by reference.

Particularly preferred protecting groups include Alloc (allyloxycarbonyl), Troc (2,2,2-Trichloroethyl carbonate), Teoc [2-(Trimethylsilyl)ethoxycarbony], BOC (tert-butyloxycarbonyl), Doc (2,4-dimethylpent-3-yloxycarbonyl), Hoc (cyclohexyloxy-carbonyl), TcBOC (2,2,2-trichloro-tert-butyloxycarbonyl), Fmoc (9-fluorenylmethyloxycarbonyl), 1-Adoc (1-Adamantyloxycarbonyl) and 2-Adoc (2-adamantyloxycarbonyl).

### Hydroxyl Protecting Groups

Hydroxyl protecting groups are well known in the art, a large number of suitable groups are described on pages 16 to 366 of Wuts, P. G. M. and Greene, T. W., Protective Groups in Organic Synthesis, 4^{th} Edition, Wiley-Interscience, 2007, and in P. Kocienski, Protective Groups, 3rd Edition (2005) which are incorporated herein by reference.

Classes of particular interest include silyl ethers, methyl ethers, alkyl ethers, benzyl ethers, esters, benzoates, carbonates, and sulfonates. Particularly preferred protecting groups include THP (tetrahydropyranyl ether).

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “antibody” is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgGi, IgG2, IgG3, IgG4, IgAi, and IgA2. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, J¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

By “co-administering” is meant intravenously administering two (or more) drugs during the same administration, rather than sequential infusions of the two or more drugs. Generally, this will involve combining the two (or more) drugs into the same IV bag prior to co-administration thereof.

A drug that is administered “concurrently” with one or more other drugs is administered during the same treatment cycle, on the same day of treatment as the one or more other drugs, and, optionally, at the same time as the one or more other drugs. For instance, for cancer therapies given every 3 weeks, the concurrently administered drugs are each administered on day-1 of a 3-week cycle.

A “chemotherapeutic agent” refers to a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammail and calicheamicin omegali (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethyl ornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g., celecoxib or etoricoxib), proteosome inhibitor (e.g., PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors; serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARFMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releaseing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

“Drug”, “drug substance”, “active pharmaceutical ingredient”, and the like, refer to a compound (e.g., compounds of Formula (I) and compounds specifically named above) that may be used for treating a subject in need of treatment.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “epitope” refers to the particular site on an antigen molecule to which an antibody binds.

The “epitope 4D5” or “4D5 epitope” or “4D5” is the region in the extracellular domain of HER2 to which the antibody 4D5 (ATCC CRL 10463) and trastuzumab bind. This epitope is close to the transmembrane domain of HER2, and within domain IV of HER2. To screen for antibodies which bind to the 4D5 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 4D5 epitope of HER2 (e.g. any one or more residues in the region from about residue 550 to about residue 610, inclusive, of HER2 (SEQ ID NO: 39).

The “epitope 2C4” or “2C4 epitope” is the region in the extracellular domain of HER2 to which the antibody 2C4 binds. In order to screen for antibodies which bind to the 2C4 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 2C4 epitope of HER2. Epitope 2C4 comprises residues from domain II in the extracellular domain of HER2. The 2C4 antibody and pertuzumab bind to the extracellular domain of HER2 at the junction of domains I, II and III (Franklin et al. Cancer Cell 5:317-328 (2004)). Anti-HER2 murine antibody 7C2 binds to an epitope in domain I of HER2. See, e.g., PCT Publication No. WO 98/17797. This epitope is distinct from the epitope bound by trastuzumab, which binds to domain IV of HER2, and the epitope bound by pertuzumab, which binds to domain II of HER2. By binding domain IV, trastuzumab disrupts ligand-independent HER2-HER3 complexes, thereby inhibiting downstream signaling (e.g. PI3K/AKT). In contrast, pertuzumab binding to domain II prevents ligand-driven HER2 interaction with other HER family members (e.g. HER3, HER1 or HER4), thus also preventing downstream signal transduction. Binding of MAb 7C2 to domain I does not result in interference of trastuzumab or pertuzumab binding to domains IV and II, respectively, thereby offering the potential of combining a MAb 7C2 ADC with trastuzumab, trastuzumab emtansine (T-DM-1), and/or pertuzumab. Murine antibody 7C2, 7C2.B9, is described in PCT Publication No. WO 98/17797. An anti-HER2 7C2 humanized antibody is disclosed in WO2016/040723 A1.

“Excipient” refers to any substance that may influence the bioavailability of a drug, but is otherwise pharmacologically inactive.

The term “Fe region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of HI, 50-58 of H2, and 95-102 of H3. (See Almagro and Fransson, Front. Biosci. 13: 1619-1633 (2008).) Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); non-steroidal anti-inflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, including SOLU-MEDROL® methylprednisolone sodium succinate, and dexamethasone; dihydrofolate reductase inhibitors such as methotrexate (oral or subcutaneous); anti-malarial agents such as chloroquine and hydroxychloroquine; sulfasalazine; leflunomide; cytokine or cytokine receptor antibodies including anti-interferon-alpha, -beta, or -gamma antibodies, antitumor necrosis factor (TNF)-alpha antibodies (infliximab (REMICADE®) or adalimumab), anti-TNF-alpha immunoadhesin (etanercept), anti-TNF-beta antibodies, anti-interleukin-2 (IL-2) antibodies and anti-IL-2 receptor antibodies, and anti-interleukin-6 (IL-6) receptor antibodies and antagonists (such as ACTEMRA™ (tocilizumab)); anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187); streptokinase; transforming growth factor-beta (TGF-beta); streptodornase; RNA or DNA from the host; FK506; RS-61443; chlorambucil; deoxyspergualin; rapamycin; T-cell receptor (Cohen et al, U.S. Pat. No. 5,114,721); T-cell receptor fragments (Offner et al, Science, 251: 430-432 (1991); WO 90/11294; Ianeway, Nature, 341: 482 (1989); and WO 91/01133); BAFF antagonists such as BAFF antibodies and BR3 antibodies and zTNF4 antagonists (for review, see Mackay and Mackay, Trends Immunol, 23: 113-5 (2002) and see also definition below); biologic agents that interfere with T cell helper signals, such as anti-CD40 receptor or anti-CD40 ligand (CD 154), including blocking antibodies to CD40-CD40 ligand (e.g., Durie et al, Science, 261: 1328-30 (1993); Mohan et al, J. Immunol, 154: 1470-80 (1995)) and CTLA4-Ig (Finck et al, Science, 265: 1225-7 (1994)); and T-cell receptor antibodies (EP 340,109) such as T10B9. Some preferred immunosuppressive agents herein include cyclophosphamide, chlorambucil, azathioprine, leflunomide, MMF, or methotrexate.

An “isolated antibody” is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “HER2,” as used herein, refers to any native, mature HER2 which results from processing of a HER2 precursor protein in a cell. The term includes HER2 from any vertebrate source, including mammals such as primates (e.g. humans and cynomolgus monkeys) and rodents (e.g., mice and rats), unless otherwise indicated.

The term also includes naturally occurring variants of HER2, e.g., splice variants or allelic variants. The amino acid sequence of an exemplary human HER2 precursor protein, with signal sequence (with signal sequence, amino acids 1-22) is shown in SEQ ID NO: 64. The amino acid sequence of an exemplary mature human HER2 is amino acids 23-1255 of SEQ ID NO: 64.

The term “HER2-positive cell” refers to a cell that expresses HER2 on its surface. The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program

The term “PD-1 axis binding antagonist” refers to a molecule that inhibits the interaction of a PD-1 axis binding partner with either one or more of its binding partner, so as to remove T-cell dysfunction resulting from signaling on the PD-1 signaling axis—with a result being to restore or enhance T-cell function (e.g., proliferation, cytokine production, target cell killing). As used herein, a PD-1 axis binding antagonist includes a PD-1 binding antagonist, a PD-L1 binding antagonist and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, polypeptides, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is MDX-1106 (nivolumab) described herein. In another specific aspect, a PD-1 binding antagonist is MK-3475 (lambrolizumab) described herein. In another specific aspect, a PD-1 binding antagonist is CT-o11 (pidilizumab) described herein. In another specific aspect, a PD-1 binding antagonist is AMP-224 described herein.

The term “PD-L1 binding antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, polypeptides, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signalling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific aspect, an anti-PD-L1 antibody is YW243.55. S70 described herein. In another specific aspect, an anti-PD-L1 antibody is MDX-1105 described herein. In still another specific aspect, an anti-PD-L1 antibody is MPDL3280A described herein. In still another specific aspect, an anti-PD-L1 antibody is MEDI4736 described herein.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, polypeptides, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

A “fixed” or “flat” dose of a therapeutic agent herein refers to a dose that is administered to a human patient without regard for the weight (WT) or body surface area (BSA) of the patient. The fixed or flat dose is therefore not provided as a mg/kg dose or a mg/m² dose, but rather as an absolute amount of the therapeutic agent.

A “loading” dose herein generally comprises an initial dose of a therapeutic agent administered to a patient, and is followed by one or more maintenance dose(s) thereof. Generally, a single loading dose is administered, but multiple loading doses are contemplated herein. Usually, the amount of loading dose(s) administered exceeds the amount of the maintenance dose(s) administered and/or the loading dose(s) are administered more frequently than the maintenance dose(s), so as to achieve the desired steady-state concentration of the therapeutic agent earlier than can be achieved with the maintenance dose(s).

A “maintenance” dose herein refers to one or more doses of a therapeutic agent administered to the patient over a treatment period. Usually, the maintenance doses are administered at spaced treatment intervals, such as approximately every week, approximately every 2 weeks, approximately every 3 weeks, or approximately every 4 weeks, preferably every 3 weeks.

“Infusion” or “infusing” refers to the introduction of a drug-containing solution into the body through a vein for therapeutic purposes. Generally, this is achieved via an intravenous (IV) bag.

An “intravenous bag” or “IV bag” is a bag that can hold a solution which can be administered via the vein of a patient. In one embodiment, the solution is a saline solution (e.g. about 0.9% or about 0.45% NaCl). Optionally, the IV bag is formed from polyolefin or polyvinal chloride.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

A “free cysteine amino acid” refers to a cysteine amino acid residue which has been engineered into a parent antibody, has a thiol functional group (—SH), and is not paired as an intramolecular or intermolecular disulfide bridge.

The term “or salts, solvates, isomers or tautomers thereof” means that salts, solvates, isomeric or tautomeric forms of the shown structure are also included. Mixtures thereof, means that mixture of these forms may be present, for example, the compounds of the invention may include both a tautomeric form and a salt. Suitably, these forms are pharmaceutically acceptable salts, solvates, isomers or tautomers thereof.

“Pharmaceutically acceptable” substances refers to those substances which are within the scope of sound medical judgment suitable for use in contact with the tissues of subjects without undue toxicity, irritation, allergic response, and the like, commensurate with a reasonable benefit-to-risk ratio, and effective for their intended use.

“Pharmaceutical composition” refers to the combination of one or more drug substances and one or more excipients.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer. The term “polypeptide” is used herein to refer to any amino acid polymer comprised of two or more amino acid residues linked via peptide bonds. The term “peptide” or “oligopeptide” typically refers to short polypeptides, e.g. those comprising between 2 and 20 amino acids.

“Protein” refers to large polypeptides. Proteins may comprise one or more long chains of amino acid residues, i.e. one or more long polypeptides.

As used herein, “solvate” refers to a complex of variable stoichiometry formed by a solute (e.g. formulas (1)-(1) (A), (B), (C), (D), or any other compound herein or a salt thereof) and a solvent. Pharmaceutically acceptable solvates may be formed for crystalline compounds wherein solvent molecules are incorporated into the crystalline lattice during crystallization. The incorporated solvent molecules can be water molecules or non-aqueous molecules, such as but not limited to, ethanol, isopropanol, dimethyl sulfoxide, acetic acid, ethanolamine, and ethyl acetate molecules.

The term “subject” as used herein refers to a human or non-human mammal. Examples of non-human mammals include livestock animals such as sheep, horses, cows, pigs, goats, rabbits and deer; and companion animals such as cats, dogs, rodents, and horses.

“Therapeutically effective amount” of a drug refers to the quantity of the drug or composition that is effective in treating a subject and thus producing the desired therapeutic, ameliorative, inhibitory or preventative effect. The therapeutically effective amount may depend on the weight and age of the subject and the route of administration, among other things.

“Treating” refers to reversing, alleviating, inhibiting the progress of, or preventing a disorder, disease or condition to which such term applies, or to reversing, alleviating, inhibiting the progress of, or preventing one or more symptoms of such disorder, disease or condition.

“Treatment” refers to the act of “treating”, as defined immediately above.

As used herein the term “comprising” means “including at least in part of” and is meant to be inclusive or open ended. When interpreting each statement in this specification that includes the term “comprising”, features, elements and/or steps other than that or those prefaced by the term may also be present. Related terms such as “comprise” and “comprises” are to be interpreted in the same manner.

The term “consisting essentially of” limits the scope of a claim to the specified materials or steps “and those that do not materially affect the basic and novel characteristic(s)” of the claimed invention. When the phrase “consisting essentially of” appears in a clause of the body of a claim, rather than immediately following the preamble, it limits only the element set forth in that clause.

The term “consisting of” excludes any element, step, or ingredient not specified in the claim; “consisting of” defined as “closing the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole. It should be understood that while various embodiments in the specification are presented using “comprising” language, under various circumstances, a related embodiment is also described using “consisting essentially of” or “consisting of” language.

### A

A is a group selected from:

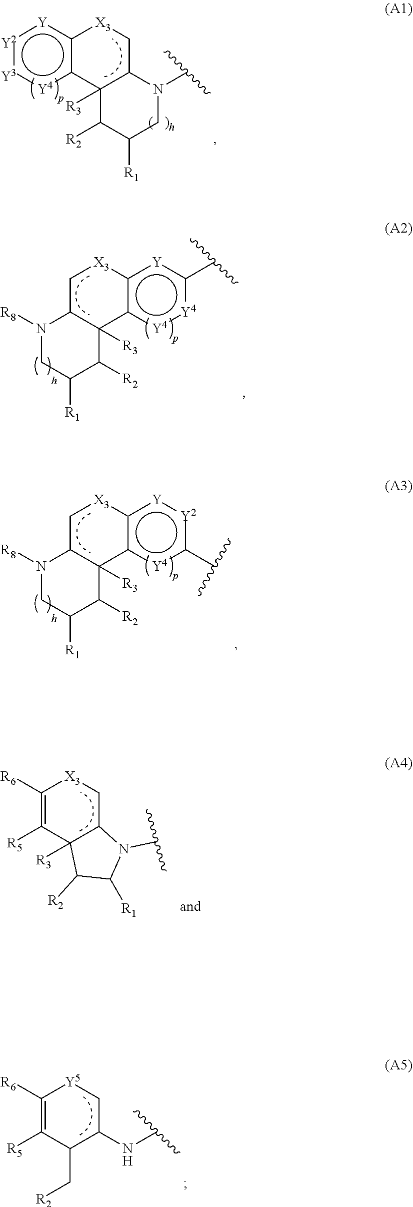

The ring containing Y in (A1), (A2) and (A3) is an aromatic ring and, because of the limitations on the substituents, is either a 6-membered aryl ring (when p is 1) or is a 5-membered heteroaryl ring (when p is 0).

Thus, when p is 1 then (A1), (A2) and (A3) may be represented by (A6), (A7) and (A8):

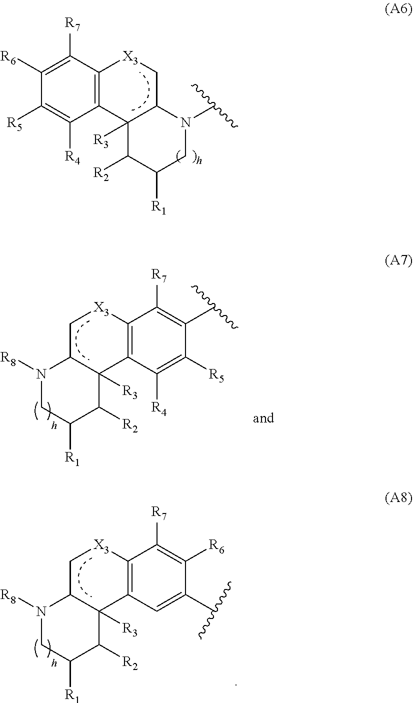

When p is 0 then (A1), (A2) and (A3) may be represented by (A9), (A10), (A11), (A12) and (A13):

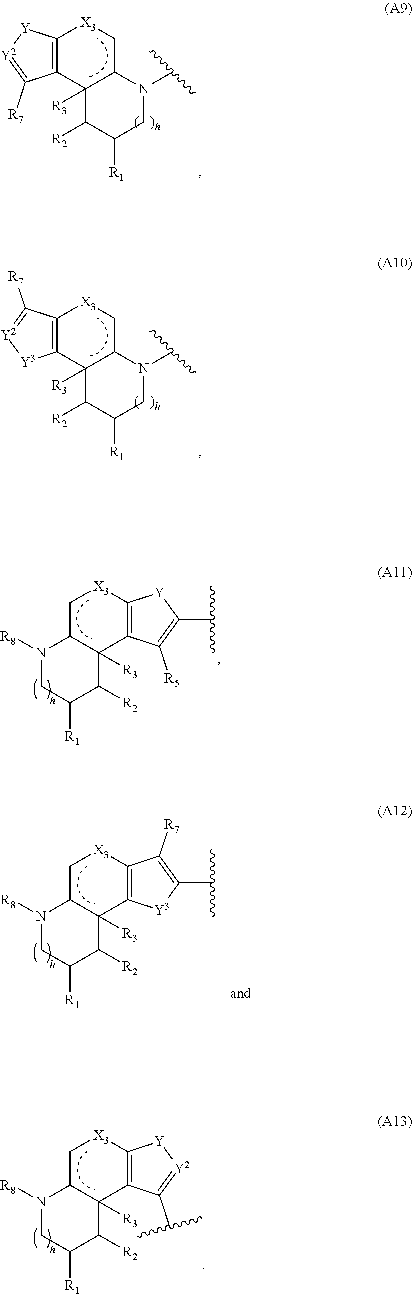

Suitably A is selected from (A1), (A4), (A5), (A6), (A9) and (A10).

In (A1), (A2), (A3), (A4) and (A5) when X₃ or Y⁵ is C═O with the carbon forming part of the ring, then [symbol] represents an α,β-unsaturated double bond conjugated with the C═O such that (A1), (A2), (A3), (A4) and (A5) are represented by (A14), (A15), (A16), (A17) and (A18) respectively:

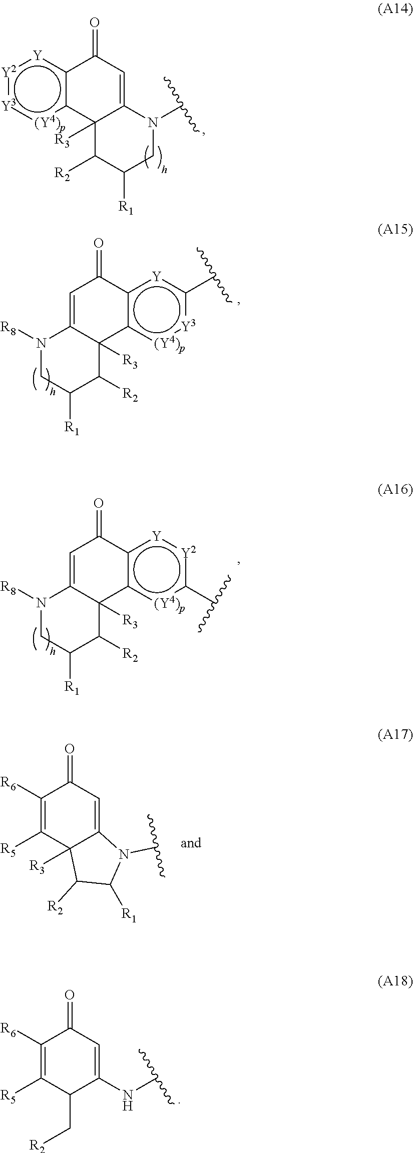

In (A1), (A2), (A3), (A4) and (A5) when X₃ is C—OH or Y⁵ is C—OH or C—NH₂ then [symbol] represents the double bonds of an aromatic 6-membered ring and R₃ is absent such that (A1), (A2), (A3), (A4) and (A5) are represented by (A19), (A19.1), (A20), (A20.1), (A21), (A21.1), (A22), (A22.1), (A23) and (A24):

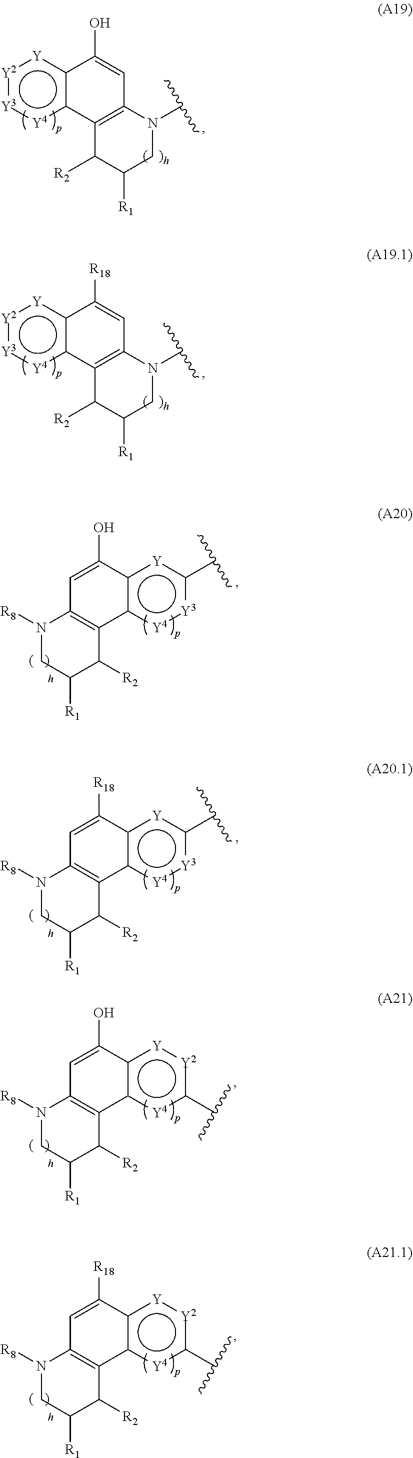

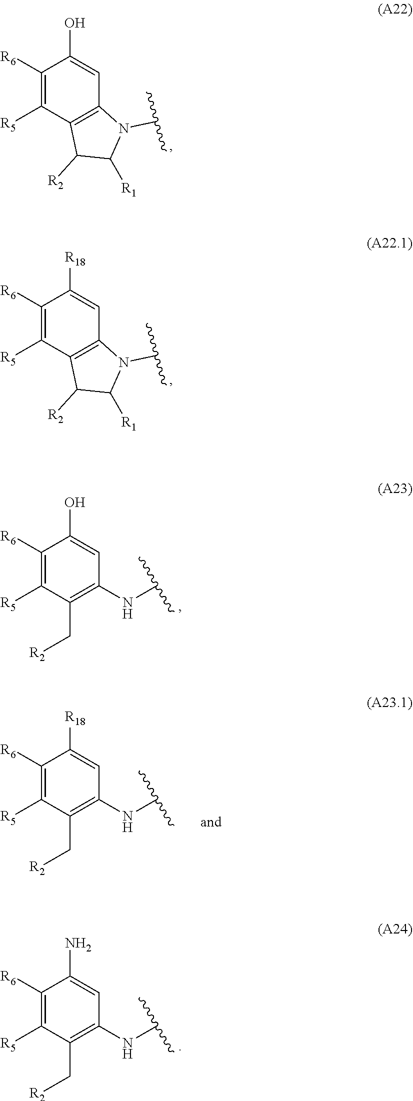

Suitably A is selected from (A4), (A5), (A6), (A7), (A8), (A9), (A10), (A11), (A12), (A13), (A14), (A15), (A16), (A17), (A18), (A19), (A19.1), (A20), (A20.1), (A21), (A21.1), (A22), (A22.1), (A23) and (A24).

Suitably A is selected from (A1), (A2), (A3) and (A4). Suitably A is selected from (A1), (A2) and (A3)

Suitably A is (A1). Suitably, (A1) is selected from:

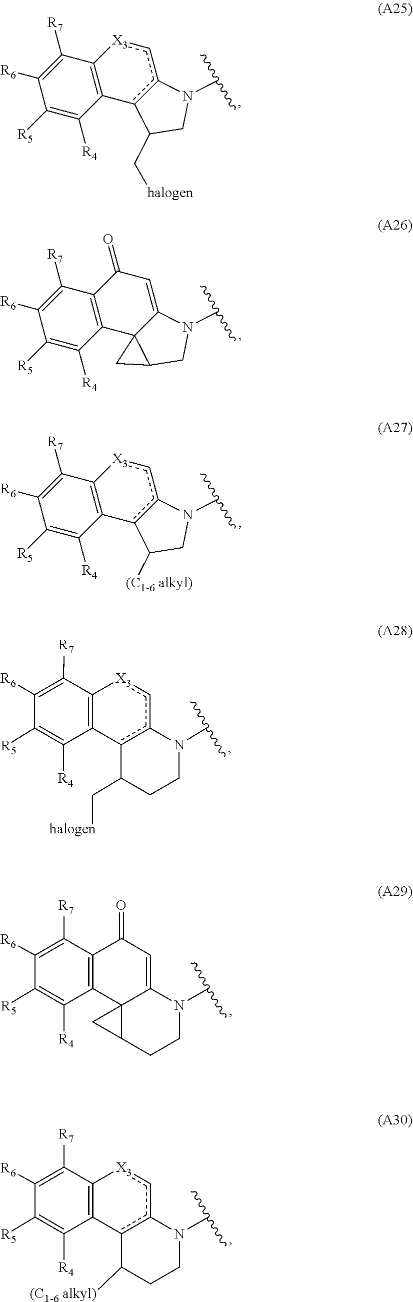

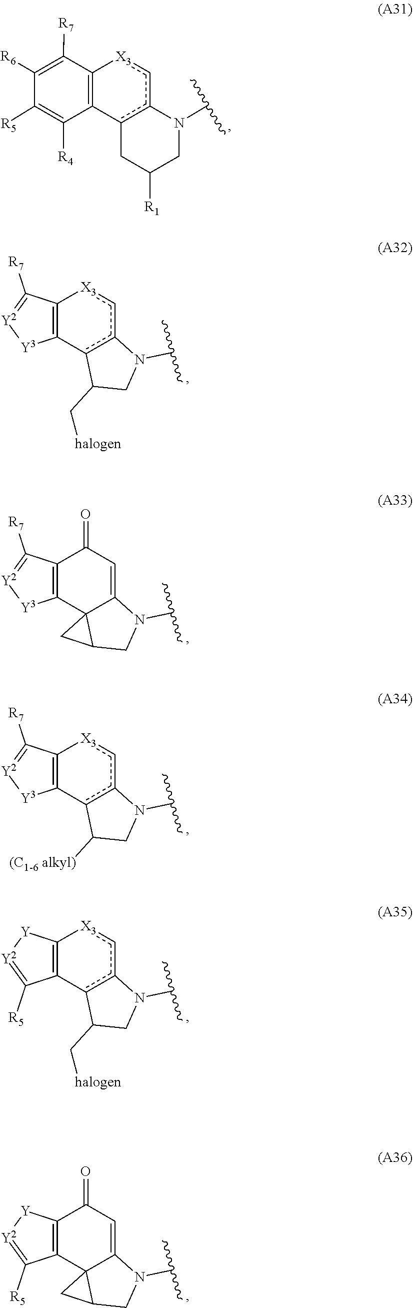

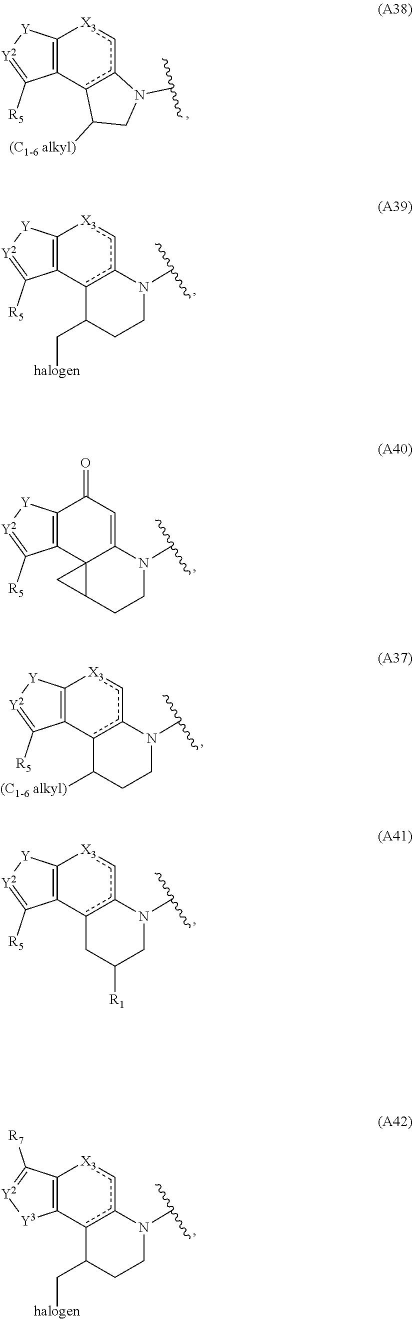

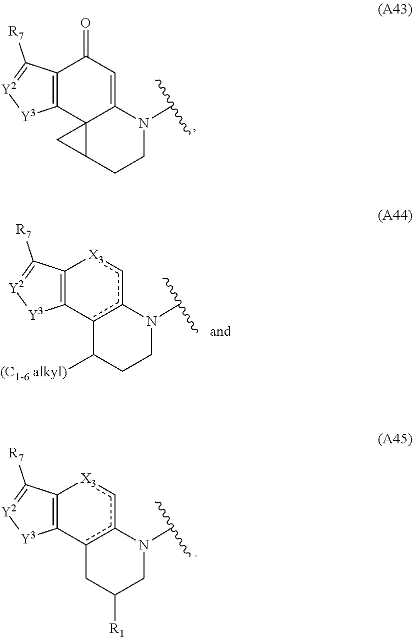

When p is 1, suitably, (A1) is (A25), (A26), (A28), (A29) or (A31). More suitably, when p is 1, (A1) is (A25), (A26), (A28) or (A29). More suitably, (A1) is (A25) or (A26).

When p is 1, suitably, (A1) is (A32), (A33), (A35), (A36), (A38), (A39), (A41), (A42), (A43) or (A45). More suitably, when p is 0, (A1) is (A32), (A33), (A35), (A36), (A38), (A39), (A42) or (A43). More suitably, when p is 0, (A1) is (A32), (A33), (A35) or (A36).

In some aspects, suitably, A is (A25), (A26), (A28), (A29), (A32), (A33), (A35), (A36), (A38), (A39), (A42) or (A43).

In some aspects, suitably A is (A2). Suitably, (A2) is selected from:

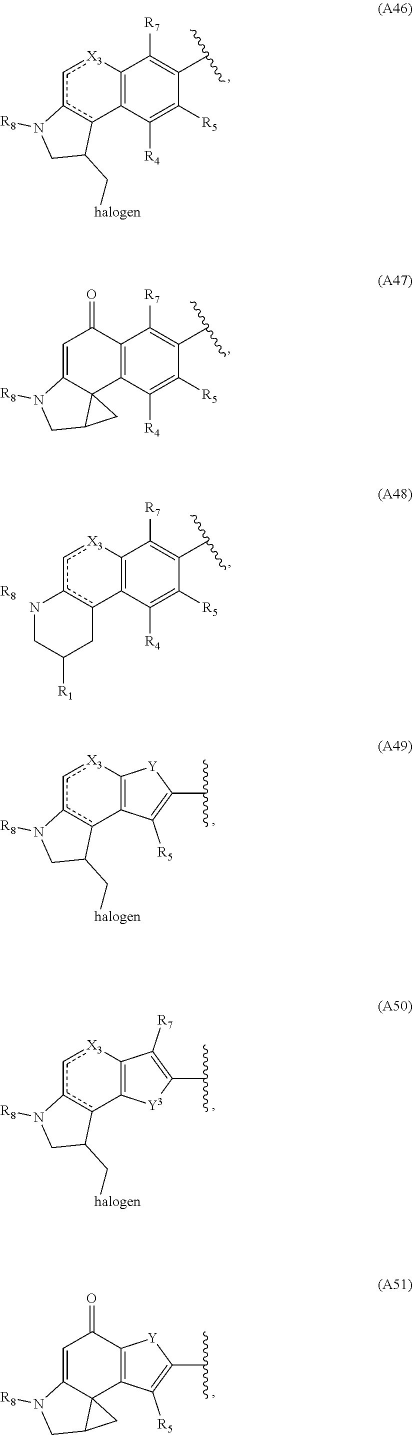

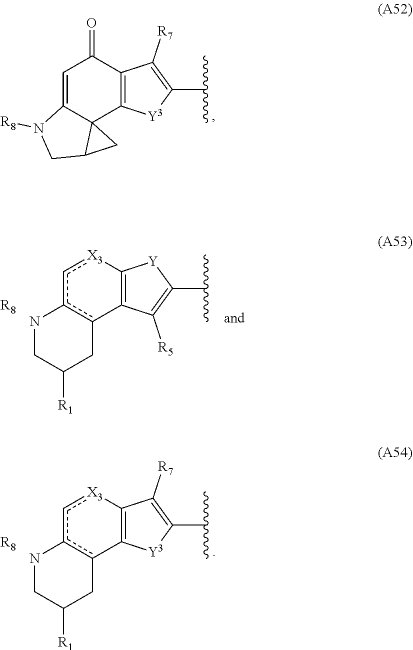

More suitably, (A2) is (A46), (A47), (A49), (A50), (A51) or (A52).

In some aspects, suitably A is (A3). Suitably, (A3) is selected from:

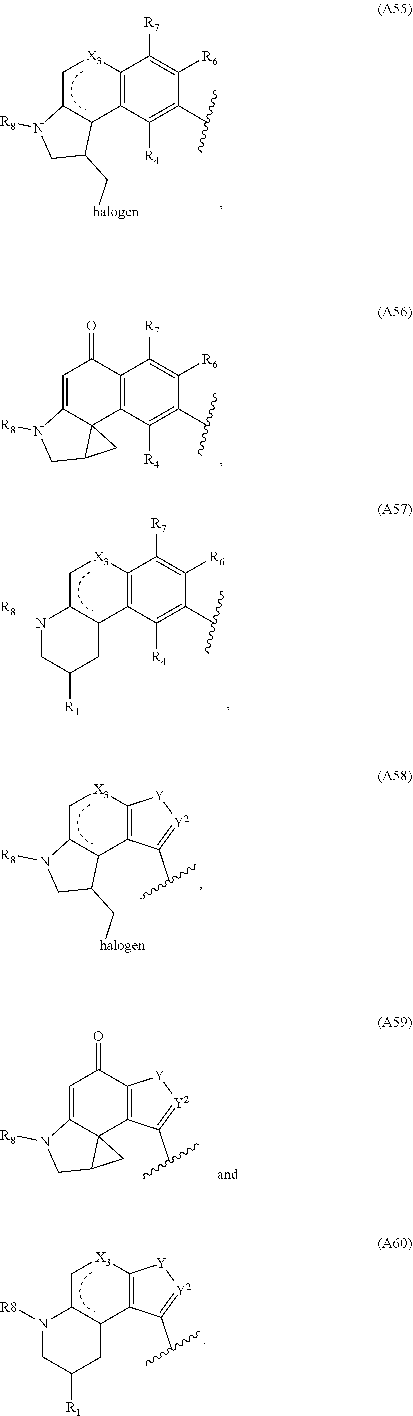

More suitably, (A3) is (A55), (A56), (A58) or (A59).

In some aspects, suitably A is (A4). Suitably, (A4) is:

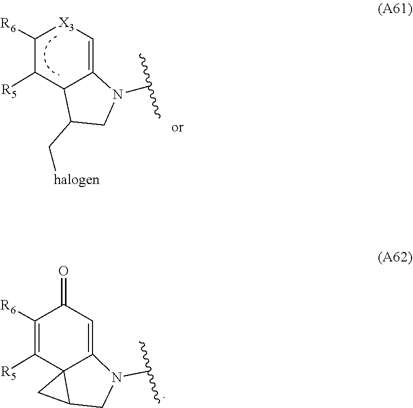

In some aspects, suitably A is (A5). Suitably, (A5) is:

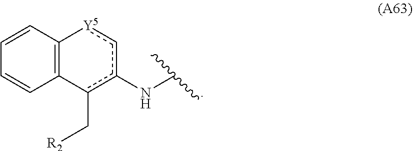

In one aspect, more suitably, A is

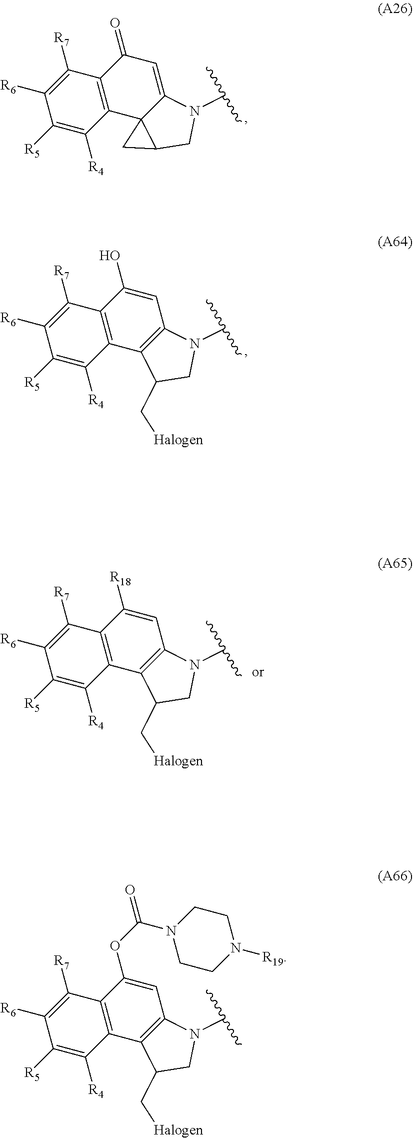

In one aspect, more suitably, A is

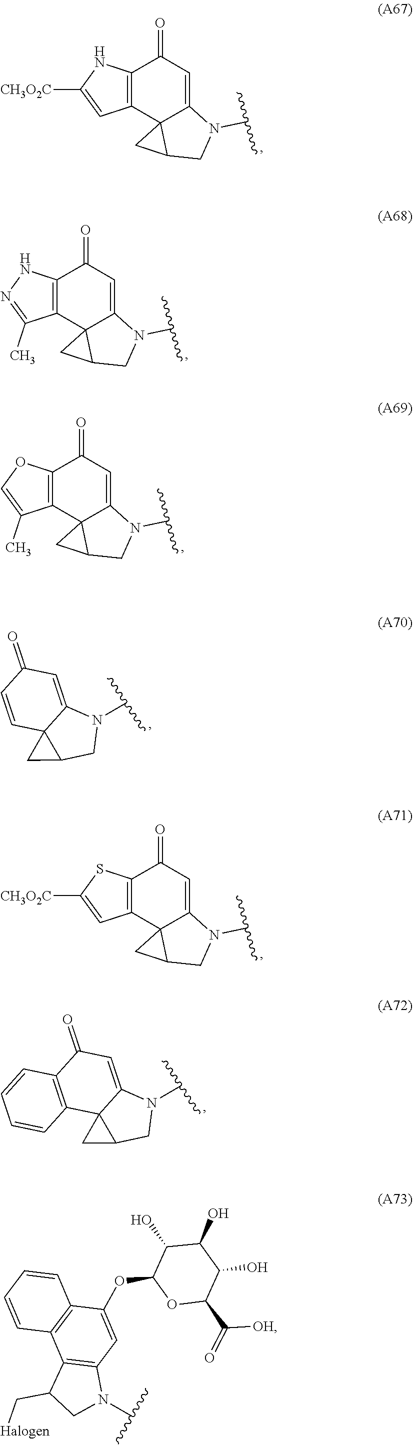

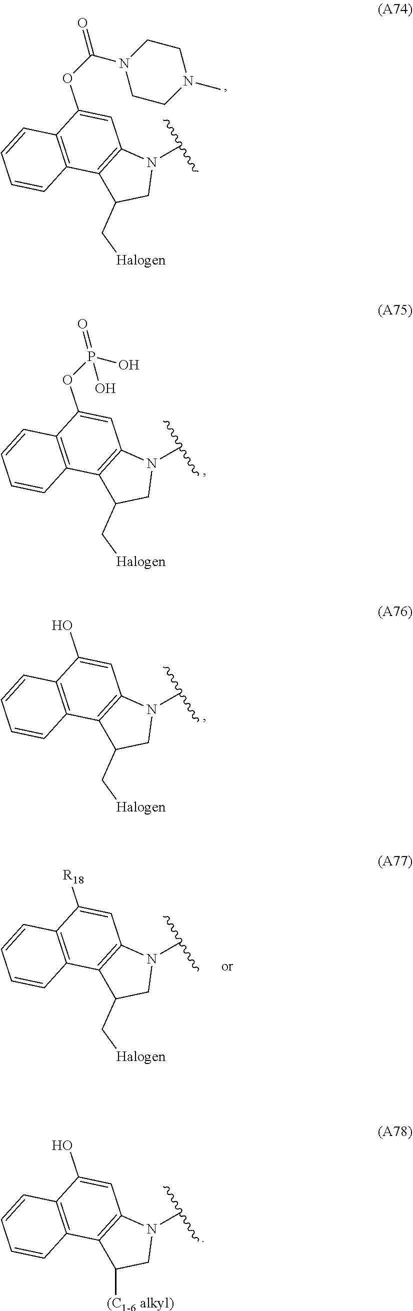

More suitably, A is (A72), (A73), (A74), (A75), (A76), (A77) or (A78). More suitably, A is (A73), (A74), (A75), (A76), (A77) or (A78). More suitably, A is (A73), (A74), (A75), (A76) or (A77).

More suitably, in one aspect, A is:

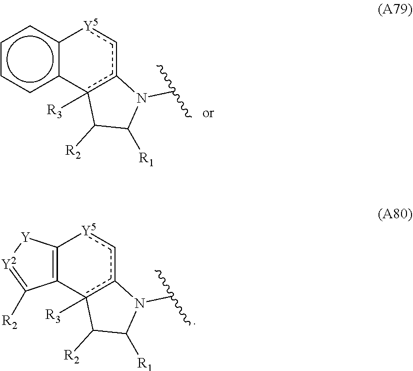

Suitably, in another aspect, A is:

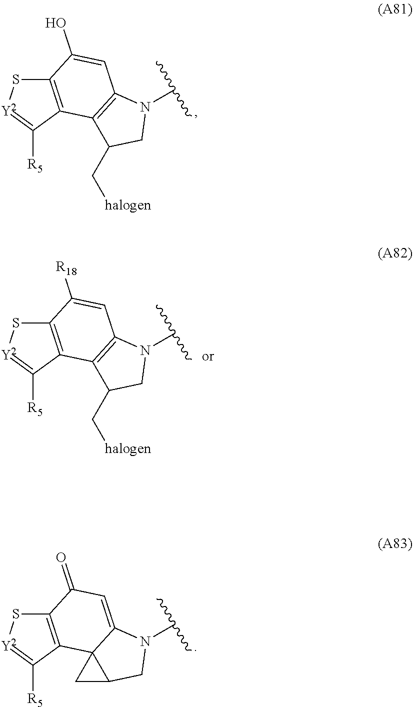

### X₁

Suitably, X₁ is selected from O, S, NR₂₁, CR₂₁R₂₂, CR₂₁R₂₂O, C(═O), C(═O)NR₂₁, NR₂₁C(═O), O—C(O) and C(O)—O or is absent.

Suitably, X₁ is selected from O, S, NR₂₁, CR₂₁R₂₂, C(═O), C(═O)NR₂₈ and NR₂₁C(═O) or is absent. Hence, X₁ may be an amide that links group A to group SP in either direction. Thus, when X₁ is selected as C(═O)NR₂₈ then A is linked to SP as follows: A-C(═O)NR₂₈—SP—X₂—B, whereas when X₁ is NR₂₁C(═O) then A is linked to SP as follows: A-NR₂₁C(═O)—SP—X₂—B.

Suitably, X₁ is selected from C(═O), C(═O)NH and NHC(═O) or is absent.

Most suitably, when A is (A1) then X₁ is C(═O).

Most suitably, when A is (A2) then X₁ is NHC(═O).

### SP

Suitably, SP is an amino acid.

Suitably, SP is a peptide chain. Suitably a peptide chain has 2, 3, 4, 5, 6, 7, 8, 9, 10, 11 or 12 amino acids.

Suitably, SP is a paraformaldehyde chain. Suitably a paraformaldehyde chain has 1, 2, 3, 4, 5, 6, 7, 8, 9, 10, 11, 12, 13, 14, 15, 16, 17, 18, 19, 20, 21, 22, 23 or 24 units (CH₂O).

Suitably, SP is a polyethylene glycol chain. Suitably a polyethylene glycol chain has 1, 2, 3, 4, 5, 6, 7, 8, 9, 10, 11 or 12 polyethylene glycol units (CH₂CH₂O).

Suitably, SP is a paraformaldehyde chain —(CH₂O)₁₋₂₄—, a polyethylene glycol chain —(CH₂CH₂O)₁₋₁₂— or —(CH₂)ₘ-Y⁶—(CH₂)ₙ—.

In a more suitable aspect, SP is —(CH₂)ₘ-Y⁶—(CH₂)ₙ—.

Suitably, Y⁶ is selected from —(CH₂)_{z}—, phenylene, napthalenylene, pyrrolylene, N-methylpyrrolylene, furanylene, thiophenylene, imidazolylene, N-methylimidazolylene, oxazolylene, thiazolylene, indolylene, N-methylindolylene, benzofuranylene, benzothiophenylene, benzimidazolylene, N-methylbenzoimidazolylene, benzooxazolylene, benzothiazolylene, cyclopentylene, cyclohexylene cycloheptylene, cyclopentenylene, cyclohexenylene, cycloheptenylene, pyrrolidinylene, pyrrolinylene, piperidinylene and morpholinylene and the Y⁶ group is optionally substituted with up to three independently selected optional C₁₋₆ alkyl, OC₁₋₆ alkyl, OCH₂Ph and R₂₀ groups.

In some aspects, suitably, Y⁶ is unsubstituted.

In other aspects, suitably, Y⁶ is substituted with 1, 2 or 3 independently selected C₁₋₆ alkyl, OC₁₋₆ alkyl, OCH₂Ph, R₂₀ groups.

In one aspect, more suitably, Y⁶ is substituted with one R₂₀ group. More suitably, Y⁶ is substituted with a (CH₂)ⱼ—OH, (CH₂)ⱼ—NR₂₇R₂₈, or K₁—R* group. More suitably, Y⁶ is substituted with an OH, NH₂, or K₁—R* group.

Suitably, SP is selected from —CH₂O—, —CH₂O—CH₂O—, —CH₂O—CH₂O—CH₂O—, —CH₂CH₂O—, —CH₂CH₂O—CH₂CH₂O—, —CH₂CH₂O—CH₂CH₂O—CH₂CH₂O—, —(CH₂)ₘ—(CH₂)_{z}—(CH₂)ₙ—,

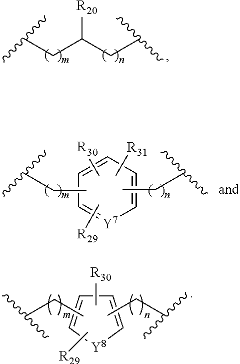

The aromatic rings above are drawn without specifying the positions of any of the R₂₉, R₃₀ or R₃₁ groups, and the two groups (shown by bonds that end in a zig-zag line) where the aromatic ring is attached to the rest of the molecule. Hence, these groups may be present on any position of the aromatic ring except for Y⁷ or Y⁸ (as positioning a group, such as R₂₉ at Y⁷ or Y⁸ would not meet the valence requirements).

More suitably, SP is selected from —CH₂CH₂O—CH₂CH₂O—, —(CH₂)ₘ—(CH₂)_{z}—(CH₂)ₙ—,

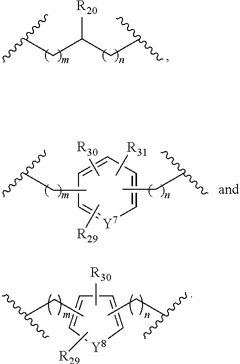

In one aspect,

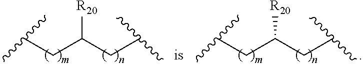

In another aspect, suitably

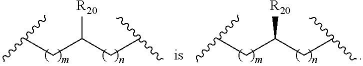

More suitably,

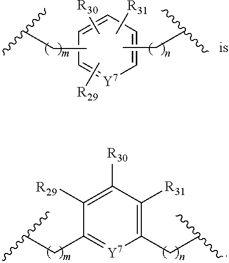

More suitably, SP is selected from —CH₂CH₂O—CH₂CH₂O—, —(CH₂)ₘ—(CH₂)_{z}—(CH₂)ₙ—,

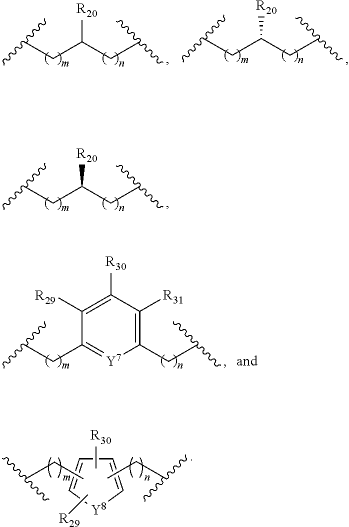

In one aspect,

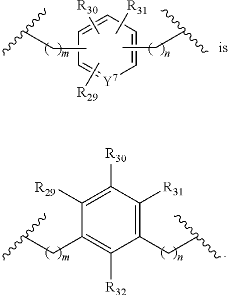

### X₂

Suitably, X₂ is selected from O, S, NR₂₃, CR₂₃R₂₄, CR₂₃R₂₄O, C(═O), C(═O)NR₂₃, NR₂₄C(═O), O—C(O) and C(O)—O or is absent.

Suitably, X₂ is selected from O, S, CR₃₀R₃₁, C(═O), C(═O)NR₃₀, NR₃₀C(═O) or is absent. Hence, X₂ may be an amide that links group SP to group B in either direction. Thus, when X₂ is selected as C(═O)NR₃₀ then SP is linked to B as follows: A-X₁-L-C(═O)NR₃₀—B, whereas when X₂ is NR₃₀C(═O) then SP is linked to B as follows: A-X₁-L-NR₃₀C(═O)—B.

Suitably, X₂ is selected from O, CR₃₀R₃₁, C(═O) or is absent.

Most suitably, when B is (B1) then X₂ is O.

Most suitably, when B is (B2) then X₂ is CR₃₀R₃₁ or is absent.

### B

The compounds of formula (I) comprise a group B:

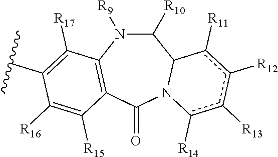

wherein the dotted lines indicate the optional presence of one or more double bonds in the C-ring.

Suitably, no double bond are present in the C-ring of group B and the dotted lines represent single bonds.

If one double bond is present it may be situated between any one of C1 and C2, C2 and C3, and C3 and C4. If two double bonds are present they are situated between C1 and C2, and C3 and C4.

In one aspect, B is (B1) and has a double bond between C1 and C2; or (B2) which has a double bond between C2 and C3; or (B3) which has a double bond between C3 and C4; or (B4) which has a double bond between C1 and C2 and a second double bond between C3 and C4. Hence, in this aspect B is:

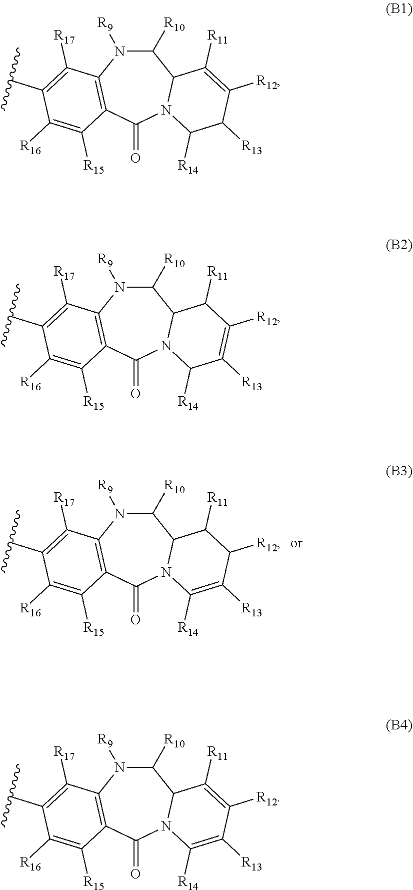

Most suitably, in some aspects, B contains a double bond in the C-ring. More suitably, B has a double bond between C2 and C3, i.e. B is (B2).

Suitably, B is:

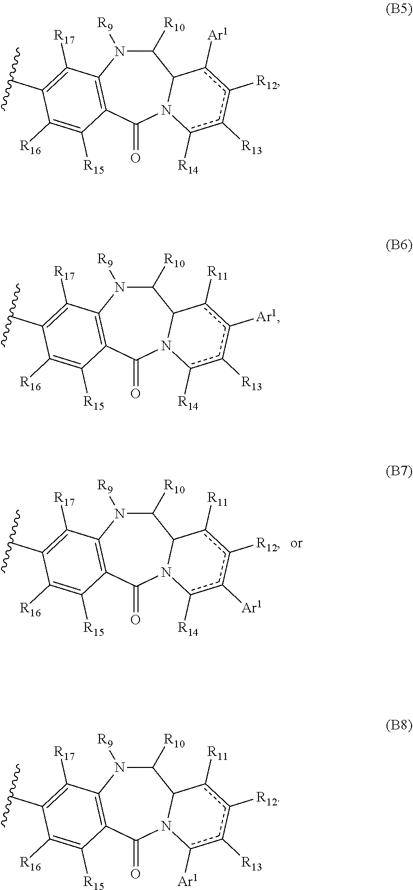

More suitably, B is (B6).

In a one aspect, (aa) one of R₁₁, R₁₂, R₁₃ and R₁₄ is E and another of R₁₁, R₁₂, R₁₃ and R₁₄ is an optionally substituted Ar¹ group. In this aspect B is:

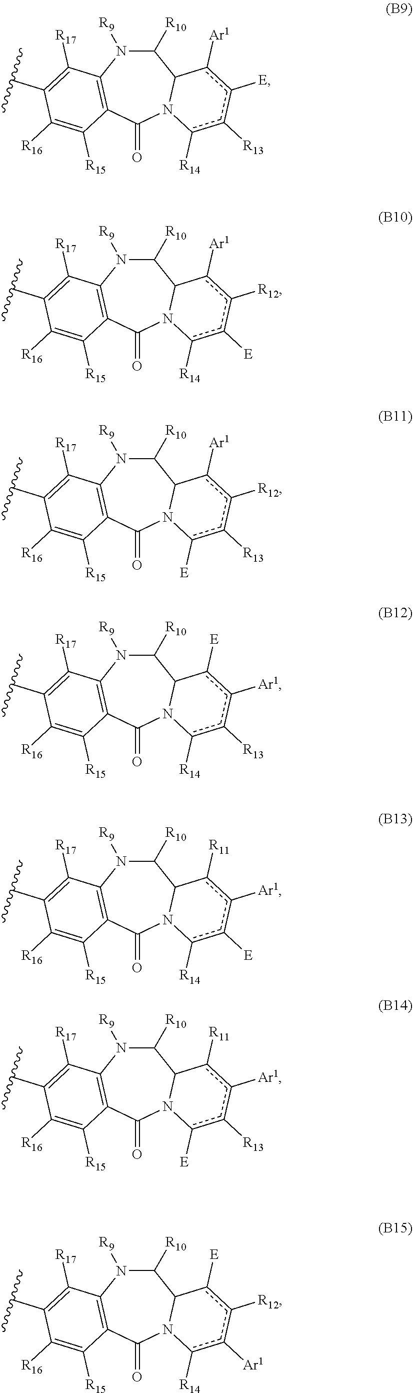

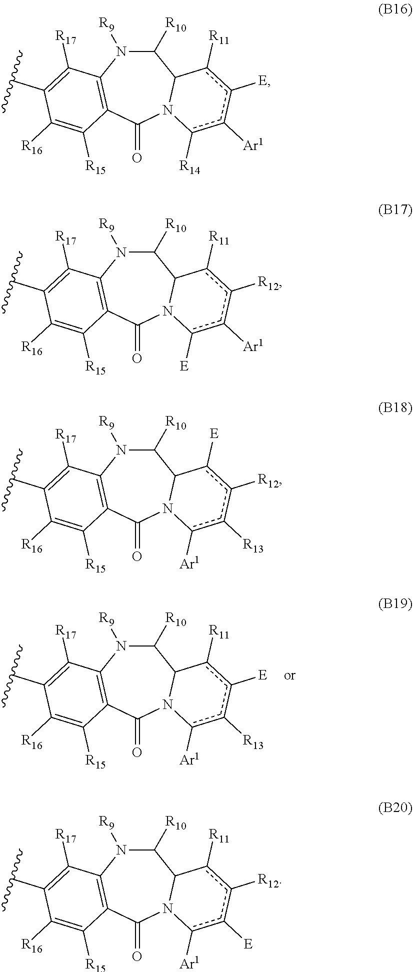

More suitably, in this aspect B is (B12), (B13) or (B14).

In another aspect, (ab) one of R₁₁, R₁₂, R₁₃ and R₁₄ is an RX group. In this aspect B is:

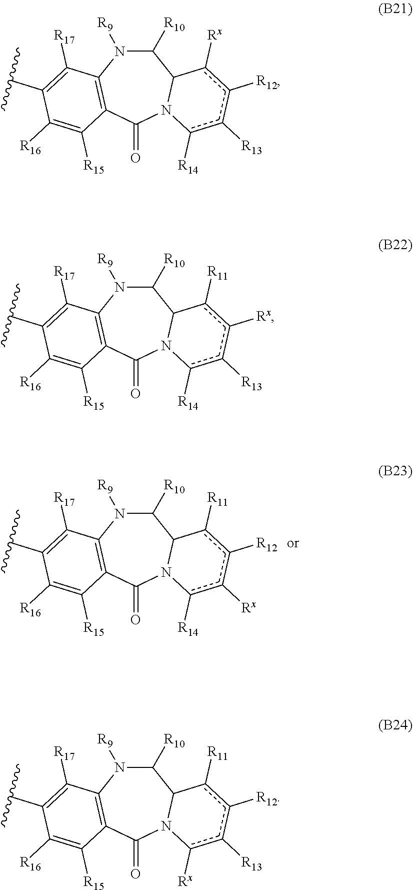

More suitably, in this aspect B is (B22).

More suitably, B is:

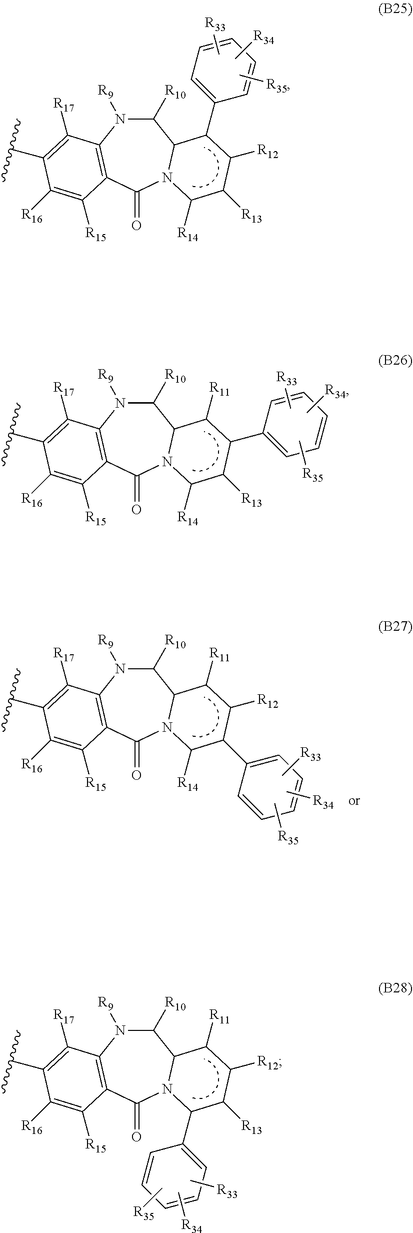

wherein R₃₃, R₃₄ and R₃₅ are each independently selected from are each independently selected from H, E, C₁₋₆ alkyl, OC₁₋₆ alkyl, OCH₂Ph, R₂₀ and R₂₅.

More suitably, B is (B10).

More suitably, B is

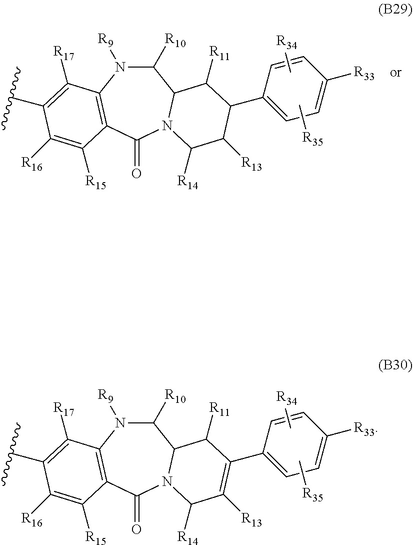

More suitably, B is (B30).

The polycyclic group B contains a stereocenter at the C12a position, as shown below:

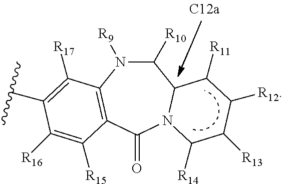

In one aspect, B, (B1), (B2), (B3), (B4), (B5), (B6), (B7), (B8), (B9), (B10), (B11), (B12), (B13), (B14), (B15), (B16), (B17), (B18), (B19), (B20), (B21), (B22), (B23), (B24), (B25), (B26), (B27), (B28), (B29) or (B30) is a racemic mixture at the the C12a position.

Suitably, in another aspect, B, (B1), (B2), (B3), (B4), (B5), (B6), (B7), (B8), (B9), (B10), (B11), (B12), (B13), (B14), (B15), (B16), (B17), (B18), (B19), (B20), (B21), (B22), (B23), (B24), (B25), (B26), (B27), (B28), (B29) or (B30) has R-stereochemistry at the C12a position. Hence, in this aspect B may be represented by:

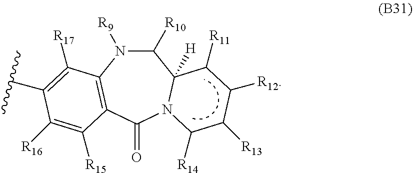

More suitably, in another aspect, B, (B1), (B2), (B3), (B4), (B5), (B6), (B7), (B8), (B9), (B10), (B11), (B12), (B13), (B14), (B15), (B16), (B17), (B18), (B19), (B20), (B21), (B22), (B23), (B24), (B25), (B26), (B27), (B28), (B29) or (B30) has S-stereochemistry at the C12a position. Hence, in this aspect B may be represented by:

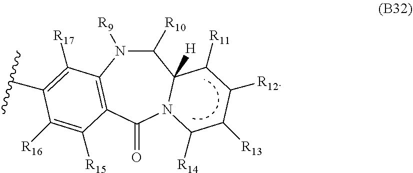

In another aspect (ac), one of R₁₁, R₁₂, R₁₃ and R₁₄ is R^{Y}, wherein R^{Y} is a 5- or 6-membered cyclic, heterocyclic, or heteroaryl ring optionally substituted with up to two groups independently selected from C₁₋₆ alkyl, OC₁₋₆ alkyl, OCH₂Ph, E, R₂₀ and R₂₅. In this aspect, suitably R^{Y} is an optionally substituted saturated or unsaturated 5-membered heterocyclic ring with at least one heteroatom selected from S, O or N. Suitable examples of such optionally substituted 5-membered heterocyclic rings (each with one heteroatom) include optionally substituted: furan, tetrahydrofuran, thiophene, tetrahydrothiophene, pyrrole, or pyrrolidine.

More suitably, R^{Y} is an optionally substituted 5-membered heterocyclic ring selected from furanyl, thiophenyl, or pyrryl. Most suitably, R^{Y} is thiophenyl.

### Ar¹

Suitably, the Ar¹ is a phenyl, biphenyl, indenyl, naphthalenyl or C₅₋₁₀ heteroaryl group; wherein the Ar¹ group is optionally substituted.

Suitably, the Ar¹ is a phenyl, biphenyl, indenyl, naphthalenyl, pyrrolyl, furanyl, thiophenyl, pyridinyl, oxazolyl, isoxazolyl, isoxazinyl, oxadiazolyl (e.g. 1-oxa-2,3-diazolyl, 1-oxa-2,4-diazolyl, 1-oxa-2,5-diazolyl, 1-oxa-3,4-diazolyl), oxatriazolyl, thiazolyl, isothiazolyl, imidazolyl, pyrazolyl, pyridazinyl, pyrimidinyl, pyrazinyl, triazolyl, triazinyl, tetrazolyl, benzoxazolyl, benzisoxazolyl, benzothiazolyl, benzimidazolyl, indazolyl, benzodioxolyl, benzothiadiazolyl, benzotriazolyl, purinyl(e.g., adeninyl, guaninyl), pteridinyl, cinnolinyl, naphthyridinyl, phthalazinyl, quinazolinyl and quinoxalinyl; wherein the Ar¹ group is optionally substituted.

Suitably, the Ar¹ is a phenyl, pyrrolyl, furanyl, thiophenyl, pyridinyl, oxazolyl, isoxazolyl, thiazolyl, isothiazolyl, imidazolyl, pyrazolyl, triazolyl, tetrazolyl, pyrimidinyl, pyrazinyl, benzoxazolyl, benzisoxazolyl, benzothiazolyl, benzimidazolyl, indazolyl, cinnolinyl, naphthyridinyl, phthalazinyl, quinazolinyl and quinoxalinyl; wherein the Ar¹ group is optionally substituted.

The Ar¹ group is optionally substituted with 1, 2 or 3 optional groups independently selected from C₁₋₆ alkyl, OC₁₋₆ alkyl, OCH₂Ph, E, R₂₀ and R₂₅.

Suitably, in other aspects, the Ar¹ group comprises 1, 2 or 3 groups independently selected from C₁₋₆ alkyl, OC₁₋₆ alkyl, OCH₂Ph, E, R₂₀ and R₂₅. More suitably, the Ar¹ group comprises 1, 2 or 3 groups independently selected from C₁₋₆ alkyl, OC₁₋₆ alkyl, OCH₂Ph and E.

Suitably, in some aspects, the Ar¹ group comprises 1, 2 or 3 groups independently selected from C₁₋₆ alkyl, OC₁₋₆ alkyl and OCH₂Ph.

Suitably, the Ar¹ group comprises 1, 2 or 3 groups independently selected from C₁₋₆ alkyl.

Suitably, the Ar¹ group comprises 1, 2 or 3 groups independently selected from OC₁₋₆ alkyl.

Suitably, the Ar¹ group comprises 1, 2 or 3 R₂₀ groups.

Suitably, the Ar¹ group comprises 1, 2 or 3 R₂₅ groups.

More suitably, the Ar¹ group comprises 1, 2 or 3 E groups.

More suitably, in the above aspects, the Ar¹ group is optionally substituted with 1 or 2 optional groups.

More suitably, the Ar¹ group is not optionally substituted.

### E

In one aspect, suitably, each E is independently selected from S(O)₂—NR₂₅R₂₆, S(O)₂—OH, CH₂CH₂[OCH₂CH₂]_{w}R₂₅ and E¹.

In another aspect, suitably, each E is (CH₂)ⱼ—S(O)₂—OH. More suitably, each E is S(O)₂—OH.

In another aspect, suitably, each E is CH₂CH₂[OCH₂CH₂]_{w}R₂₅.

In another aspect, suitably, each E is E¹.

In another aspect, suitably each E is (CH₂)ⱼ—S(O)₂—NR₂₅R₂₆. Suitably, each E is S(O)₂—NHR₂₅. Suitably, each E is S(O)₂—NH₂, S(O)₂—NH—CH₃ or S(O)₂—NHR₂O. More suitably, each E is S(O)₂—NH₂, S(O)₂—NH—CH₃ or S(O)₂—NH—K₁—R*. More suitably, each E is S(O)₂—NH—CH₃.

### E¹

Each E¹ group comprises at least two heteroatoms (i.e. 2, 3, 4, 5, 6, 7, 8, 9 or 10 heteroatoms) which may be present as ring heteroatoms or as heteroatoms in the optional substituents. For example, a hexose typically comprises one ring heteroatom (an oxygen) and 5 heteroatoms in the hydroxyl or CH₂OH groups.

More suitably, each E¹ is independently selected from pentose; hexose; C₅₋₆ heterocyclyl comprising at least two ring heteroatoms; C₅₋₁₀ heteroaryl group comprising at least two ring heteroatoms; C₅₋₁₀ heteroaryl group substituted with a C₅₋₆ heteroaryl or a C₅₋₆ heterocyclyl group; and phenyl substituted with a C₅₋₆ heteroaryl comprising at least two ring heteroatoms or a C₅₋₆ heterocyclyl group comprising at least two ring heteroatoms; wherein each E¹ group may be independently optionally substituted with 1, 2 or 3 optional groups independently selected from OC₁₋₆ alkyl, OCH₂Ph, R₂₀ and R₂₇.

In one aspect, each E¹ is an optionally substituted pentose.

Suitably, each pentose is independently selected from optionally substituted arabinose, lyxose, ribose and xylose.

In another aspect, each E¹ is an optionally substituted hexose.

Suitably, each hexose is independently selected from optionally substituted allose, altrose, glucose, mannose, gulose, idose, galactose and talose. Suitably, each hexose is independently selected from optionally substituted glucose, mannose and galactose.

Suitably, the hexose is

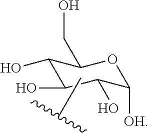

In another aspect, each E¹ is an optionally substituted C₅₋₆ heterocyclyl. Suitably, each optionally substituted C₅₋₆ heterocyclyl comprises at least two ring heteroatoms.

Suitably, each optionally substituted C₅₋₆ heterocyclyl is independently selected from optionally substituted morpholinyl, piperazinyl, dioxolanyl and dioxanyl.

Suitably, each C₅₋₆ heterocyclyl is independently selected from

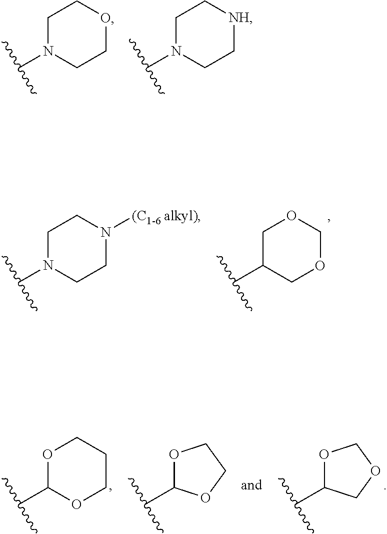

In another aspect, each E¹ is an optionally substituted C₅₋₁₀ heteroaryl group. Suitably, each optionally substituted C₅₋₁₀ heteroaryl comprises at least two ring heteroatoms.

Suitably, each optionally substituted C₅₋₁₀ heteroaryl is independently selected from oxazolyl, isoxazolyl, isoxazinyl, oxadiazolyl (e.g. 1-oxa-2,3-diazolyl, 1-oxa-2,4-diazolyl, 1-oxa-2,5-diazolyl, 1-oxa-3,4-diazolyl), oxatriazolyl, thiazolyl, isothiazolyl, imidazolyl, pyrazolyl, pyridazinyl, pyrimidinyl, pyrazinyl, triazolyl, triazinyl, tetrazolyl, benzoxazolyl, benzisoxazolyl, benzothiazolyl, benzimidazolyl, indazolyl, benzodioxolyl, benzothiadiazolyl, benzotriazolyl, purinyl(e.g., adeninyl, guaninyl), pteridinyl, cinnolinyl, naphthyridinyl, phthalazinyl, quinazolinyl and quinoxalinyl.

More suitably, each optionally substituted C₅₋₁₀ heteroaryl is independently selected from oxazolyl, isoxazolyl, thiazolyl, isothiazolyl, imidazolyl, pyrazolyl, triazolyl, tetrazolyl, pyrimidinyl, pyrazinyl, benzoxazolyl, benzisoxazolyl, benzothiazolyl, benzimidazolyl, indazolyl, cinnolinyl, naphthyridinyl, phthalazinyl, quinazolinyl and quinoxalinyl.

Suitably, each optionally substituted C₅₋₁₀ heteroaryl is independently selected from:

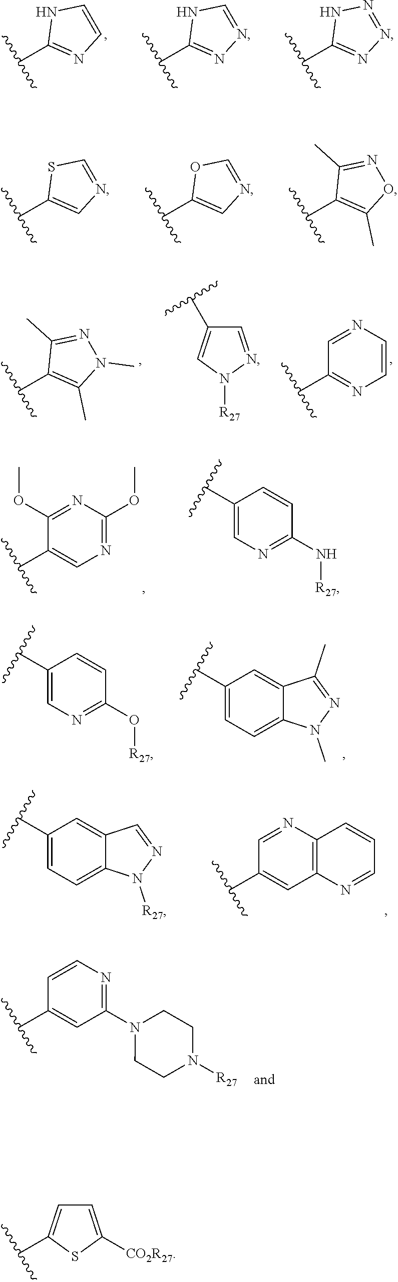

In some aspects, suitably, the optionally substituted C₅₋₁₀ heteroaryl is an optionally substituted C₁₀ heteroaryl group. Suitably, each optionally substituted C₁₀ heteroaryl group is selected from optionally substituted cinnolinyl, naphthyridinyl, phthalazinyl, quinazolinyl and quinoxalinyl.

Suitably, each optionally substituted C₅₋₁₀ heteroaryl group is an optionally substituted naphthyridinyl.

Suitably, each optionally substituted C₅₋₁₀ heteroaryl group is:

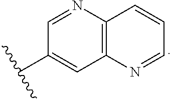 [SMILES: *c1cnc2cccnc2c1]

In another aspect, each E¹ is a C₅₋₁₀ heteroaryl group substituted with a C₅₋₆ heteroaryl or a C₅₋₆ heterocyclyl group wherein each E¹ group may be optionally substituted.

Suitably, each C₅₋₁₀ heteroaryl group is independently selected from pyrrolyl, furanyl, thiophenyl, pyridinyl, oxazolyl, isoxazolyl, isoxazinyl, oxadiazolyl (e.g. 1-oxa-2,3-diazolyl, 1-oxa-2,4-diazolyl, 1-oxa-2,5-diazolyl, 1-oxa-3,4-diazolyl), oxatriazolyl, thiazolyl, isothiazolyl, imidazolyl, pyrazolyl, pyridazinyl, pyrimidinyl, pyrazinyl, triazolyl, triazinyl, tetrazolyl, benzoxazolyl, benzisoxazolyl, benzothiazolyl, benzimidazolyl, indazolyl, benzodioxolyl, benzothiadiazolyl, benzotriazolyl, purinyl(e.g., adeninyl, guaninyl), pteridinyl, cinnolinyl, naphthyridinyl, phthalazinyl, quinazolinyl and quinoxalinyl wherein the C₅₋₁₀ heteroaryl group is substituted with a C₅₋₆ heteroaryl or a C₅₋₆ heterocyclyl group; and e each E¹ group may be independently optionally substituted.

Suitably, each C₅₋₁₀ heteroaryl group is independently selected from pyrrolyl, furanyl, thiophenyl, pyridinyl, oxazolyl, isoxazolyl, isoxazinyl, oxadiazolyl (e.g. 1-oxa-2,3-diazolyl, 1-oxa-2,4-diazolyl, 1-oxa-2,5-diazolyl, 1-oxa-3,4-diazolyl), oxatriazolyl, thiazolyl, isothiazolyl, imidazolyl, pyrazolyl, pyridazinyl, pyrimidinyl, pyrazinyl, triazolyl, triazinyl, tetrazolyl, benzoxazolyl, benzisoxazolyl, benzothiazolyl, benzimidazolyl, indazolyl, benzodioxolyl, benzothiadiazolyl, benzotriazolyl, purinyl(e.g., adeninyl, guaninyl), pteridinyl, cinnolinyl, naphthyridinyl, phthalazinyl, quinazolinyl and quinoxalinyl wherein (i) the C₅₋₁₀ heteroaryl group is substituted with a C₅₋₆ heteroaryl selected from oxazolyl, thizolyl, imidazolyl, pyrazolyl, triazolyl, tetrazolyl, pyrazolyl, pyridazinyl, pyrimidinyl and pyrazinyl; or (ii) the C₅₋₁₀ heteroaryl group is substituted with a C₅₋₆ heterocyclyl group selected from morpholinyl, piperazinyl, dioxolanyl and dioxany; and each E¹ group may be independently optionally substituted.

Suitably, each C₅₋₁₀ heteroaryl group is independently selected from pyrrolyl, furanyl, thiophenyl and pyridinyl substituted with a C₅₋₆ heteroaryl or a C₅₋₆ heterocyclyl group; and independently optionally further substituted.

More suitably, each C₅₋₁₀ heteroaryl group is independently selected from pyrrolyl, furanyl, thiophenyl and pyridinyl substituted with a C₅₋₆ heteroaryl selected from oxazolyl, thizolyl, imidazolyl, pyrazolyl, triazolyl, tetrazolyl, pyrazolyl, pyridazinyl, pyrimidinyl and pyrazinyl; or a C₅₋₆ heterocyclyl group selected from morpholinyl, piperazinyl, dioxolanyl and dioxanyl; and each E¹ group may be independently optionally further substituted.

Suitably, each C₅₋₁₀ heteroaryl group substituted with a C₅₋₆ heteroaryl or a C₅₋₆ heterocyclyl group is:

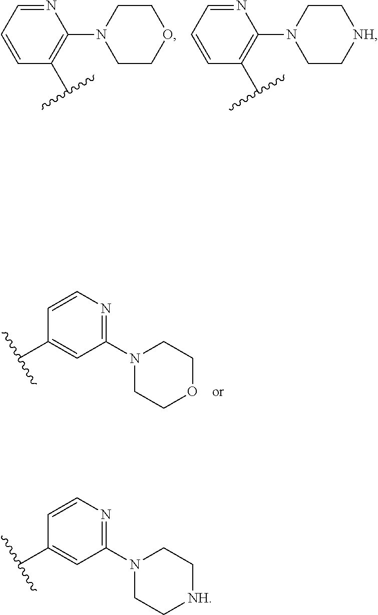

In another aspect, each E¹ is a phenyl substituted with a C₅₋₆ heteroaryl or a C₅₋₆ heterocyclyl group wherein each E¹ group may be independently optionally substituted.

Suitably, each phenyl is substituted with a C₅₋₆ heteroaryl selected from oxazolyl, thizolyl, imidazolyl, pyrazolyl, triazolyl, tetrazolyl, pyrazolyl, pyridazinyl, pyrimidinyl and pyrazinyl; or a C₅₋₆ heterocyclyl group selected from morpholinyl, piperazinyl, dioxolanyl and dioxanyl; and independently optionally further substituted.

Suitably, each phenyl substituted with a C₅₋₆ heteroaryl or a C₅₋₆ heterocyclyl group is:

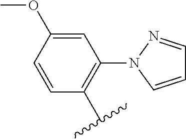 [SMILES: *c1ccc(OC)cc1-n1cccn1]

Suitably, in some aspects, each E¹ contains no optional substituents.

Suitably, in some aspects, each E¹ independently comprises 1, 2 or 3 optional groups independently selected from C₁₋₆ alkyl, OC₁₋₆ alkyl, OCH₂Ph, phenyl and R₂₀. Suitably, in another aspect, each E¹ independently comprises an R₂₀ group. Suitably, in another aspect, each E¹ independently comprises 1, 2 or 3 optional groups independently selected from C₁₋₆ alkyl, OC₁₋₆ alkyl, and OCH₂Ph. More suitably, each E¹ independently comprises 1, 2 or 3 optional groups independently selected from CH₃, CH₂CH₃, OCH₃ and OCH₂CH₃.

Suitably, in some aspects, an E¹ independently comprises 1, 2 or 3 optional groups independently selected from C₁₋₆ alkyl, OC₁₋₆ alkyl, OCH₂Ph, phenyl and R₂₀. Suitably, in another aspect, an E¹ independently comprises an R₂₀ group. Suitably, in another aspect, an E¹ independently comprises 1, 2 or 3 optional groups independently selected from C₁₋₆ alkyl, OC₁₋₆ alkyl, and OCH₂Ph. More suitably, an E¹ independently comprises 1, 2 or 3 optional groups independently selected from CH₃, CH₂CH₃, OCH₃ and OCH₂CH₃.

### Suitable Compounds

Suitably the compound of formula (I) is a compound of formula (II):

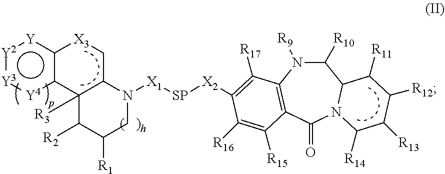

or salts, solvates, isomers or tautomers thereof.

Suitably the compound of formula (I) is a compound of formula (III):

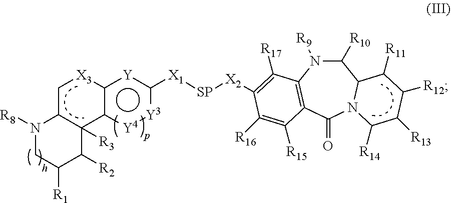

or salts, solvates, isomers or tautomers thereof.

Suitably the compound of formula (I) is a compound of formula (IV):

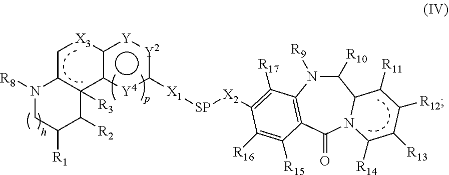

or salts, solvates, isomers or tautomers thereof.

Suitably the compound of formula (I) is a compound of formula (V):

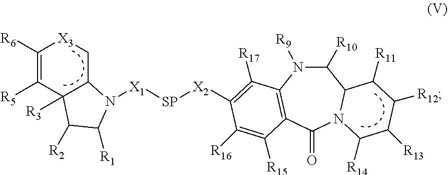

or salts, solvates, isomers or tautomers thereof.

Suitably the compound of formula (I) is a compound of formula (VI):

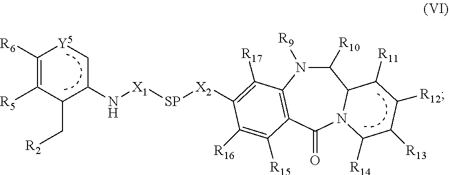

or salts, solvates, isomers or tautomers thereof.

In some aspects, the compound of formula (I) is a compound of formula (II), (III), (IV), (V), (VI) or salts, solvates and tautomers thereof.

More suitably, the compound of formula (I) is a compound of formula (VII),

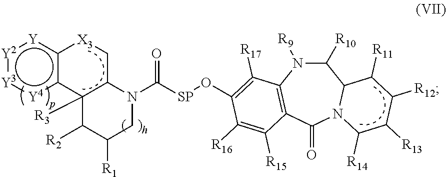

or salts, solvates, isomers or tautomers thereof.

More suitably, the compound of formula (I) is a compound of formula (VIII),

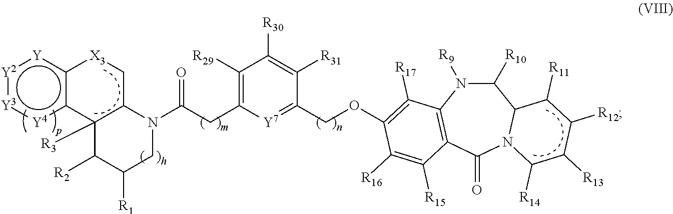

or salts, solvates, isomers or tautomers thereof.

More suitably, in one aspect, the compound of formula (I) is a compound of formula (IX),

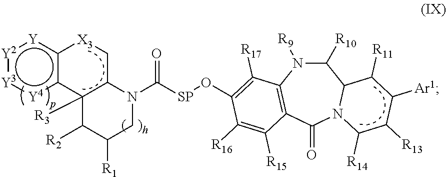

or salts, solvates, isomers or tautomers thereof.

More suitably, in one aspect, the compound of formula (I) is:

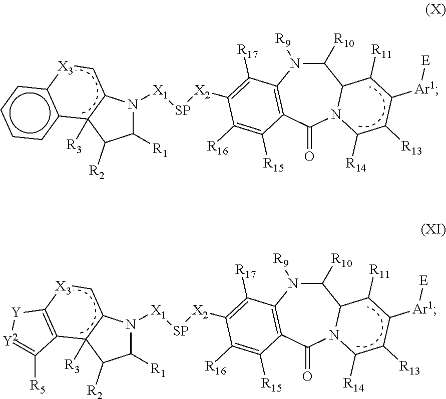

or salts, solvates, isomers or tautomers thereof.

Suitably the compound of formula (I) is a compound of formula (XI) or salts, solvates, isomers or tautomers thereof. More suitably, the compound of formula (I) is a compound of formula (X) or salts, solvates, isomers or tautomers thereof.

More suitably, in one aspect, the compound of formula (I) is:

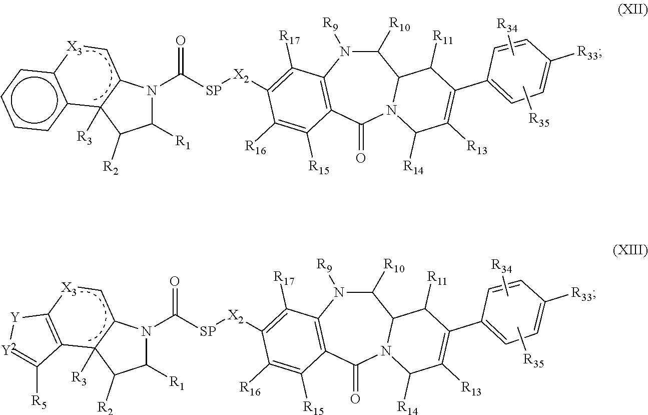

or salts, solvates, isomers or tautomers thereof.

Suitably the compound of formula (I) is a compound of formula (XIII) or salts, solvates, isomers or tautomers thereof. More suitably, the compound of formula (I) is a compound of formula (XII) or salts, solvates, isomers or tautomers thereof.

In one aspect, the compound of formula (I), (II), (III), (IV), (V), (VI), (VII), (VIII), (IX), (X), (XI), (XII) or (XIII) is a racemic mixture at the the C12a position of the B polycyclic group.

Suitably, in another aspect, formula (I), (II), (III), (IV), (V), (VI), (VII), (VIII), (IX), (X), (XI), (XII) or (XIII) has R-stereochemistry at the C12a position of the B polycyclic group.

More suitably, in another aspect, formula (I), (II), (III), (IV), (V), (VI), (VII), (VIII), (IX), (X), (XI), (XII) or (XIII) has S-stereochemistry at the C12a position of the B polycyclic group.

### X₃

X₃ is selected from C═O; C—OH; and C—R₁₈ wherein R₁₈ is a prodrug moiety comprising carbonyl, carbamoyl, glycosyl, O-amino, O-acylamino, para-aminobenzyl ether, peptidyl or phosphate groups. Hence, the C of these groups C═O; C—OH; and C—R₁₈ is a carbon of the ring system of (A1), (A2), (A3) and (A4) in which X₃ appears and the groups ═O; —OH; and —R₁₈ are substituent groups attached to the ring carbon. For example, for (A1) the X₃ groups C═O; C—OH; and C—R₁₈ result in structures such that A is:

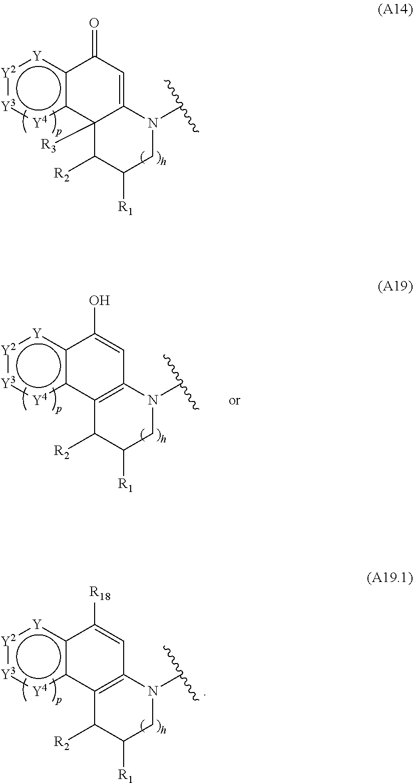

In one aspect, suitably, X₃ is selected from C═O and C—OH.

In an alternative aspect, suitably, X₃ is C—R₁₈.

### R₁

Suitably, R₁ is selected from H, F, Cl, Br and I. More suitably, R₁ is selected from H and Cl. More suitably, R₁ is H.

In one aspect, R₁ is selected from F, Cl, Br and I. More suitably in this aspect, R₁ is Cl.

R₂ and R₃

In one aspect, R₂ is —CH₂-halogen and R₃ is H or is absent. Suitably in this aspect R₂ is selected from —CH₂—F, —CH₂—Cl, —CH₂—Br and —CH₂—I. More suitably, R₂ is selected from —CH₂—Cl and —CH₂—Br. Most suitably, R₂ is-CH₂—Cl.

In another aspect, R₂ is C₁₋₆ alkyl and R₃ is H or is absent. Suitably in this aspect, R₂ is methyl, ethyl, propyl.

In another aspect, R₂ and R₃ together with the carbon atoms to which they are attached form a cyclopropyl ring.

More suitably, R₂ is —CH₂-halogen and R₃ is H or is absent; or R₂ and R₃ together with the carbon atoms to which they are attached form a cyclopropyl ring.

In one aspect, R₃ is H.

In another aspect, R₃ is absent.

### Y

In some aspects, Y is selected from N—R₁₉, O and S. In these aspects, more suitably Y is selected from N—R₁₉ and O. In an alternative, suitably, Y is S. Most suitably, Y is N—R₁₉.

In alternative aspect, Y is C—R₇.

### Y²

In some aspects, Y² is selected from C—R₆ and N. More suitable Y² is C—R₆.

### Y³

In some aspects, Y³ is selected from N—R₁₉, O and S. In these aspects, more suitably Y³ is selected from N—R₁₉ and O. Most suitably, Y³ is N—R₁₉.

### Y⁴

Suitably, Y⁴ is CH.

### Y⁵

Y⁵ is selected from C═O; C—OH; C—NH₂; and C—R₁₈ wherein R₁₈ is a prodrug moiety comprising carbonyl, carbamoyl, glycosyl, O-amino, O-acylamino, para-aminobenzyl ether, peptidyl or phosphate groups. Hence, the C of these groups C═O; C—OH; C—NH₂; and C—R₁₈ is a carbon of the ring system of (A5) in which Y⁵ appears and the groups ═O; —OH; and —R₁₈ are substituent groups attached to the ring carbon. Thus, for (A5) the Y⁵ groups C═O; C—OH; C—NH₂ and C—R₁₈ result in A being (A18), (A22), (A23) or

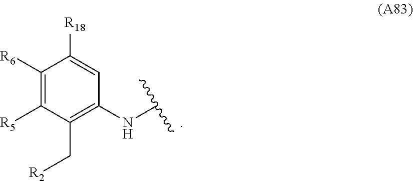

Suitably, Y⁵ is selected C═O; C—OH and C—NH₂.

More suitably, Y⁵ is C—OH.

### Y⁶

Suitably, Y⁶ is selected from —(CH₂)_{z}—, arylene and heteroarylene and the Y⁶ group is optionally substituted.

Suitably, Y⁶ is selected from —(CH₂)_{z}—, phenylene, pyridinylene, pyrrolylene, pyridylene, furanylene, thiphenylene and the Y⁶ group is optionally substituted.

In some aspects, the Y⁶ group is substituted with 1, 2 or 3 independently selected R₂₀ groups

Suitably, Y⁶ is selected from —CH₂—, —CH(R₂₀)—, —CH₂—CH₂—, —CH₂—CH₂—CH₂—, —CH₂—CH₂—CH₂—CH₂—, —CH₂—CH₂—CH₂—CH₂—CH₂—,

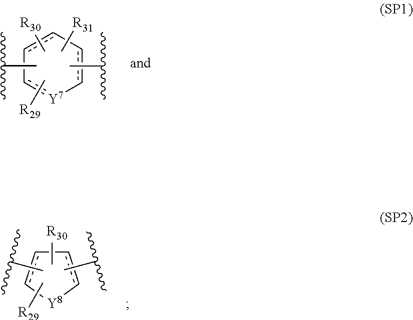

wherein Y⁷ is selected from C—R₃₂ and N;
Y⁸ is selected from N—R₂₅, O and S; and
R₂₉, R₃₀ and R₃₁ are independently selected from H, C₁₋₆ alkyl, OC₁₋₆ alkyl, OCH₂Ph and R₂₀.

Suitably, Y⁶ is —CH₂—, —CH(R₂₀)—, —CH₂—CH₂—, —CH₂—CH₂—CH₂—, —CH₂—CH₂—CH₂—CH₂—, —CH₂—CH₂—CH₂—CH₂—CH₂—,

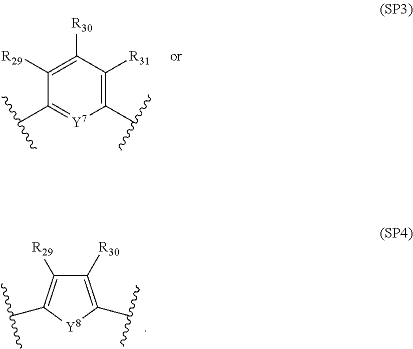

Suitably, Y⁶ is —CH₂—, —CH(R₂₀)—, —CH₂—CH₂—, —CH₂—CH₂—CH₂—, —CH₂—CH₂—CH₂—CH₂—, —CH₂—CH₂—CH₂—CH₂—CH₂— or

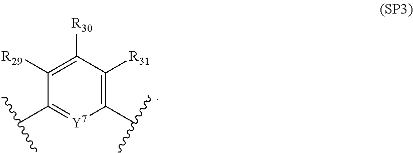

Suitably, Y⁶ is —CH₂—, —CH₂—CH₂—, —CH₂—CH₂—CH₂—, —CH₂—CH₂—CH₂—CH₂—, —CH₂—CH₂—CH₂—CH₂—CH₂— or

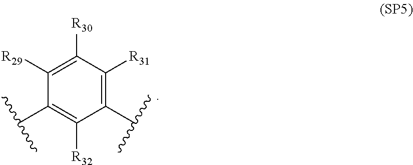

### Y⁷

Y⁷ is selected from C—R₃₂ and N.

In one aspect, Y⁷ is C—R₃₂; suitably, Y⁷ is CH.

In another aspect, Y⁷ is N.

### Y⁸

Y⁸ is selected from N—R₂₅, O and S.

Suitably, Y⁸ is N—R₂₅; more suitably, Y⁸ is selected from N—H and N—CH₃.

R₄, R₅, R₆ and R₇

In the aspects where one of R₄ and R₅, R₅ and R₆, or R₆ and R₇ together with the carbon atoms to which they are attached form a 6-membered aryl, or a 5- or 6-membered cyclic, heterocyclic, or heteroaryl ring optionally substituted with up to three independently selected optional R₂₀ groups, groups (A1)-(A5) contain a further fused ring (not drawn). In these aspects, then the remaining groups (from R₄, R₅, R₆ and R₇) that do not form the further fused ring are each independently selected from the normal specified list of groups, i.e. from H, C₁₋₆ alkyl, OC₁₋₆ alkyl, CO₂H, CO₂C₁₋₆ alkyl, OCH₂Ph and R₂₀. For example, where A is (A1), p is 1 and R₅ and R₆ together with the carbon atoms to which they are attached form a 6-membered aryl ring the structure of the group A can be shown as follows:

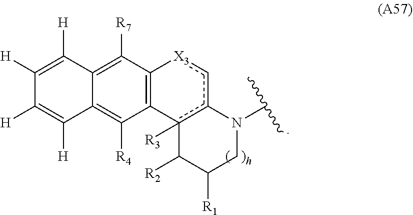

Groups R₄ and R₇ do not form the further fused ring and so are each independently selected from the normal specified list of groups for R₄, R₅, R₆ and R₇, i.e. from H, C₁₋₆ alkyl, OC₁₋₆ alkyl, CO₂H, CO₂C₁₋₆ alkyl, OCH₂Ph and R₂₀. In addition, the H groups shown on the further fused ring of (A57) may be optionally substituted with up to three independently selected optional R₂₀ groups.

More suitably, R₄, R₅, R₆ and R₇ are each independently selected from H, C₁₋₆ alkyl, OC₁₋₆ alkyl, CO₂H, CO₂C₁₋₆ alkyl, OCH₂Ph and R₂₀. In some aspects, one of R₄, R₅, R₆ and R₇ is R₂₀; and the remaining of R₄, R₅, R₆ and R₇ are each independently selected from H, C₁₋₆ alkyl, OC₁₋₆ alkyl, CO₂H, CO₂C₁₋₆ alkyl and OCH₂Ph.

In some aspects, more suitably R₄, R₅, R₆ and R₇ are each independently selected from H, C₁₋₆ alkyl, OC₁₋₆ alkyl, CO₂H, CO₂C₁₋₆ alkyl and OCH₂Ph.

More suitably, R₄ is selected from H, C₁₋₆ alkyl, OC₁₋₆ alkyl, CO₂H, CO₂C₁₋₆ alkyl and OCH₂Ph. More suitably, R₄ is selected from H, CH₃, CH₂CH₃, OCH₃, OCH₂CH₃, CO₂CH₃ and CO₂CH₂CH₃. More suitably, R₄ is H.

More suitably, R₅ is selected from H, C₁₋₆ alkyl, OC₁₋₆ alkyl, CO₂H, CO₂C₁₋₆ alkyl and OCH₂Ph. More suitably, R₅ is selected from H, CH₃, CH₂CH₃, OCH₃, OCH₂CH₃, CO₂CH₃ and CO₂CH₂CH₃. More suitably, R₅ is H.

More suitably, R₆ is selected from H, C₁₋₆ alkyl, OC₁₋₆ alkyl, CO₂H, CO₂C₁₋₆ alkyl and OCH₂Ph. More suitably, R₆ is selected from H, CH₃, CH₂CH₃, OCH₃, OCH₂CH₃, CO₂CH₃ and CO₂CH₂CH₃. More suitably, R₆ is H.

More suitably, R₇ is selected from H, C₁₋₆ alkyl, OC₁₋₆ alkyl, CO₂H, CO₂C₁₋₆ alkyl and OCH₂Ph. More suitably, R₇ is selected from H, CH₃, CH₂CH₃, OCH₃, OCH₂CH₃, CO₂CH₃ and CO₂CH₂CH₃. More suitably, R₇ is H.

### R₈

Suitably, R₈ is selected from H and R₂₀.

In some aspects, R₈ is selected from selected from H, C₁₋₆ alkyl, OC₁₋₆ alkyl, OCH₂Ph and nitrogen protecting groups. More suitably, R₈ is selected from selected from H and C₁₋₆ alkyl. More suitably, R₈ is selected from selected from H, CH₃ and CH₂CH₃.

R₉ and R₁₀

Suitably, (i) R₉ and R₁₀ together form a double bond.

Suitably, (ii) R₉ is H and R₁₀ is OH.

Suitably, (iii) R₉ is H and R₁₀ is OCH₃ or OCH₂CH₃.

Suitably, (iv) R₉ is selected from OH, SO₃H, nitrogen protecting groups, methyl, ethyl, OCH₃, OCH₂CH₃, OCH₂Ph, (CH₂)ₛ—CO₂H, (CH₂)ₛ—CO₂CH₃, (CH₂)ₛ—CO₂CH₂CH₃, K₁—R*, O—(CH₂)ₜ—NH₂, O—(CH₂)ₜ—NH—CH₃, (CH₂)ₛ—NH₂, (CH₂)ₛ—NH—CH₃, C(═O)—NH—(CH₂)ₜ—NH₂, C(═O)—NH—(CH₂)ₜ—NH—CH₃, C(═O)—NH—C₆H₄—(CH₂)ₛ—H, C(═O)—NH—(CH₂)ₜ—C(═NH)NH₂ and C(═O)—NH—(CH₂)ₜ—C(═NH)NH—CH₃ and R₁₀ is H. More suitably (iv), R₉ is selected from OH, SO₃H, methyl, ethyl, OCH₃, OCH₂CH₃, CO₂H, CO₂CH₃, CO₂CH₂CH₃, K₁—R*, O—(CH₂)ₜ—NH₂ and (CH₂)ₛ—NH₂ and R₁₀ is H. More suitably (iv), R₉ is SO₃H or K₁—R*, and R₁₀ is H.

In some aspects, R₉ is SO₃H and the compound of formula (I) is a salt thereof. Suitably, in this aspect, R₉ is SO₃H and the compound of formula (I) is an alkali metal salt thereof (AM)⁺; hence, in this aspect, R₉ may be written as SO₃⁻(AM)⁺. Suitably, R₉ is SO₃H and the compound of formula (I) is an alkali metal salt thereof chosen from L1⁺, Na⁺ and K⁺. More suitably, R₉ is SO₃H and the compound of formula (I) is a Na⁺ salt thereof; hence, in this aspect, R₉ may be written as SO₃⁻Na⁺.

Suitably, (iv) R₉ is H and R₁₀ is oxo or H.

More suitably, R₉ and R₁₀ are either (i), (ii), (iii) or (iv). More suitably, R₉ and R₁₀ are either (i), (ii) or (iii).

R₁₁, R₁₂, R₁₃ and R₁₄

For the options where any of R₁₁, R₁₂, R₁₃ and R₁₄ are each independently selected from ═CH₂, ═CH—(CH₂)ₛ—CH₃, ═CH—(CH₂)ₛ—R₂₅ and ═O, the carbon of the C-ring to which it is attached cannot have an optional double bond in order for the valence requirements of the molecule to be met. For example, if R₁₁ is ═CH₂ and is positioned at the C1 position of the C-ring adjacent to the fused carbon of the C-ring, and R₁₂ and R₁₃ are each H then the resulting B group may be represented as:

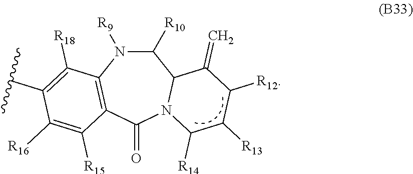

Suitably, one of R₁₁, R₁₂, R₁₃ and R₁₄ is Ar¹.

Suitably, the remaining of R₁₁, R₁₂, R₁₃ and R₁₄ are each independently selected from H, E, R₂₀, R₂₅, ═CH—(CH₂)S—R₂₅, (CH₂)S—OR₂₅, (CH₂)S—CO₂R₂₅, (CH₂)ₛ—NR₂₅R₂₆, O—(CH₂)ₜ—NR₂₅R₂₆, NH—C(O)—R₂₅, O—(CH₂)ₜ—NH—C(O)—R₂₅, O—(CH₂)ₜ—C(O)—NH—R₂₅, (CH₂)S—SO₂R₂₅, O—SO₂R₂₅, (CH₂)ₛ—C(O)R₂₅ and (CH₂)ₛ—C(O)NR₂₅R₂₆.

Suitably, the remaining of R₁₁, R₁₂, R₁₃ and R₁₄ are each independently selected from H, E, R₂₀, R₂₅, (CH₂)S—OR₂₅, (CH₂)ₛ—CO₂R₂₅, (CH₂)ₛ—NR₂₅R₂₆, O—(CH₂)ₜ—NR₂₅R₂₆, NH—C(O)—R₂₅, O—(CH₂)ₜ—NH—C(O)—R₂₅, O—(CH₂)ₜ—C(O)—NH—R₂₅, (CH₂)ₛ—C(O)R₂₅ and (CH₂)ₛ—C(O)NR₂₅R₂₆.

Suitably at least one of the remaining of R₁₁, R₁₂, R₁₃ and R₁₄ is H. Suitably, at least two of the remaining of R₁₁, R₁₂, R₁₃ and R₁₄ are H.

In some aspects, at least one of R₁₁, R₁₂, R₁₃ and R₁₄ is E. Suitably, 1, 2 or 3 of R₁₁, R₁₂, R₁₃ and R₁₄ is E.

Suitably, in aspect (ab) R^{X} is -E¹-Z¹—Ar¹ where the Ar¹ group is optionally further substituted with 1 or 2 optional groups independently selected from C₁₋₆ alkyl, OC₁₋₆ alkyl, OCH₂Ph, E, R₂₀ and R₂₅.

More suitably, in aspect (ab) R^{X} is —Ar¹—Z¹-E where the Ar¹ group is optionally further substituted with 1 or 2 optional groups independently selected from C₁₋₆ alkyl, OC₁₋₆ alkyl, OCH₂Ph, E, R₂₀ and R₂₅.

In aspect (ab) suitably, the remaining of R₁₁, R₁₂, R₁₃ and R₁₄ are H or E.

Suitably, in aspect (ab) suitably, 1, 2 or 3 of the remaining of R₁₁, R₁₂, R₁₃ and R₁₄ are H.

Suitably, in some aspects, R₁₁ is H. Suitably, in alternative aspects, R₁₁ is E. Suitably, in alternative aspects, R₁₁ is Ar¹.

Suitably, in some aspects, R₁₂ is H. Suitably, in alternative aspects, R₁₂ is E. Suitably, in alternative aspects, R₁₂ is Ar¹

Suitably, in some aspects, R₁₃ is H. Suitably, in alternative aspects, R₁₃ is E. Suitably, in alternative aspects, R₁₃ is Ar¹

Suitably, in some aspects, R₁₄ is H. Suitably, in alternative aspects, R₁₄ is E. Suitably, in alternative aspects, R₁₄ is Ar¹

### R^{X}

When R^{X} is —Ar¹—Z¹-E, then suitably, R^{X} is —Ar¹—NR₂₆-E; —Ar¹—C(═O)—O-E; —Ar¹—O-E; or —Ar¹-E. Suitably, R^{X} is —Ar¹-E.

When R^{X} is -E¹-Z¹—Ar¹; then suitably, R^{X} is -E¹-NR₂₆—Ar¹; -E¹-C(═O)—O—Ar¹; -E¹-O—Ar¹; or -E¹-Ar¹. Suitably, R^{X} is -E¹-Ar¹.

In some aspects, R^{X} is —Ar¹-E or -E¹-Ar¹. More suitably, R^{X} is —Ar¹-E.

For the above aspects of R^{X}, Ar¹ is optionally further substituted with 1 or 2 optional groups independently selected from C₁₋₆ alkyl, OC₁₋₆ alkyl, OCH₂Ph, E, R₂₀ and R₂₅.

### Z¹

Suitably, Z¹ is NH, NCH₃, C(═O)—O, O or is absent. Suitably, Z¹ is NH, C(═O)—O, O or is absent. More suitably, Z¹ is absent.

R₁₅, R₁₆, R¹⁷ and R₁₈

Suitably, R₁₅, R₁₆ R₁₇ and R₁₈ are each independently selected from H and R₂₀.

Suitably, R₁₅, R₁₆ R₁₇ and R₁₈ are each independently selected from H, (CH₂)ⱼ—OH, methyl, ethyl, OCH₃, OCH₂CH₃, OCH₂Ph, CO₂H, CO₂CH₃, CO₂CH₂CH₃, O—(CH₂)ₜ—NH₂ and (CH₂)ₛ—NH₂.

More suitably, R₁₅, R₁₆ R₁₇ and R₁₈ are each independently selected from H, (CH₂)ⱼ—OH, OCH₃, OCH₂CH₃, OCH₂Ph and (CH₂)ₛ—NH₂.

More suitably, R₁₅ is H.

More suitably, R₁₆ is OCH₃.

More suitably, R₁₇ is OCH₃.

More suitably, R₁₈ is H.

R₁₉, R₂₁, R₂₂, R₂₃, R₂₄, R₂₆ and R₂₈

Suitably each R₁₉, R₂₁, R₂₂, R₂₃, R₂₄, R₂₆ and R₂₈ is independently selected from H, methyl, ethyl, n-propyl, i-propyl, n-butyl, s-butyl, i-butyl and t-butyl.

Suitably each R₁₉, R₂₁, R₂₂, R₂₃, R₂₄, R₂₆ and R₂₈ is independently selected from H, methyl, and ethyl. More suitably each R₁₉, R₂₁, R₂₂, R₂₃, R₂₄, R₂₆ and R₂₈ is independently selected from H and methyl.

### R₂₀

Suitably, each R₂₀ is independently selected from (CH₂)ⱼ—OH, (CH₂)ⱼ—CO₂R₂₇, C(O)R₂₇, O—(CH₂)ₖ—NR₂₇R₂₈, K₁—R*, (CH₂)ⱼ—NR₂₇R₂₈, C(═O)—NH—(CH₂)ₖ—NR₂₇R₂₈, C(═O)—NH—C₆H₄—(CH₂)ⱼ—R₂₇ and C(═O)—NH—(CH₂)ₖ—C(═NH)NR₂₇R₂₈.

Suitably, each R₂₀ is independently selected from (CH₂)ⱼ—OH, (CH₂)ⱼ—CO₂H, (CH₂)ⱼ—CO₂CH₃, (CH₂)ⱼ—CO₂CH₂CH₃, C(O)H, C(O)CH₃, C(O)Ph, K₁—R*, O—(CH₂)ₖ—NH₂, O—(CH₂)ₖ—NH—CH₃, (CH₂)ⱼ—NH₂, (CH₂)ⱼ—NH—CH₃, C(═O)—NH—(CH₂)ₖ—NH₂, C(═O)—NH—(CH₂)ₖ—NH—CH₃, C(═O)—NH—C₆H₄—(CH₂)ⱼ—H, C(═O)—NH—(CH₂)ₖ—C(═NH)NH₂ and C(═O)—NH—(CH₂)ₖ—C(═NH)NH—CH₃.

More suitably, each R₂₀ is independently selected from (CH₂)ⱼ—OH, CO₂H, CO₂CH₃, CO₂CH₂CH₃, K₁—R*, O—(CH₂)ₖ—NH₂ and (CH₂)ⱼ—NH₂.

Suitably, one R₂₀ group is selected from K₁—R*, O—(CH₂)ₖ—NR₂₇R₂₈, (CH₂)ⱼ—NR₂₇R₂₈, C(═O)—NH—(CH₂)ₖ—NR₂₇R₂₈; C(═O)—NH—C₆H₄—(CH₂)ⱼ—R₂₇ and C(═O)—NH—(CH₂)ₖ—C(═NH)NR₂₇R₂₈; and the remaining R₂₀ groups are each independently selected from (CH₂)ⱼ—OH and (CH₂)ⱼ—CO₂R₂₇.

More suitably, one R₂₀ group is selected from K₁—R*, O—(CH₂)ₖ—NH₂, O—(CH₂)ₖ—NH—CH₃, (CH₂)ⱼ—NH₂, (CH₂)ⱼ—NH—CH₃, C(═O)—NH—(CH₂)ₖ—NH₂, C(═O)—NH—(CH₂)ₖ—NH—CH₃, C(═O)—NH—C₆H₄—(CH₂)ⱼ—H, C(═O)—NH—(CH₂)ₖ—C(═NH)NH₂ and C(═O)—NH—(CH₂)ₖ—C(═NH)NH—CH₃; and the remaining R₂₀ groups are each independently selected from (CH₂)ⱼ—OH, (CH₂)ⱼ—CO₂H, (CH₂)ⱼ—CO₂CH₃ and (CH₂)ⱼ—CO₂CH₂CH₃.

More suitably, one R₂₀ group is selected from O—(CH₂)ₖ—NH₂ and (CH₂)ⱼ—NH₂; and the remaining R₂₀ groups are each independently selected from (CH₂)ⱼ—OH, CO₂H, CO₂CH₃, CO₂CH₂CH₃.

More suitably, the compound of formula (I) comprises at least one R₂₀ group. More suitably, the compound of formula (I) comprises 1, 2, 3, 4, 5 or 6 R₂₀ groups.

More suitably, in some aspects the compound of formula (I) contain only a single R₂₀ group.

Suitably, R₂₀ is selected from (CH₂)ⱼ—OH, (CH₂)ⱼ—CO₂R₂₇, K₁—R*, O—(CH₂)ₖ—NR₂₇R₂₈, (CH₂)ⱼ—NR₂₇R₂₈, C(═O)—NH—(CH₂)ₖ—NR₂₇R₂₈; C(═O)—NH—C₆H₄—(CH₂)ⱼ—R₂₇ and C(═O)—NH—(CH₂)ₖ—C(═NH)NR₂₇R₂₈.

Suitably, R₂₀ is selected from (CH₂)ⱼ—OH, (CH₂)ⱼ—CO₂H, (CH₂)ⱼ—CO₂CH₃, (CH₂)ⱼ—CO₂CH₂CH₃, K₁—R*, O—(CH₂)ₖ—NH₂, O—(CH₂)ₖ—NH—CH₃, (CH₂)ⱼ—NH₂, (CH₂)ⱼ—NH—CH₃, C(═O)—NH—(CH₂)ₖ—NH₂, C(═O)—NH—(CH₂)ₖ—NH—CH₃, C(═O)—NH—C₆H₄—(CH₂)ⱼ—H, C(═O)—NH—(CH₂)ₖ—C(═NH)NH₂ and C(═O)—NH—(CH₂)ₖ—C(═NH)NH—CH₃.

More suitably, R₂₀ is selected from (CH₂)ⱼ—OH, K₁—R*, O—(CH₂)ₖ—NH₂ and (CH₂)ⱼ—NH₂.

In some aspects, suitable, R₂₀ is (CH₂)ⱼ—OH.

In some aspects, more suitably, one R₂₀ group is K₁—R*. More suitably, in this aspect, the compound of formula (I) comprise a single R₂₀ group.

In some aspects, suitable, R₂₀ is (CH₂)ⱼ—NH₂.

Suitably, in alternative aspects, R₂₀ groups are absent from the compound of formula (I). Hence, in such aspects there are no R₂₀ groups.

### R₂₅

Suitably, each R₂₅ is selected from C₅₋₉ heteroaryl, C₆₋₁₆ heteroarylalkyl, phenyl, benzyl and phenethyl; wherein the heteroaryl, heteroarylalkyl, phenyl and aralkyl groups are optionally substituted with 1, 2 or 3 independently selected optional C₁₋₆ alkyl, OC₁₋₆ alkyl and R₂₀ groups.

Suitably, each R₂₅ is selected from H, C₁₋₁₂ alkyl, N-methylpyrrolyl, furanyl, thiophenyl, N-methylimidazolyl, oxazolyl, thiazolyl, pyridyl, indolyl, N-methylindolyl, benzofuranyl, benzothiophenyl, benzimidazolyl, N-methylbenzoimidazolyl, benzooxazolyl, benzothiazolyl, pyrrol-3-ylmethyl, pyrrol-4-ylmethyl, pyrrolyl, pyrazolyl, imidazolyl, triazolyl, tetrazolyl, thiazolinyl, oxazinyl, isoxazolyl, pyrazinyl, pyrimidinyl, indazolyl, imidazol-2-ylmethyl, imidazol-4-ylmethyl, thiophen-3-ylmethyl, furan-3-ylmethyl, phenyl, benzyl and phenethyl; wherein the heteroaryl, heteroarylalkyl, phenyl and aralkyl groups are optionally substituted with 1, 2 or 3 independently selected optional C₁₋₆ alkyl, OC₁₋₆ alkyl and R₂₀ groups.

Suitably, each R₂₅ is selected from H, C₁₋₆ alkyl, N-methylpyrrolyl, furanyl, thiophenyl, N-methylimidazolyl, oxazolyl, thiazolyl, pyridyl, indolyl, N-methylindolyl, benzofuranyl, benzothiophenyl, benzimidazolyl, N-methylbenzoimidazolyl, benzooxazolyl, benzothiazolyl, pyrrol-3-ylmethyl, pyrrol-4-ylmethyl, pyrrolyl, pyrazolyl, imidazolyl, triazolyl, tetrazolyl, thiazolinyl, oxazinyl, isoxazolyl, pyrazinyl, pyrimidinyl, indazolyl, imidazol-2-ylmethyl, imidazol-4-ylmethyl, thiophen-3-ylmethyl, furan-3-ylmethyl, phenyl, benzyl and phenethyl; wherein the heteroaryl, heteroarylalkyl, phenyl and aralkyl groups are optionally substituted with 1, 2 or 3 independently selected optional C₁₋₆ alkyl, OC₁₋₆ alkyl and R₂₀ groups.

Suitably, each R₂₅ is selected from H, methyl, ethyl, n-propyl, i-propyl, n-butyl, s-butyl, i-butyl, t-butyl, N-methylpyrrolyl, furanyl, thiophenyl, N-methylimidazolyl, oxazolyl, thiazolyl, pyridyl, indolyl, N-methylindolyl, benzofuranyl, benzothiophenyl, benzimidazolyl, N-methylbenzoimidazolyl, benzooxazolyl, benzothiazolyl, pyrrol-3-ylmethyl, pyrrol-4-ylmethyl, pyrrolyl, pyrazolyl, imidazolyl, triazolyl, tetrazolyl, thiazolinyl, oxazinyl, isoxazolyl, pyrazinyl, pyrimidinyl, indazolyl, imidazol-2-ylmethyl, imidazol-4-ylmethyl, thiophen-3-ylmethyl, furan-3-ylmethyl, phenyl, benzyl and phenethyl optionally substituted with 1, 2 or 3 independently selected optional C₁₋₆ alkyl, OC₁₋₆ alkyl and R₂₀ groups.

Suitably, in one aspect, each R₂₅ is selected from H, methyl, ethyl, n-propyl, i-propyl, n-butyl, s-butyl, i-butyl, t-butyl, N-methylpyrrolyl, furanyl, thiophenyl, N-methylimidazolyl, oxazolyl, thiazolyl, pyridyl, indolyl, N-methylindolyl, benzofuranyl, benzothiophenyl, benzimidazolyl, N-methylbenzoimidazolyl, benzooxazolyl, benzothiazolyl, phenyl, benzyl and phenethyl optionally substituted with 1, 2 or 3 independently selected optional C₁₋₆ alkyl, OC₁₋₆ alkyl and R₂₀ groups.

Suitably, in another aspect, each R₂₅ is selected from:

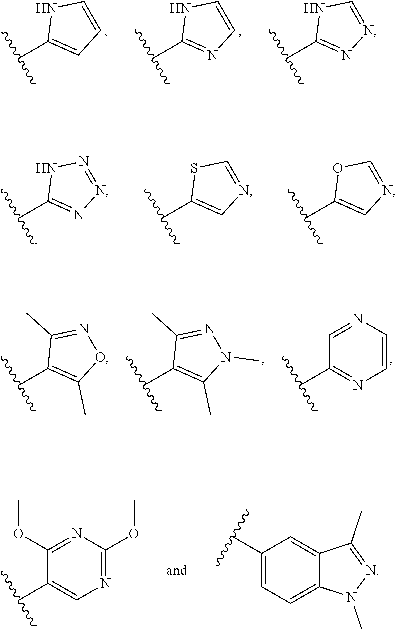

In some embodiments, each R₂₅ is selected from H, methyl, ethyl, n-propyl, i-propyl, n-butyl, s-butyl, i-butyl and t-butyl.

Suitably, in some aspects, each R₂₅ contains no optional groups.

Suitably, in some aspects, each R₂₅ group independently comprises 1, 2 or 3 optional groups independently selected from C₁₋₆ alkyl, OC₁₋₆ alkyl and R₂₀. Suitably, in another aspect, each R₂₅ group independently comprises an R₂₀ group. Suitably, in another aspect, each R₂₅ group independently comprises 1, 2 or 3 optional groups independently selected from C₁₋₆ alkyl and OC₁₋₆ alkyl. More suitably, each R₂₅ group independently comprises 1, 2 or 3 optional groups independently selected from CH₃, CH₂CH₃, OCH₃ and OCH₂CH₃.

Suitably, in some aspects, an R₂₅ group independently comprises 1, 2 or 3 optional groups independently selected from C₁₋₆ alkyl, OC₁₋₆ alkyl and R₂₀. Suitably, in another aspect, an R₂₅ group independently comprises an R₂₀ group. Suitably, in another aspect, an R₂₅ group independently comprises 1, 2 or 3 optional groups independently selected from C₁₋₆ alkyl and OC₁₋₆ alkyl. More suitably, an R₂₅ group independently comprises 1, 2 or 3 optional groups independently selected from CH₃, CH₂CH₃, OCH₃ and OCH₂CH₃.

### R₂₇

In some aspects, an R₂₇ is independently selected from H and C₁₋₆ alkyl. In other aspects, each R₂₇ is independently selected from H and C₁₋₆ alkyl. Suitably, in such aspects, the and C₁₋₆ alkyl is independently selected from methyl, ethyl, n-propyl, i-propyl, n-butyl, s-butyl, i-butyl and t-butyl.

In another aspect, an R₂₇ is independently selected from C₅₋₂₀ aryl and C₆₋₂₆ aralkyl. Suitably, an R₂₇ is independently selected from phenyl, biphenyl, indenyl, naphthalenyl, benzyl and phenethyl.

Suitably, each R₂₇ is independently selected from H, methyl, ethyl, n-propyl, i-propyl, n-butyl, s-butyl, i-butyl and t-butyl, phenyl, biphenyl, indenyl, naphthalenyl, benzyl and phenethyl.

R₂₉, R₃₀, and R₃₂

R₂₉, R₃₀, R₃₁ and R₃₂ are each independently selected from H, C₁₋₆ alkyl, OC₁₋₆ alkyl, OCH₂Ph and R₂₀.

Suitably, R₂₉, R₃₀, R₃₁ and R₃₂ are each independently selected from H, (CH₂)ⱼ—OH, methyl, ethyl, OCH₃, OCH₂CH₃, OCH₂Ph, CO₂H, CO₂CH₃, CO₂CH₂CH₃, K₁—R*, O—(CH₂)ₜ—NH₂ and (CH₂)ₛ—NH₂.

More suitably, R₂₉, R₃₀, R₃₁ and R₃₂ are each independently selected from H, (CH₂)ⱼ—OH, OCH₃, OCH₂CH₃, OCH₂Ph, K₁—R* and (CH₂)ₛ—NH₂.

In one aspect, more suitably, R₃₀ is selected from (CH₂)ⱼ—OH, K₁—R*, and (CH₂)ₛ—NH₂; and R₂₉, R₃₁ and R₃₂ are H.

More suitably, R₂₉ is H.

More suitably, R₃₀ is H.

More suitably, R₃₁ is H.

More suitably, R₃₂ is H.

In some aspects, one of R₂₉, R₃₀, R₃₁ and R₃₂ is selected from OH, K₁—R*, O—(CH₂)ₖ—NR₂₇R₂₈, (CH₂)ⱼ—NR₂₇R₂₈, (CH₂)ⱼ—CO₂R₂₇, C(═O)—NH—(CH₂)ₖ—NR₂₇R₂₈; C(═O)—NH—C₆H₄—(CH₂)ⱼ—R₂₇ and C(═O)—NH—(CH₂)ₖ—C(═NH)NR₂₇R₂₈; and the remaining of R₂₉, R₃₀, R₃₁ and R₃₂ are each independently selected from H, C₁₋₆ alkyl, OC₁₋₆ alkyl and OCH₂Ph.

In some aspects, one of R₂₉, R₃₀, R₃₁ and R₃₂ is selected from OH, K₁—R*, O—(CH₂)_{g}-NR₂₆R₂₇, (CH₂)_{f}-NR₂₆R₂₇, C(═O)—NH—(CH₂)_{g}-NR₂₆R₂₇, C(═O)—NH—C₆H₄—(CH₂)_{f}-R₂₀ and C(═O)—NH—(CH₂)_{g}-C(═NH)NR₂₆R₂₇; and the remaining of R₂₉, R₃₀, R₃₁ and R₃₂ are H.

R₃₃, R₃₄ and R₃₅.

R₃₃, R₃₄ and R₃₅ are each independently selected from H, E, C₁₋₆ alkyl, OC₁₋₆ alkyl, OCH₂Ph, R₂₀ and R₂₅.

Suitably, R₃₃, R₃₄ and R₃₅ are each independently selected from H, E, (CH₂)ⱼ—OH, methyl, ethyl, OCH₃, OCH₂CH₃, OCH₂Ph, CO₂H, CO₂CH₃, CO₂CH₂CH₃, K₁—R*, O—(CH₂)ₜ—NH₂ and (CH₂)ₛ—NH₂.

More suitably, in some aspects, 1, 2 or 3 of R₃₃, R₃₄ and R₃₅ is E. More suitably, one of R₃₃, R₃₄ and R₃₅ is E.

More suitably, R₃₃, R₃₄ and R₃₅ are each independently selected from H, E, (CH₂)ⱼ—OH, OCH₃, OCH₂CH₃, OCH₂Ph, K₁—R* and (CH₂)ₛ—NH₂.

More suitably, R₃₃ is H.

More suitably, R₃₄ is H.

More suitably, R₃₅ is H.

In some aspects, one of R₃₃, R₃₄ and R₃₅ is R₂₀ and the remaining of R₃₃, R₃₄ and R₃₅ are each independently selected from H, E, C₁₋₆ alkyl, OC₁₋₆ alkyl and OCH₂Ph.

In some aspects, one of R₃₃, R₃₄ and R₃₅ is selected from O—(CH₂)_{g}—NR₂₆R₂₇, (CH₂)_{f}—NR₂₆R₂₇, C(═O)—NH—(CH₂)_{g}—NR₂₆R₂₇, C(═O)—NH—C₆H₄—(CH₂)_{f}—R₂₀ and C(═O)—NH—(CH₂)_{g}—C(═NH)NR₂₆R₂₇; and the remaining of R₃₃, R₃₄ and R₃₅ are H.

### Combinations

Suitably, at least one of R₄, R₅, R₆, R₇, R₈, R₉, R₁₁, R₁₂, R₁₃, R₁₄, R₁₅, R₁₆ and R₁₇ is selected from H, C₁₋₆ alkyl, OC₁₋₆ alkyl and OCH₂Ph; suitably, at least two, three, four, five, six, seven, eight, nine, ten, eleven, twelve or thirteen of R₄, R₅, R₆, R₇, R₈, R₉, R₁₁, R₁₂, R₁₃, R₁₄, R₁₅, R₁₆ and R₁₇ are selected from H, C₁₋₆ alkyl, OC₁₋₆ alkyl and OCH₂Ph.

Suitably, at least one of R₄, R₅, R₆, R₇, R₈, R₉, R₁₁, R₁₂, R₁₃, R₁₄, R₁₅, R₁₆ and R₁₇; suitably, at least two, three, four, five, six, seven, eight, nine, ten, eleven, twelve or thirteen of R₅, R₆, R₈, R₉, R₁₁, R₁₂, R₁₃, R₁₆ and R₁₇ are H.

In some aspects, suitably, one of R₄, R₅, R₆, R₇, R₈, R₉, R₁₁, R₁₂, R₁₃, R₁₄, R₁₅, R₁₆ and R₁₇ is R₂₀. Suitably the remaining of R₄, R₅, R₆, R₇, R₈, R₉, R₁₁, R₁₂, R₁₃, R₁₄, R₁₅, R₁₆ and R₁₇ are selected from H, C₁₋₆ alkyl, OC₁₋₆ alkyl and OCH₂Ph.

In some aspects, suitably, at least one of R₄, R₅, R₆, R₇, R₈, R₉, R₁₁, R₁₂, R₁₃, R₁₄, R₁₅, R₁₆, R₁₇, R₂₉, R₃₀, R₃₁ and R₃₂ is selected from H, C₁₋₆ alkyl, OC₁₋₆ alkyl and OCH₂Ph; suitably, at least two, three, five, six, seven, eight, nine, ten, eleven, twelve, thirteen, fourteen, fifteen, sixteen or seventeen of R₄, R₅, R₆, R₇, R₈, R₉, R₁₁, R₁₂, R₁₃, R₁₄, R₁₅, R₁₆, R₁₇, R₂₉, R₃₀, R₃₁ and R₃₂ are selected from H, C₁₋₆ alkyl, OC₁₋₆ alkyl and OCH₂Ph.

In some aspects, suitably, at least one of R₄, R₅, R₆, R₇, R₈, R₉, R₁₁, R₁₂, R₁₃, R₁₄, R₁₅, R₁₆, R₁₇, R₂₉, R₃₀, R₃₁ and R₃₂ is H; suitably, at least two, three, five, six, seven, eight, nine, ten, eleven, twelve, thirteen, fourteen, fifteen, sixteen or seventeen of R₄, R₅, R₆, R₇, R₈, R₉, R₁₁, R₁₂, R₁₃, R₁₄, R₁₅, R₁₆, R₁₇, R₁₈, R₂₉, R₃₀, R₃₁ and R₃₂ are H.

In some aspects, suitably, at least one R₄, R₅, R₆, R₇, R₈, R₉, R₁₁, R₁₂, R₁₃, R₁₄, R₁₅, R₁₆, R₁₇, R₂₉, R₃₀, R₃₁ and R₃₂ is R₂₀. Suitably the remaining of R₄, R₅, R₆, R₇, R₈, R₉, R₁₁, R₁₂, R₁₃, R₁₄, R₁₅, R₁₆, R₁₇, R₂₉, R₃₀, R₃₁ and R₃₂ are selected from H, C₁₋₆ alkyl, OC₁₋₆ alkyl and OCH₂Ph.

h

In some aspects, h is 1. More suitably, in other aspects, h is 0.

j

Each j is an integer independently selected from 0 to 6; that is each j is independently 0, 1, 2, 3, 4, 5 or 6.

In some aspects, j is 1, 2, 3, 4, 5 or 6.

Suitably, each j is an integer independently selected from 0 to 5; suitably independently selected from 0 to 4; suitably independently selected from 0 to 3; suitably independently selected from 0 to 2; suitably independently selected from 0 to 1.

In some aspects, j is 0.

k

Each k is an integer independently selected from 1 to 6; that is each k is independently selected from 1, 2, 3, 4, 5 and 6.

Suitably, each k is an integer independently selected from 1 to 5; suitably independently selected from 1 to 4; suitably independently selected from 1 to 3; suitably independently selected from 1 to 2.

In some aspects, k is 1.

m

m is an integer selected from 0 to 12; that is m is selected from 0, 1, 2, 3, 4, 5, 6, 7, 8, 9, 10, 11 and 12.

In some aspects, m is 1, 2, 3, 4, 5, 6, 7, 8, 9, 10, 11 or 12.

Suitably, m is an integer selected from 0 to 11; suitably selected from 0 to 10; suitably selected from 0 to 9; suitably selected from 0 to 8; suitably selected from 0 to 7; suitably selected from 0 to 6; suitably selected from 0 to 5; suitably selected from 0 to 4; suitably selected from 0 to 3; suitably selected from 0 to 2; suitably selected from 0 to 1.

In some aspects, m is 0.

n

n is an integer selected from 0 to 12; that is n is selected from 0, 1, 2, 3, 4, 5, 6, 7, 8, 9, 10, 11 and 12.

In some aspects, n is 1, 2, 3, 4, 5, 6, 7, 8, 9, 10, 11 or 12.

Suitably, n is an integer selected from 0 to 11; suitably selected from 0 to 10; suitably selected from 0 to 9; suitably selected from 0 to 8; suitably selected from 0 to 7; suitably selected from 0 to 6; suitably selected from 0 to 5; suitably selected from 0 to 4; suitably selected from 0 to 3; suitably selected from 0 to 2; suitably selected from 0 to 1.

In some aspects, n is 1.

p

In some aspects, p is 1. More suitably, in other aspects, p is 0.

s

Each s is an integer independently selected from 0 to 6; that is each s is independently selected from 0, 1, 2, 3, 4, 5 and 6.

In some aspects, s is 1, 2, 3, 4, 5 or 6.

Suitably, each s is an integer independently selected from 0 to 5; suitably independently selected from 0 to 4; suitably independently selected from 0 to 3; suitably independently selected from 0 to 2.

In some aspects, s is 0 or 1. More suitably, s is 0.

t

Each t is an integer independently selected from 1 to 6; that is each t is independently selected from 1, 2, 3, 4, 5 and 6.

Suitably, each t is an integer independently selected from 1 to 5; suitably independently selected from 1 to 4; suitably independently selected from 1 to 3; suitably independently selected from 1 to 2.

In some aspects, t is 1.

z

Each z is an integer selected from 1 to 5; that is each z is selected from 1, 2, 3, 4 and 5.

Suitably, each z is an integer selected from 1 to 4; suitably, selected from 1 to 3; suitably, selected from 1 to 2.

In some aspects, z is 1.

### Prodrug Moiety R₁₈

A prodrug moiety is a masked form of an active drug that needs to be transformed before exhibiting its pharmacological action. Typically, such moieties are designed to be activated after an enzymatic or chemical reaction once they have been administered into the body. Activation of prodrugs typically involves the elimination of the prodrug moiety to release the drug. Prodrugs are considered to be inactive or at least significantly less active than the released drugs.

Several prodrug moieties are known for group A, such as CPI or CBI groups, in compounds of formula (I). In particular, prodrug moieties containing carbonyl, carbamoyl, glycosyl, O-amino, O-acylamino, para-aminobenzyl ether, peptidyl or phosphate groups have been reported in Wolff, I., et al, Clin. Cancer Res. 1996, 2, 1717-1723; Wang, Y., et al, Bioorg. Med. Chem. 2006, 14, 7854-7861; Tietze, L. F., et al, J. Med. Chem. 2009, 52, 537-543; Jin, W., et al, J. Am. Chem. Soc. 2007, 129, 15391-15397; Jeffrey, et al., J. Med. Chem. 2005, 48, 1344-1358; Boger, D. L., et al, Synthesis 1999, 1505-1509; Tercel, M., et al., J. Org. Chem. 1999, 64, 5946-5953; Nagamura, S., et al, Bioorg. Med. Chem. 1997, 5, 623-630; and Zhao, R. Y. et al, J. Med. Chem. 2011, 55, 766-782.

Suitably, where the prodrug moiety R₁₈ comprises a glycosyl group that glycosyl group is a glucoside or a glucuronide. That is the glycosyl group is derived from a glucose or a glucuronic acid group.

Suitably, in some aspects the prodrug moiety R₁₈ also comprises a linker.

Suitably, the prodrug moiety R₁₈ is-O—NHR₁₉, —O—NR₁₉Boc, —O—P(O)(OH)₂, —O—NHSO₂R₁₉, —O—C(═O)—NR′R″, —O—NHC(O)C(CH₃)₃, —O—NHCO₂R₁₉, —NHCONH₂,

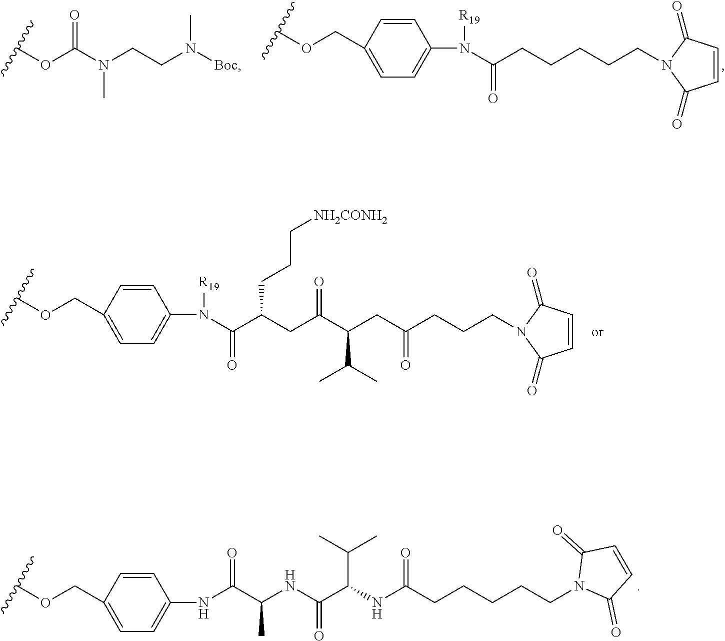

wherein R′ and R″ together with the nitrogen to which they are attached form a 5- or 6-membered heterocyclic ring optionally substituted with 1, 2 or 3 C₁₋₆ alkyl groups; and wherein each AA is an independently selected amino acid.

Hence, the —(CH₂)₁₋₁₀— linker consists of 1, 2, 3, 4, 5, 6, 7, 8, 9 or 10 CH₂ units. Suitably, such linkers consist of 3, 4, 5, 6 or 7 CH₂ units.

Hence, the -[AA]₂₋₁₂-is a peptide group consisting of 2, 3, 4, 5, 6, 7, 8, 9, 10, 11 or 12 amino acid units. Suitably, this peptide group consist of 2, 3, 4, 5, 6, 7 or 8 amino acid units.

More suitably, the prodrug moiety R₁₈ is-O—NH₂, —O—NHCH₃, —O—P(O)(OH)₂, —O—NHBoc, —O—NCH₃Boc, —O—NHSO₂CH₃, —O—NHC(O)C(CH₃)₃, —O—NHCO₂CH₃, —NHCONH₂,

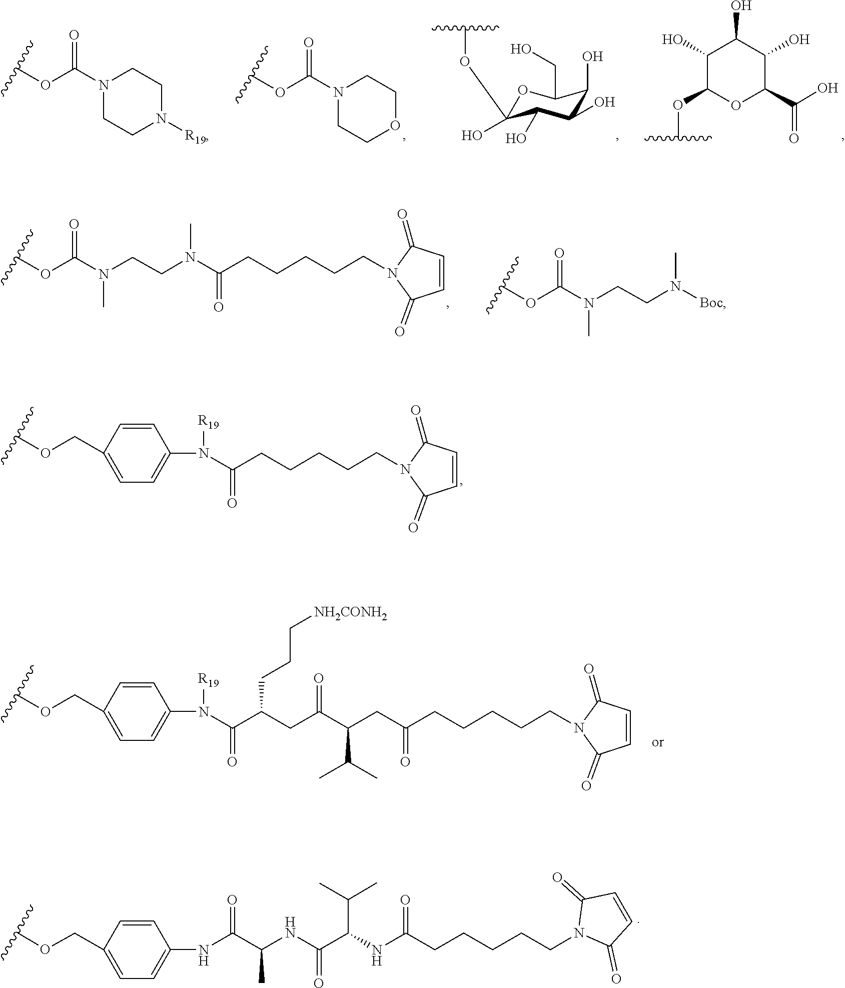

More suitably, the prodrug moiety is:

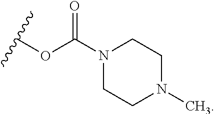 [SMILES: *OC(=O)N1CCN(C)CC1]

### R′ and R″

Suitably, R′ and R″ together with the nitrogen to which they are attached form a 6-membered heterocyclic ring optionally substituted with 1, 2 or 3 C₁₋₆ alkyl groups. Suitably, R′ and R″ together with the nitrogen to which they are attached form an optionally substituted morpholinyl or piperazinyl ring.

More suitably, R′ and R″ together with the nitrogen to which they are attached form:

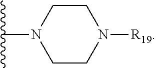

More suitably, R′ and R″ together with the nitrogen to which they are attached form:

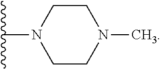 [SMILES: *N1CCN(C)CC1]

### K₁

Linker K₁ is a bond or is a moiety having 1-200 nonhydrogen atoms selected from C, N, O, S, or halogen, and optionally incorporates alkyl, ether, oxo, carboxyl, carboxamide, carboxamidyl, ester, urethanyl, branched, cyclic, unsaturated, amino acid, heterocyclyl, aryl or heteroaryl moieties. Linker K₁ may be unbranched or branched, flexible or rigid, short or long and may incorporate any combination of moieties as deemed useful. In some embodiments, at least a portion of the linker K₁ may have a polyalkylene oxide polymeric region, which may enhance solubility of the compound of formula (I) or (II). In some embodiments, the linker K₁ may have a repeating unit of ethylene glycol, and may have a number of repeating ethylene glycol units of about 1 to about 25, or any number therebetween. In some embodiments, K₁ may include about 3 to about 20, about 4 to about 15, about 5 to about 12 or about 6 to about 10 ethylene glycol units. In some embodiments, at least a portion of Linker K₁ may include one or more amino acid moieties which may provide enhanced solubility for the compound of formula (I) or (II) or may provide amino acid sequences to enhance target binding, enhance compatibility with a targeting agent, or enhance target binding recognition. In other embodiments, the linker K₁ may include one or more amino acid moieties that provide a suitable substrate motif for a protease. When a set of amino acid moieties are incorporated into the linker K₁ that provide a substrate motif specific for a selected protease, the cytotoxic drug compound of formula (I) or (II) may be released from a target bound conjugate to provide localized cytotoxic effects. In other embodiments, the linker K₁ may include an alkylene chain. Suitably, the alkylene chain is 1, 2, 3, 4, 5, 6, 7, 8, 9, 10, 11 or 12 carbons in length; and suitably the alkylene chain comprises —CH₂— groups. Such substrate motifs are known in the art and may be incorporated into the linker 1₂ as desired to provide selective release from the target bound conjugate. This selectivity can be based on known presence of a desired protease within the localized delivery region of the conjugate drug. Other polymeric types of moieties may be incorporated in the linker K₁, such as polyacids, polysaccharides, or polyamines. Other moieties such as substituted aromatic or heteroaromatic moieties may be used to enhance rigidity or provide synthetically accessible sites on substituents therein for linking to reactive moieties or to the compound of formula (I) or (II).

For example, the linker K₁ can include ethylene glycol repeating units, and/or an amino acid sequence. In some embodiments, linker K₁ comprises the formula:

—[CH₂CH₂O]₀₋₅₀—X_{AA}—

wherein X_{AA} is an amino acid sequence.

Any suitable number of ethylene glycol units can be used in the linker L of the present invention. For example, the linker K₁ can include 1, 2, 3, 4, 5, 6, 7, 8, 9, 10, 11, 12, 15, 16, 19, 20, 23, 24, 35, 36, 37, 48, 49, or more ethylene glycol units. In some embodiments, the linker K₁ can include 8 ethylene glycol units. Several commercially available ethylene glycol groups (polyethylene glycol, PEG) are suitable in the linker K₁, such as H₂N-dPEG®₈-C(O)OH, having a discrete (“d”) polyethylene glycol having 8 ethylene glycol repeating units. Other discrete PEG units are commercially available and known to one of skill in the art, such as by Advanced ChemTech, In some embodiments, the linker K₁ comprises the formula:

—HN-PEG-C(O)—X_{AA}—

wherein PEG has 1-50 ethylene glycol units, and X_{AA} is an amino acid sequence.

In another example, the linker K₁ can include an alkylene chain, and/or an amino acid sequence. In some embodiments, linker K₁ comprises the formula:

—[CH₂]₀₋₁₂—X_{AA}—

wherein X_{AA} is an amino acid sequence; and the linker K₁ can include 0, 1, 2, 3, 4, 5, 6, 7, 8, 9, 10, 11 or 12 —CH₂— units.

In some embodiments, the linker K₁ comprises the formula:

—HN-PEG₈-C(O)-Val-Ala-

wherein PEG₈ has 8 ethylene glycol units.

The linker K₁ can also include a variety of other connecting groups that connect the ethylene glycol portion to the amino acid sequence, or connect the ethylene glycol or amino acid sequence to R*, or the compound of formula (I) or (II). For example, the amino acid sequence can be connected to the compound of formula (I) or (II) via a 4-amino benzyl carboxylate group. Suitably, in some embodiments, the ethylene glycol portion ca be directly linked to R*. In some embodiments, the linker K₁ comprises the formula:

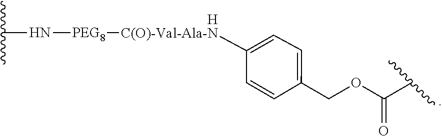

Suitably, for the above embodiment, the HN group is directly linked to R*.

More suitably, the linker K₁ is:

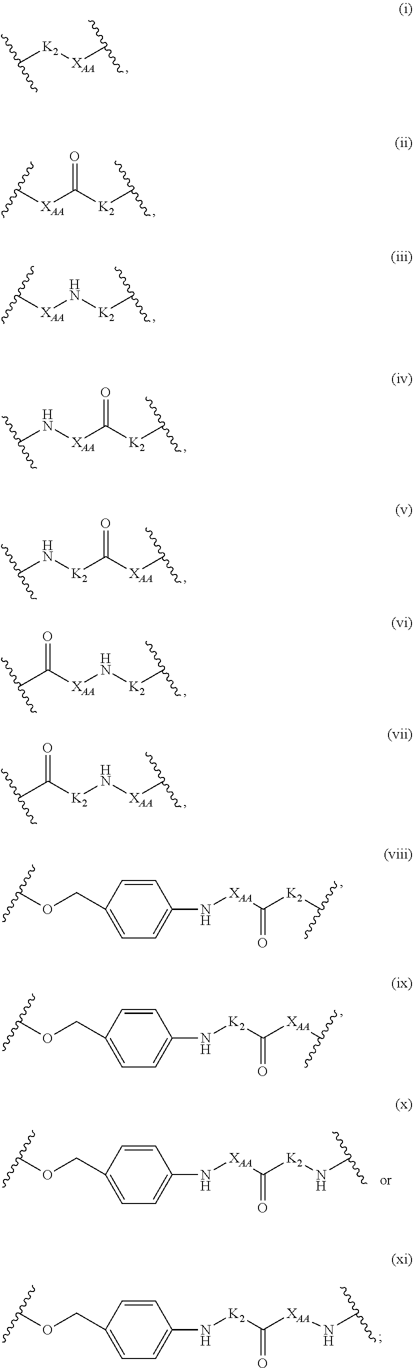

wherein X_{AA} is an amino acid sequence; and K₂ is —[CH₂CH₂O]₀₋₅₀— or —[CH₂]₀₋₁₂—. The linker K₁ may be attached to R* and the rest of the compound of formula (I) in either direction. More suitably, the linker K₁ is (i), (ii), (iii), (iv), (vi), (viii) or (ix).

More suitably, the linker K₁ is (iv):

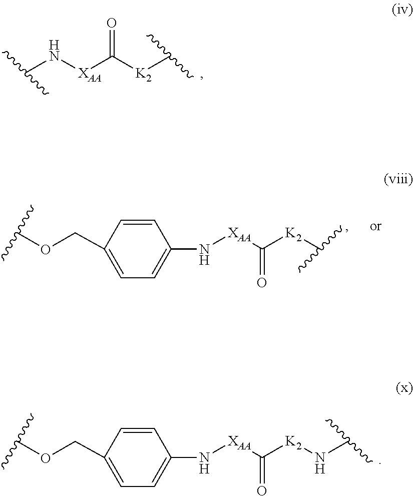

More suitably, L² can include 1, 2, 3, 4, 5, 6, 7, 8, 9, 10, 11, 12, 15, 16, 19, 20, 23, 24, 35, 36, 37, 48, 49 or 50 ethylene glycol units.

More suitably, L² can include 0, 1, 2, 3, 4, 5, 6, 7, 8, 9, 10, 11 or 12 —CH₂— units.

Suitably, for the above embodiment, the HN group is directly linked to R*.

The amino acid portion of the linker K₁ can include any suitable number of amino acid moieties, as described above. For example, the amino acid sequence X_{AA} can include from 1 to 100 amino acid moieties, or from 1 to 10 amino acid moieties, or from 1 to 5 amino acid moieties. Suitably, the linker K₁ can include 1, 2, 3, 4, 5, 6, 7, 8, 9, 10, or more amino acid moieties. Suitably, the linker K₁ includes 2 amino acid moieties.

More suitably, the linker K₁ includes the amino acid sequence Val-Ala.

More suitably, the amino acid sequence X_{AA} is:

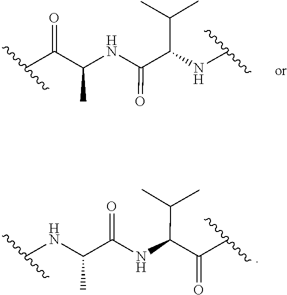 [SMILES: *N[C@H](C(=O)N[C@@H](C)C(*)=O)C(C)C ; *N[C@@H](C)C(=O)N[C@H](C(*)=O)C(C)C]

More suitably, the amino acid sequence X_{AA} is:

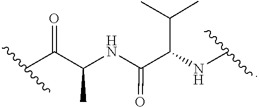 [SMILES: *N[C@H](C(=O)N[C@@H](C)C(*)=O)C(C)C]

### R*

R* is an azide, alkyne, bisulfone, carbohydrazide, hydrazine, hydroxylamine, iodoacetamide, isothiocyanate, maleimide, phosphine, pyrridopyridazine, semihydrazide, succinimidyl ester, sulfodichlorophenol ester, sulfonyl halide, sulfosuccinimidyl ester, 4-sulfotetrafluorophenyl ester, tetrafluorophenyl ester, thiazole, R₂₀, O—(CH₂)ₖ—NR₂₆R₂₆, NHNH₂, or is a targeting agent wherein the targeting agent is selected from a protein, a portion of a protein, a polypeptide, a nucleic acid, a hormone, an antibody or an antibody fragment.

Hence, R* is a reactive moiety capable of reacting with a targeting agent, or is a targeting agent. Where R* is a reactive moiety it can react with functional groups such as aldehdes, amines, disulfides, ketones thiols in the targeting agent, or in Staudinger reactions, Pictet-Spengler reactions and/or Click-type chemistry with the targeting agent. For some reactive moieties suitable coupling reagents are used to react the reactive moiety with a targeting agent, e.g. where R* is a carboxylic acid [when R_{A} is (CH₂)ⱼ—CO₂R₂₆] carbodiimide coupling reagents may be used.

Suitably, R* is an azide, alkynes, bisulfone, carbohydrazide, hydroxylamine, iodoacetamide, isothiocyanate, maleimide, phosphine, semihydrazide, succinimidyl ester and sulfonyl halide, R₂₀ or is a targeting agent.

More suitably, in some aspects, R* is maleimide:

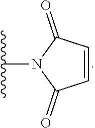 [SMILES: *N1C(=O)C=CC1=O]

In one aspect, suitably, R* is an azide, alkynes, bisulfone, carbohydrazide, hydroxylamine, iodoacetamide, isothiocyanate, maleimide, phosphine, semihydrazide, succinimidyl ester and sulfonyl halide or R₂₀.

A number of other chemistries are known for attachment of compounds to antibodies. U.S. Pat. No. 7,595,292 (Brocchini et al.) refers to linkers that form thioesters with the sulfurs in a disulfide bond of an antibody. U.S. Pat. No. 7,985,783 (Carico et al.) refers to the introduction of aldehyde residues into antibodies, which are used to couple compounds to the antibody.

In another aspect, R* is a targeting agent wherein the targeting agent is selected from a protein, a portion of a protein, a peptide, a nucleic acid, a hormone, an antibody or an antibody fragment. The targeting agent may bind to a tumor-associated antigen, a cancer-stem-cell associated antigen or a viral antigen.

Suitably, the targeting agent is selected from a protein, a portion of a protein, a polypeptide, a nucleic acid, an antibody or an antibody fragment. More suitably, the targeting agent is an antibody or an antibody fragment. More suitably, the targeting agent is an antibody.

In various embodiments, the targeting agent may bind to a target selected from an acute myeloid leukemia (AML M4) cell, an acute promyelocytic leukemia cell, an acute lymphoblastic leukemia cell, an acute lymphocytic leukemia cell, a chronic lymphocytic leukemia cell, a chronic myeloid leukemia cell, a chronic T-cell lymphocytic leukemia, a myelodysplasia syndromic cell, a multiple myeloma cell, a prostate carcinoma cell, a renal cell adenocarcinoma cell, a pancreatic adenocarcinoma cell, a lung carcinoma cell or a gastric adenocarcinoma cell, a gastric adenocarcinoma cell, a breast cancer cell, a colon cancer cell, a melanoma cell, a thyroid cancer cell, an ovarian cancer cell, a bladder cancer cell, a liver cancer cell, a head and neck cancer cell, an esophageal cancer cell, a hodgkin lymphoma cell, a non-hodgkin lymphoma cell, a mesothelioma cell, a neuroblastoma cell, a neuroendocrine tumor cell, a neurofibromatosis type 1 (NF1) cell, a neurofibromatosis type 2 (NF2) or an osteosarcoma cell.

### Other Features

Suitably, the compound of formula (I) is selected with the proviso that when —K₁—R* is present in the compound of formula (I), there is only one —K₁—R* group present.

In some embodiments, —K₁—R* is absent from the compound of formula (I).

Suitably, the compound of formula (I) contains only one primary or secondary amine.

Suitably, the compound of formula (I) contains only one primary amine, secondary amine or —K₁—R* group.

Suitably, the compound of formula (I) contains only one primary amine, secondary amine, R₂₀, or —K₁—R* group.

### Other Aspects

In some aspects, the compound of formula (I) is selected with the proviso that when R₂ is C₁₋₆ alkyl that R₉ and R₁₀ are selected from options (i), (ii), (iii) or (iv). When R₂ is C₁₋₆ alkyl then the moiety A of the compound of formula (I) will not alkylate DNA. In such aspects, the options for R₉ and R₁₀ are limited to those that ensure that the moiety B of the compound of formula (I) does alkylate with DNA. An example of a compound that falls within this proviso is:

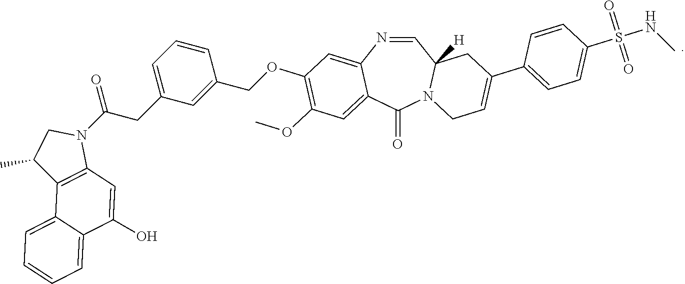

In some aspects, the compound of formula (I) is selected with the proviso that when (v) R₉ is H or C₁₋₆ alkyl, and R₁₀ is oxo or H; then either R₂ is selected from —CH₂-halogen and H, and R₃ is H; or R₂ and R₃ together with the carbon atoms to which they are attached form a cyclopropyl ring. When option (v) applies then the moiety B of the compound of formula (I) will not alkylate DNA. In such aspects, the options for R₂ are limited to those that ensure that the moiety A of the compound of formula (I) does alkylate with DNA. An example of a compound that falls within this proviso is shown below:

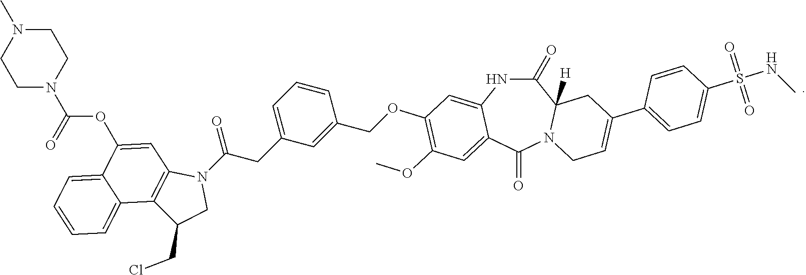 [SMILES: CNS(=O)(=O)c1ccc(C2=CCN3C(=O)c4cc(OC)c(OCc5cccc(CC(=O)N6C[C@@H](CCl)c7c6cc(OC(=O)N6CCN(C)CC6)c6ccccc76)c5)cc4NC(=O)[C@@H]3C2)cc1]

In this compound R₉ is H, R₁₀ is oxo and R₂ is —CH₂—Cl.

### Specific Structures

Suitably, the compound of formula (I) is:

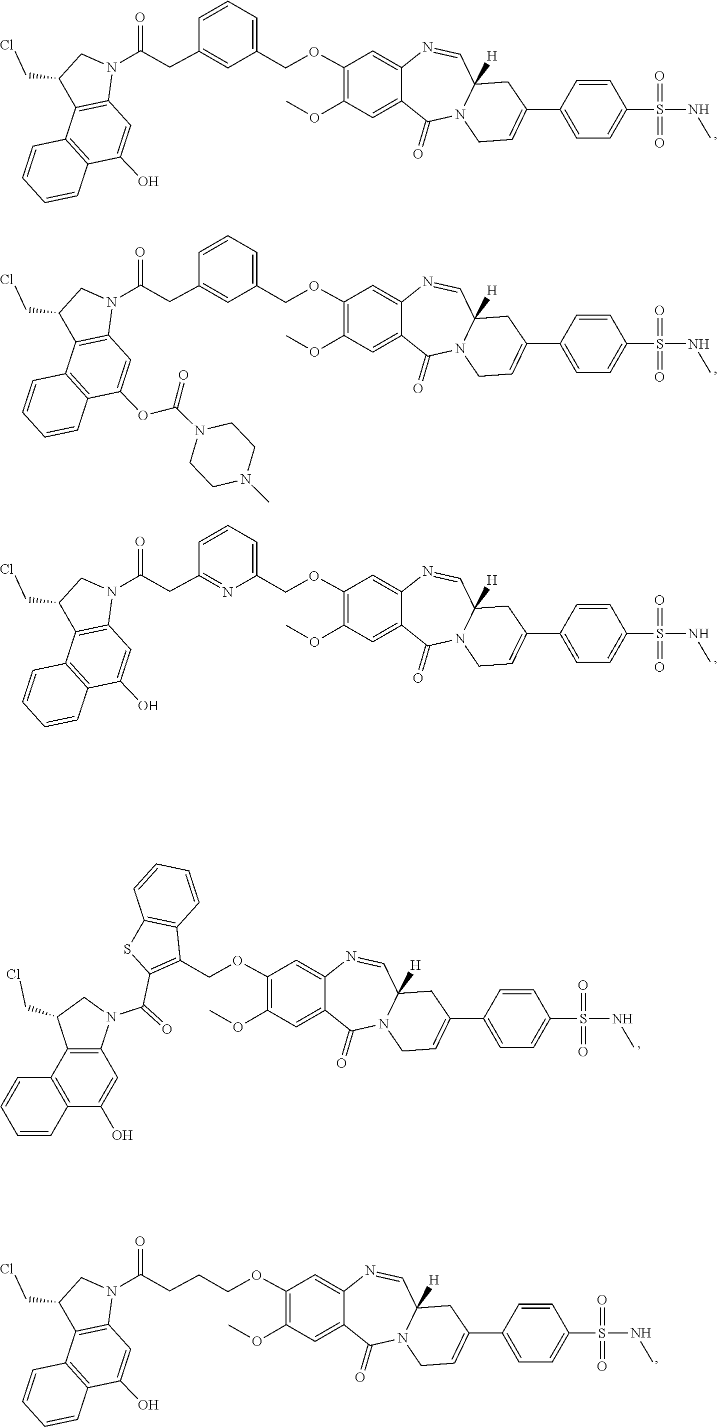

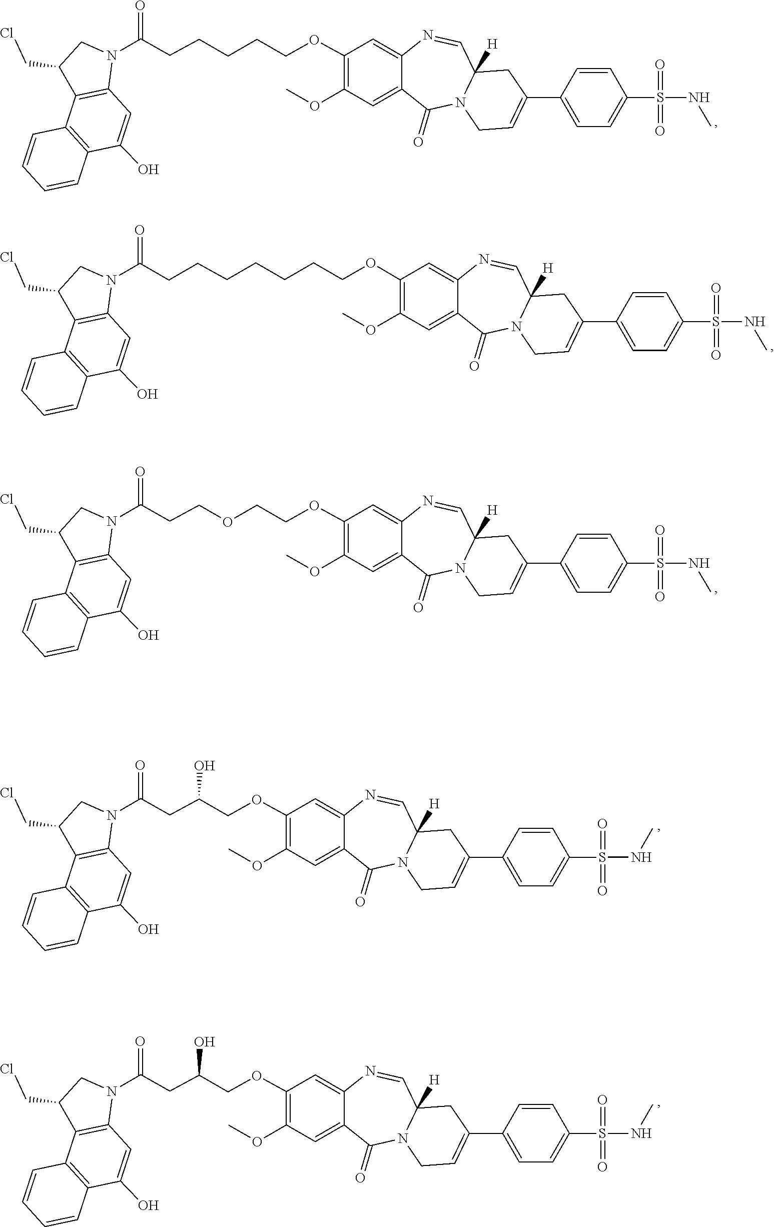

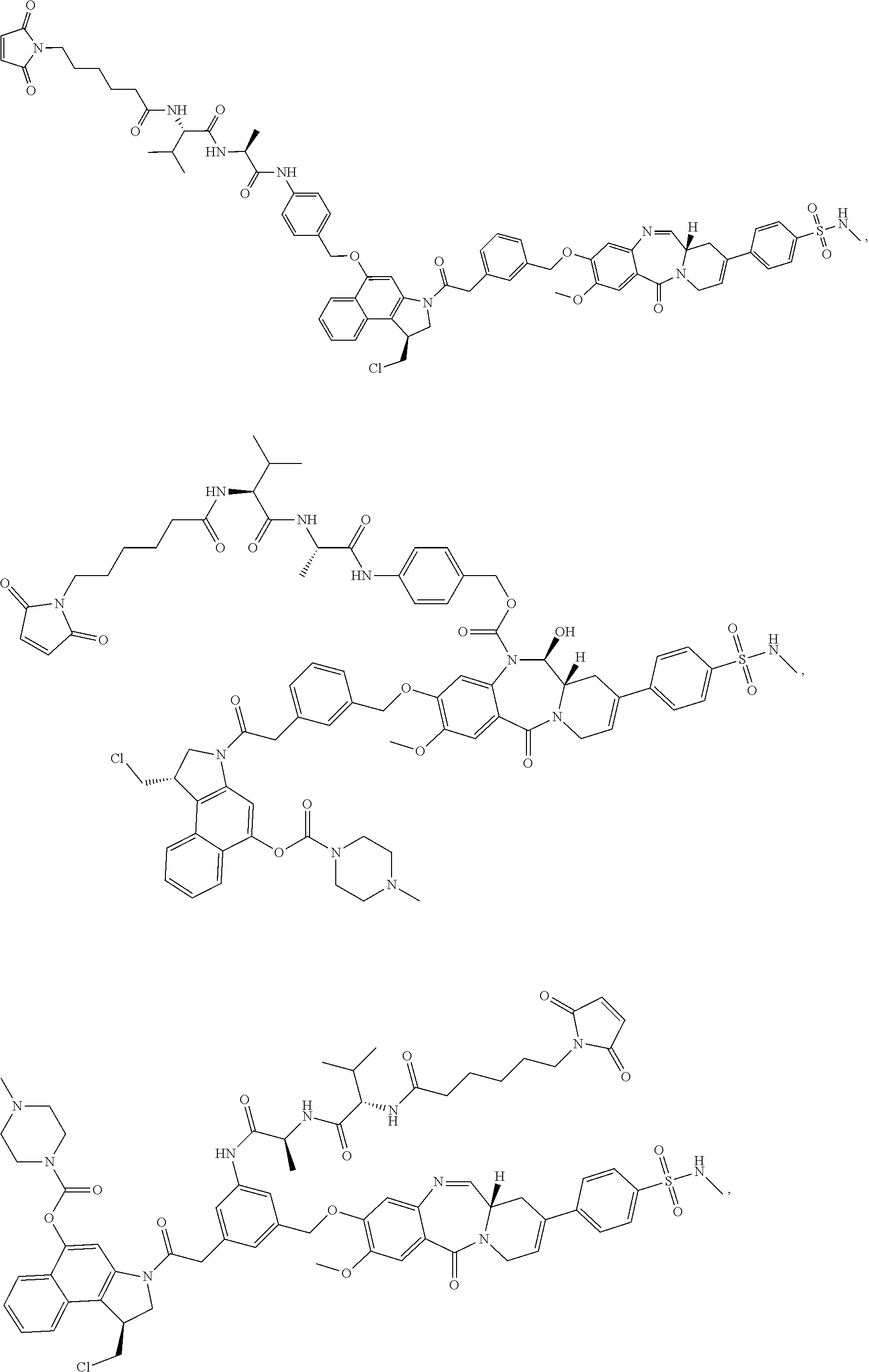

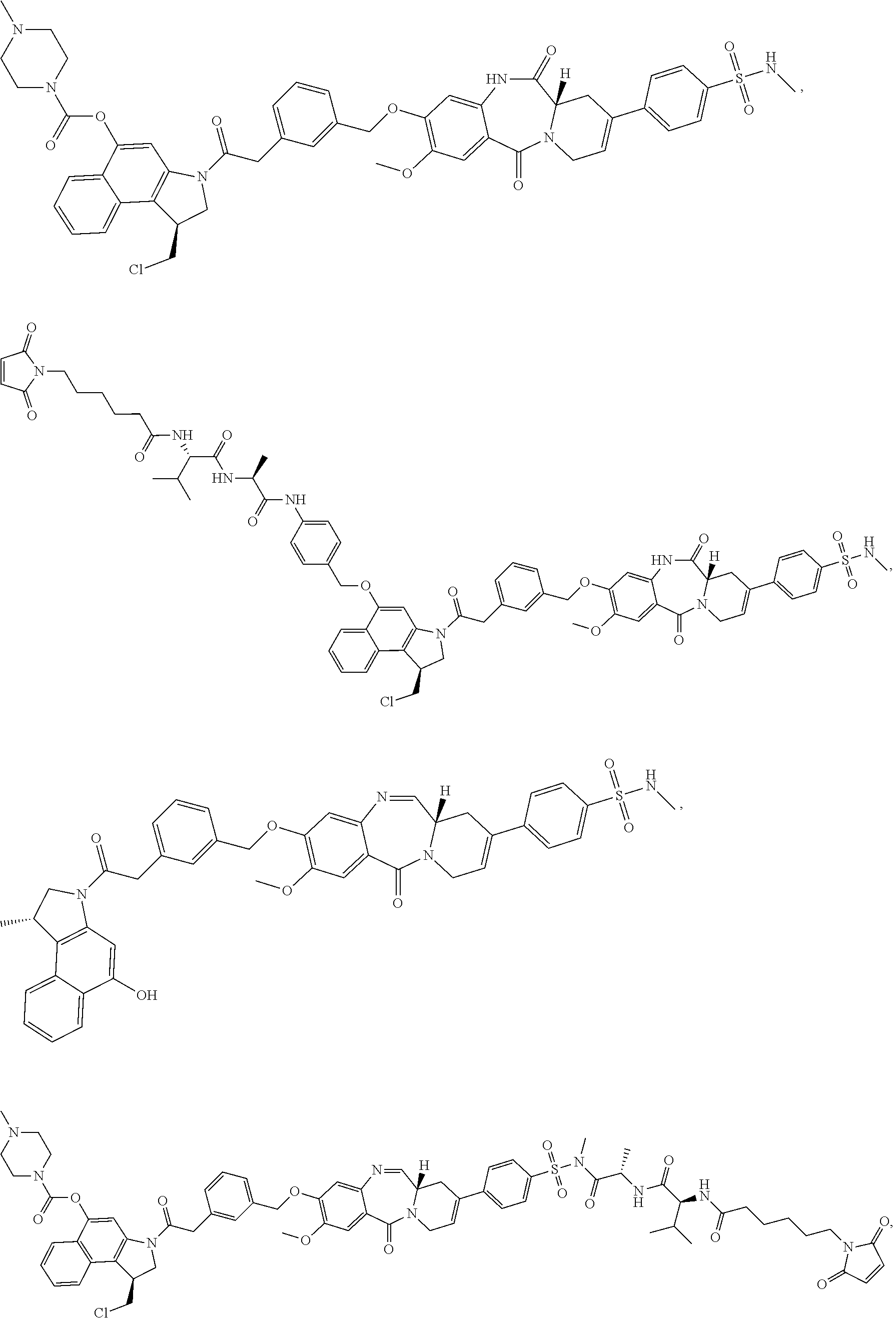

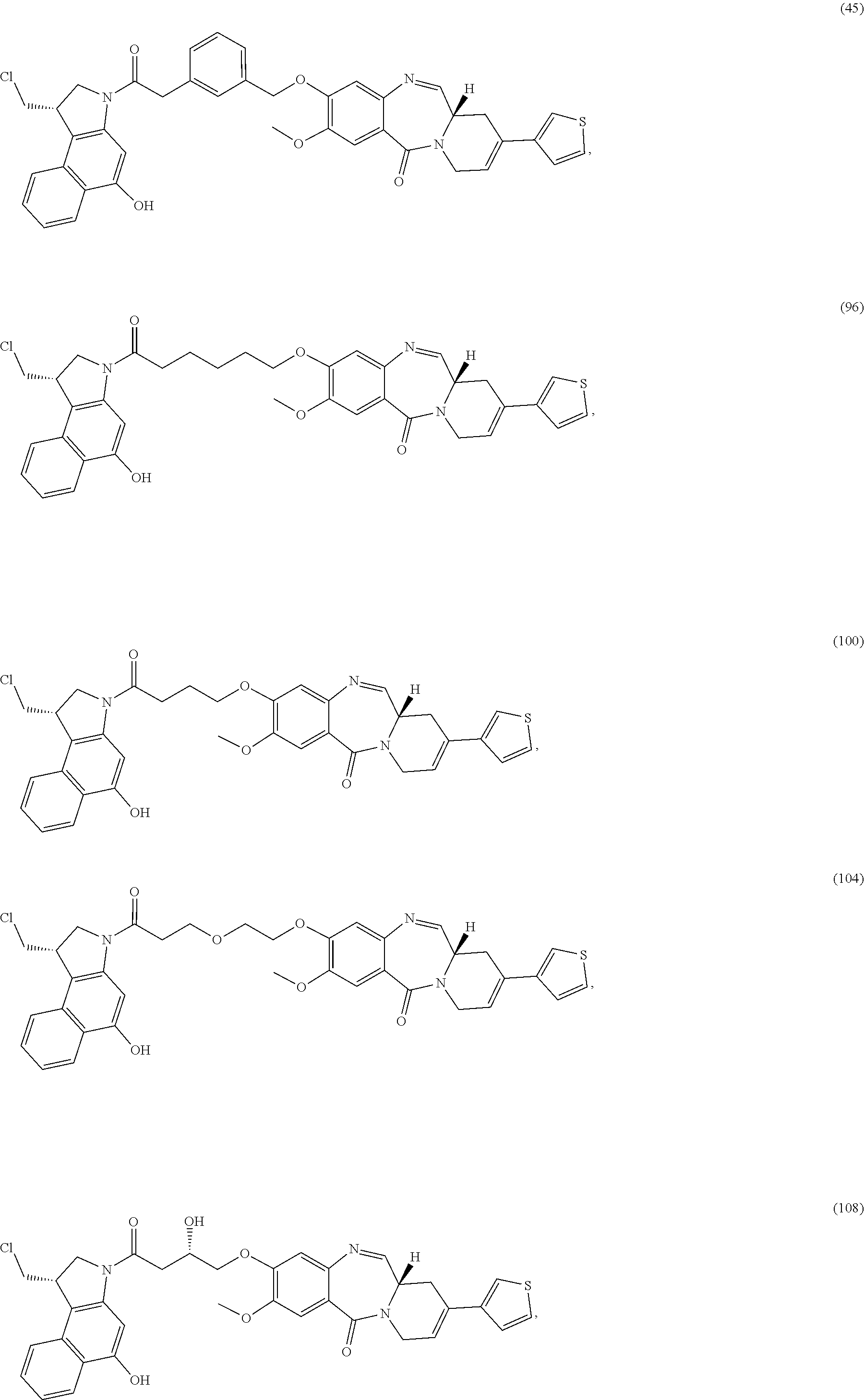 [SMILES: COc1cc2c(cc1OCc1cccc(CC(=O)N3C[C@@H](CCl)c4c3cc(O)c3ccccc43)c1)N=C[C@@H]1CC(c3ccsc3)=CCN1C2=O ; COc1cc2c(cc1OCCCCCC(=O)N1C[C@@H](CCl)c3c1cc(O)c1ccccc31)N=C[C@@H]1CC(c3ccsc3)=CCN1C2=O ; COc1cc2c(cc1OCCCC(=O)N1C[C@@H](CCl)c3c1cc(O)c1ccccc31)N=C[C@@H]1CC(c3ccsc3)=CCN1C2=O ; COc1cc2c(cc1OCCOCCC(=O)N1C[C@@H](CCl)c3c1cc(O)c1ccccc31)N=C[C@@H]1CC(c3ccsc3)=CCN1C2=O ; COc1cc2c(cc1OC[C@@H](O)CC(=O)N1C[C@@H](CCl)c3c1cc(O)c1ccccc31)N=C[C@@H]1CC(c3ccsc3)=CCN1C2=O]

or salts, solvates, isomers or tautomers thereof.

### Applications

The compound of formula (I) or salts, solvates, isomers or tautomers thereof, or a pharmaceutical compositions comprising such compounds of formula (I) find application as a medicament.

The invention finds application in the treatment of a proliferative disease, a bacterial infection, a malarial infection and inflammation.

In certain aspects a method of treating a disease or condition selected from a proliferative disease, a bacterial infection, a malarial infection and inflammation is provided, the method comprising administering to a subject a therapeutically effective amount of a compound of the formula (I) or salts, solvates, isomers or tautomers thereof or a composition comprising a compound of formula (I) or pharmaceutically acceptable salts, solvates, tautomers, stereoisomers or mixtures thereof.

In certain aspects a method of treating a disease or condition selected from proliferative diseases, bacterial infections, malaria and inflammation is provided, the method comprising administering to a subject a therapeutically effective amount of a targeted conjugate comprising a compound of the formula (I) or pharmaceutically acceptable salts, solvates, tautomers, stereoisomers or mixtures thereof.

In certain aspects a method of treating a proliferative disease is provided, the method comprising administering to a subject a therapeutically effective amount of an antibody-drug conjugate comprising a compound of the formula (I) or pharmaceutically acceptable salts, solvates, tautomers, stereoisomers or mixtures thereof.

The term “proliferative disease” refers to an unwanted or uncontrolled cellular proliferation of excessive or abnormal cells which is undesired, such as, neoplastic or hyperplastic growth, whether in vitro or in vivo. Examples of proliferative conditions include, but are not limited to, benign, pre-malignant, and malignant cellular proliferation, including but not limited to, neoplasms and tumours (e.g. histocytoma, glioma, astrocyoma, osteoma), cancers (e.g. lung cancer, small cell lung cancer, hepatocellular cancer, gastric or stomach cancer including gastrointestinal cancer, bowel cancer, colon cancer, hepatoma, breast cancer, glioblastoma, cervical cancer, ovarian cancer, oesophageal [or esophageal] cancer, oral cancer, prostate cancer, testicular cancer, liver cancer, rectal cancer, colorectal cancer, endometrial or uterine carcinoma, uterine cancer, salivary gland carcinoma, kidney or renal cancer, prostate cancer, vulval cancer, thyroid cancer, hepatic carcinoma, anal carcinoma, penile carcinoma, head and neck cancer, bladder cancer, pancreas cancer, brain cancer, sarcoma, osteosarcoma, Kaposi's sarcoma, melanoma), leukemias, psoriasis, bone diseases, fibroproliferative disorders (e.g. of connective tissues), and atherosclerosis. Suitably the proliferative disease is selected from bladder cancer, bone cancer, bowel cancer, brain cancer, breast cancer, cervical cancer, colon cancer, head and neck cancer, leukemia, liver cancer, lung cancer, lymphoma, melanoma, oesophageal cancer, oral cancer, ovarian cancer, pancreatic cancer, prostate cancer, rectal cancer, renal cancer, retinoblastoma, sarcoma, skin cancer, stomach cancer, testicular cancer, thyroid cancer and uterine cancer. Suitably the proliferative disease is selected from breast cancer and cervical cancer.

Suitably, the proliferative disease is selected from bladder cancer, bone cancer, bowel cancer, brain cancer, breast cancer, cervical cancer, colon cancer, head and neck cancer, leukemia, liver cancer, lung cancer, lymphoma, melanoma, oesophageal cancer, oral cancer, ovarian cancer, pancreatic cancer, prostate cancer, rectal cancer, renal cancer, retinoblastoma, sarcoma, skin cancer, stomach cancer, testicular cancer, thyroid cancer and uterine cancer.

Any type of cell may be treated, including but not limited to, bone, eye, head and neck, lung, gastrointestinal (including, e.g. mouth, oesophagus, bowel, colon), breast (mammary), cervix, ovarian, uterus, prostate, liver (hepatic), kidney (renal), bladder, pancreas, brain, and skin.

A skilled person is readily able to determine whether or not a candidate compound treats a proliferative condition for any particular cell type.

Suitably subjects are human, livestock animals and companion animals.

In a further aspect, the compound of formula (I) or pharmaceutically acceptable salts, solvates, tautomers, stereoisomers or mixtures thereof, may be linked, either directly or indirectly, to a targeting agent (e.g., a protein, a portion of a protein, a polypeptide, a nucleic acid, a hormone, an antibody or an antibody fragment, etc.) to provide a targeted conjugate. The target conjugates of the present disclosure may contain one or multiple compounds of formula (I) (or pharmaceutically acceptable salts, solvates, tautomers, stereoisomers or mixtures thereof). A variety of target conjugates are known in the art and may be used with a compound of formula (I) or pharmaceutically acceptable salts, solvates, tautomers, stereoisomers or mixtures thereof. For example, in a particular aspect the target conjugate is an antibody-drug conjugate, wherein one or more compounds of formula (I) are linked, directly or indirectly, to the antibody. Therefore, the compound of formula (I) or pharmaceutically acceptable salts, solvates, tautomers, stereoisomers or mixtures thereof, may be used as a payload on a targeted conjugate.

Suitably, a compound of formula (I) or pharmaceutically acceptable salts, solvates, tautomers, stereoisomers or mixtures thereof, for use as a drug in a targeted conjugate is prepared by attaching a compound of formula (I) or salts, solvates, isomers or tautomers thereof to a targeting agent, either directly or via an optional linker group. Suitably, the compound of formula (I) or pharmaceutically acceptable salts, solvates, tautomers, stereoisomers or mixtures thereof, is attached to a targeting agent via a linker group. Suitably, the targeted conjugate is for use in the treatment of a disease, more specifically of a proliferative disease. Suitably, the drug may be attached by any suitable functional group that it contains to the targeting agent either directly or via a linker group. Typically, the drug contains, or can be modified to contain, one or more functional groups such as amine, hydroxyl or carboxylic acid groups for attaching the drug to the targeting agent either directly or via a linker group. In some aspects, one or more atoms or groups of the compound of formula (I) may be eliminated during the attachment of the drug to the antibody. In some aspects, the targeting agent binds to a cell surface receptor or a tumor-associated antigen. In some aspects, the targeting agent is an antibody. In some aspects, the targeting agent is an antibody fragment. In some aspects, the targeting agent is a hormone. In some aspects, the targeting agent is a protein. In some aspects, the targeting agent is a polypeptide. In some aspects, the targeting agent is a small molecule (for example, folic acid). Suitably, the targeting agent is selected from a protein, a portion of a protein, a polypeptide, a nucleic acid, an antibody or an antibody fragment. More suitably, the targeting agent is an antibody or an antibody fragment. More suitably, the targeting agent is an antibody.

Suitably, the present invention relates to a compound of formula (I) or pharmaceutically acceptable salts, solvates, tautomers, stereoisomers or mixtures thereof, for use in preparing a targeting conjugate (e.g. an antibody-drug conjugate). Suitably, a compound of formula (I) or pharmaceutically acceptable salts, solvates, tautomers, stereoisomers or mixtures thereof, may be used directly to prepare a targeting conjugate when a compound of formula (I) or pharmaceutically acceptable salts, solvates, tautomers, stereoisomers or mixtures thereof, contains one or more functional groups such as amine, hydroxyl or carboxylic acid groups for attaching the drug to the targeting agent either directly or via a linker group. Suitably, a compound of formula (I) or pharmaceutically acceptable salts, solvates, tautomers, stereoisomers or mixtures thereof, may be used in preparing a targeting conjugate by being modified to contain one or more functional groups such as amine, hydroxyl or carboxylic acid groups for attaching the drug to the targeting agent either directly or via a linker group. Suitably, a compound of formula (I) or pharmaceutically acceptable salts, solvates, tautomers, stereoisomers or mixtures thereof, may be used in preparing a targeting conjugate by being modified to contain one or more linker groups, wherein the targeting agent (such as an antibody) is attached to the drug through one or more linker groups. Therefore, the present invention provides for a compound of formula (I) further comprising one or more linker groups or pharmaceutically acceptable salts, solvates, tautomers, stereoisomers or mixtures thereof. Suitably, a compound of formula (I) further comprises 1, 2 or 3 linker groups. Suitably, a compound of formula (I) further comprises 1 or 2 linker groups. Suitably, a compound of formula (I) further comprise 1 linker group. In some aspects, one or more atoms or groups (such as H atoms or hydroxyl groups) of the compound of formula (I) may be eliminated during the attachment of the drug to the targeting agent (such as an antibody) or the attachment of the linker to the drug or the modification of the drug to contain one or more functional groups (such as amine, hydroxyl or carboxylic acid groups) for attaching the drug to the antibody either directly or via a linker group. In some aspects, where the compound of formula (I) further comprises a linker group that is attached to the rest of the compound of formula (I) by eliminating one or more atoms or groups (such as H atom or atoms or hydroxyl groups) from an R_{A} group or by eliminating the R₇ group from a N—R₇ group.

Suitably such linker groups may comprise from 1-200 non-hydrogen atoms selected from C, N, O, S or halogen and may be branched, cyclic and/or unsaturated and, optionally, such linker groups may incorporate ether, oxo, carboxamidyl, urethanyl, heterocyclyl, aryl, heteroaryl, azide, alkyne, bisulfone, carbohydrazide, hydrazine, hydroxylamine, iodoacetamide, isothiocyanate, maleimide, phosphine, pyrridopyridazine, R_{A}, semihydrazide, succinimidyl ester, sulfodichlorophenol ester, sulfonyl halide, sulfosuccinimidyl ester, 4-sulfotetrafluorophenyl ester, tetrafluorophenyl ester and thiazole moieties.

The compounds of formula (I) find application as payloads for antibodies or antibody fragments. The compounds of formula (I) readily allow conjugation to antibodies or antibody fragments.

In some aspects, the present invention relates to the treatment of a bacterial infection in a subject.

In some aspects, the compounds of formula (I) or pharmaceutically acceptable salts, solvates, tautomers, stereoisomers or mixtures thereof, are broad spectrum agents capable of treating a bacterial infection caused by Gram-positive bacteria and/or Gram-negative bacteria and/or atypical bacteria.

Suitably the bacterial infection is caused by at least one bacterium selected from the genera Enterococcus, Staphylococcus, Streptococcus, Bacillus, Acinetobacter, Burkholderia, Coxiella, Francisella, Yersina, Klebsiella, Escherichia, Enterobacter and Pseudomonas.

Suitably the bacterial infection is caused by at least one bacterium selected from the genera Enterococcus, Staphylococcus, Acinetobacter, Burkholderia, Klebsiella, Escherichia, Enterobacter and Pseudomonas.

Suitably the bacterial infection is caused by at least one bacterium selected from Enterococcus faeculis, Enterococcus faecium, Staphylococcus aureus, Streptococcus pyogenes, Streptococcus pneumoniae, Streptococcus agalactiae, Bacillus anthracis, Bacillus cereus, Bacillus subtilis, Haemophilus influenzae, Acinetobacter baumannii, Burkholderia multivorans, Burkholderia cenocepacia, Burkholderia cepacia, Burkholderia mallei, Burkholderia pseudomallei, Coxiella burnetii, Citrobacter freundii, Escherichia coli, Enterobacter cloacae, Enterobacter aerogenes, Francisella tularensis, Yersina pestis, Klebsiella pneumoniae, Serratia marcesens, Salmonella typhi, Salmonella typhimurum, Stenotrophomonas maltophilia, Pseudomonas aeruginosa and Neisseria gonorrhoeae.

More suitably the bacterial infection is caused by at least one bacterium selected from Enterococcus faeculis, Enterococcus faecium, Staphylococcus aureus, Acinetobacter baumannii, Burkholderia multivorans, Burkholderia cenocepacia, Burkholderia cepacia, Klebsiella pneumonia and Pseudomonas aeruginosa.

In some embodiments, the bacterial infection is caused by Gram-positive bacteria selected from Enterococcus faeculis, Enterococcus faecium, Staphylococcus aureus, Streptococcus pyogenes, Streptococcus pneumoniae, Streptococcus agalactiae, Bacillus anthracis, Bacillus cereus and Bacillus subtilis.

In some embodiments, the infection is caused by Gram-negative bacteria, such as Haemophilus influenzae, Acinetobacter baumannii, Burkholderia multivorans, Burkholderia cenocepacia, Burkholderia cepacia, Burkholderia mallei, Burkholderia pseudomallei, Coxiella burnetii, Citrobacter freundii, Escherichia coli, Enterobacter cloacae, Enterobacter aerogenes, Francisella tularensis, Yersina pestis, Klebsiella pneumoniae, Pseudomonas aeruginosa and Neisseria gonorrhoeae.

In some embodiments, the bacterial infection is caused by drug-resistant bacteria. Such drug-resistant bacteria are bacteria that are resistant to one or more antibacterials other than the compounds of formula (I) described herein. The language “resistance” and “antibacterial resistance” “drug-resistant” refers to bacteria that are able to survive exposure to one or more antibacterial drugs. In some embodiments, the drug-resistant bacteria include Streptococcus pyogenes, Streptococcus agalactiae, Streptococcus pneumoniae (including penicillin-resistant Streptococcus pneumoniae), Staphylococcus aureus (including vancomycin-resistant Staphylococcus aureus (VRSA)), methicillin-resistant Staphylococcus aureus (MRSA) (including hospital-acquired MRSA, community acquired MRSA and coagulase negative staphylocci), Acinetobacter baumannii, Burkholderia multivorans, Burkholderia cenocepacia, Burkholderia cepacia, Klebsiella pneumoniae Pseudomonas aeruginosa and Neisseria gonorrhoeae (including penicillin-resistant Neisseria gonorrhoeae).

In some embodiments, the drug-resistant bacteria is a multiple drug resistant bacteria. The language “multiple drug resistant bacteria” includes bacteria that is resistant to two or more of antibiotics typically used for the treatment of such bacterial infections, for example, tetracycline, penicillin, cephalosporins (e.g., ceftriazone or cefixime), glycopeptides (e.g. vancomycin), quinolones (e.g., norfloxacin, ciprofloxacin or ofloxacin), co-trimoxazole, sulfonamides, aminoglycosides (e.g., kanamycin or gentamicin) and macrolides (e.g., azithromycin).

One of ordinary skill in the art is readily able to determine whether or not a candidate compound treats a bacterial infection by, for example, assays (such as those described in the examples) which may be used to determine the activity of a particular compound.

In some aspects, the present invention relates to the treatment of malaria in a subject.

In some aspects, the present invention relates to the treatment of inflammation in a subject.

### Linker Group

A linker is a bifunctional compound which can be used to link a drug and a targeting moiety (e.g., an antibody) to form a targeted drug conjugate (e.g., an antibody-drug conjugate) or targeting conjugate. Such conjugates are useful in the treatment of disease as a drug (e.g., a cytotoxic agent) may be delivered to a cell through recognition of an antigen.

In one aspect, a second section of the linker group is introduced which has a second reactive site (e.g., an electrophilic group) that is reactive to an opposing group (e.g., a nucleophilic group) present on a targeting agent such as an antibody. Useful nucleophilic groups on an antibody include, but are not limited to, sulfhydryl, hydroxyl and amino groups. In this instance, the heteroatom of the nucleophilic group of an antibody is reactive to an electrophilic group on a linker group and forms a covalent bond to that linker group. The electrophilic group then provides a site of attachment for the linker-payload or linker-drug, and can include the disulfide bridges of the antibody (i.e., a stochastic conjugation) or a residue containing an electrophilic group (either synthetic or naturally-occurring) introduced to the antibody to allow efficient conjugation (i.e., site-specific conjugation).

In another aspect, a linker group has a reactive site which has a nucleophilic group that is reactive to an electrophilic group present on an antibody. Electrophilic groups on an antibody include, but are not limited to, aldehyde and ketone carbonyl groups. The heteroatom of a nucleophilic group of a linker group can react with an electrophilic group on an antibody and form a covalent bond to the antibody. Nucleophilic groups in this respect may include, but are not limited to, hydrazide, oxime, amino, hydrazine, thiosemicarbazone, hydrazine carboxylate, and arylhydrazide. The electrophilic group on an antibody provides a convenient site for attachment to a linker group. For a more comprehensive list of linking technologies, please see Jain, N.; Smith, S. W.; Ghone, S.; Tomczuk, B., Current ADC Linker Chemistry. Pharmaceutical Research 2015, 32 (11), 3526-3540.

Linkers can either be cleavable or non-cleavable, with cleavable linkers normally represented by combinations of amino acids. The list of cleavable linkers includes, but is not limited to, valine-citruline, valine-alanine and any combination of two to eight amino acids. A self-immolative unit (e.g., a PAB spacer) can be included to assist with clean cleavage, and optionally hydrophilic groups (e.g., PEG) can be added to increase hydrophilicity of the construct. In some aspects, more suitably, the linker group comprises a self-immolative unit. A range of self immolative units are known in the art [30] and have been described in, for example, U.S. Pat. No. 7,754,681, European Patent Publication No. 0624377.

A variety of suitable linker groups are known in the art and may be used as described herein. For example, the maleimide methodology is routinely used as a method to attach antibodies to drug compounds by providing a linker attached to the drug with a terminal maleimide group. In addition, methodologies using diarylcyclooctyne moieties (such as, but not limited to, DBCO, dibenzylcyclooctyne) are known in the art. Diarylcyclooctynes react with stable azides to provide attachment via the formation of stable triazoles. Diarylcyclooctynes are thermostable with very narrow and specific reactivity toward azides, resulting in almost quantitative yields of stable triazoles. Furthermore, the reaction does not require a cytotoxic Cu(I) catalyst (that is toxic to most organisms) and thus, prevents its use in many biological systems. Still further, alkoxyamine methodologies are also alternatives in the art. For site-specific conjugation of the drug to the antibody, the antibodies may comprise a “tag” (which may be proprietary) that will react with a diarylcyclooctyne (for example DBCO), an alkyoxyamine and/or maleimide group to attach the antibody to the drug. The tag in some instances may be a mutated amino acid. Suitably linker groups incorporating the various groups described above are available in the art.

### Antibody Drug Conjugates

Antibody therapy has been established for the targeted treatment of patients with cancer, immunological and angiogenic disorders (Carter, P. (2006) Nature Reviews Immunology 6:343-357). The use of antibody-drug conjugates (ADC), i.e. immunoconjugates, for the local delivery of cytotoxic or cytostatic agents, i.e. drugs to kill or inhibit tumor cells in the treatment of cancer, targets delivery of the drug moiety to tumors, and intracellular accumulation therein, whereas systemic administration of these unconjugated drug agents may result in unacceptable levels of toxicity to normal cells (Xie et al (2006) Expert. Opin. Biol. Ther. 6(3):281-291; Kovtun ef a/(2006) Cancer Res. 66(6):3214-3121; Law et al (2006) Cancer Res. 66(4):2328-2337; Wu et al (2005) Nature Biotech. 23(9): 1 137-1 145; Lambert J. (2005) Current Opin. in Pharmacol. 5:543-549; Hamann P. (2005) Expert Opin. Ther. Patents 15(9): 1087-1 103; Payne, G. (2003) Cancer Cell 3:207-212; Trail ef a/(2003) Cancer Immunol. Immunother. 52:328-337; Syrigos and Epenetos (1999) Anticancer Research 19:605-614).

Maximal efficacy with minimal toxicity is sought thereby. Efforts to design and refine ADC have focused on the selectivity of monoclonal antibodies (mAbs) as well as drug mechanism of action, drug-linking, drug/antibody ratio (loading), and drug-releasing properties (Junutula, et al., 2008b Nature Biotech., 26(8):925-932; Doman et al., (2009) Blood 114(13):2721-2729; U.S. Pat. Nos. 7,521,541; 7,723,485; WO2009/052249; McDonagh (2006) Protein Eng. Design & Sel. 19(7): 299-307; Doronina et al., (2006) Bioconj. Chem. 17:114-124; Erickson et al., (2006) Cancer Res. 66(8): 1-8; et al., (2005) Clin. Cancer Res. 1 1:843-852; Jeffrey et al., (2005) J. Med. Chem. 48:1344-1358; Hamblett et al., (2004) Clin. Cancer Res. 10:7063-7070). Drug moieties may impart their cytotoxic and cytostatic effects by mechanisms including tubulin binding, DNA binding, proteasome and/or topoisomerase inhibition. Some cytotoxic drugs tend to be inactive or less active when conjugated to large antibodies or protein receptor ligands.

In some aspects, the present invention relates to a compound of formula (I) or pharmaceutically acceptable salts, solvates, tautomers, stereoisomers or mixtures thereof, for use as a drug in an antibody-drug conjugate. Suitably, a compound of formula (I) or pharmaceutically acceptable salts, solvates, tautomers, stereoisomers or mixtures thereof, for use as a drug in an antibody-drug conjugate is prepared by attaching a compound of formula (I) or salts, solvates, isomers or tautomers thereof to an antibody, either directly or via an optional linker group. Suitably, the compound of formula (I) or pharmaceutically acceptable salts, solvates, tautomers, stereoisomers or mixtures thereof, is attached to an antibody via a linker group. Suitably, the antibody-drug conjugate is for use in for treatment of a disease, more specifically of a proliferative disease. Suitably, the drug may be attached by any suitable functional group that it contains to the antibody either directly or via a linker group. Typically, the drug contains, or can be modified to contain, one or more functional groups such as amine, hydroxyl or carboxylic acid groups for attaching the drug to the antibody either directly or via a linker group. In some aspects, the antibody of the antibody drug conjugate is an antibody fragment, such as, but not limited to a single chain antibody. In some aspects, one or more atoms or groups of the compound of formula (I) may be eliminated during the attachment of the drug to the antibody. In some aspects, the antibody binds to a cell surface receptor or a tumor-associated antigen.

In some aspects, the present invention relates to the use of a compound of formula (I) or pharmaceutically acceptable salts, solvates, tautomers, stereoisomers or mixtures thereof, as a drug in an antibody-drug conjugate. Suitably, the use of a compound of formula (I) or pharmaceutically acceptable salts, solvates, tautomers, stereoisomers or mixtures thereof, as a drug in an antibody-drug conjugate is accomplished by attaching a compound of formula (I) or salts, solvates, isomers or tautomers thereof to an antibody, either directly or via an optional linker group. Suitably, the compound of formula (I) or salts, solvates, isomers or tautomers thereof, is attached to an antibody via a linker group. Suitably, the antibody-drug conjugate is for use in for treatment of a disease, more specifically of a proliferative disease. Suitably, the drug may be attached by any suitable functional group that it contains to the antibody either directly or via a linker group. Typically, the drug contains, or can be modified to contain, one or more functional groups such as amine, hydroxyl or carboxylic acid groups for attaching the drug to the antibody either directly or via a linker group. In some aspects, the antibody of the antibody drug conjugate is an antibody fragment, such as, but not limited to a single chain antibody. In some aspects, one or more atoms or groups of the compound of formula (I) may be eliminated during the attachment of the drug to the antibody. In some aspects, the antibody binds to a cell surface receptor or a tumor-associated antigen.

The substituent groups of the compounds of formula (I) may interact with DNA sequences and may be selected so as to target specific sequences. Hence, when the substituent groups are tailored in this way, the compounds of formula (I) find application in targeted chemotherapy.

### Antibody and Antibody Fragments

The term “antibody” specifically covers monoclonal antibodies, polyclonal antibodies, dimers, multimers, multispecific antibodies (e.g., bispecific antibodies), intact antibodies and antibody fragments, so long as they exhibit the desired biological activity, for example, the ability to bind a desired antigen on a target cell or tissue. Antibodies may be murine, human, humanized, chimeric, or derived from other species. An antibody is a protein generated by the immune system that is capable of recognizing and binding to a specific antigen. (Janeway, C, Travers, P., Walport, M., Shlomchik (2001) Immuno Biology, 5th Ed., Garland Publishing, New York). A target antigen generally has numerous binding sites, also called epitopes, recognized by CDRs on the antibody. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. lgG1, lgG2, lgG3, lgG4, lgA1 and lgA2) or subclass, or allotype (e.g. human G1 m1, G1 m2, G1 m3, non-G1 m1 [that, is any allotype other than G1 m1], G1 m17, G2m23, G3m21, G3m28, G3m1 1, G3m5, G3m13, G3m14, G3m10, G3m15, G3m16, G3m6, G3m24, G3m26, G3m27, A2m1, A2m2, Km1, Km2 and Km3) of immunoglobulin molecule. The immunoglobulins can be derived from any species, including human, murine, or rabbit origin.

As used herein, “binds an epitope” is used to mean the antibody binds an epitope with a higher affinity than a non-specific partner such as Bovine Serum Albumin (BSA, Genbank accession no. CAA76847, version no. CAA76847.1 Gl:3336842, record update date: Jan. 7, 2011 02:30 PM). In some embodiments the antibody binds an epitope with an association constant (Ka) at least 2, 3, 4, 5, 10, 20, 50, 100, 200, 500, 1000, 2000, 5000, 10⁴, 10⁵ or 10⁶-fold higher than the antibody's association constant for BSA, when measured at physiological conditions.

The term “antibody fragment” refers to a portion of a full length antibody, for example, the antigen binding or variable region thereof. Examples of antibody fragments include Fab, Fab′, F(ab′)2, and scFv fragments; diabodies; linear antibodies; fragments produced by a Fab expression library, anti-idiotypic (anti-Id) antibodies, CDR (complementary determining region), single-chain antibody molecules; and multispecific antibodies formed from antibody fragments and epitope-binding fragments of any of the above which immunospecifically bind to target antigens, such as, for example, cancer cell antigens, viral antigens or microbial antigens., The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant or epitope on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The antibodies, including monoclonal antibodies, herein specifically include “chimeric” antibodies in which a portion of the antibody structure, for example the heavy and/or light chain, is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences. An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1 q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., lgG1, lgG2, lgG3, lgG4, IgA, and lgA2. The heavy-chain constant domains that correspond to the different classes of antibodies are called α, δ, ε, γ, and μ, respectively. The subunit structures and three-dimensional configurations of different classes of immunoglobulins are well known.

The antibodies disclosed herein may be modified. For example, to make them less immunogenic to a human subject. This may be achieved using any of a number of techniques familiar to the person skilled in the art, such as humanisation.

### Tumor-Associated Antigens:

(1) BMPR1B (bone morphogenetic protein receptor-type IB, Genbank accession no. NM_001203)
ten Dijke, P., et al Science 264 (5155): 101-104 (1994), Oncogene 14 (11): 1377-1382 (1997); WO2004063362 (Claim 2); WO2003042661 (Claim 12); US2003134790-A1 (Page 38-39); WO2002102235 (Claim 13; Page 296); WO2003055443 (Page 91-92); WO200299122 (Example 2; Page 528-530); WO2003029421 (Claim 6); WO2003024392 (Claim 2; FIG. 112); WO200298358 (Claim 1; Page 183); WO200254940 (Page 100-101); WO200259377 (Page 349-350); WO200230268 (Claim 27; Page 376); WO200148204 (Example; FIG. 4) NP_001194 bone morphogenetic protein receptor, type IB/pid=NP_001194.1—Cross-references: MIM:603248; NP_001194.1; AY065994
(2) E16 (LAT1, SLC7A5, Genbank accession no. NM_003486)
Biochem. Biophys. Res. Commun. 255 (2), 283-288 (1999), Nature 395 (6699):288-291 (1998), Gaugitsch, H. W., et al (1992) J. Biol. Chem. 267 (16): 11267-11273); WO2004048938 (Example 2); WO2004032842 (Example TV); WO2003042661 (Claim 12); WO2003016475 (Claim 1); WO200278524 (Example 2); WO200299074 (Claim 19; Page 127-129); WO200286443 (Claim 27; Pages 222, 393); WO2003003906 (Claim 10; Page 293); WO200264798 (Claim 33; Page 93-95); WO200014228 (Claim 5; Page 133-136); US2003224454 (FIG. 3); WO2003025138 (Claim 12; Page 150); NP_003477 solute carrier family 7 (cationic amino acid transporter, y+ system), member 5/pid=NP_003477.3—Homo sapiens; Cross-references: MIM:600182; NP_003477.3; NM_015923; NM_003486_1
(3) STEAP1 (six transmembrane epithelial antigen of prostate, Genbank accession no. NM_012449)
Cancer Res. 61 (15), 5857-5860 (2001), Hubert, R. S., et al (1999) Proc. Natl. Acad. Sci. U.S.A. 96 (25): 14523-14528); WO2004065577 (Claim 6); WO2004027049 (FIG. 1L); EP1394274 (Example 11); WO2004016225 (Claim 2); WO2003042661 (Claim 12); US2003157089 (Example 5); US2003185830 (Example 5); US2003064397 (FIG. 2); WO200289747 (Example 5; Page 618-619); WO2003022995 (Example 9; FIG. 13A, Example 53; Page 173, Example 2; FIG. 2A); NP_036581 six transmembrane epithelial antigen of the prostate; Cross-references: MIM:604415; NP_036581.1; NM_012449_1
(4) 0772P (CA125, MUC16, Genbank accession no. AF361486)
J. Biol. Chem. 276 (29):27371-27375 (2001)); WO2004045553 (Claim 14); WO200292836 (Claim 6; FIG. 12); WO200283866 (Claim 15; Page 116-121); US2003124140 (Example 16); U.S. Pat. No. 798,959; Cross-references: GI:34501467; AAK74120.3; AF361486_1
(5) MPF (MPF, MSLN, SMR, megakaryocyte potentiating factor, mesothelin, Genbank accession no. NM_005823) Yamaguchi, N., et al Biol. Chem. 269 (2), 805-808 (1994), Proc. Natl. Acad. Sci. U.S.A. 96 (20): 11531-11536 (1999), Proc. Natl. Acad. Sci. U.S.A. 93 (1): 136-140 (1996), J. Biol. Chem. 270 (37):21984-21990 (1995)); WO2003101283 (Claim 14); (WO2002102235 (Claim 13; Page 287-288); WO2002101075 (Claim 4; Page 308-309); WO200271928 (Page 320-321); WO9410312 (Page 52-57); Cross-references: MIM:601051; NP_005814.2; NM_005823_1
(6) Napi2b (Napi3b, NAPI-3B, NPTIIb, SLC34A2, solute carrier family 34
(sodium phosphate), member 2, type II sodium-dependent phosphate transporter 3b, Genbank accession no. NM_006424) J. Biol. Chem. 277 (22): 19665-19672 (2002), Genomics 62 (2):281-284 (1999), Feild, J. A., et al (1999) Biochem. Biophys. Res. Commun. 258 (3):578-582); WO2004022778 (Claim 2); EP1394274 (Example 11); WO2002102235 (Claim 13; Page 326); EP875569 (Claim 1; Page 17-19); WO200157188 (Claim 20; Page 329); WO2004032842 (Example IV); WO200175177 (Claim 24; Page 139-140); Cross-references: MIM:604217; NP_006415.1; NM_006424_1
(7) Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B, Genbank accession no. AB040878) Nagase T., et al (2000) DNA Res. 7 (2): 143-150); WO2004000997 (Claim 1); WO2003003984 (Claim 1); WO200206339 (Claim 1; Page 50); WO200188133 (Claim 1; Page 41-43, 48-58); WO2003054152 (Claim 20); WO2003101400 (Claim 11); Accession: Q9P283; EMBL; AB040878; BAA95969.1. Genew; HGNC: 10737;
(8) PSCA hlg (2700050C12Rik, C530008O16Rik, RIKEN cDNA 2700050C12, RIKEN cDNA 2700050C12 gene, Genbank accession no. AY358628); Ross et al (2002) Cancer Res. 62:2546-2553; US2003129192 (Claim 2); US2004044180 (Claim 12); US2004044179 (Claim 11); US2003096961 (Claim 11); US2003232056 (Example 5); WO2003105758 (Claim 12); US2003206918 (Example 5); EP1347046 (Claim 1); WO2003025148 (Claim 20); Cross-references: GI:37182378; AAQ88991.1; AY358628_1
(9) ETBR (Endothelin type B receptor, Genbank accession no. AY275463); Nakamuta M., et al Biochem. Biophys. Res. Commun. 177, 34-39, 1991; Ogawa Y., et al Biochem. Biophys. Res. Commun. 178, 248-255, 1991; Arai H., et al Jpn. Circ. J. 56, 1303-1307, 1992; Arai H., et al J. Biol. Chem. 268, 3463-3470, 1993; Sakamoto A., Yanagisawa M., et al Biochem. Biophys. Res. Commun. 178, 656-663, 1991; Elshourbagy N. A., et al J. Biol. Chem. 268, 3873-3879, 1993; Haendler B., et al J. Cardiovasc. Pharmacol. 20, S1-S4, 1992; Tsutsumi M., et al Gene 228, 43-49, 1999; Strausberg R. L., et al Proc. Natl. Acad. Sci. U.S.A. 99, 16899-16903, 2002; Bourgeois C, et al J. Clin. Endocrinol. Metab. 82, 3116-3123, 1997; Okamoto Y., et al Biol. Chem. 272, 21589-21596, 1997; Verheij J. B., et al Am. J. Med. Genet. 108, 223-225, 2002; Hofstra R. M. W., et al Eur. J. Hum. Genet. 5, 180-185, 1997; Puffenberger E. G., et al Cell 79, 1257-1266, 1994; Attie T., et al, Hum. Mol. Genet. 4, 2407-2409, 1995; Auricchio A., et al Hum. Mol. Genet. 5:351-354, 1996; Amiel J., et al Hum. Mol. Genet. 5, 355-357, 1996; Hofstra R. M. W., et al Nat. Genet. 12, 445-447, 1996; Svensson P J., et al Hum. Genet. 103, 145-148, 1998; Fuchs S., et al Mol. Med. 7, 115-124, 2001; Pingault V., et al (2002) Hum. Genet. 111, 198-206; WO2004045516 (Claim 1); WO2004048938 (Example 2); WO2004040000 (Claim 151); WO2003087768 (Claim 1); WO2003016475 (Claim 1); WO2003016475 (Claim 1); WO200261087 (FIG. 1); WO2003016494 (FIG. 6); WO2003025138 (Claim 12; Page 144); WO200198351 (Claim 1; Page 124-125); EP522868 (Claim 8; FIG. 2); WO200177172 (Claim 1; Page 297-299); US2003109676; U.S. Pat. No. 6,518,404 (FIG. 3); U.S. Pat. No. 5,773,223 (Claim 1a; Col 31-34); WO2004001004;
(10) MSG783 (RNF124, hypothetical protein FLJ20315, Genbank accession no. NM_017763);

### WO2003104275 (Claim 1); WO2004046342 (Example 2); WO2003042661 (Claim 12); WO2003083074 (Claim 14; Page 61); WO2003018621 (Claim 1); WO2003024392 (Claim 2; FIG. 93); WO200166689 (Example 6); Cross-references: LocusID: 54894; NP_060233.2; NM_017763_1

(11) STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein, Genbank accession no. AF455138)
Lab. Invest. 82 (11): 1573-1582 (2002); WO2003087306; US2003064397 (Claim 1; FIG. 1); WO200272596 (Claim 13; Page 54-55); WO200172962 (Claim 1; FIG. 4B); WO2003104270 (Claim 11); WO2003104270 (Claim 16); US2004005598 (Claim 22); WO2003042661 (Claim 12); US2003060612 (Claim 12; FIG. 10); WO200226822 (Claim 23; FIG. 2); WO200216429 (Claim 12; FIG. 10); Cross-references: GI:22655488; AAN04080.1; AF455138_1
(12) TrpM4 (BR22450, FLJ20041, TRPM4, TRPM4B, transient receptor potential cation channel, subfamily M, member 4, Genbank accession no. NM_017636) Xu, X. Z., et al Proc. Natl. Acad. Sci. U.S.A. 98 (19): 10692-10697 (2001), Cell 109 (3):397-407 (2002), J. Biol. Chem. 278 (33):30813-30820 (2003); US2003143557 (Claim 4); WO200040614 (Claim 14; Page 100-103); WO200210382 (Claim 1; FIG. 9A); WO2003042661 (Claim 12); WO200230268 (Claim 27; Page 391); US2003219806 (Claim 4); WO200162794 (Claim 14; FIG. 1A-D); Cross-references: MIM:606936; NP_060106.2; NM_017636_1
(13) CRIPTO (CR, CR1, CRGF, CRIPTO, TDGF1, teratocarcinoma-derived growth factor, Genbank accession no. NP_003203 or NM_003212)
Ciccodicola, A., et al EMBO J. 8 (7): 1987-1991 (1989), Am. J. Hum. Genet. 49 (3):555-565 (1991); US2003224411 (Claim 1); WO2003083041 (Example 1); WO2003034984 (Claim 12); WO200288170 (Claim 2; Page 52-53); WO2003024392 (Claim 2; FIG. 58); WO200216413 (Claim 1; Page 94-95, 105); WO200222808 (Claim 2; FIG. 1); U.S. Pat. No. 5,854,399 (Example 2; Col 17-18); U.S. Pat. No. 5,792,616 (FIG. 2); Cross-references: MIM: 187395; NP_003203.1; NM_003212_1
(14) CD21 (CR2 (Complement receptor 2) or C3DR (C3d/Epstein Barr virus receptor) or Hs.73792 Genbank accession no. M26004)
Fujisaku et al (1989) J. Biol. Chem. 264 (4):2118-2125); Weis J. J., et al J. Exp. Med. 167, 1047-1066, 1988; Moore M., et al Proc. Natl. Acad. Sci. U.S.A. 84, 9194-9198, 1987; Barel M., et al Mol. Immunol. 35, 1025-1031, 1998; Weis J. J., et al Proc. Natl. Acad. Sci. U.S.A. 83, 5639-5643, 1986; Sinha S. K., et al (1993) J. Immunol. 150, 5311-5320; WO2004045520 (Example 4); US2004005538 (Example 1); WO2003062401 (Claim 9); WO2004045520 (Example 4); WO9102536 (FIGS. 9.1-9.9); WO2004020595 (Claim 1); Accession: P20023; Q13866; Q14212; EMBL; M26004; AAA35786.1.
(15) CD79b (CD79B, CD79β, IGb (immunoglobulin-associated beta), B29, Genbank accession no. NM_000626 or 11038674)
Proc. Natl. Acad. Sci. U.S.A. (2003) 100 (7):4126-4131, Blood (2002) 100 (9):3068-3076, Muller et al (1992) Eur. J. Immunol. 22 (6): 1621-1625); WO2004016225 (claim 2, FIG. 140); WO2003087768, US2004101874 (claim 1, page 102); WO2003062401 (claim 9); WO200278524 (Example 2); US2002150573 (claim 5, page 15); U.S. Pat. No. 5,644,033; WO2003048202 (claim 1, pages 306 and 309); WO 99/558658, U.S. Pat. No. 6,534,482 (claim 13, FIG. 17A/B); WO200055351 (claim 11, pages 1145-1146); Cross-references: MIM: 147245; NP_000617.1; NM_000626_1
(16) FcRH2 (IFGP4, IRTA4, SPAP1A (SH2 domain containing phosphatase anchor protein 1a), SPAP1B, SPAP1C, Genbank accession no. NM_030764, AY358130) Genome Res. 13 (10):2265-2270 (2003), Immunogenetics 54 (2):87-95 (2002), Blood 99 (8):2662-2669 (2002), Proc. Natl. Acad. Sci. U.S.A. 98 (17):9772-9777 (2001), Xu, M. J., et al (2001) Biochem. Biophys. Res. Commun. 280 (3):768-775; WO2004016225 (Claim 2); WO2003077836; WO200138490 (Claim 5; FIG. 18D-1-18D-2); WO2003097803 (Claim 12); WO2003089624 (Claim 25); Cross-references: MIM:606509; NP_110391.2; NM_030764_1
(17) HER2 (ErbB2, Genbank accession no. M11730)
Coussens L., et al Science (1985) 230(4730): 1132-1139); Yamamoto T., et al Nature 319, 230-234, 1986; Semba K., et al Proc. Natl. Acad. Sci. U.S.A. 82, 6497-6501, 1985; Swiercz J. M., et al J. Cell Biol. 165, 869-880, 2004; Kuhns J. J., et al J. Biol. Chem. 274, 36422-36427, 1999; Cho H.-S., et al Nature 421, 756-760, 2003; Ehsani A, et al (1993) Genomics 15, 426-429; WO2004048938 (Example 2); WO2004027049 (FIG. 1I); WO2004009622; WO2003081210; WO2003089904 (Claim 9); WO2003016475 (Claim 1); US2003118592; WO2003008537 (Claim 1); WO2003055439 (Claim 29; FIG. 1 A-B); WO2003025228 (Claim 37; FIG. 5C); WO200222636 (Example 13; Page 95-107); WO200212341 (Claim 68; FIG. 7); WO200213847 (Page 71-74); WO200214503 (Page 114-117); WO200153463 (Claim 2; Page 41-46); WO200141787 (Page 15); WO200044899 (Claim 52; FIG. 7); WO200020579 (Claim 3; FIG. 2); U.S. Pat. No. 5,869,445 (Claim 3; Col 31-38); WO9630514 (Claim 2; Page 56-61); EP1439393 (Claim 7); WO2004043361 (Claim 7); WO2004022709; WO200100244 (Example 3; FIG. 4); Accession: P04626; EMBL; M11767; AAA35808.1. EMBL; M11761; AAA35808.1.
(18) NCA (CEACAM6, Genbank accession no. M18728);
Barnett T., et al Genomics 3, 59-66, 1988; Tawaragi Y., et al Biochem. Biophys. Res. Commun. 150, 89-96, 1988; Strausberg R. L., et al Proc. Natl. Acad. Sci. U.S.A. 99: 16899-16903, 2002; WO2004063709; EP 1439393 (Claim 7); WO2004044178 (Example 4); WO2004031238; WO2003042661 (Claim 12); WO200278524 (Example 2); WO200286443 (Claim 27; Page 427); WO200260317 (Claim 2); Accession: P40199; Q14920; EMBL; M29541; AAA59915.1. EMBL; M18728;
(19) MDP (DPEP1, Genbank accession no. BC017023)
Proc. Natl. Acad. Sci. U.S.A. 99 (26): 16899-16903 (2002); WO2003016475 (Claim 1); WO200264798 (Claim 33; Page 85-87); JP05003790 (FIG. 6-8); Wo9946284 (FIG. 9); Cross-references: MIM: 179780; AAH17023.1; BC017023_1
(20) IL20Rα (IL20Rα, ZCYTOR7, Genbank accession no. AF 184971);
Clark H. F., et al Genome Res. 13, 2265-2270, 2003; Mungall A. J., et al Nature 425, 805-811, 2003; Blumberg H., et al Cell 104, 9-19, 2001; Dumoutier L., et al J. Immunol. 167, 3545-3549, 2001; Parrish-Novak J., et al J. Biol. Chem. 277, 47517-47523, 2002; Pletnev S., et al (2003) Biochemistry 42: 12617-12624; Sheikh F., et al (2004) J. Immunol. 172, 2006-2010; EP1394274 (Example 11); US2004005320 (Example 5); WO2003029262 (Page 74-75); WO2003002717 (Claim 2; Page 63); WO200222153 (Page 45-47); US2002042366 (Page 20-21); WO200146261 (Page 57-59); WO200146232 (Page 63-65); Wo9837193 (Claim 1; Page 55-59); Accession: Q9UHF4; Q6UWA9; Q96SH8; EMBL; AF 184971; AAF01320.1.
(21) Brevican (BCAN, BEHAB, Genbank accession no. AF229053)
Gary S. C., et al Gene 256, 139-147, 2000; Clark H. F., et al Genome Res. 13, 2265-2270, 2003; Strausberg R. L., et al Proc. Natl. Acad. Sci. U.S.A. 99, 16899-16903, 2002; US2003186372 (Claim 11); US2003186373 (Claim 11); US2003119131 (Claim 1; FIG. 52); US2003119122 (Claim 1; FIG. 52); US2003119126 (Claim 1); US2003119121 (Claim 1; FIG. 52); US2003119129 (Claim 1); US2003119130 (Claim 1); US2003119128 (Claim 1; FIG. 52); US2003119125 (Claim 1); WO2003016475 (Claim 1); WO200202634 (Claim 1);
(22) EphB2R (DRT, ERK, Hek5, EPHT3, Tyro5, Genbank accession no. NM_004442) Chan, J. and Watt, V. M., Oncogene 6 (6), 1057-1061 (1991) Oncogene 10 (5):897-905 (1995), Annu. Rev. Neurosci. 21:309-345 (1998), Int. Rev. Cytol. 196: 177-244 (2000); WO2003042661 (Claim 12); WO200053216 (Claim 1; Page 41); WO2004065576 (Claim 1); WO2004020583 (Claim 9); WO2003004529 (Page 128-132); WO200053216 (Claim 1; Page 42); Cross-references: MIM: 600997; NP_004433.2; NM_004442_1
(23) ASLG659 (B7h, Genbank accession no. AX092328)
US20040101899 (Claim 2); WO2003104399 (Claim 11); WO2004000221 (FIG. 3); US2003165504 (Claim 1); US2003124140 (Example 2); US2003065143 (FIG. 60); WO2002102235 (Claim 13; Page 299); US2003091580 (Example 2); WO200210187 (Claim 6; FIG. 10); WO200194641 (Claim 12; FIG. 7b); WO200202624 (Claim 13; FIG. 1A-1B); US2002034749 (Claim 54; Page 45-46); WO200206317 (Example 2; Page 320-321, Claim 34; Page 321-322); WO200271928 (Page 468-469); WO200202587 (Example 1; FIG. 1); WO200140269 (Example 3; Pages 190-192); WO200036107 (Example 2; Page 205-207); WO2004053079 (Claim 12); WO2003004989 (Claim 1); WO200271928 (Page 233-234, 452-453); WO 0116318;
(24) PSCA (Prostate stem cell antigen precursor, Genbank accession no. AJ297436) Reiter R. E., et al Proc. Natl. Acad. Sci. U.S.A. 95, 1735-1740, 1998; Gu Z., et al Oncogene 19, 1288-1296, 2000; Biochem. Biophys. Res. Commun. (2000) 275(3):783-788; WO2004022709; EP1394274 (Example 11); US2004018553 (Claim 17); WO2003008537 (Claim 1); WO200281646 (Claim 1; Page 164); WO2003003906 (Claim 10; Page 288); WO200140309 (Example 1; FIG. 17); US2001055751 (Example 1; FIG. 1b); WO200032752 (Claim 18; FIG. 1); WO9851805 (Claim 17; Page 97); Wo9851824 (Claim 10; Page 94); WO9840403 (Claim 2; FIG. 1B); Accession: 043653; EMBL; AF043498; AAC39607.1.
(25) GEDA (Genbank accession No. AY260763);
AAP14954 lipoma HMGIC fusion-partner-like protein/pid=AAP14954.1 —Homo sapiens Species: Homo sapiens (human)

### WO2003054152 (Claim 20); WO2003000842 (Claim 1); WO2003023013 (Example 3, Claim 20); US2003194704 (Claim 45); Cross-references: GI:30102449; AAP14954.1; AY260763_1

(26) BAFF-R (B cell-activating factor receptor, BLyS receptor 3, BR3, Genbank accession No. AF116456); BAFF receptor/pid=NP_443177.1—Homo sapiens Thompson, J. S., et al Science 293 (5537), 2108-2111 (2001); WO2004058309; WO2004011611; WO2003045422 (Example; Page 32-33); WO2003014294 (Claim 35; FIG. 6B); WO2003035846 (Claim 70; Page 615-616); WO200294852 (Col 136-137); WO200238766 (Claim 3; Page 133); WO200224909 (Example 3; FIG. 3); Cross-references: MIM:606269; NP_443177.1; NM_052945_1; AF132600
(27) CD22 (B-cell receptor CD22-B isoform, BL-CAM, Lyb-8, Lyb8, SIGLEC-2, FLJ22814, Genbank accession No. AK026467);
Wilson et al (1991) J. Exp. Med. 173: 137-146; WO2003072036 (Claim 1; FIG. 1); Cross-references: MIM: 107266; NP_001762.1; NM_001771_1
(28) CD79a (CD79A, CD79a, immunoglobulin-associated alpha, a B cell-specific protein that covalently interacts with Ig beta (CD79B) and forms a complex on the surface with Ig M molecules, transduces a signal involved in B-cell differentiation), pI: 4.84, MW: 25028 TM: 2 [P] Gene Chromosome: 19q13.2, Genbank accession No. NP_001774.10)
WO2003088808, US20030228319; WO2003062401 (claim 9); US2002150573 (claim 4, pages 13-14); Wo9958658 (claim 13, FIG. 16); WO9207574 (FIG. 1); U.S. Pat. No. 5,644,033; Ha et al (1992) J. Immunol. 148(5): 1526-1531; Mueller et al (1992) Eur. J. Biochem. 22: 1621-1625; Hashimoto et al (1994) Immunogenetics 40(4):287-295; Preud'homme et al (1992) Clin. Exp. Immunol. 90(1): 141-146; Yu et al (1992) J. Immunol. 148(2) 633-637; Sakaguchi et al (1988) EMBO J. 7(11):3457-3464;
(29) CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia); 372 aa, pI: 8.54 MW: 41959 TM: 7 [P] Gene Chromosome: 1 1q23.3, Genbank accession No. NP_001707.1)
WO2004040000; WO2004015426; US2003105292 (Example 2); U.S. Pat. No. 6,555,339 (Example 2); WO200261087 (FIG. 1); WO200157188 (Claim 20, page 269); WO200172830 (pages 12-13); WO200022129 (Example 1, pages 152-153, Example 2, pages 254-256); Wo9928468 (claim 1, page 38); U.S. Pat. No. 5,440,021 (Example 2, col 49-52); Wo9428931 (pages 56-58); Wo9217497 (claim 7, FIG. 5); Dobner et al (1992) Eur. J. Immunol. 22:2795-2799; Barella et al (1995) Biochem. J. 309:773-779;
(30) HLA-DOB (Beta subunit of MHC class II molecule (la antigen) that binds peptides and presents them to CD4+T lymphocytes); 273 aa, pI: 6.56 MW: 30820 TM: 1 [P] Gene Chromosome: 6p21.3, Genbank accession No. NP_002111.1)
Tonnelle et al (1985) EMBO J. 4(11):2839-2847; Jonsson et al (1989) Immunogenetics 29(6):411-413; Beck et al (1992) J. Mol. Biol. 228:433-441; Strausberg et al (2002) Proc. Natl. Acad. Sci USA 99: 16899-16903; Servenius et al (1987) J. Biol. Chem. 262:8759-8766; Beck et al (1996) J. Mol. Biol. 255: 1-13; Naruse et al (2002) Tissue Antigens 59:512-519; Wo9958658 (claim 13, FIG. 15); US6153408 (Col 35-38); U.S. Pat. No. 5,976,551 (col 168-170); US6011146 (col 145-146); Kasahara et al (1989) Immunogenetics 30(1):66-68; Larhammar et al (1985) J. Biol. Chem. 260(26): 14111-14119;
(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), pI: 7.63, MW: 47206 TM: 1 [P] Gene Chromosome: 17p13.3, Genbank accession No. NP_002552.2)
Le et al (1997) FEBS Lett. 418(1-2): 195-199; WO2004047749; WO2003072035 (claim 10); Touchman et al (2000) Genome Res. 10: 165-173; WO200222660 (claim 20); WO2003093444 (claim 1); WO2003087768 (claim 1); WO2003029277 (page 82);
(32) CD72 (B-cell differentiation antigen CD72, Lyb-2) PROTEIN SEQUENCE Full maeaity . . . tafrfpd (1 . . . 359; 359 aa), pI: 8.66, MW: 40225 TM: 1 [P] Gene Chromosome: 9p13.3, Genbank accession No. NP_001773.1)
WO2004042346 (claim 65); WO2003026493 (pages 51-52, 57-58); WO200075655 (pages 105-106); Von Hoegen et al (1990) J. Immunol. 144(12):4870-4877; Strausberg et al (2002) Proc. Natl. Acad. Sci USA 99: 16899-16903;
(33) LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosis); 661 aa, pI: 6.20, MW: 74147 TM: 1 [P] Gene Chromosome: 5q12, Genbank accession No. NP_005573.1)
US2002193567; WO9707198 (claim 11, pages 39-42); Miura et al (1996) Genomics 38(3):299-304; Miura et al (1998) Blood 92:2815-2822; WO2003083047; Wo9744452 (claim 8, pages 57-61); WO200012130 (pages 24-26);
(34) FcRH1 (Fc receptor-like protein 1, a putative receptor for the immunoglobulin Fe domain that contains C2 type Ig-like and ITAM domains, may have a role in B-lymphocyte differentiation); 429 aa, pI: 5.28, MW: 46925 TM: 1 [P] Gene Chromosome: 1q21-1q22, Genbank accession No. NP_443170.1) WO2003077836; WO200138490 (claim 6, FIG. 18E-1-18-E-2); Davis et al (2001) Proc. Natl. Acad. Sci USA 98(17):9772-9777; WO2003089624 (claim 8); EP1347046 (claim 1); WO2003089624 (claim 7);
(35) IRTA2 (Immunoglobulin superfamily receptor translocation associated 2, a putative immunoreceptor with possible roles in B cell development and lymphomagenesis; deregulation of the gene by translocation occurs in some B cell malignancies); 977 aa, pI: 6.88 MW: 106468 TM: 1 [P] Gene Chromosome: 1q21, Genbank accession No. Human: AF343662, AF343663, AF343664, AF343665, AF369794, AF397453, AK090423, AK090475, AL834187, AY358085; Mouse: AK089756, AY158090, AY506558; NP_112571.1 WO2003024392 (claim 2, FIG. 97); Nakayama et al (2000) Biochem. Biophys. Res. Commun. 277(1): 124-127; WO2003077836; WO200138490 (claim 3, FIG. 18B-1-18B-2);
(36) TENB2 (TMEFF2, tomoregulin, TPEF, HPP1, TR, putative transmembrane proteoglycan, related to the EGF/heregulin family of growth factors and follistatin); 374 aa, NCBI Accession: AAD55776, AAF91397, AAG49451, NCBI RefSeq: NP_057276; NCBI Gene: 23671; OMIM: 605734; SwissProt Q9UIK5; Genbank accession No. AF179274; AY358907, CAF85723, CQ782436
WO2004074320 (SEQ ID NO 810); JP2004113151 (SEQ ID NOS 2, 4, 8); WO2003042661 (SEQ ID NO 580); WO2003009814 (SEQ ID NO 411); EP1295944 (pages 69-70); WO200230268 (page 329); WO200190304 (SEQ ID NO 2706); US2004249130; US2004022727; WO2004063355; US2004197325; US2003232350; US2004005563; US2003124579; Horie et al (2000) Genomics 67: 146-152; Uchida et al (1999) Biochem. Biophys. Res. Commun. 266:593-602; Liang et al (2000) Cancer Res. 60:4907-12; Glynne-Jones et al (2001) Int J Cancer. October 15; 94(2): 178-84;
(37) PMEL17 (silver homolog; SILV; D12S53E; PMEL17; SI; SIL); ME20; gp100) BC001414; BT007202; M32295; M77348; NM_006928; McGlinchey, R. P. et al (2009) Proc. Natl. Acad. Sci. U.S.A. 106 (33), 13731-13736; Kummer, M. P. et al (2009) J. Biol. Chem. 284 (4), 2296-2306;
(38) TMEFF1 (transmembrane protein with EGF-like and two follistatin-like domains 1; Tomoregulin-1); H7365; C9orf2; C9ORF2; U19878; X83961; NM_080655; NM_003692; Harms, P. W. (2003) Genes Dev. 17 (21), 2624-2629; Gery, S. et al (2003) Oncogene 22 (18):2723-2727;
(39) GDNF-Ra1 (GDNF family receptor alpha 1; GFRA1; GDNFR; GDNFRA; RETL1; TRNR1; RET1L; GDNFR-alpha1; GFR-ALPHA-1); U95847; BC014962; NM_145793 NM_005264; Kim, M. H. et al (2009) Mol. Cell. Biol. 29 (8), 2264-2277; Treanor, J. J. et al (1996) Nature 382 (6586):80-83;
(40) Ly6E (lymphocyte antigen 6 complex, locus E, Ly67, RIG-E, SCA-2, TSA-l); NP_002337.1; NM_002346.2; de Nooij-van Dalen, A G. et al (2003) Int. J. Cancer 103 (6), 768-774; Zammit, D. J. et al (2002) Mol. Cell. Biol. 22 (3):946-952; WO 2013/17705;
(41) TMEM46 (shisa homolog 2 (Xenopus laevis); SHISA2); NP_001007539.1; NM_001007538.1; Furushima, K. et al (2007) Dev. Biol. 306 (2), 480-492; Clark, H. F. et al (2003) Genome Res. 13 (10):2265-2270;
(42) Ly6G6D (lymphocyte antigen 6 complex, locus G6D; Ly6-D, MEGTl); NP_067079.2; NM_021246.2; Mallya, M. et al (2002) Genomics 80 (1): 113-123; Ribas, G. et al (1999) J. Immunol. 163 (1):278-287;
(43) LGR5 (leucine-rich repeat-containing G protein-coupled receptor 5; GPR49, GPR67); NP_003658.1; NM_003667.2; Salanti, G. et al (2009) Am. J. Epidemiol. 170 (5):537-545; Yamamoto, Y. et al (2003) Hepatology 37 (3):528-533;
(44) RET (ret proto-oncogene; MEN2A; HSCR1; MEN2B; MTC1; PTC; CDHF12; Hs.168114; RET51; RET-ELE1); NP_066124.1; NM_020975.4; Tsukamoto, H. et al (2009) Cancer Sci. 100 (10): 1895-1901; Narita, N. et al (2009) Oncogene 28 (34):3058-3068;
(45) LY6K (lymphocyte antigen 6 complex, locus K; LY6K; HSJ001348; FLJ35226); NP_059997.3; NM_017527.3; Ishikawa, N. et al (2007) Cancer Res. 67 (24): 11601-11611; de Nooij-van Dalen, A G. et al (2003) Int. J. Cancer 103 (6):768-774;
(46) GPR19 (G protein-coupled receptor 19; Mm.4787); NP_006134.1; NM_006143.2; Montpetit, A. and Sinnett, D. (1999) Hum. Genet. 105 (1-2): 162-164; O'Dowd, B. F. et al (1996) FEBS Lett. 394 (3):325-329;
(47) GPR54 (KISS1 receptor; KISS1R; GPR54; HOT7T175; AXOR12); NP_115940.2; NM 032551.4; Navenot, J. M. et al (2009) Mol. Pharmacol. 75 (6): 1300-1306; Hata, K. et al (2009) Anticancer Res. 29 (2):617-623;
(48) ASPHD1 (aspartate beta-hydroxylase domain containing 1; LOC253982); NP_859069.2; NM_181718.3; Gerhard, D. S. et al (2004) Genome Res. 14 (10B):2121-2127;
(49) Tyrosinase (TYR; OCAIA; OCA1A; tyrosinase; SHEP3); NP_000363.1; NM_000372.4; Bishop, D. T. et al (2009) Nat. Genet. 41 (8):920-925; Nan, H. et al (2009) Int. J. Cancer 125 (4): 909-917;
(50) TMEM118 (ring finger protein, transmembrane 2; RNFT2; FLJ14627); NP_001103373.1; NM 001109903.1; Clark, H. F. et al (2003) Genome Res. 13 (10):2265-2270; Scherer, S. E. et al (2006) Nature 440 (7082):346-351 (51) GPR172A (G protein-coupled receptor 172A; GPCR41; FLJ11856; D15Ertd747e); NP_078807.1; NM_024531.3; Ericsson, T. A. et al (2003) Proc. Natl. Acad. Sci. U.S.A. 100 (11):6759-6764; Takeda, S. et al (2002) FEBS Lett. 520 (1-3):97-101.
(52) CD33, a member of the sialic acid binding, immunoglobulin-like lectin family, is a 67-kDa glycosylated transmembrane protein. CD33 is expressed on most myeloid and monocytic leukemia cells in addition to committed myelomonocytic and erythroid progenitor cells. It is not seen on the earliest pluripotent stem cells, mature granulocytes, lymphoid cells, or nonhematopoietic cells (Sabbath et al., (1985) J. Clin. Invest. 75:756-56; Andrews et al., (1986) Blood 68: 1030-5). CD33 contains two tyrosine residues on its cytoplasmic tail, each of which is followed by hydrophobic residues similar to the immunoreceptor tyrosine-based inhibitory motif (ITIM) seen in many inhibitory receptors.
(53) CLL-1 (CLEC12A, MICL, and DCAL2), encodes a member of the C-type lectin/C-type lectin-like domain (CTL/CTLD) superfamily. Members of this family share a common protein fold and have diverse functions, such as cell adhesion, cell-cell signalling, glycoprotein turnover, and roles in inflammation and immune response. The protein encoded by this gene is a negative regulator of granulocyte and monocyte function. Several alternatively spliced transcript variants of this gene have been described, but the full-length nature of some of these variants has not been determined. This gene is closely linked to other CTL/CTLD superfamily members in the natural killer gene complex region on chromosome 12p13 (Drickamer K (1999) Curr. Opin. Struct. Biol. 9 (5):585-90; van Rhenen A, et al., (2007) Blood 110 (7):2659-66; Chen C H, et al. (2006) Blood 107 (4): 1459-67; Marshall A S, et al. (2006) Eur. J. Immunol. 36 (8):2159-69; Bakker A B, et al (2005) Cancer Res. 64 (22):8443-50; Marshall A S, et al (2004) J. Biol. Chem. 279 (15): 14792-802). CLL-1 has been shown to be a type II transmembrane receptor comprising a single C-type lectin-like domain (which is not predicted to bind either calcium or sugar), a stalk region, a transmembrane domain and a short cytoplasmic tail containing an ITIM motif.

### Anti-CD22 Antibodies

In certain embodiments, the anti-CD22 antibodies of an ADC comprises three light chain hypervariable regions (HVR-L1, HVR-L2 and HVR-L3) and three heavy chain hypervariable regions (HVR-H1, HVR-H2 and HVR-H3), according to U.S. Pat. No. 8,226,945:

HVR-L1
(SEQ ID NO: 1)
RSSQSIVHSVGNTFLE
HVR-L2
(SEQ ID NO: 2)
KVSNRFS
HVR-L3
(SEQ ID NO: 3)
FQGSQFPYT
HVR-H1
(SEQ ID NO: 4)
GYEFSRSWMN
HVR-H2
(SEQ ID NO: 5)
GRIYPGDGDTNYSGKFKG
HVR-H3
(SEQ ID NO: 6)
DGSSWDWYFDV

### Anti-Ly6E Antibodies

In certain embodiments, an ADC comprises anti-Ly6E antibodies. Lymphocyte antigen 6 complex, locus E (Ly6E), also known as retinoic acid induced gene E (RIG-E) and stem cell antigen 2 (SCA-2). It is a GPI linked, 131 amino acid length, ˜8.4 kDa protein of unknown function with no known binding partners. It was initially identified as a transcript expressed in immature thymocyte, thymic medullary epithelial cells in mice (Mao, et al. (1996) Proc. Natl. Acad. Sci. U.S.A. 93:5910-5914). In some embodiments, the invention provides an immunoconjugate comprising an anti-Ly6E antibody described in PCT Publication No. WO 2013/177055.

In some embodiments, the invention provides an antibody-drug conjugate comprising an anti-Ly6E antibody comprising at least one, two, three, four, five, or six HVRs selected from (a) HVR-H1 comprising the amino acid sequence of SEQ ID NO: 12; (b) HVR-H2 comprising the amino acid sequence of SEQ ID NO: 13; (c) HVR-H3 comprising the amino acid sequence of SEQ ID NO: 14; (d) HVR-L1 comprising the amino acid sequence of SEQ ID NO: 9; (e) HVR-L2 comprising the amino acid sequence of SEQ ID NO: 10; and (f) HVR-L3 comprising the amino acid sequence of SEQ ID NO: 11.

In one aspect, the invention provides an antibody-drug conjugate comprising an antibody that comprises at least one, at least two, or all three VH HVR sequences selected from (a) HVR-H1 comprising the amino acid sequence of SEQ ID NO: 12; (b) HVR-H2 comprising the amino acid sequence of SEQ ID NO: 13; and (c) HVR-H3 comprising the amino acid sequence of SEQ ID NO: 14. In a further embodiment, the antibody comprises (a) HVR-H1 comprising the amino acid sequence of SEQ ID NO: 12; (b) HVR-H2 comprising the amino acid sequence of SEQ ID NO: 13; and (c) HVR-H3 comprising the amino acid sequence of SEQ ID NO: 14.

In another aspect, the invention provides an antibody-drug conjugate comprising an antibody that comprises at least one, at least two, or all three VL HVR sequences selected from (a) HVR-L1 comprising the amino acid sequence of SEQ ID NO: 9; (b) HVR-L2 comprising the amino acid sequence of SEQ ID NO: 10; and (c) HVR-L3 comprising the amino acid sequence of SEQ ID NO: 11. In one embodiment, the antibody comprises (a) HVR-L1 comprising the amino acid sequence of SEQ ID NO: 9; (b) HVR-L2 comprising the amino acid sequence of SEQ ID NO: 10; and (c) HVR-L3 comprising the amino acid sequence of SEQ ID NO: 11.

In another aspect, an antibody-drug conjugate of the invention comprises an antibody comprising (a) a VH domain comprising at least one, at least two, or all three VH HVR sequences selected from (i) HVR-H1 comprising the amino acid sequence of SEQ ID NO: 12, (ii) HVR-H2 comprising the amino acid sequence of SEQ ID NO: 13, and (iii) HVR-H3 comprising an amino acid sequence selected from SEQ ID NO: 14; and (b) a VL domain comprising at least one, at least two, or all three VL HVR sequences selected from (i) HVR-L1 comprising the amino acid sequence of SEQ ID NO: 9, (ii) HVR-L2 comprising the amino acid sequence of SEQ ID NO: 10, and (c) HVR-L3 comprising the amino acid sequence of SEQ ID NO: 11.

In another aspect, the invention provides an antibody-drug conjugate comprising an antibody that comprises (a) HVR-H1 comprising the amino acid sequence of SEQ ID NO: 12; (b) HVR-H2 comprising the amino acid sequence of SEQ ID NO: 13; (c) HVR-H3 comprising the amino acid sequence of SEQ ID NO: 14; (d) HVR-L1 comprising the amino acid sequence of SEQ ID NO: 9; (e) HVR-L2 comprising the amino acid sequence of SEQ ID NO: 10; and (f) HVR-L3 comprising the amino acid sequence of SEQ ID NO: 11.

In any of the above embodiments, an anti-Ly6E antibody of an antibody-drug conjugate is humanized. In one embodiment, an anti-Ly6E antibody comprises HVRs as in any of the above embodiments, and further comprises a human acceptor framework, e.g. a human immunoglobulin framework or a human consensus framework.

In another aspect, an anti-Ly6E antibody of an antibody-drug conjugate comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 8. In certain embodiments, a VH sequence having at least 90%, 91%, 92%, 93%, 94%, 95%, 96%, 97%, 98%, or 99% identity to the amino acid sequence of SEQ ID NO: 8 contains substitutions (e.g., conservative substitutions), insertions, or deletions relative to the reference sequence, but an anti-Ly6E antibody comprising that sequence retains the ability to bind to Ly6E. In certain embodiments, a total of 1 to 10 amino acids have been substituted, inserted and/or deleted in SEQ ID NO: 8. In certain embodiments, a total of 1 to 5 amino acids have been substituted, inserted and/or deleted in SEQ ID NO: 8. In certain embodiments, substitutions, insertions, or deletions occur in regions outside the HVRs (i.e., in the FRs). Optionally, the anti-Ly6E antibody comprises the VH sequence of SEQ ID NO: 8, including post-translational modifications of that sequence. In a particular embodiment, the VH comprises one, two or three HVRs selected from: (a) HVR-H1 comprising the amino acid sequence of SEQ ID NO: 12, (b) HVR-H2 comprising the amino acid sequence of SEQ ID NO: 13, and (c) HVR-H3 comprising the amino acid sequence of SEQ ID NO: 14.

In another aspect, an anti-Ly6E antibody of an antibody-drug conjugate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 7. In certain embodiments, a VL sequence having at least 90%, 91%, 92%, 93%, 94%, 95%, 96%, 97%, 98%, or 99% identity to the amino acid sequence of SEQ ID NO:7 contains substitutions (e.g., conservative substitutions), insertions, or deletions relative to the reference sequence, but an anti-Ly6E antibody comprising that sequence retains the ability to bind to Ly6E. In certain embodiments, a total of 1 to 10 amino acids have been substituted, inserted and/or deleted in SEQ ID NO: 7. In certain embodiments, a total of 1 to 5 amino acids have been substituted, inserted and/or deleted in SEQ ID NO: 7. In certain embodiments, the substitutions, insertions, or deletions occur in regions outside the HVRs (i.e., in the FRs). Optionally, the anti-Ly6E antibody comprises the VL sequence of SEQ ID NO: 7, including post-translational modifications of that sequence. In a particular embodiment, the VL comprises one, two or three HVRs selected from (a) HVR-L1 comprising the amino acid sequence of SEQ ID NO: 9; (b) HVR-L2 comprising the amino acid sequence of SEQ ID NO: 10; and (c) HVR-L3 comprising the amino acid sequence of SEQ ID NO: 11.

In another aspect, an antibody-drug conjugate comprising an anti-Ly6E antibody is provided, wherein the antibody comprises a VH as in any of the embodiments provided above, and a VL as in any of the embodiments provided above.

In one embodiment, an antibody-drug conjugate is provided, wherein the antibody comprises the VH and VL sequences in SEQ ID NO: 8 and SEQ ID NO: 7, respectively, including post-translational modifications of those sequences.

In a further aspect, provided herein are antibody-drug conjugate comprising antibodies that bind to the same epitope as an anti-Ly6E antibody provided herein. For example, in certain embodiments, an immunoconjugate is provided comprising an antibody that binds to the same epitope as an anti-Ly6E antibody comprising a VH sequence of SEQ ID NO: 8 and a VL sequence of SEQ ID NO: 7, respectively.

In a further aspect of the invention, an anti-Ly6E antibody of an antibody-drug conjugate according to any of the above embodiments is a monoclonal antibody, including a human antibody. In one embodiment, an anti-Ly6E antibody of an antibody-drug conjugate is an antibody fragment, e.g., a Fv, Fab, Fab′, scFv, diabody, or F(ab′)₂ fragment. In another embodiment, the antibody is a substantially full length antibody, e.g., an IgG1 antibody, IgG2a antibody or other antibody class or isotype as defined herein. In some embodiments, an immunconjugate (ADC) comprises an anti-Ly6E antibody comprising a heavy chain and a light chain comprising the amino acid sequences of SEQ ID NO: 16 and 15, respectively.

Table of Ly6E Antibody Sequences

| SEQ | | |
|---|---|---|
| ID | | |
| NO | Description | Sequence |
| 7 | anti-Ly6E | DIQMTQSPSS LSASVGDRVT |
| | antibody | ITCSASQGIS NYLNWYQQKP |
| | hu9B12 v12 | GKTVKLLIYY TSNLHSGVPS |
| | light chain | RFSGSGSGTD YTLTISSLQP |
| | variable | EDFATYYCQQ YSELPWTFGQ |
| | region | GTKVEIK |
| 8 | anti-Ly6E | EVQLVESGPA LVKPTQTLTL |
| | antibody | TCTVSGFSLT |
| | hu9B12 v12 | GYSVNWIRQPPGKAL |
| | heavy chain | EWLGMIWGDG STDYNSALKS |
| | variable | RLTISKDTSK NQWLTMTNM |
| | region | DPVDTATYYC ARDYYFNYAS |
| | | WFAYWGQGTL VTVSS |
| 9 | anti-Ly6E | SASQGISNYLN |
| | antibody | |
| | hu9B12 v12 | |
| | HVR-L1 | |
| 10 | anti-Ly6E | YTSNLHS |
| | antibody | |
| | hu9B12 v12 | |
| | HVR-L2 | |
| 11 | anti-Ly6E | QQYSELPWT |
| | antibody | |
| | hu9B12 v12 | |
| | HVR-L3 | |
| 12 | anti-Ly6E | GFSLTGYSVN |
| | antibody | |
| | hu9B12 v12 | |
| | HVR-Hl | |
| 13 | anti-Ly6E | MIYVGDGSTDY NSALKS |
| | antibody | |
| | hu9B12 v12 | |
| | HVR-H2 | |
| 14 | anti-Ly6E | DYYVNYASWFAY |
| | antibody | |
| | hu9B12 v12 | |
| | HVR-H3 | |
| 15 | anti-Ly6E | DIQMTQSPSS LSASVGDRYT |
| | antibody | ITCSASQGIS NYLNWYQQKP |
| | hu9B12 v12 | GKTYKLLIYY TSNLHSGVPS |
| | K149C kappa | RFSGSGSGTD YTLTISSLQP |
| | light chain | EDFATYYCQQ YSELPWTFGQ |
| | | GTKVEIK RTVAAPSVFIF |
| | | PPSDEQLKSG TASVVCLLNN |
| | | FYPREAKVQW CVDNALQSGN |
| | | SQESVTEQDS KDSTYSLSST |
| | | LTLSKADYEK HKVYACEVTH |
| | | QGLSSPYTKS FNRGEC |
| 16 | anti-Ly6E | EVQLVESGPA LVKPTQTLTL |
| | antibody | TCTVSGFSLT GYSVNWIRQP |
| | hu9B12 v12 | PGKALEWLGM IWGDGSTDYN |
| | IgG1 heavy | SALKSRLTIS |
| | chain | KDTSKNQVVL |
| | | TMTNMDPVDT ATYYCARDYY |
| | | FNYASWFAYW |
| | | GQGTLVTVSS |
| | | ASTKGPSVFP LAPSSKSTSG |
| | | GTAALGCLVK DYFPEPVTVS |
| | | WNSGALTSGV HTFPAVLQSS |
| | | GLYSLSSVVT VPSSSLGTQT |
| | | YICNVNHKPS NTKYDKKYEP |
| | | KSCDKTHTCP PCPAPELLGG |
| | | PSVFLFPPKP KDTLMISRTP |
| | | EVTCVVVDVS HEDPEVKFNW |
| | | YVDGVEVHNA KTKPREEQYN |
| | | STYRVVSVLT VLHQDWLNGK |
| | | EYKCKVSNKA LPAPIEKTIS |
| | | KAKGQPREPQ VYTLPPSREE |
| | | MTKNQVSLTC LVKGFYPSDI |
| | | AVEWESNGQP ENNYKTTPPV |
| | | LDSDGSFFLY SKLTVDKSRW |
| | | QQGNVFSCSV MHEALHNHYT |
| | | QKSLSLSPGK |

### Anti-HER2 Antibodies

In certain embodiments, an ADC comprises anti-HER2 antibodies. In one embodiment of the invention, an anti-HER2 antibody of an ADC of the invention comprises a humanized anti-HER2 antibody, e.g., huMAb4D5-1, huMAb4D5-2, huMAb4D5-3, huMAb4D5-4, huMAb4D5-5, huMAb4D5-6, huMAb4D5-7 and huMAb4D5-8, as described in Table 3 of U.S. Pat. No. 5,821,337, which is specifically incorporated by reference herein. Those antibodies contain human framework regions with the complementarity-determining regions of a murine antibody (4D5) that binds to HER2. The humanized antibody huMAb4D5-8 is also referred to as trastuzumab, commercially available under the tradename HERCEPTIN®. In another embodiment of the invention, an anti-HER2 antibody of an ADC of the invention comprises a humanized anti-HER2 antibody, e.g., humanized 2C4, as described in U.S. Pat. No. 7,862,817. An exemplary humanized 2C4 antibody is pertuzumab, commercially available under the tradename PERJETA®.

In another embodiment of the invention, an anti-HER2 antibody of an ADC of the invention comprises a humanized 7C2 anti-HER2 antibody. A humanized 7C2 antibody is an anti-HER2 antibody.

In some embodiments, the invention provides an antibody-drug conjugate comprising an anti-HER2 antibody comprising at least one, two, three, four, five, or six HVRs selected from (a) HVR-H1 comprising the amino acid sequence of SEQ ID NO: 22; (b) HVR-H2 comprising the amino acid sequence of SEQ ID NO: 23, 27, or 28; (c) HVR-H3 comprising the amino acid sequence of SEQ ID NO: 24 or 29; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21. In some embodiments, the invention provides an antibody-drug conjugate comprising an anti-HER2 antibody comprising at least one, two, three, four, five, or six HVRs selected from (a) HVR-H1 comprising the amino acid sequence of SEQ ID NO: 22; (b) HVR-H2 comprising the amino acid sequence of SEQ ID NO: 23; (c) HVR-H3 comprising the amino acid sequence of SEQ ID NO: 24; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21.

In one aspect, the invention provides an antibody-drug conjugate comprising an antibody that comprises at least one, at least two, or all three VH HVR sequences selected from (a) HVR-H1 comprising the amino acid sequence of SEQ ID NO: 22; (b) HVR-H2 comprising the amino acid sequence of SEQ ID NO: 23, 27, or 28; and (c) HVR-H3 comprising the amino acid sequence of SEQ ID NO: 24 or 29. In one aspect, the invention provides an immunoconjugate comprising an antibody that comprises at least one, at least two, or all three VH HVR sequences selected from (a) HVR-H1 comprising the amino acid sequence of SEQ ID NO: 22; (b) HVR-H2 comprising the amino acid sequence of SEQ ID NO: 23; and (c) HVR-H3 comprising the amino acid sequence of SEQ ID NO: 24. In a further embodiment, the antibody comprises (a) HVR-H1 comprising the amino acid sequence of SEQ ID NO: 22; (b) HVR-H2 comprising the amino acid sequence of SEQ ID NO: 23, 27, or 28; and (c) HVR-H3 comprising the amino acid sequence of SEQ ID NO: 24 or 29. In a further embodiment, the antibody comprises (a) HVR-H1 comprising the amino acid sequence of SEQ ID NO: 22; (b) HVR-H2 comprising the amino acid sequence of SEQ ID NO: 23; and (c) HVR-H3 comprising the amino acid sequence of SEQ ID NO: 24.

In another aspect, the invention provides an antibody-drug conjugate comprising an antibody that comprises at least one, at least two, or all three VL HVR sequences selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21. In one embodiment, the antibody comprises (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21.

In another aspect, an antibody-drug conjugate of the invention comprises an antibody comprising (a) a VH domain comprising at least one, at least two, or all three VH HVR sequences selected from (i) HVR-H1 comprising the amino acid sequence of SEQ ID NO: 22, (ii) HVR-H2 comprising the amino acid sequence of SEQ ID NO: 23, 27, or 28, and (iii) HVR-H3 comprising an amino acid sequence selected from SEQ ID NO: 24 or 29; and (b) a VL domain comprising at least one, at least two, or all three VL HVR sequences selected from (i) HVR-L1 comprising the amino acid sequence of SEQ ID NO: 19, (ii) HVR-L2 comprising the amino acid sequence of SEQ ID NO: 20, and (c) HVR-L3 comprising the amino acid sequence of SEQ ID NO: 21. In another aspect, an antibody-drug conjugate of the invention comprises an antibody comprising (a) a VH domain comprising at least one, at least two, or all three VH HVR sequences selected from (i) HVR-H1 comprising the amino acid sequence of SEQ ID NO: 22, (ii) HVR-H2 comprising the amino acid sequence of SEQ ID NO: 23, and (iii) HVR-H3 comprising an amino acid sequence selected from SEQ ID NO: 24; and (b) a VL domain comprising at least one, at least two, or all three VL HVR sequences selected from (i) HVR-L1 comprising the amino acid sequence of SEQ ID NO: 19, (ii) HVR-L2 comprising the amino acid sequence of SEQ ID NO: 20, and (c) HVR-L3 comprising the amino acid sequence of SEQ ID NO: 21.

In another aspect, the invention provides an antibody-drug conjugate comprising an antibody that comprises (a) HVR-H1 comprising the amino acid sequence of SEQ ID NO: 22; (b) HVR-H2 comprising the amino acid sequence of SEQ ID NO: 23, 27, or 28; (c) HVR-H3 comprising the amino acid sequence of SEQ ID NO: 24 or 29; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21. In another aspect, the invention provides an antibody-drug conjugate comprising an antibody that comprises (a) HVR-H1 comprising the amino acid sequence of SEQ ID NO: 22; (b) HVR-H2 comprising the amino acid sequence of SEQ ID NO: 23; (c) HVR-H3 comprising the amino acid sequence of SEQ ID NO: 24; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21.

In any of the above embodiments, an anti-HER2 antibody of an antibody-drug conjugate is humanized. In one embodiment, an anti-HER2 antibody of an antibody-drug conjugate comprises HVRs as in any of the above embodiments, and further comprises a human acceptor framework, e.g. a human immunoglobulin framework or a human consensus framework.

In another aspect, an anti-HER2 antibody of an antibody-drug conjugate comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 18. In certain embodiments, a VH sequence having at least 90%, 91%, 92%, 93%, 94%, 95%, 96%, 97%, 98%, or 99% identity to the amino acid sequence of SEQ ID NO: 18 contains substitutions (e.g., conservative substitutions), insertions, or deletions relative to the reference sequence, but an anti-HER2 antibody comprising that sequence retains the ability to bind to HER2. In certain embodiments, a total of 1 to 10 amino acids have been substituted, inserted and/or deleted in SEQ ID NO: 18. In certain embodiments, a total of 1 to 5 amino acids have been substituted, inserted and/or deleted in SEQ ID NO: 18. In certain embodiments, substitutions, insertions, or deletions occur in regions outside the HVRs (i.e., in the FRs). Optionally, the anti-HER2 antibody comprises the VH sequence of SEQ ID NO: 18, including post-translational modifications of that sequence. In a particular embodiment, the VH comprises one, two or three HVRs selected from: (a) HVR-H1 comprising the amino acid sequence of SEQ ID NO: 22, (b) HVR-H2 comprising the amino acid sequence of SEQ ID NO: 23, and (c) HVR-H3 comprising the amino acid sequence of SEQ ID NO: 24.

In another aspect, an anti-HER2 antibody of an antibody-drug conjugate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 17. In certain embodiments, a VL sequence having at least 90%, 91%, 92%, 93%, 94%, 95%, 96%, 97%, 98%, or 99% identity to the amino acid sequence of SEQ ID NO: 17 contains substitutions (e.g., conservative substitutions), insertions, or deletions relative to the reference sequence, but an anti-HER2 antibody comprising that sequence retains the ability to bind to HER2. In certain embodiments, a total of 1 to 10 amino acids have been substituted, inserted and/or deleted in SEQ ID NO: 17. In certain embodiments, a total of 1 to 5 amino acids have been substituted, inserted and/or deleted in SEQ ID NO: 17. In certain embodiments, the substitutions, insertions, or deletions occur in regions outside the HVRs (i.e., in the FRs). Optionally, the anti-HER2 antibody comprises the VL sequence of SEQ ID NO: 17, including post-translational modifications of that sequence. In a particular embodiment, the VL comprises one, two or three HVRs selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21.

In another aspect, an antibody-drug conjugate comprising an anti-HER2 antibody is provided, wherein the antibody comprises a VH as in any of the embodiments provided above, and a VL as in any of the embodiments provided above.

In one embodiment, an antibody-drug conjugate comprising an antibody is provided, wherein the antibody comprises the VH and VL sequences in SEQ ID NO: 18 and SEQ ID NO: 17, respectively, including post-translational modifications of those sequences.

In one embodiment, an antibody-drug conjugate comprising an antibody is provided, wherein the antibody comprises the humanized 7C2.v2.2.LA (hu7C2) K149C kappa light chain sequence of SEQ ID NO: 30

In one embodiment, an antibody-drug conjugate comprising an antibody is provided, wherein the antibody comprises the Hu7C2 A118C IgG1 heavy chain sequence of SEQ ID NO: 31

In a further aspect, provided herein are antibody-drug conjugates comprising antibodies that bind to the same epitope as an anti-HER2 antibody provided herein. For example, in certain embodiments, an immunoconjugate is provided, comprising an antibody that binds to the same epitope as an anti-HER2 antibody comprising a VH sequence of SEQ ID NO: 18 and a VL sequence of SEQ ID NO: 17, respectively.

In a further aspect of the invention, an anti-HER2 antibody of an antibody-drug conjugate according to any of the above embodiments is a monoclonal antibody, including a human antibody. In one embodiment, an anti-HER2 antibody of an immunoconjugate is an antibody fragment, e.g., a Fv, Fab, Fab′, scFv, diabody, or F(ab′)₂ fragment. In another embodiment, an immunoconjugate comprises an antibody that is a substantially full length antibody, e.g., an IgGl antibody, IgG2a antibody or other antibody class or isotype as defined herein.

Table of humanized 7C2 anti-HER2
antibody sequences

| SEQ | | |
|---|---|---|
| ID | | |
| NO | Description | Sequence |
| 17 | Humanized | DIVMTQSPDS LAVSLGERAT INCRASQSVS |
| | 7C2.V2.2.LA | GSRFTYMHWY QQKPGQPPKL LIKYASILES |
| | (“hu7C2”) | GVPDRFSGSG SGTDFTLTIS SLQAEDVAVY |
| | light | YCQHSWEIPP WTFGQGTKVE IK |
| | chain | |
| | variable | |
| | region | |
| 18 | Humanized | EVQLVQSGAE VKKPGASVKV SCKASGYSFT |
| | 7C2.V2.2.LA | GYWMNWVRQA PGQGLEWIGM IHPLDAEIRA |
| | (“hu7C2”) | NQKFRDRVTI TVDTSTSTAY LELSSLRSED |
| | heavy | TAVYYCARGT YDGGFEYWGQ GTLVTVSS |
| | chain | |
| | variable | |
| | region | |
| 19 | hu7C2 | RASQSVSGSRFTYMH |
| | HVR-L1 | |
| 20 | hu7C2 HVR- | YASILES |
| | L2 | |
| 21 | hu7C2 HVR- | QHSWEIPPWT |
| | L3 | |
| 22 | hu7C2 HVR- | GYWMN |
| | H1 | |
| 23 | hu7C2 HVR- | MIHPLDAEIRANQKFRD |
| | H2 | |
| 24 | hu7C2 HVR- | GTYDGGFEY |
| | H3 | |
| 25 | Humanized | DIVMTQSPDS LAVSLGERAT INCRASQSVS |
| | 7C2.V2. | GSRFTYMHWY QQKPGQPPKL LIKYASILES |
| | 2.LA | GVPDRFSGSG SGTDFTLTIS SLQAEDVAVY |
| | (hu7C2) | YCQHSWEIPP WTFGQGTKVE IKRTVAAPSV |
| | kappa | FIFPPSDEQL KSGTASVVCL LNNFYPREAK |
| | light | VQWKVDNALQ SGNSQESVTE QDSKDSTYSL |
| | chain | SSTLTLSKAD YEKHKVYACE VTHQGLSSPV |
| | | TKSFNRGEC |
| 26 | Humanized | EVQLVQSGAE VKKPGASVKV SCKASGYSFT |
| | 7C2.V2. | GYWMNWVRQA PGQGLEWIGM IHPLDAEIRA |
| | 2.LA | NQKFRDRVTI TVDTSTSTAY LELSSLRSED |
| | (hu7C2) | TAVYYCARGT YDGGFEYWGQ GTLVTVSSAS |
| | IgG1 | TKGPSVFPLA PSSKSTSGGT AALGCLVKDY |
| | heavy | FPEPVTVSWN SGALTSGVHT FPAVLQSSGL |
| | chain | YSLSSVVTVP SSSLGTQTYI CNVNHKPSNT |
| | | KVDKKVEPKS CDKTHTCPPC PAPELLGGPS |
| | | VFLFPPKPKD TLMISRTPEV TCVVVDVSHE |
| | | DPEVKFNWYV DGVEVHNAKT KPREEQYNST |
| | | YRVVSVLTVL HQDWLNGKEY KCKVSNKALP |
| | | APIEKTISKA KGQPREPQVY TLPPSREEMT |
| | | KNQVSLTCLV KGFYPSDIAV EWESNGQPEN |
| | | NYKTTPPVLD SDGSFFLYSK LTVDKSRWQQ |
| | | GNVFSCSVMH EALHNHYTQK SLSLSPGK |
| 27 | Hu7C2. | MIHPMDSEIRANQKFRD |
| | V2.1.S53M | |
| | HVR-H2 | |
| 28 | Hu7C2. | MIHPLDSEIRANQKFRD |
| | V2.1.S53L | |
| | HVR-H2 | |
| 29 | Hu7C2. | GTYDGGFKY |
| | V2.1.E101K | |
| | HVR-H3 | |
| 30 | Humanized | DIVMTQSPDS LAVSLGERAT INCRASQSVS |
| | 7C2.V2. | GSRFTYMHWT QQKPGQPPKL LIKYASILES |
| | 2.LA | GVPDRFSGSG SGTDFTLTIS SLQAEDVAVY |
| | (hu7C2) | YCQHSWEIPP WTFGQGTKVE IKRTVAAPSV |
| | K149C | FIFPPSDEQL KSGTASVVCL LNNFYPREAK |
| | kappa | VQWCVDNALQ SGNSQESYTE QDSKDSTYSL |
| | light | SSTLTLSKAD YEKHKVYACE YTHQGLSSPV |
| | chain | TKSFNRGEC |
| 31 | Humanized | EVQLVQSGAE VKKPGASVKV SCKASGYSFT |
| | 7C2.V2. | GYWMNWVRQA PGQGLEWIGM IHPLDAEIRA |
| | 2.LA | NQKFRDRVTI TVDTSTSTAY LELSSLRSED |
| | (hu7C2) | TAVYYCARGT YDGGFEYWGQ GTLYTVSSCS |
| | A118C | TKGPSVFPLA PSSKSTSGGT AALGCLVKDY |
| | IgG1 | FPEPYTVSWN SGALTSGVHT FPAVLQSSGL |
| | heavy | YSLSSVVTVP SSSLGTQTYI CNVNHKPSNT |
| | chain | KVDKKVEPKS CDKTHTCPPC PAPELLGGPS |
| | | VFLFPPKPKD TLMISRTPEV TCVVVDVSHE |
| | | DPEVKFNWYV DGVEVHNAKT KPREEQYNST |
| | | YRVVSVLTVL HQDWLNGKEY KCKVSNKALP |
| | | APIEKTISKA KGQPREPQYT TLPPSREEMT |
| | | KNQVSLTCLV KGFYPSDIAV EWESNGQPEN |
| | | NYKTTPPVLD SDGSFFLYSK LTVDKSRWQQ |
| | | GNVFSCSVMH EALHNHYTQK SLSLSPGK |

### Anti-MUC16 Antibodies

In certain embodiments, an ADC comprises anti-MUC16 antibodies.

In some embodiments, the invention provides an antibody-drug conjugate comprising an anti-MUC16 antibody comprising at least one, two, three, four, five, or six HVRs selected from (a) HVR-H1 comprising the amino acid sequence of SEQ ID NO: 35; (b) HVR-H2 comprising the amino acid sequence of SEQ ID NO: 36; (c) HVR-H3 comprising the amino acid sequence of SEQ ID NO: 37; (d) HVR-L1 comprising the amino acid sequence of SEQ ID NO: 32; (e) HVR-L2 comprising the amino acid sequence of SEQ ID NO: 33 and (f) HVR-L3 comprising the amino acid sequence of SEQ ID NO: 34.

In one aspect, the invention provides an antibody-drug conjugate comprising an antibody that comprises at least one, at least two, or all three VH HVR sequences selected from (a) HVR-H1 comprising the amino acid sequence of SEQ ID NO: 35; (b) HVR-H2 comprising the amino acid sequence of SEQ ID NO: 36; (c) HVR-H3 comprising the amino acid sequence of SEQ ID NO: 37. In a further embodiment, the antibody comprises (a) HVR-H1 comprising the amino acid sequence of SEQ ID NO: 35; (b) HVR-H2 comprising the amino acid sequence of SEQ ID NO: 36; (c) HVR-H3 comprising the amino acid sequence of SEQ ID NO: 37.

In another aspect, the invention provides an antibody-drug conjugate comprising an antibody that comprises at least one, at least two, or all three VL HVR sequences selected from (a) HVR-L1 comprising the amino acid sequence of SEQ ID NO: 32; (b) HVR-L2 comprising the amino acid sequence of SEQ ID NO: 33; and (c) HVR-L3 comprising the amino acid sequence of SEQ ID NO: 34. In one embodiment, the antibody comprises (a) HVR-L1 comprising the amino acid sequence of SEQ ID NO: 32; (b) HVR-L2 comprising the amino acid sequence of SEQ ID NO: 33; and (c) HVR-L3 comprising the amino acid sequence of SEQ ID NO: 34.

In another aspect, an antibody-drug conjugate of the invention comprises an antibody comprising (a) a VH domain comprising at least one, at least two, or all three VH HVR sequences selected from (i) HVR-H1 comprising the amino acid sequence of SEQ ID NO: 35, (ii) HVR-H2 comprising the amino acid sequence of SEQ ID NO: 36, and (iii) HVR-H3 comprising an amino acid sequence selected from SEQ ID NO: 37; and (b) a VL domain comprising at least one, at least two, or all three VL HVR sequences selected from (i) HVR-L1 comprising the amino acid sequence of SEQ ID NO: 32, (ii) HVR-L2 comprising the amino acid sequence of SEQ ID NO: 33, and (c) HVR-L3 comprising the amino acid sequence of SEQ ID NO: 34.

In another aspect, the invention provides an antibody-drug conjugate comprising an antibody that comprises (a) HVR-H1 comprising the amino acid sequence of SEQ ID NO: 35 (b) HVR-H2 comprising the amino acid sequence of SEQ ID NO: 36; (c) HVR-H3 comprising the amino acid sequence of SEQ ID NO: 37; (d) HVR-L1 comprising the amino acid sequence of SEQ ID NO: 32; (e) HVR-L2 comprising the amino acid sequence of SEQ ID NO: 33; and (f) HVR-L3 comprising the amino acid sequence of SEQ ID NO: 34.

In any of the above embodiments, an anti-MUC16 antibody of an antibody-drug conjugate is humanized. In one embodiment, an anti-MUC16 antibody comprises HVRs as in any of the above embodiments, and further comprises a human acceptor framework, e.g. a human immunoglobulin framework or a human consensus framework.

In another aspect, an anti-MUC16 antibody of an antibody-drug conjugate comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 39. In certain embodiments, a VH sequence having at least 90%, 91%, 92%, 93%, 94%, 95%, 96%, 97%, 98%, or 99% identity to the amino acid sequence of SEQ ID NO: 39 contains substitutions (e.g., conservative substitutions), insertions, or deletions relative to the reference sequence, but an anti-MUC16 antibody comprising that sequence retains the ability to bind to MUC16. In certain embodiments, a total of 1 to 10 amino acids have been substituted, inserted and/or deleted in SEQ ID NO: 39. In certain embodiments, a total of 1 to 5 amino acids have been substituted, inserted and/or deleted in SEQ ID NO: 39. In certain embodiments, substitutions, insertions, or deletions occur in regions outside the HVRs (i.e., in the FRs). Optionally, the anti-MUC16 antibody comprises the VH sequence of SEQ ID NO: 39, including post-translational modifications of that sequence. In a particular embodiment, the VH comprises one, two or three HVRs selected from: (a) HVR-H1 comprising the amino acid sequence of SEQ ID NO: 35, (b) HVR-H2 comprising the amino acid sequence of SEQ ID NO: 36, and (c) HVR-H3 comprising the amino acid sequence of SEQ ID NO: 37.

In another aspect, an anti-MUC16 antibody of an antibody-drug conjugate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 38. In certain embodiments, a VL sequence having at least 90%, 91%, 92%, 93%, 94%, 95%, 96%, 97%, 98%, or 99% identity to the amino acid sequence of SEQ ID NO:38 contains substitutions (e.g., conservative substitutions), insertions, or deletions relative to the reference sequence, but an anti-MUC16 antibody comprising that sequence retains the ability to bind to MUC16. In certain embodiments, a total of 1 to 10 amino acids have been substituted, inserted and/or deleted in SEQ ID NO: 38. In certain embodiments, a total of 1 to 5 amino acids have been substituted, inserted and/or deleted in SEQ ID NO: 38. In certain embodiments, the substitutions, insertions, or deletions occur in regions outside the HVRs (i.e., in the FRs). Optionally, the anti-MUC16 antibody comprises the VL sequence of SEQ ID NO: 38, including post-translational modifications of that sequence. In a particular embodiment, the VL comprises one, two or three HVRs selected from (a) HVR-L1 comprising the amino acid sequence of SEQ ID NO: 32; (b) HVR-L2 comprising the amino acid sequence of SEQ ID NO: 33; and (c) HVR-L3 comprising the amino acid sequence of SEQ ID NO: 34.

In another aspect, an antibody-drug conjugate comprising an anti-MUC16 antibody is provided, wherein the antibody comprises a VH as in any of the embodiments provided above, and a VL as in any of the embodiments provided above.

In one embodiment, an antibody-drug conjugate is provided, wherein the antibody comprises the VH and VL sequences in SEQ ID NO: 39 and SEQ ID NO: 38, respectively, including post-translational modifications of those sequences.

In a further aspect, provided herein are antibody-drug conjugate comprising antibodies that bind to the same epitope as an anti-MUC16 antibody provided herein. For example, in certain embodiments, an immunoconjugate is provided comprising an antibody that binds to the same epitope as an anti-MUC16 antibody comprising a VH sequence of SEQ ID NO: 39 and a VL sequence of SEQ ID NO: 38, respectively.

In a further aspect of the invention, an anti-MUC16 antibody of an antibody-drug conjugate according to any of the above embodiments is a monoclonal antibody, including a human antibody. In one embodiment, an anti-MUC16 antibody of an antibody-drug conjugate is an antibody fragment, e.g., a Fv, Fab, Fab′, scFv, diabody, or F(ab′)₂ fragment. In another embodiment, the antibody is a substantially full length antibody, e.g., an IgG1 antibody, IgG2a antibody or other antibody class or isotype as defined herein.

Table of MUC16 Antibody Sequences

| SEQ | | |
|---|---|---|
| ID | Descrip- | |
| NO | tion | Sequence |
| 32 | Anti- | KASDLIHNWLA |
| | Mucl6 | |
| | antibody | |
| | HVR-L1 | |
| 33 | Anti- | YGATSLET |
| | Mucl6 | |
| | antibody | |
| | HVR-L2 | |
| 34 | Anti- | QQYWTTPFT |
| | Mucl6 | |
| | antibody | |
| | HVR-L3 | |
| 35 | Anti- | GYSITNDYAW N |
| | Mucl6 | |
| | antibody | |
| | HVR-H1 | |
| 36 | Anti- | GYISYSGYTT YNPSLKS |
| | Mucl6 | |
| | antibody | |
| | HVR-H2 | |
| 37 | Anti- | ARWASGLDY |
| | Mucl6 | |
| | antibody | |
| | HVR-H3 | |
| 38 | Anti- | DIQMTQSPSS LSASVGDRYT ITCKASDLIH |
| | Mucl6 | NWLAWYQQKP GKAPKLLIYG ATSLETGVPS |
| | antibody | RFSGSGSGTD FTLTISSLQP EDFATYYCQQ |
| | light | YWTTPFTFGQ GTKVEIKR |
| | chain | |
| | variable | |
| | region | |
| 39 | Anti- | EVQLVESGGG LVQPGGSLRL SCAASGYSIT |
| | Mucl6 | NDYAWNWVRQ APGKGLEWVG YISYSGYTTY |
| | antibody | NPSLKSRFTI SRDTSKNTLY LQMNSLRAED |
| | heavy | TAVYYCARWA SGLDYWGQGT LYTVSS |
| | chain | |
| | variable | |
| | region | |

### Anti-STEAP-1 Antibodies

In certain embodiments, an ADC comprises anti-STEAP-1 antibodies.

In some embodiments, the invention provides an antibody-drug conjugate comprising an anti-STEAP-1 antibody comprising at least one, two, three, four, five, or six HVRs selected from (a) HVR-H1 comprising the amino acid sequence of SEQ ID NO: 40; (b) HVR-H2 comprising the amino acid sequence of SEQ ID NO: 41; (c) HVR-H3 comprising the amino acid sequence of SEQ ID NO: 42; (d) HVR-L1 comprising the amino acid sequence of SEQ ID NO: 43; (e) HVR-L2 comprising the amino acid sequence of SEQ ID NO: 44 and (f) HVR-L3 comprising the amino acid sequence of SEQ ID NO: 45.

In one aspect, the invention provides an antibody-drug conjugate comprising an antibody that comprises at least one, at least two, or all three VH HVR sequences selected from (a) HVR-H1 comprising the amino acid sequence of SEQ ID NO: 40; (b) HVR-H2 comprising the amino acid sequence of SEQ ID NO: 41; (c) HVR-H3 comprising the amino acid sequence of SEQ ID NO: 42. In a further embodiment, the antibody comprises (a) HVR-H1 comprising the amino acid sequence of SEQ ID NO: 40; (b) HVR-H2 comprising the amino acid sequence of SEQ ID NO: 41; (c) HVR-H3 comprising the amino acid sequence of SEQ ID NO: 42.

In another aspect, the invention provides an antibody-drug conjugate comprising an antibody that comprises at least one, at least two, or all three VL HVR sequences selected from (a) HVR-L1 comprising the amino acid sequence of SEQ ID NO: 43; (b) HVR-L2 comprising the amino acid sequence of SEQ ID NO: 44; and (c) HVR-L3 comprising the amino acid sequence of SEQ ID NO: 45. In one embodiment, the antibody comprises (a) HVR-L1 comprising the amino acid sequence of SEQ ID NO: 43; (b) HVR-L2 comprising the amino acid sequence of SEQ ID NO: 44; and (c) HVR-L3 comprising the amino acid sequence of SEQ ID NO: 45.

In another aspect, an antibody-drug conjugate of the invention comprises an antibody comprising (a) a VH domain comprising at least one, at least two, or all three VH HVR sequences selected from (i) HVR-H1 comprising the amino acid sequence of SEQ ID NO: 40, (ii) HVR-H2 comprising the amino acid sequence of SEQ ID NO: 41, and (iii) HVR-H3 comprising an amino acid sequence selected from SEQ ID NO: 42; and (b) a VL domain comprising at least one, at least two, or all three VL HVR sequences selected from (i) HVR-L1 comprising the amino acid sequence of SEQ ID NO: 43, (ii) HVR-L2 comprising the amino acid sequence of SEQ ID NO: 44, and (c) HVR-L3 comprising the amino acid sequence of SEQ ID NO: 45.

In another aspect, the invention provides an antibody-drug conjugate comprising an antibody that comprises (a) HVR-H1 comprising the amino acid sequence of SEQ ID NO: 40 (b) HVR-H2 comprising the amino acid sequence of SEQ ID NO: 41; (c) HVR-H3 comprising the amino acid sequence of SEQ ID NO: 42; (d) HVR-L1 comprising the amino acid sequence of SEQ ID NO: 43; (e) HVR-L2 comprising the amino acid sequence of SEQ ID NO: 44; and (f) HVR-L3 comprising the amino acid sequence of SEQ ID NO: 45.

In any of the above embodiments, an anti-STEAP-1 antibody of an antibody-drug conjugate is humanized. In one embodiment, an anti-STEAP-1 antibody comprises HVRs as in any of the above embodiments, and further comprises a human acceptor framework, e.g. a human immunoglobulin framework or a human consensus framework.

In another aspect, an anti-STEAP-1 antibody of an antibody-drug conjugate comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 46. In certain embodiments, a VH sequence having at least 90%, 91%, 92%, 93%, 94%, 95%, 96%, 97%, 98%, or 99% identity to the amino acid sequence of SEQ ID NO: 46 contains substitutions (e.g., conservative substitutions), insertions, or deletions relative to the reference sequence, but an anti-STEAP-1 antibody comprising that sequence retains the ability to bind to STEAP-1. In certain embodiments, a total of 1 to 10 amino acids have been substituted, inserted and/or deleted in SEQ ID NO: 46.

In certain embodiments, a total of 1 to 5 amino acids have been substituted, inserted and/or deleted in SEQ ID NO: 46. In certain embodiments, substitutions, insertions, or deletions occur in regions outside the HVRs (i.e., in the FRs). Optionally, the anti-STEAP-1 antibody comprises the VH sequence of SEQ ID NO: 46, including post-translational modifications of that sequence. In a particular embodiment, the VH comprises one, two or three HVRs selected from: (a) HVR-H1 comprising the amino acid sequence of SEQ ID NO: 40, (b) HVR-H2 comprising the amino acid sequence of SEQ ID NO: 41, and (c) HVR-H3 comprising the amino acid sequence of SEQ ID NO: 42.

In another aspect, an anti-STEAP-1 antibody of an antibody-drug conjugate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 47. In certain embodiments, a VL sequence having at least 90%, 91%, 92%, 93%, 94%, 95%, 96%, 97%, 98%, or 99% identity to the amino acid sequence of SEQ ID NO: 47 contains substitutions (e.g., conservative substitutions), insertions, or deletions relative to the reference sequence, but an anti-STEAP-1 antibody comprising that sequence retains the ability to bind to STEAP-1. In certain embodiments, a total of 1 to 10 amino acids have been substituted, inserted and/or deleted in SEQ ID NO: 47 In certain embodiments, a total of 1 to 5 amino acids have been substituted, inserted and/or deleted in SEQ ID NO: 47. In certain embodiments, the substitutions, insertions, or deletions occur in regions outside the HVRs (i.e., in the FRs). Optionally, the anti-STEAP-1 antibody comprises the VL sequence of SEQ ID NO: 47, including post-translational modifications of that sequence. In a particular embodiment, the VL comprises one, two or three HVRs selected from (a) HVR-L1 comprising the amino acid sequence of SEQ ID NO: 43; (b) HVR-L2 comprising the amino acid sequence of SEQ ID NO: 44; and (c) HVR-L3 comprising the amino acid sequence of SEQ ID NO: 45.

In another aspect, an antibody-drug conjugate comprising an anti-STEAP-1 antibody is provided, wherein the antibody comprises a VH as in any of the embodiments provided above, and a VL as in any of the embodiments provided above.

In one embodiment, an antibody-drug conjugate is provided, wherein the antibody comprises the VH and VL sequences in SEQ ID NO: 46 and SEQ ID NO: 47, respectively, including post-translational modifications of those sequences.

In a further aspect, provided herein are antibody-drug conjugate comprising antibodies that bind to the same epitope as an anti-STEAP-1 antibody provided herein. For example, in certain embodiments, an immunoconjugate is provided comprising an antibody that binds to the same epitope as an anti-STEAP-1 antibody comprising a VH sequence of SEQ ID NO: 46 and a VL sequence of SEQ ID NO: 47, respectively.

In a further aspect of the invention, an anti-STEAP-1 antibody of an antibody-drug conjugate according to any of the above embodiments is a monoclonal antibody, including a human antibody. In one embodiment, an anti-STEAP-1 antibody of an antibody-drug conjugate is an antibody fragment, e.g., a Fv, Fab, Fab′, scFv, diabody, or F(ab′)₂ fragment. In another embodiment, the antibody is a substantially full length antibody, e.g., an IgGl antibody, IgG2a antibody or other antibody class or isotype as defined herein.

Table of STEAP Antibody Sequences

| SEQ | | |
|---|---|---|
| ID | Descrip- | |
| NO | tion | Sequence |
| 40 | Anti- | GYSITSDYAW N |
| | STEAP-1 | |
| | HVR-H1 | |
| 41 | Anti- | GYISNSGSTS YNPSLKS |
| | STEAP-1 | |
| | HVR-H2 | |
| 42 | Anti- | ERNYDYDDYY YAMDY |
| | STEAP-1 | |
| | HVR-H3 | |
| 43 | Anti- | KSSQSLLYRS NQKNYLA |
| | STEAP-1 | |
| | HVR-L1 | |
| 44 | Anti- | WASTRES |
| | STEAP-1 | |
| | HVR-L2 | |
| 45 | Anti- | QQYYNYPRT |
| | STEAP-1 | |
| | HVR-L3 | |
| 46 | Anti- | EVQLVESGGG LVQPGGSLRL SCAVSGYSIT |
| | STEAP-1 | SDYAWNWVRQ APGKGLEWVG YISNSGSTSY |
| | heavy | NPSLKSRFTI SRDTSKNTLY LQMNSLRAED |
| | chain | TAVYYCARER NYDYDDYYYA MDYWGQGTLV |
| | variable | TVSS |
| | region | |
| 47 | Anti- | DIQMTQSPSS LSASVGDRVT ITCKSSQSLL |
| | STEAP-1 | YRSNQKNYLA WYQQKPGKAP KLLIYWASTR |
| | light | ESGVPSRFSG SGSGTDFTLT ISSLQPEDFA |
| | chain | TYYCQQYYNY PRTFGQGTKV EIK |
| | variable | |
| | region | |

### Anti-NaPi2b Antibodies

In certain embodiments, an ADC comprises anti-NaPi2b antibodies. In some embodiments, the invention provides an antibody-drug conjugate comprising an anti-NaPi2b antibody comprising at least one, two, three, four, five, or six HVRs selected from (a) HVR-H1 comprising the amino acid sequence of SEQ ID NO: 48; (b) HVR-H2 comprising the amino acid sequence of SEQ ID NO: 49; (c) HVR-H3 comprising the amino acid sequence of SEQ ID NO: 50; (d) HVR-L1 comprising the amino acid sequence of SEQ ID NO: 51; (e) HVR-L2 comprising the amino acid sequence of SEQ ID NO: 52 and (f) HVR-L3 comprising the amino acid sequence of SEQ ID NO: 53.

In one aspect, the invention provides an antibody-drug conjugate comprising an antibody that comprises at least one, at least two, or all three VH HVR sequences selected from (a) HVR-H1 comprising the amino acid sequence of SEQ ID NO: 48; (b) HVR-H2 comprising the amino acid sequence of SEQ ID NO: 49; (c) HVR-H3 comprising the amino acid sequence of SEQ ID NO: 50. In a further embodiment, the antibody comprises (a) HVR-H1 comprising the amino acid sequence of SEQ ID NO: 48; (b) HVR-H2 comprising the amino acid sequence of SEQ ID NO: 49; (c) HVR-H3 comprising the amino acid sequence of SEQ ID NO: 50.

In another aspect, the invention provides an antibody-drug conjugate comprising an antibody that comprises at least one, at least two, or all three VL HVR sequences selected from (a) HVR-L1 comprising the amino acid sequence of SEQ ID NO: 51; (b) HVR-L2 comprising the amino acid sequence of SEQ ID NO: 52; and (c) HVR-L3 comprising the amino acid sequence of SEQ ID NO: 53. In one embodiment, the antibody comprises (a) HVR-L1 comprising the amino acid sequence of SEQ ID NO: 51; (b) HVR-L2 comprising the amino acid sequence of SEQ ID NO: 52; and (c) HVR-L3 comprising the amino acid sequence of SEQ ID NO: 53.

In another aspect, an antibody-drug conjugate of the invention comprises an antibody comprising (a) a VH domain comprising at least one, at least two, or all three VH HVR sequences selected from (i) HVR-H1 comprising the amino acid sequence of SEQ ID NO: 48, (ii) HVR-H2 comprising the amino acid sequence of SEQ ID NO: 49, and (iii) HVR-H3 comprising an amino acid sequence selected from SEQ ID NO: 50; and (b) a VL domain comprising at least one, at least two, or all three VL HVR sequences selected from (i) HVR-L1 comprising the amino acid sequence of SEQ ID NO: 51, (ii) HVR-L2 comprising the amino acid sequence of SEQ ID NO: 52, and (c) HVR-L3 comprising the amino acid sequence of SEQ ID NO: 53.

In another aspect, the invention provides an antibody-drug conjugate comprising an antibody that comprises (a) HVR-H1 comprising the amino acid sequence of SEQ ID NO: 48 (b) HVR-H2 comprising the amino acid sequence of SEQ ID NO: 49; (c) HVR-H3 comprising the amino acid sequence of SEQ ID NO: 50; (d) HVR-L1 comprising the amino acid sequence of SEQ ID NO: 51; (e) HVR-L2 comprising the amino acid sequence of SEQ ID NO: 52; and (f) HVR-L3 comprising the amino acid sequence of SEQ ID NO: 53.

In any of the above embodiments, an anti-NaPi2b antibody of an antibody-drug conjugate is humanized. In one embodiment, an anti-NaPi2b antibody comprises HVRs as in any of the above embodiments, and further comprises a human acceptor framework, e.g. a human immunoglobulin framework or a human consensus framework.

In another aspect, an anti-NaPi2b antibody of an antibody-drug conjugate comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 54. In certain embodiments, a VH sequence having at least 90%, 91%, 92%, 93%, 94%, 95%, 96%, 97%, 98%, or 99% identity to the amino acid sequence of SEQ ID NO: 54 contains substitutions (e.g., conservative substitutions), insertions, or deletions relative to the reference sequence, but an anti-NaPi2b antibody comprising that sequence retains the ability to bind to NaPi2b. In certain embodiments, a total of 1 to 10 amino acids have been substituted, inserted and/or deleted in SEQ ID NO: 54. In certain embodiments, a total of 1 to 5 amino acids have been substituted, inserted and/or deleted in SEQ ID NO: 54. In certain embodiments, substitutions, insertions, or deletions occur in regions outside the HVRs (i.e., in the FRs). Optionally, the anti-NaPi2b antibody comprises the VH sequence of SEQ ID NO: 54, including post-translational modifications of that sequence. In a particular embodiment, the VH comprises one, two or three HVRs selected from: (a) HVR-H1 comprising the amino acid sequence of SEQ ID NO: 48, (b) HVR-H2 comprising the amino acid sequence of SEQ ID NO: 49, and (c) HVR-H3 comprising the amino acid sequence of SEQ ID NO: 50.

In another aspect, an anti-NaPi2b antibody of an antibody-drug conjugate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 55. In certain embodiments, a VL sequence having at least 90%, 91%, 92%, 93%, 94%, 95%, 96%, 97%, 98%, or 99% identity to the amino acid sequence of SEQ ID NO: 55 contains substitutions (e.g., conservative substitutions), insertions, or deletions relative to the reference sequence, but an anti-NaPi2b antibody comprising that sequence retains the ability to bind to anti-NaPi2b. In certain embodiments, a total of 1 to 10 amino acids have been substituted, inserted and/or deleted in SEQ ID NO: 55. In certain embodiments, a total of 1 to 5 amino acids have been substituted, inserted and/or deleted in SEQ ID NO: 55. In certain embodiments, the substitutions, insertions, or deletions occur in regions outside the HVRs (i.e., in the FRs). Optionally, the anti-NaPi2b antibody comprises the VL sequence of SEQ ID NO: 55, including post-translational modifications of that sequence. In a particular embodiment, the VL comprises one, two or three HVRs selected from (a) HVR-L1 comprising the amino acid sequence of SEQ ID NO: 51; (b) HVR-L2 comprising the amino acid sequence of SEQ ID NO: 52; and (c) HVR-L3 comprising the amino acid sequence of SEQ ID NO: 53.

In another aspect, an antibody-drug conjugate comprising an anti-NaPi2b antibody is provided, wherein the antibody comprises a VH as in any of the embodiments provided above, and a VL as in any of the embodiments provided above.

In one embodiment, an antibody-drug conjugate is provided, wherein the antibody comprises the VH and VL sequences in SEQ ID NO: 54 and SEQ ID NO: 55, respectively, including post-translational modifications of those sequences.

In a further aspect, provided herein are antibody-drug conjugate comprising antibodies that bind to the same epitope as an anti-NaPi2b antibody provided herein. For example, in certain embodiments, an immunoconjugate is provided comprising an antibody that binds to the same epitope as an anti-NaPi2b antibody comprising a VH sequence of SEQ ID NO: 54 and a VL sequence of SEQ ID NO: 55, respectively.

In a further aspect of the invention, an anti-NaPi2b antibody of an antibody-drug conjugate according to any of the above embodiments is a monoclonal antibody, including a human antibody. In one embodiment, an anti-NaPi2b antibody of an antibody-drug conjugate is an antibody fragment, e.g., a Fv, Fab, Fab′, scFv, diabody, or F(ab′)₂ fragment. In another embodiment, the antibody is a substantially full length antibody, e.g., an IgGl antibody, IgG2a antibody or other antibody class or isotype as defined herein.

Table of NaPi2b Antibody Sequences

| SEQ | | |
|---|---|---|
| ID | | |
| NO | Description | Sequence |
| 48 | Anti-NaPi2b | GFSFSDFAMS |
| | HVR-Hi | |
| 49 | Anti-NaPi2b | ATIGR VAFHTYYPDSMKG |
| | HVR-H2 | |
| 50 | Anti-NaPi2b | ARHRGFDVGHFDF |
| | HVR-H3 | |
| 51 | Anti-NaPi2b | RSSETL VHSSGNTYLE |
| | HVR-L1 | |
| 52 | Anti-NaPi2b | RVSNRFS |
| | HVR-L2 | |
| 53 | Anti-NaPi2b | FQGSFNPLT |
| | HVR-L3 | |
| 54 | Anti-NaPi2b | EVQLVESGGGL VQPGGSLRL |
| | heavy chain | SCAASGFSFSDFAMSWVRQAP |
| | variable region | GKGLEWVATIGRVAFHTYYPD |
| | | SMKGRFTISRDNSKNTLYLQM |
| | | NSLRAEDTAVYYCARHRGFDV |
| | | GHFDFWGQGTLVTVSS |
| 55 | Anti-NaPi2b | DIQMTQSPSSLSASVGDRVTI |
| | light chain | TCRSSETL VHSSGNTYLEWQ |
| | variable region | QKPGKAPKLLIYRVSNRFSGV |
| | | PSRFSGSGSGTDFTLTISSLQ |
| | | PEDFATYYCFQGSFNPLTFGQ |
| | | GTKVEIKR |

### Anti-CD79b Antibodies

In certain embodiments, an ADC comprises anti-CD79b antibodies. In some embodiments, the invention provides an antibody-drug conjugate comprising an anti-CD79b antibody comprising at least one, two, three, four, five, or six HVRs selected from (a) HVR-H1 comprising the amino acid sequence of SEQ ID NO: 58; (b) HVR-H2 comprising the amino acid sequence of SEQ ID NO: 59; (c) HVR-H3 comprising the amino acid sequence of SEQ ID NO: 60; (d) HVR-L1 comprising the amino acid sequence of SEQ ID NO: 61; (e) HVR-L2 comprising the amino acid sequence of SEQ ID NO: 62; and (f) HVR-L3 comprising the amino acid sequence of SEQ ID NO: 63.

In one aspect, the invention provides an antibody-drug conjugate comprising an antibody that comprises at least one, at least two, or all three VH HVR sequences selected from (a) HVR-H1 comprising the amino acid sequence of SEQ ID NO: 58; (b) HVR-H2 comprising the amino acid sequence of SEQ ID NO: 59; and (c) HVR-H3 comprising the amino acid sequence of SEQ ID NO: 60. In a further embodiment, the antibody comprises (a) HVR-H1 comprising the amino acid sequence of SEQ ID NO: 58; (b) HVR-H2 comprising the amino acid sequence of SEQ ID NO: 59; and (c) HVR-H3 comprising the amino acid sequence of SEQ ID NO: 60.

In another aspect, the invention provides an antibody-drug conjugate comprising an antibody that comprises at least one, at least two, or all three VL HVR sequences selected from (a) HVR-L1 comprising the amino acid sequence of SEQ ID NO: 61; (b) HVR-L2 comprising the amino acid sequence of SEQ ID NO: 62; and (c) HVR-L3 comprising the amino acid sequence of SEQ ID NO: 63. In one embodiment, the antibody comprises (a) HVR-L1 comprising the amino acid sequence of SEQ ID NO: 61; (b) HVR-L2 comprising the amino acid sequence of SEQ ID NO: 62; and (c) HVR-L3 comprising the amino acid sequence of SEQ ID NO: 63.

In another aspect, an antibody-drug conjugate of the invention comprises an antibody comprising (a) a VH domain comprising at least one, at least two, or all three VH HVR sequences selected from (i) HVR-H1 comprising the amino acid sequence of SEQ ID NO: 58, (ii) HVR-H2 comprising the amino acid sequence of SEQ ID NO: 59, and (iii) HVR-H3 comprising an amino acid sequence selected from SEQ ID NO: 60; and (b) a VL domain comprising at least one, at least two, or all three VL HVR sequences selected from (i) HVR-L1 comprising the amino acid sequence of SEQ ID NO: 61, (ii) HVR-L2 comprising the amino acid sequence of SEQ ID NO: 62, and (iii) HVR-L3 comprising the amino acid sequence of SEQ ID NO: 63.

In another aspect, the invention provides an antibody-drug conjugate comprising an antibody that comprises (a) HVR-H1 comprising the amino acid sequence of SEQ ID NO: 58; (b) HVR-H2 comprising the amino acid sequence of SEQ ID NO: 59; (c) HVR-H3 comprising the amino acid sequence of SEQ ID NO: 60; (d) HVR-L1 comprising the amino acid sequence of SEQ ID NO: 61; (e) HVR-L2 comprising the amino acid sequence of SEQ ID NO: 62; and (f) HVR-L3 comprising the amino acid sequence of SEQ ID NO: 63.

In any of the above embodiments, an anti-CD79b antibody of an antibody-drug conjugate is humanized. In one embodiment, an anti-CD79b antibody comprises HVRs as in any of the above embodiments, and further comprises a human acceptor framework, e.g. a human immunoglobulin framework or a human consensus framework.

In another aspect, an anti-CD79b antibody of an antibody-drug conjugate comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 56. In certain embodiments, a VH sequence having at least 90%, 91%, 92%, 93%, 94%, 95%, 96%, 97%, 98%, or 99% identity to the amino acid sequence of SEQ ID NO: 56 contains substitutions (e.g., conservative substitutions), insertions, or deletions relative to the reference sequence, but an anti-CD79b antibody comprising that sequence retains the ability to bind to CD79b. In certain embodiments, a total of 1 to 10 amino acids have been substituted, inserted and/or deleted in SEQ ID NO: 56. In certain embodiments, a total of 1 to 5 amino acids have been substituted, inserted and/or deleted in SEQ ID NO: 56. In certain embodiments, substitutions, insertions, or deletions occur in regions outside the HVRs (i.e., in the FRs). Optionally, the anti-CD79b antibody comprises the VH sequence of SEQ ID NO: 8, including post-translational modifications of that sequence. In a particular embodiment, the VH comprises one, two or three HVRs selected from: (a) HVR-H1 comprising the amino acid sequence of SEQ ID NO: 58, (b) HVR-H2 comprising the amino acid sequence of SEQ ID NO: 59, and (c) HVR-H3 comprising the amino acid sequence of SEQ ID NO: 60.

In another aspect, an anti-CD79b antibody of an antibody-drug conjugate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 57. In certain embodiments, a VL sequence having at least 90%, 91%, 92%, 93%, 94%, 95%, 96%, 97%, 98%, or 99% identity to the amino acid sequence of SEQ ID NO: 57 contains substitutions (e.g., conservative substitutions), insertions, or deletions relative to the reference sequence, but an anti-CD79b antibody comprising that sequence retains the ability to bind to CD79b. In certain embodiments, a total of 1 to 10 amino acids have been substituted, inserted and/or deleted in SEQ ID NO: 57. In certain embodiments, a total of 1 to 5 amino acids have been substituted, inserted and/or deleted in SEQ ID NO: 57. In certain embodiments, the substitutions, insertions, or deletions occur in regions outside the HVRs (i.e., in the FRs). Optionally, the anti-CD79b antibody comprises the VL sequence of SEQ ID NO: 57, including post-translational modifications of that sequence. In a particular embodiment, the VL comprises one, two or three HVRs selected from (a) HVR-L1 comprising the amino acid sequence of SEQ ID NO: 61; (b) HVR-L2 comprising the amino acid sequence of SEQ ID NO: 62; and (c) HVR-L3 comprising the amino acid sequence of SEQ ID NO: 63.

In another aspect, an antibody-drug conjugate comprising an anti-CD79b antibody is provided, wherein the antibody comprises a VH as in any of the embodiments provided above, and a VL as in any of the embodiments provided above.

In one embodiment, an antibody-drug conjugate is provided, wherein the antibody comprises the VH and VL sequences in SEQ ID NO: 56 and SEQ ID NO: 57, respectively, including post-translational modifications of those sequences.

In a further aspect, provided herein are antibody-drug conjugate comprising antibodies that bind to the same epitope as an anti-CD79b antibody provided herein. For example, in certain embodiments, an immunoconjugate is provided comprising an antibody that binds to the same epitope as an anti-CD79b antibody comprising a VH sequence of SEQ ID NO: 56 and a VL sequence of SEQ ID NO: 57, respectively.

In a further aspect of the invention, an anti-CD79b antibody of an antibody-drug conjugate according to any of the above embodiments is a monoclonal antibody, including a human antibody. In one embodiment, an anti-CD79b antibody of an antibody-drug conjugate is an antibody fragment, e.g., a Fv, Fab, Fab′, scFv, diabody, or F(ab′)₂ fragment. In another embodiment, the antibody is a substantially full length antibody, e.g., an IgGl antibody, IgG2a antibody or other antibody class or isotype as defined herein.

Table of CD79b Antibody Sequences

| SEQ | | |
|---|---|---|
| ID | Descrip- | |
| NO | tion | Sequence |
| 56 | anti-CD79b | EVQLVESGGG LVQPGGSLRL SCAASGYTFS |
| | huMA79bv28 | SYWIEWVRQA PGKGLEWIGE ILPGGGDTNY |
| | heavy chain | NEIFKGRATF SADTSKNTAY LQMNSLRAED |
| | variable | TAVYYCTRRV PIRLDYWGQG TLVTVSS |
| | region | |
| 57 | anti-CD79b | DIQLTQSPSS LSASVGDRVT ITCKASQSVD |
| | huMA79bv28 | YEGDSFLNWY QQKPGKAPKL LIYAASNLES |
| | light chain | GVPSRFSGSG SGTDFTLTIS SLQPEDFATY |
| | variable | YCQQSNEDPL TFGQGTKVEI KR |
| | region | |
| 58 | anti-CD79b | GYTFSSYWIE |
| | huMA79bv28 | |
| | HVR-Hl | |
| 59 | anti-CD79b | GEILPGGGDTNYNEIFKG |
| | huMA79bv28 | |
| | HVR-H2 | |
| 60 | anti-CD79b | TRRVPIRLDY |
| | huMA79bv28 | |
| | HVR-H3 | |
| 61 | anti-CD79b | KASQSVDYEGDSFLN |
| | huMA79bv28 | |
| | HVR-L1 | |
| 62 | anti-CD79b | AASNLES |
| | huMA79bv28 | |
| | HVR-L2 | |
| 63 | anti-CD79b | QQSNEDPLT |
| | huMA79bv28 | |
| | HVR-L3 | |

### Human HER2 Precursor Protein

Details of an exemplary human HER2 precursor protein with signal sequences is provided below

| SEQ | | |
|---|---|---|
| ID | | |
| NO | Description | Sequence |
| 64 | Exemplary | MELAALCRWG LLLALLPPGA ASTQVCTGTD |
| | human HER2 | MKLRLPASPE THLDMLRHLY QGCQVVQGNL |
| | precursor | ELTYLPTNAS LSFLQDIQEV QGYVLIAHNQ |
| | protein, | VRQVPLQRLR IVRGTQLFED NYALAVLDNG |
| | with | DPLNNITPVT GASPGGLREL QLRSLTEILK |
| | signal | GGVLIQRNPQ LCYQDTILWK DIFHKNNQLA |
| | sequence | LTLIDTNRSR ACHPCSPMCK GSRCWGESSE |
| | | DCQSLTRTVC AGGCARCKGP LPTDCCHEQC |
| | | AAGCTGPKHS DCLACLHFNH SGICELHCPA |
| | | LVTYNTDTFE SMPNPEGRYT FGASCVTACP |
| | | YNYLSTDVGS CTLVCPLHNQ EVTAEDGTQR |
| | | CEKCSKPCAR VCYGLGMEHL REVRAVTSAN |
| | | IQEFAGCKKI FGSLAFLPES FDGDPASNTA |
| | | PLQPEQLQVF ETLEEITGYL YISAWPDSLP |
| | | DLSVFQNLQV IRGRILHNGA YSLTLQGLGI |
| | | SWLGLRSLRE LGSGLALIHH NTHLCFVHTV |
| | | PWDQLFRNPH QALLHTANRP EDECVGEGLA |
| | | CHQLCARGHC WGPGPTQCVN CSQFLRGQEC |
| | | VEECRVLQGL PREYVNARHC LPCHPECQPQ |
| | | NGSVTCFGPE ADQCVACAHY KDPPFCVARC |
| | | PSGVKPDLSY MPIWKFPDEE GACQPCPINC |
| | | THSCVDLDDK GCPAEQRASP LTSIISAVVG |
| | | ILLVVVLGVV FGILIKRRQQ KIRKYTMRRL |
| | | LQETELVEPL TPSGAMPNQA QMRILKETEL |
| | | RKVKVLGSGA FGTVYKGIWI PDGENVKIPV |
| | | AIKVLRENTS PKANKEILDE AYVMAGVGSP |
| | | YVSRLLGICL TSTVQLVTQL MPYGCLLDHV |
| | | RENRGRLGSQ DLLNWCMQIA KGMSYLEDVR |
| | | LVHRDLAARN VLVKSPNHVK ITDFGLARLL |
| | | DIDETEYHAD GGKVPIKWMA LESILRRRFT |
| | | HQSDVWSYGV TVWELMTFGA KPYDGIPARE |
| | | IPDLLEKGER LPQPPICTID VYMIMVKCWM |
| | | IDSECRPRFR ELVSEFSRMA RDPQRFVVIQ |
| | | NEDLGPASPL DSTFYRSLLE DDDMGDLVDA |
| | | EEYLVPQQGF FCPDPAPGAG GMVHHRHRSS |
| | | STRSGGGDLT LGLEPSEEEA PRSPLAPSEG |
| | | AGSDVFDGDL GMGAAKGLQS LPTHDPSPLQ |
| | | RYSEDPTVPL PSETDGYVAP LTCSPQPEYV |
| | | NQPDVRPQPP SPREGPLPAA RPAGATLERP |
| | | KTLSPGKNGV VKDVFAFGGA VENPEYLTPQ |
| | | GGAAPQPHPP PAFSPAFDNL YYWDQDPPER |
| | | GAPPSTFKGT PTAENPEYLG LDVPV |

### Antibody Affinity

In certain embodiments, an antibody provided herein has a dissociation constant (Kd) of ≤1 μM, ≤100 nM, ≤50 nM, ≤10 nM, ≤5 nM, ≤1 nM, ≤0.1 nM, ≤0.01 nM, or ≤0.001 nM, and optionally is ≥10⁻¹³ M. (e.g. 10⁻⁸ M or less, e.g. from 10⁻⁸ M to 10⁻¹³ M, e.g., from 10⁻⁹ M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 pM ^{[125}I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCF T-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethyl-aminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (−0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ, See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instalments) or a 8000-series SLM-AMINCO spectrophotometer (ThermoSpectronic) with a stirred cuvette.

### Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9: 129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9: 129-134 (2003); and Hollinger et al., Proc. Natl. Acad. Sci. USA 90: 6444-6448 (1993). Triabodies and tetrabodies are also described in Hudson et al., Nat. Med. 9: 129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516).

Antibody fragments can be made by various techniques, including but not limited to proteolytic digestion of an intact antibody as well as production by recombinant host cells (e.g. E. coli or phage), as described herein.

### Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13: 1619-1633 (2008), and are further described, e.g., in Riechmann et al., Nature 332:323-329 (1988); Queen et al., Proc. Nat'l Acad. Sci. USA 86: 10029-10033 (1989); U.S. Pat. Nos. 5,821,337, 7,527,791, 6,982,321, and 7,087,409; Kashmiri et al, Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol, 151:2623 (1993)); human mature (somatically mutated) framework regions or human germline framework regions (see, e.g., Almagro and Fransson, Front. Biosci. 13: 1619-1633 (2008)); and framework regions derived from screening FR libraries (see, e.g., Baca et al., J. Biol. Chem. 272: 10678-10684 (1997) and Rosok et al., J. Biol. Chem. 271:22611-22618 (1996)).

### Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23: 1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol, 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. Methods in Molecular Biology 178: 1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, Methods in Molecular Biology 248: 161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol, 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol, 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, bispecific antibodies may bind to two different epitopes of the same target. Bispecific antibodies may also be used to localize cytotoxic agents to cells which express the target. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). The term “knob-into-hole” or “KnH” technology as used herein refers to the technology directing the pairing of two polypeptides together in vitro or in vivo by introducing a protuberance (knob) into one polypeptide and a cavity (hole) into the other polypeptide at an interface in which they interact. For example, KnHs have been introduced in the Fc:Fc binding interfaces, CL:CH1 interfaces or VH/VL interfaces of antibodies (see, e.g., US 2011/0287009, US2007/0178552, WO 96/027011, WO 98/050431, Zhu et al., 1997, Protein Science 6:781-788, and WO2012/106587). In some embodiments, KnHs drive the pairing of two different heavy chains together during the manufacture of multispecific antibodies. For example, multispecific antibodies having KnH in their Fc regions can further comprise single variable domains linked to each Fc region, or further comprise different heavy chain variable domains that pair with similar or different light chain variable domains. KnH technology can be also be used to pair two different receptor extracellular domains together or any other polypeptide sequences that comprises different target recognition sequences (e.g., including affibodies, peptibodies and other Fc fusions).

The term “knob mutation” as used herein refers to a mutation that introduces a protuberance (knob) into a polypeptide at an interface in which the polypeptide interacts with another polypeptide. In some embodiments, the other polypeptide has a hole mutation.

The term “hole mutation” as used herein refers to a mutation that introduces a cavity (hole) into a polypeptide at an interface in which the polypeptide interacts with another polypeptide. In some embodiments, the other polypeptide has a knob mutation.

A brief nonlimiting discussion is provided below.

A “protuberance” refers to at least one amino acid side chain which projects from the interface of a first polypeptide and is therefore positionable in a compensatory cavity in the adjacent interface (i.e. the interface of a second polypeptide) so as to stabilize the heteromultimer, and thereby favor heteromultimer formation over homomultimer formation, for example. The protuberance may exist in the original interface or may be introduced synthetically (e.g., by altering nucleic acid encoding the interface). In some embodiments, nucleic acid encoding the interface of the first polypeptide is altered to encode the protuberance. To achieve this, the nucleic acid encoding at least one “original” amino acid residue in the interface of the first polypeptide is replaced with nucleic acid encoding at least one “import” amino acid residue which has a larger side chain volume than the original amino acid residue. It will be appreciated that there can be more than one original and corresponding import residue. The side chain volumes of the various amino residues are shown, for example, in Table 1 of US2011/0287009. A mutation to introduce a “protuberance” may be referred to as a “knob mutation.”

In some embodiments, import residues for the formation of a protuberance are naturally occurring amino acid residues selected from arginine (R), phenylalanine (F), tyrosine (Y) and tryptophan (W). In some embodiments, an import residue is tryptophan or tyrosine. In some embodiment, the original residue for the formation of the protuberance has a small side chain volume, such as alanine, asparagine, aspartic acid, glycine, serine, threonine or valine.

A “cavity” refers to at least one amino acid side chain which is recessed from the interface of a second polypeptide and therefore accommodates a corresponding protuberance on the adjacent interface of a first polypeptide. The cavity may exist in the original interface or may be introduced synthetically (e.g. by altering nucleic acid encoding the interface). In some embodiments, nucleic acid encoding the interface of the second polypeptide is altered to encode the cavity. To achieve this, the nucleic acid encoding at least one “original” amino acid residue in the interface of the second polypeptide is replaced with DNA encoding at least one “import” amino acid residue which has a smaller side chain volume than the original amino acid residue. It will be appreciated that there can be more than one original and corresponding import residue. In some embodiments, import residues for the formation of a cavity are naturally occurring amino acid residues selected from alanine (A), serine (S), threonine (T) and valine (V). In some embodiments, an import residue is serine, alanine or threonine. In some embodiments, the original residue for the formation of the cavity has a large side chain volume, such as tyrosine, arginine, phenylalanine or tryptophan. A mutation to introduce a “cavity” may be referred to as a “hole mutation.”

The protuberance is “positionable” in the cavity which means that the spatial location of the protuberance and cavity on the interface of a first polypeptide and second polypeptide respectively and the sizes of the protuberance and cavity are such that the protuberance can be located in the cavity without significantly perturbing the normal association of the first and second polypeptides at the interface. Since protuberances such as Tyr, Phe and Trp do not typically extend perpendicularly from the axis of the interface and have preferred conformations, the alignment of a protuberance with a corresponding cavity may, in some instances, rely on modeling the protuberance/cavity pair based upon a three-dimensional structure such as that obtained by X-ray crystallography or nuclear magnetic resonance (NMR). This can be achieved using widely accepted techniques in the art.

In some embodiments, a knob mutation in an IgGl constant region is T366W (EU numbering). In some embodiments, a hole mutation in an IgGl constant region comprises one or more mutations selected from T366S, L368A and Y407V (EU numbering). In some embodiments, a hole mutation in an IgGl constant region comprises T366S, L368A and Y407V (EU numbering).

In some embodiments, a knob mutation in an IgG4 constant region is T366W (EU numbering). In some embodiments, a hole mutation in an IgG4 constant region comprises one or more mutations selected from T366S, L368A, and Y407V (EU numbering). In some embodiments, a hole mutation in an IgG4 constant region comprises T366S, L368A, and Y407V (EU numbering).

Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol, 148(5): 1547-1553 (1992)); using “diabody” technology for making bispecific antibody fragments (see, e.g., Hollinger et al., Proc. Natl Acad. Sci. USA, 90:6444-6448 (1993)); and using single-chain Fv (sFv) dimers (see, e.g. Gruber et al., J. Immunol, 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to the target as well as another, different antigen (see, US 2008/0069820, for example).

### Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### Substitution, Insertion, and Deletion Variants

In certain embodiments, antibody variants having one or more amino acid substitutions are provided. Sites of interest for substitutional mutagenesis include the HVRs and FRs. Conservative substitutions are shown below in a Table of conservative substitutions under the heading of “preferred substitutions.” More substantial changes are provided in the Table under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

Table of conservative substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; | Ile |
| | Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:

(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207: 179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178: 1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244: 1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%). The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108; US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336: 1239-1249 (2004); Yamane-Ohnuki et al. Biotech. Bioeng. 87: 614 (2004). Examples of cell lines capable of producing defucosylated antibodies include Led 3 CHO cells deficient in protein fucosylation (Ripka et al. Arch. Biochem. Biophys. 249:533-545 (1986); US Pat Appl No US 2003/0157108; and WO 2004/056312, especially at Example 11), and knockout cell lines, such as alpha-1,6-fucosyl transferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgGl, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82: 1499-1502 (1985); 5,821,337 (see Bruggemann, M. et al., J. Exp. Med. 166: 1351-1361 (1987)).

Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202: 163 (1996); Cragg, M. S. et al., Blood 101: 1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12): 1759-1769 (2006)).

In some embodiments, one or more amino acid modifications may be introduced into the Fc portion of the antibody provided herein in order to increase IgG binding to the neonatal Fc receptor. In certain embodiments, the antibody comprises the following three mutations according to EU numbering: M252Y, S254T, and T256E (the “YTE mutation”) (U.S. Pat. No. 8,697,650; see also Dall'Acqua et al., Journal of Biological Chemistry 281(33):23514-23524 (2006). In certain embodiments, the YTE mutation does not affect the ability of the antibody to bind to its cognate antigen. In certain embodiments, the YTE mutation increases the antibody's serum half-life compared to the native (i.e., non-YTE mutant) antibody. In some embodiments, the YTE mutation increases the serum half-life of the antibody by 3-fold compared to the native (i.e., non-YTE mutant) antibody. In some embodiments, the YTE mutation increases the serum half-life of the antibody by 2-fold compared to the native (i.e., non-YTE mutant) antibody. In some embodiments, the YTE mutation increases the serum half-life of the antibody by 4-fold compared to the native (i.e., non-YTE mutant) antibody. In some embodiments, the YTE mutation increases the serum half-life of the antibody by at least 5-fold compared to the native (i.e., non-YTE mutant) antibody. In some embodiments, the YTE mutation increases the serum half-life of the antibody by at least 10-fold compared to the native (i.e., non-YTE mutant) antibody. See, e.g., U.S. Pat. No. 8,697,650; see also Dall'Acqua et al., Journal of Biological Chemistry 281(33):23514-23524 (2006).

In certain embodiments, the YTE mutant provides a means to modulate antibody-dependent cell-mediated cytotoxicity (ADCC) activity of the antibody. In certain embodiments, the YTEO mutant provides a means to modulate ADCC activity of a humanized IgG antibody directed against a human antigen. See, e.g., U.S. Pat. No. 8,697,650; see also Dall'Acqua et al., Journal of Biological Chemistry 281(33):23514-23524 (2006).

In certain embodiments, the YTE mutant allows the simultaneous modulation of serum half-life, tissue distribution, and antibody activity (e.g., the ADCC activity of an IgG antibody). See, e.g., U.S. Pat. No. 8,697,650; see also Dall'Acqua et al., Journal of Biological Chemistry 281(33):23514-23524 (2006).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In certain embodiments, the proline at position 329 (EU numbering) (P329) of a wild-type human Fc region is substituted with glycine or arginine or an amino acid residue large enough to destroy the proline sandwich within the Fc/Fc gamma receptor interface, that is formed between the P329 of the Fe and tryptophane residues W87 and W110 of FcgRIII (Sondermann et al., Nature 406, 267-273 (20 Jul. 2000)). In a further embodiment, at least one further amino acid substitution in the Fc variant is S228P, E233P, L234A, L235A, L235E, N297A, N297D, or P331S and still in another embodiment said at least one further amino acid substitution is L234A and L235A of the human IgGl Fc region or S228P and L235E of the human IgG4 Fc region, all according to EU numbering (U.S. Pat. No. 8,969,526 which is incorporated by reference in its entirety).

In certain embodiments, a polypeptide comprises the Fc variant of a wild-type human IgG Fc region wherein the polypeptide has P329 of the human IgG Fc region substituted with glycine and wherein the Fc variant comprises at least two further amino acid substitutions at L234A and L235A of the human IgGl Fc region or S228P and L235E of the human IgG4 Fc region, and wherein the residues are numbered according to the EU numbering (U.S. Pat. No. 8,969,526 which is incorporated by reference in its entirety). In certain embodiments, the polypeptide comprising the P329G, L234A and L235A (EU numbering) substitutions exhibit a reduced affinity to the human FcγRIIIA and FcγRIIA, for down-modulation of ADCC to at least 20% of the ADCC induced by the polypeptide comprising the wildtype human IgG Fc region, and/or for down-modulation of ADCP (U.S. Pat. No. 8,969,526 which is incorporated by reference in its entirety).

In a specific embodiment the polypeptide comprising an Fc variant of a wildtype human Fc polypeptide comprises a triple mutation: an amino acid substitution at position Pro329, a L234A and a L235A mutation according to EU numbering (P329/LALA) (U.S. Pat. No. 8,969,526 which is incorporated by reference in its entirety). In specific embodiments, the polypeptide comprises the following amino acid substitutions: P329G, L234A, and L235A according to EU numbering.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).) In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

### Cysteine Engineered Antibody Variants

In certain embodiments, it may be desirable to create cysteine engineered antibodies, e.g., a “THIOMAB™” or TDC, in which one or more residues of an antibody are substituted with cysteine residues. In particular embodiments, the substituted residues occur at sites of the antibody that are available for conjugation. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: K149 (Kabat numbering) of the light chain; V205 (Kabat numbering) of the light chain; A118 (EU numbering) of the heavy chain; A140 (EU numbering) of the heavy chain; L174 (EU numbering) of the heavy chain; Y373 (EU numbering) of the heavy chain; and 5400 (EU numbering) of the heavy chain Fc region. In specific embodiments, the antibodies described herein comprise the HC-A140C (EU numbering) cysteine substitution. In specific embodiments, the antibodies described herein comprise the LC-K149C (Kabat numbering) cysteine substitution. In specific embodiments, the antibodies described herein comprise the HC-A118C (EU numbering) cysteine substitution. Cysteine engineered antibodies may be generated as described, e.g., in U.S. Pat. No. 7,521,541.

In certain embodiments, the antibody comprises one of the following heavy chain cysteine substitutions:

| Chain | | EU Mutation | Kabat Mutation |
|---|---|---|---|
| (HC/LC) | Residue | Site # | Site # |
| HC | T | 114 | 110 |
| HC | A | 140 | 136 |
| HC | L | 174 | 170 |
| HC | L | 179 | 175 |
| HC | T | 187 | 183 |
| HC | T | 209 | 205 |
| HC | V | 262 | 258 |
| HC | G | 371 | 367 |
| HC | Y | 373 | 369 |
| HC | E | 382 | 378 |
| HC | S | 424 | 420 |
| HC | N | 434 | 430 |
| HC | Q | 438 | 434 |

In certain embodiments, the antibody comprises one of the following light chain cysteine substitutions:

| Chain | | EU Mutation | Kabat Mutation |
|---|---|---|---|
| (HC/LC) | Residue | Site # | Site # |
| LC | I | 106 | 106 |
| LC | R | 108 | 108 |
| LC | R | 142 | 142 |
| LC | K | 149 | 149 |
| LC | V | 205 | 205 |

A nonlimiting exemplary hu7C2.v2.2.LA light chain (LC) K149C THIOMAB™ has the heavy chain and light chain amino acid sequences of SEQ ID NOs: 26 and 30, respectively. A nonlimiting exemplary hu7C2.v2.2.LA heavy chain (HC) A118C THIOMAB™ has the heavy chain and light chain amino acid sequences of SEQ ID NOs: 31 and 25, respectively.

### Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1, 3, 6-trioxane, ethylene/maleic anhydride copolymer, polyaminoacids (either homopolymers or random copolymers), and dextran or poly(n-vinyl pyrrolidone)polyethylene glycol, propylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### Recombinant Methods and Compositions

Antibodies may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0 Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fe effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N T, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22: 1409-1414 (2004), and L1 et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977); baby hamster kidney cells (BHK); mouse Sertoli cells (TM4 cells as described, e.g., in Mather, Biol. Reprod. 23:243-251 (1980); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Administration & Dose

Compounds of formula I may be administered alone or in combination with one or another or with one or more pharmacologically active compounds which are different from the compounds of formula I.

Compounds of the invention may suitably be combined with various components to produce compositions of the invention. Suitably the compositions are combined with a pharmaceutically acceptable carrier or diluent to produce a pharmaceutical composition (which may be for human or animal use). Suitable carriers and diluents include isotonic saline solutions, for example phosphate-buffered saline. Useful pharmaceutical compositions and methods for their preparation may be found in standard pharmaceutical texts. See, for example, Handbook for Pharmaceutical Additives, 3rd Edition (eds. M. Ash and I. Ash), 2007 (Synapse Information Resources, Inc., Endicott, New York, USA) and Remington: The Science and Practice of Pharmacy, 21st Edition (ed. D. B. Troy) 2006 (Lippincott, Williams and Wilkins, Philadelphia, USA) which are incorporated herein by reference.

The compounds of the invention may be administered by any suitable route. Suitably the compounds of the invention will normally be administered orally or by any parenteral route, in the form of pharmaceutical preparations comprising the active ingredient, optionally in the form of a non-toxic organic, or inorganic, acid, or base, addition salt, in a pharmaceutically acceptable dosage form.

The compounds of the invention, their pharmaceutically acceptable salts, and pharmaceutically acceptable solvates of either entity can be administered alone but will generally be administered in admixture with a suitable pharmaceutical excipient diluent or carrier selected with regard to the intended route of administration and standard pharmaceutical practice.

For example, the compounds of the invention or salts or solvates thereof can be administered orally, buccally or sublingually in the form of tablets, capsules (including soft gel capsules), ovules, elixirs, solutions or suspensions, which may contain flavouring or colouring agents, for immediate-, delayed-, modified-, sustained-, controlled-release or pulsatile delivery applications. The compounds of the invention may also be administered via fast dispersing or fast dissolving dosages forms.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethyl cellulose (HPMC), hydroxypropylcellulose (HPC), sucrose, gelatin and acacia. Additionally, lubricating agents such as magnesium stearate, stearic acid, glyceryl behenate and tale may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol.

The compounds of the invention can also be administered parenterally, for example, intravenously, intra-arterially, or they may be administered by infusion techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Suitably formulation of the invention is optimised for the route of administration e.g. oral, intravenously, etc.

Administration may be in one dose, continuously or intermittently (e.g. in divided doses at appropriate intervals) during the course of treatment. Methods of determining the most effective means and dosage are well known to a skilled person and will vary with the formulation used for therapy, the purpose of the therapy, the target cell(s) being treated, and the subject being treated. Single or multiple administrations can be carried out with the dose level and the dose regimen being selected by the treating physician, veterinarian, or clinician.

Depending upon the disorder and patient to be treated, as well as the route of administration, the compositions may be administered at varying doses. For example, a typical dosage for an adult human may be 100 ng to 25 mg (suitably about 1 micro g to about 10 mg) per kg body weight of the subject per day.

Suitably guidance may be taken from studies in test animals when estimating an initial dose for human subjects. For example when a particular dose is identified for mice, suitably an initial test dose for humans may be approx. 0.5× to 2× the mg/Kg value given to mice.

### Other Forms

Unless otherwise specified, included in the above are the well known ionic, salt, solvate, and protected forms of these substituents. For example, a reference to carboxylic acid (—COOH) also includes the anionic (carboxylate) form (—COO—), a salt or solvate thereof, as well as conventional protected forms. Similarly, a reference to an amino group includes the protonated form (—N+HR¹R²), a salt or solvate of the amino group, for example, a hydrochloride salt, as well as conventional protected forms of an amino group. Similarly, a reference to a hydroxyl group also includes the anionic form (—O—), a salt or solvate thereof, as well as conventional protected forms.

### Isomers, Salts and Solvates

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; alpha- and beta-forms; axial and equatorial forms; boat-, chair-, twist-, envelope-, and half chair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH.

A reference to a class of structures may well include structurally isomeric forms falling within that class (e.g. C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not apply to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof.

Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

In some embodiments, the compound of formula (I) and salts and solvates thereof, comprises pharmaceutically acceptable salts of the compounds of formula (I).

Compounds of formula (I), which include compounds specifically named above, may form salts, solvates (such as hydrates), isomers or tautomers. Suitably, these are pharmaceutically acceptable salts, solvates, isomers or tautomers. These salts include nontoxic acid addition salts (including di-acids) and base salts.

If the compound is cationic, or has a functional group which may be cationic (e.g. —NH₂ may be —NH₃+), then an acid addition salt may be formed with a suitable anion. Examples of suitable inorganic anions include, but are not limited to, those derived from the following inorganic acids hydrochloric acid, nitric acid, nitrous acid, phosphoric acid, sulfuric acid, sulphurous acid, hydrobromic acid, hydroiodic acid, hydrofluoric acid, phosphoric acid and phosphorous acids. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfonate, naphthylate, 2-napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate, tosylate, trifluoroacetate and xinofoate salts.

For example, if the compound is anionic, or has a functional group which may be anionic (e.g. —COOH may be —COO—), then a base salt may be formed with a suitable cation. Examples of suitable inorganic cations include, but are not limited to, metal cations, such as an alkali or alkaline earth metal cation, ammonium and substituted ammonium cations, as well as amines. Examples of suitable metal cations include sodium (Na⁺) potassium (K⁺), magnesium (Mg²⁺), calcium (Ca²⁺), zinc (Zn²⁺), and aluminum (Al³⁺). Examples of suitable organic cations include, but are not limited to, ammonium ion (i.e. NH⁴⁺) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺. Examples of suitable amines include arginine, N,N′-dibenzylethylene-diamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine. For a discussion of useful acid addition and base salts, see S. M. Berge et al., J. Pharm. Sci. (1977) 66:1-19; see also Stahl and Wermuth, Handbook of Pharmaceutical Salts: Properties, Selection, and Use (2011)

Pharmaceutically acceptable salts may be prepared using various methods. For example, one may react a compound of formula (I) with an appropriate acid or base to give the desired salt. One may also react a precursor of the compound of formula (I) with an acid or base to remove an acid- or base-labile protecting group or to open a lactone or lactam group of the precursor. Additionally, one may convert a salt of the compound of formula (I) to another salt through treatment with an appropriate acid or base or through contact with an ion exchange resin. Following reaction, one may then isolate the salt by filtration if it precipitates from solution, or by evaporation to recover the salt. The degree of ionization of the salt may vary from completely ionized to almost non-ionized.

It may be convenient or desirable to prepare, purify, and/or handle a corresponding solvate of the active compound. The term “solvate” describes a molecular complex comprising the compound and one or more pharmaceutically acceptable solvent molecules (e.g., EtOH). The term “hydrate” is a solvate in which the solvent is water. Pharmaceutically acceptable solvates include those in which the solvent may be isotopically substituted (e.g., D₂O, acetone-d6, DMSO-d6).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will typically be observed.

### BRIEF DESCRIPTION OF THE DRAWINGS

Embodiments of the present study will now be described further with reference to the accompanying drawings, in which:

FIG. 1 is a visual representation of data presented in Table 2 (UPLC data showing the retention times of the compounds analysed) (seco);

FIG. 2 is a visual representation of data presented in Table 2 (UPLC data showing the retention times of the compounds analysed) (spiro).

### DESCRIPTION OF THE EMBODIMENTS

### General Remarks

3-(Bromomethyl)-benzeneacetic acid methyl ester was purchased from Beta Pharma Scientific Inc. (USA). All other reagents and solvents were purchased from Fluorochem Ltd. (UK), Sigma-Aldrich Ltd. (Merck KGaA, Germany), VWR Ltd. (Avantor Inc., USA), Fischer Scientific, Inc. (USA), and other standard commercial suppliers, and used as purchased. Anhydrous reactions were carried out under an inert atmosphere of argon using anhydrous solvents which were used as purchased, without further drying. Thin Layer Chromatography (TLC) was performed on silica gel aluminium plates (Merck 60, F₂₅₄), and flash column chromatography was carried out either manually, using silica gel (Merck 9385, 230-400 mesh ASTM, 40-63 μM) (whilst monitoring by TLC: UV (254 nm), or using a Biotage Isolera Dalton 2000 (automated mass-directed flash chromatography system). All Nuclear Magnetic Resonance (NMR) spectra were obtained at room temperature either using a Bruker DPX400 or a Varian Mercury Vx Agilent 400 MHz spectrometer, for which chemical shifts are expressed in ppm relative to the solvent and coupling constants are expressed in Hz. Microwave reactions were carried out on a Biotage Initiator+ microwave synthesizer. High Resolution Mass Spectrometry (HRMS) was performed on a Thermo Scientific-Exactive HCD Orbitrap Mass Spectrometer. Yields refer to isolated material (homogeneous by TLC and NMR) unless otherwise stated and names are assigned according to IUPAC nomenclature. Liquid Chromatography Mass Spectrometry (LCMS) analysis Methods A-C were performed on a Waters Alliance 2695 with water (A) and acetonitrile (B) comprising the mobile phases. Formic acid (0.1%) was added to both acetonitrile and water to ensure acidic conditions throughout the analysis. Function type: Diode array (535 scans). Column type: Monolithic C18 50×4.60 mm. Mass spectrometry data were collected using a Waters Micromass ZQ instrument coupled to the HPLC with a Waters 2996 PDA. Waters Micromass ZQ parameters used were: Capillary (kV), 3.38; Cone (V), 35; Extractor (V), 3.0; Source temperature (° C.), 100; De-solvation Temperature (° C.), 200; Cone flow rate (L/h), 50; De-solvation flow rate (L/h), 250. Gradient conditions are described as follows.

Method A (10 min): from 95% A/5% B to 50% B over 3 min. Then from 50% B to 80% B over 2 min. Then from 80% B to 95% B over 1.5 min and held constant for 1.5 min. This was then reduced to 5% B over 0.2 min and maintained to 5% B for 1.8 min. The flow rate was 0.5 mL/min, 200 μL was split via a zero dead volume T piece which passed into the mass spectrometer. The wavelength range of the UV detector was 220-400 nm.

Method B (5 min): from 95% A/5% B to 90% B over 3 min. Then from 90% B to 95% B over 0.5 min and held constant for 1 min. This was then reduced to 5% B over 0.5 min. The flow rate was 1.0 mL/min, 100 μL was split via a zero dead volume T piece which passed into the mass spectrometer. The wavelength range of the UV detector was 220-500 nm.

Method C (5 min): from 95% A/5% B, which was increased to 90% B over 3 min and to 95% B over a further 0.5 min. The gradient was then held at 95% B for 1 min and then returned to 5% B over 0.5 min. The total duration of the run was 5 minutes and the solvent flow rate was 1 mL/min, 100 μL was split via a zero dead volume T piece which passed into the mass spectrometer. The wavelength range of the UV detector was 220-500 nm.

Liquid Chromatography Mass Spectrometry (LCMS) analysis Methods D-G were performed on a Shimadzu LC-20AD series, Binary Pump, Diode Array Detector. Column type: Agilent Poroshell 120 EC-C18, 2.7 μm, 4.6×50 mm. Mobile phase: A: 0.05% formic acid in water (v/v); B: 0.05% formic acid in acetonitrile (v/v). Flow Rate: 1 mL/min at 25° C. Detector: 214 nm, 254 nm. Gradient stop time: 5 min. MS: 2020, Quadrupole LC/MS, Ion Source: API-ESI, TIC: 100-1300 m/z, Drying gas flow: 15 L/min, Nebulizer pressure: 1.5 L/min, Drying gas temperature: 250° C., Vcap: 4500V. Sample preparation: samples were dissolved in methanol at 1-10 μg/mL, then filtered through a 0.22 μm filter membrane. Injection volume: 1-10 μL. Gradient conditions are described as follows.

Method D (5 min): 20% A/80% B for 0.5 min, which was increased to 100% B over 3.5 min, then held at 100% B for 0.5 min. This was then returned to 20% A/80% B for 0.5 min.

Method E (5 min): 50% A/50% B for 0.5 min, which was increased to 100% B over 3.5 min, then held at 100% B for 0.5 min. This was then returned to 50% A/50% B for 0.5 min.

Method F (5 min): 85% A/15% B for 0.5 min, which was increased to 100% B over 3.5 min, then held at 100% B for 0.5 min. This was then returned to 85% A/15% B for 0.5 min.

Method G (5 min): 97% A/3% B for 0.5 min, which was increased to 30% A/70% B over 3.5 min, then to 100% B over 0.5 min. This was then returned to 97% A/3% B for 0.5 min.

Optical rotations were measured on an SGWzz-1 automatic Polarimeter (Shanghai Shen Guang Instrument Co., Ltd.)

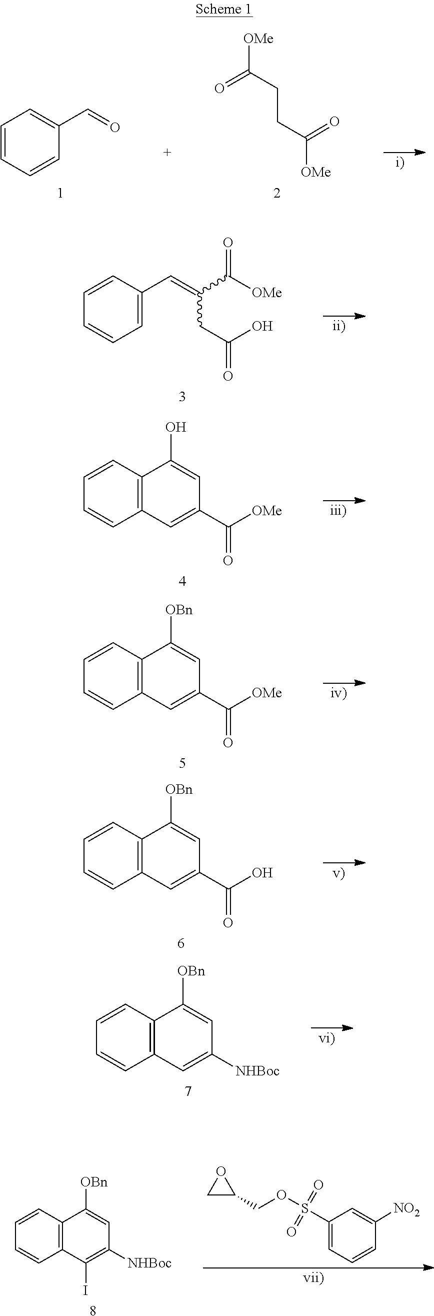

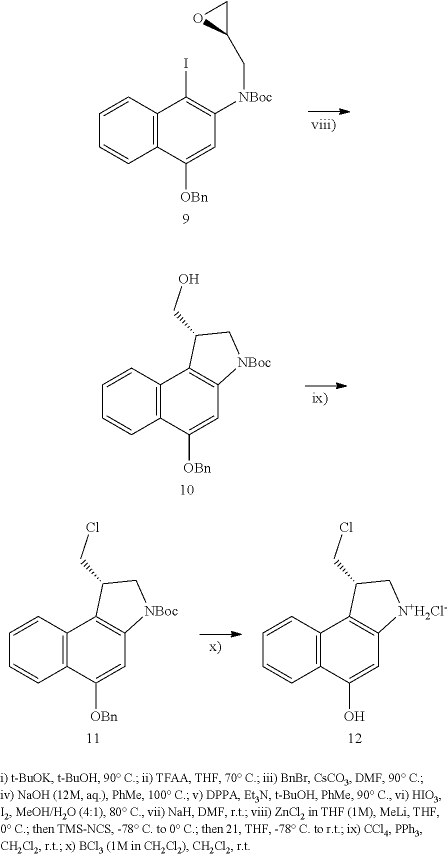

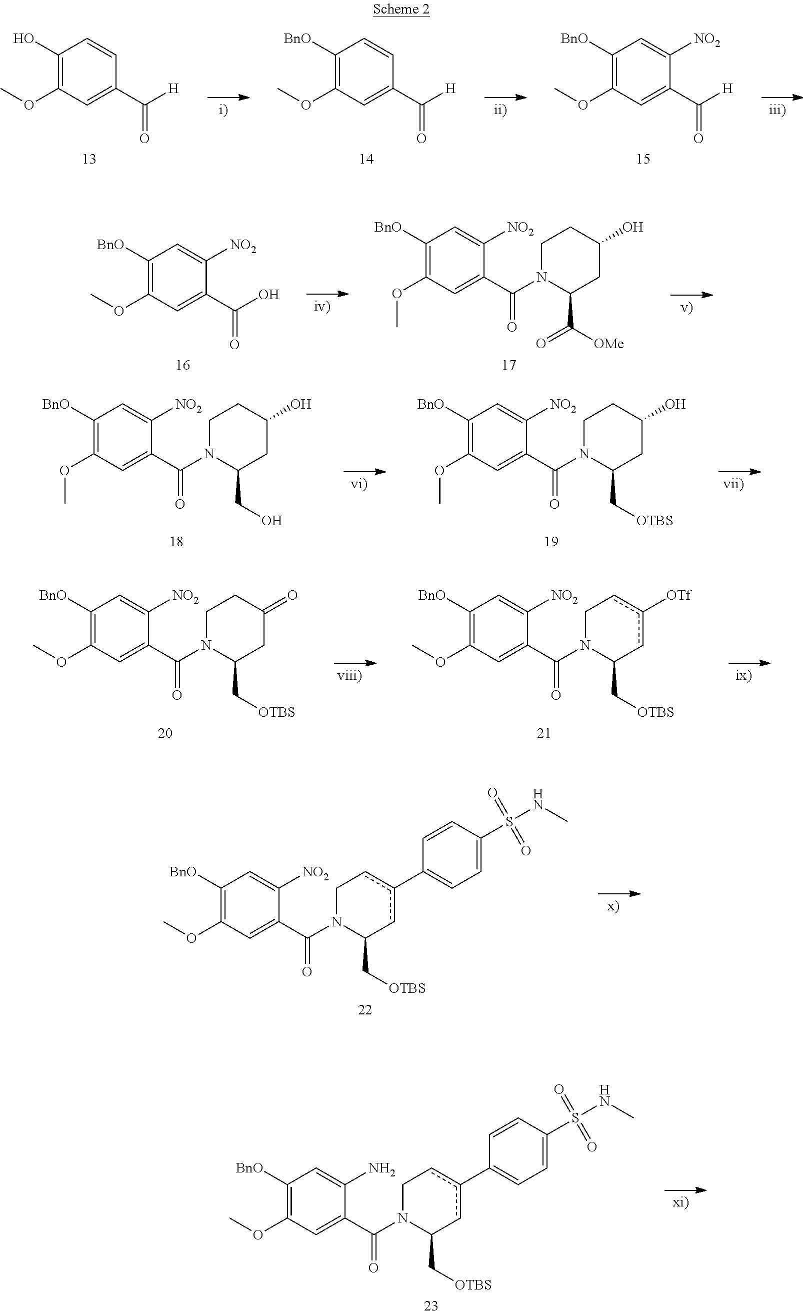

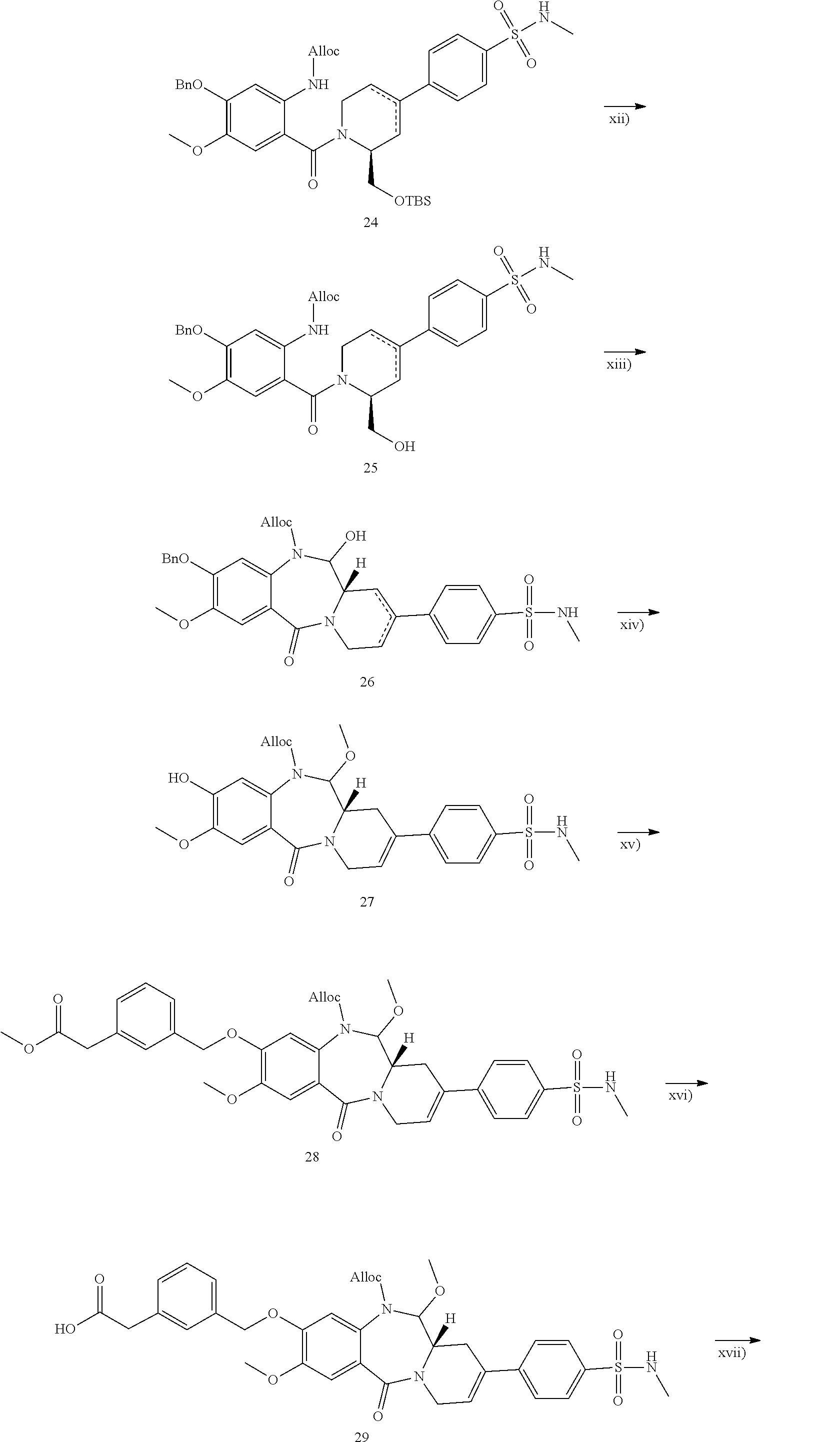

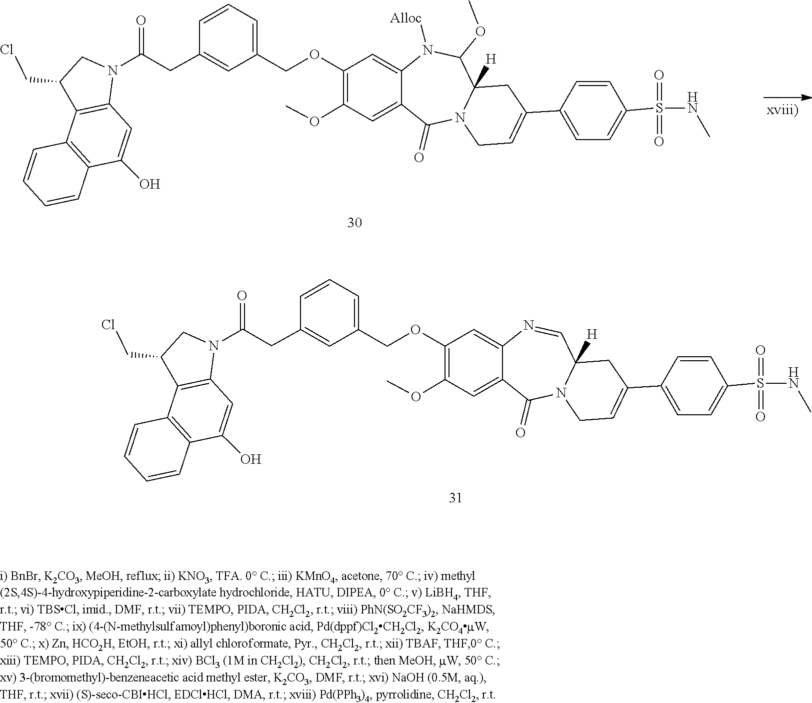

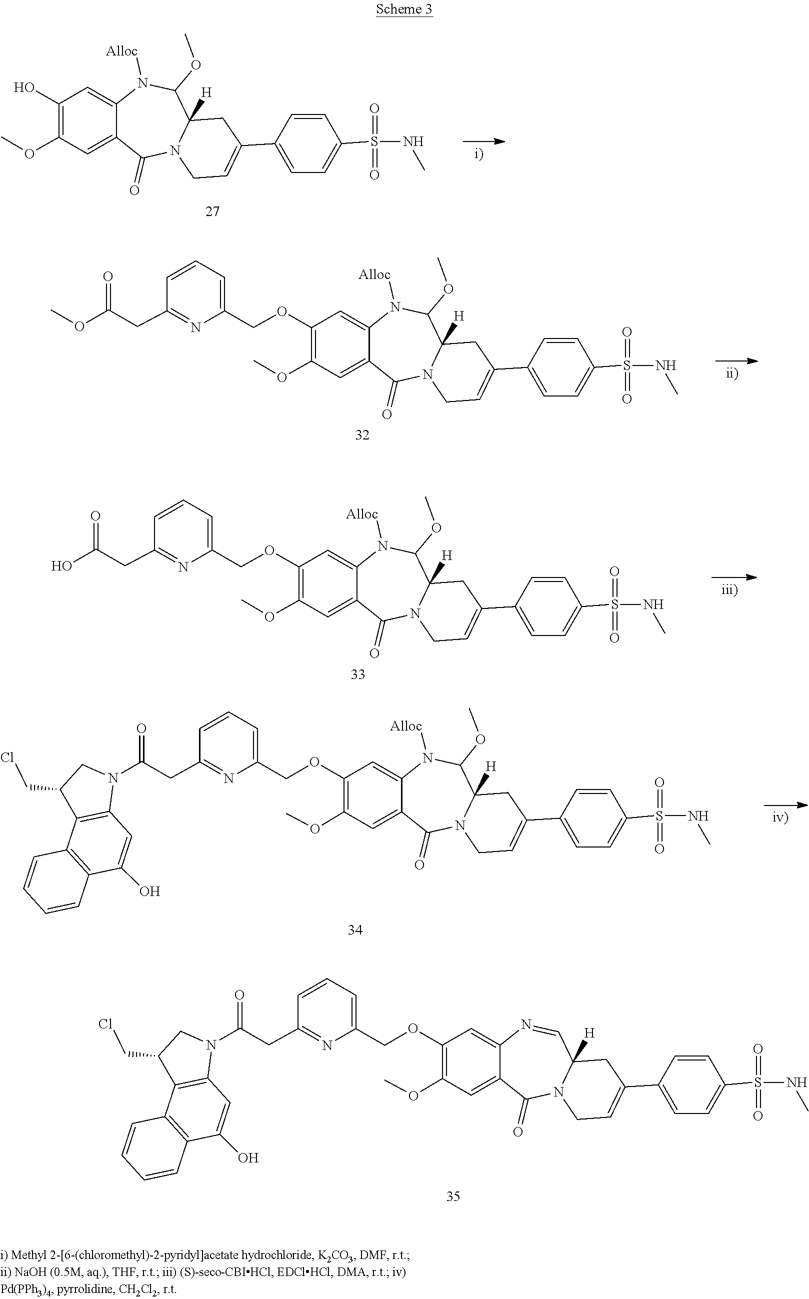

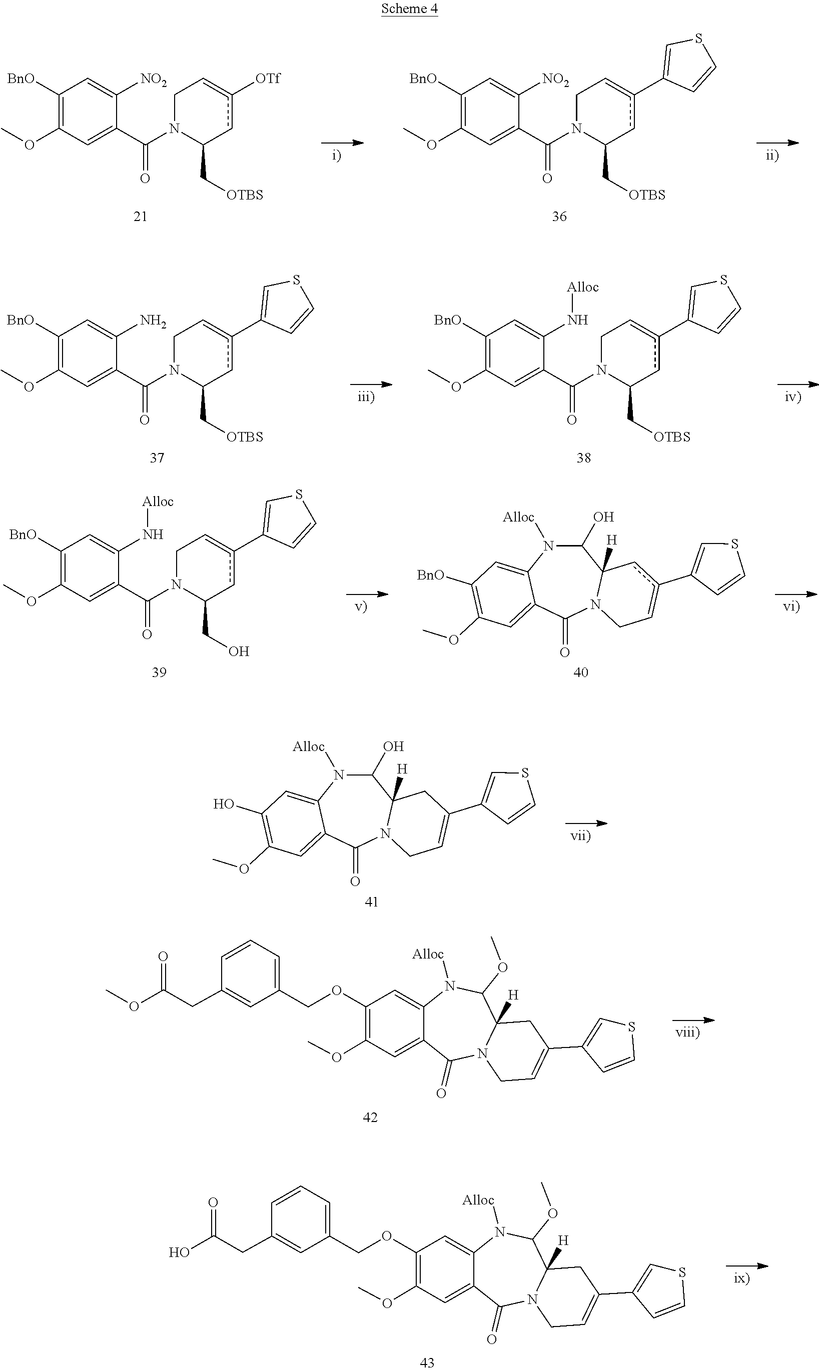

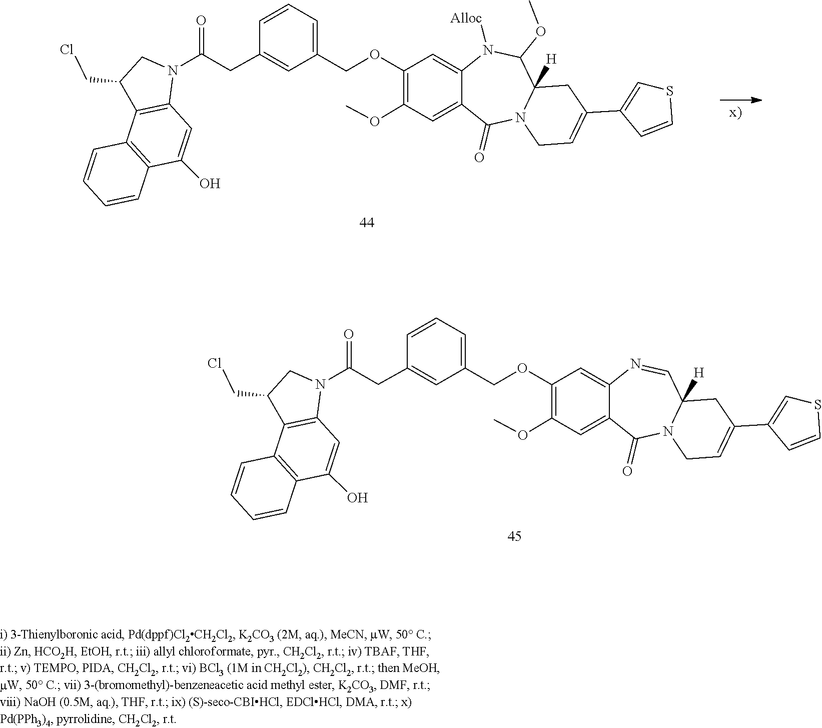

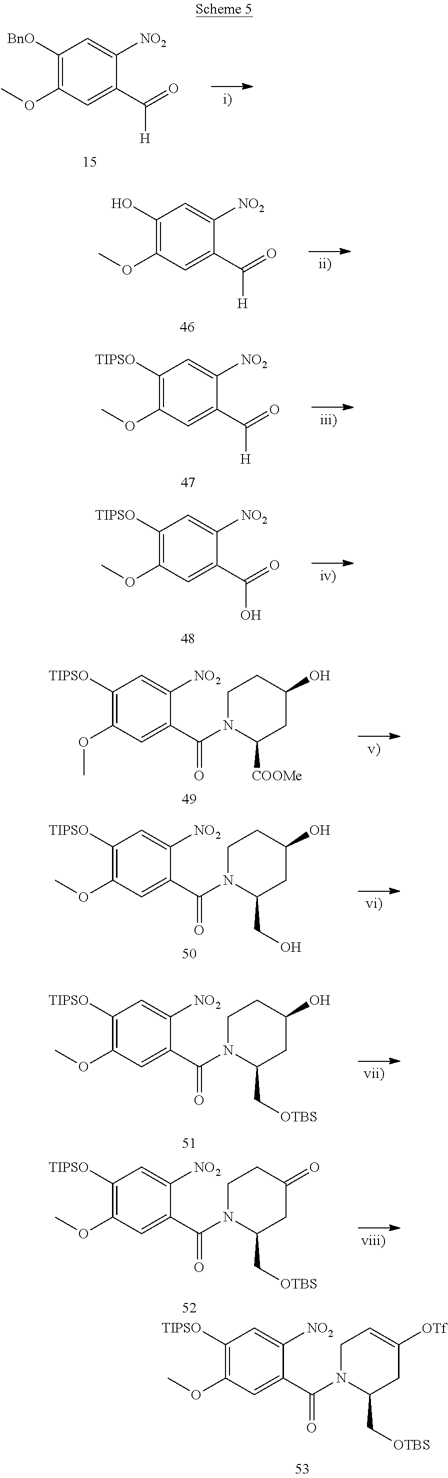

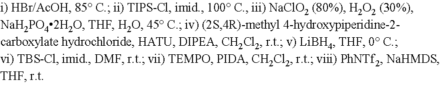

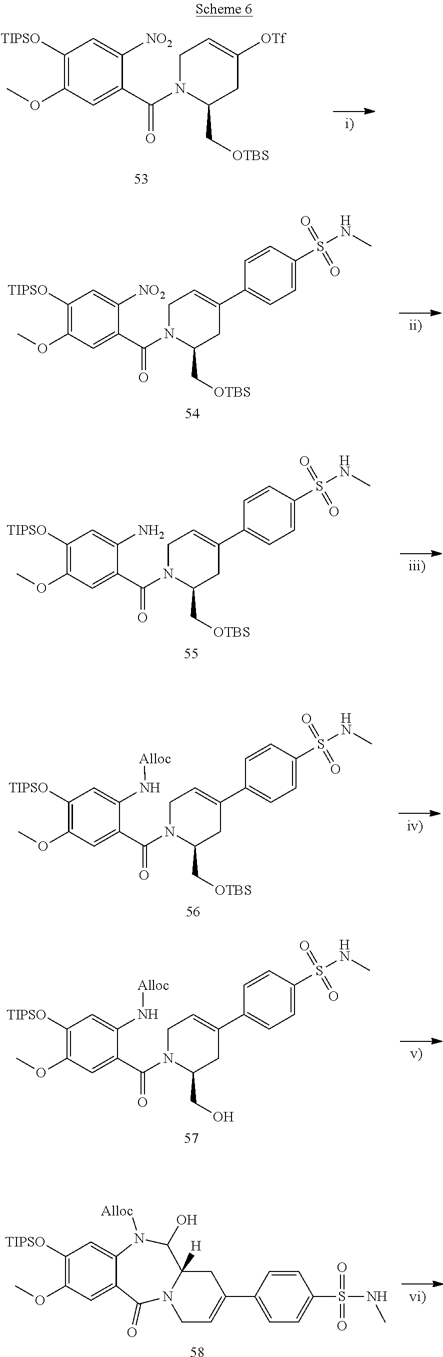

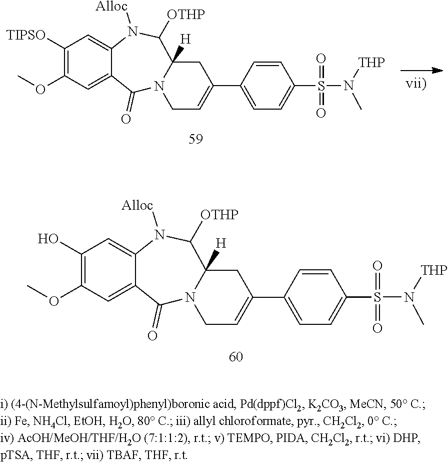

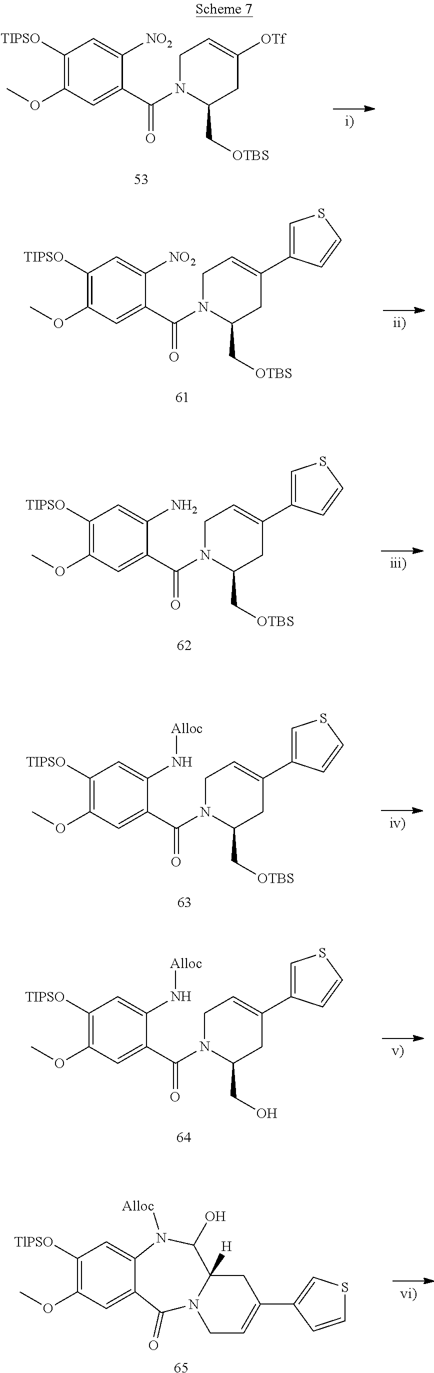

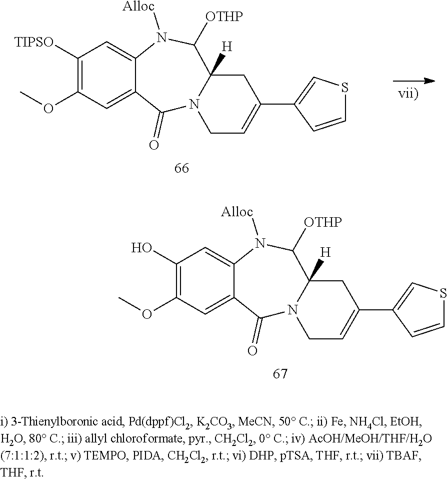

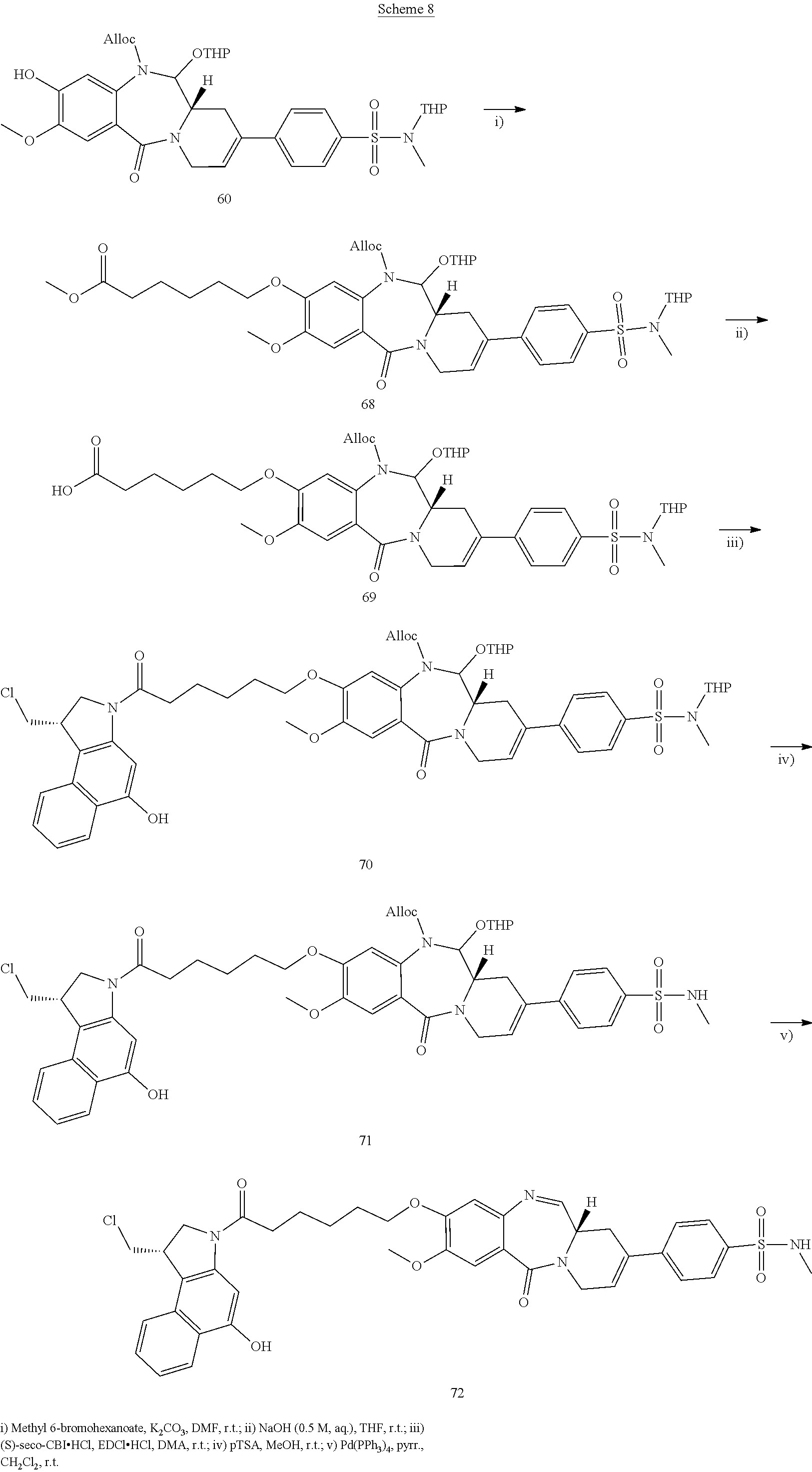

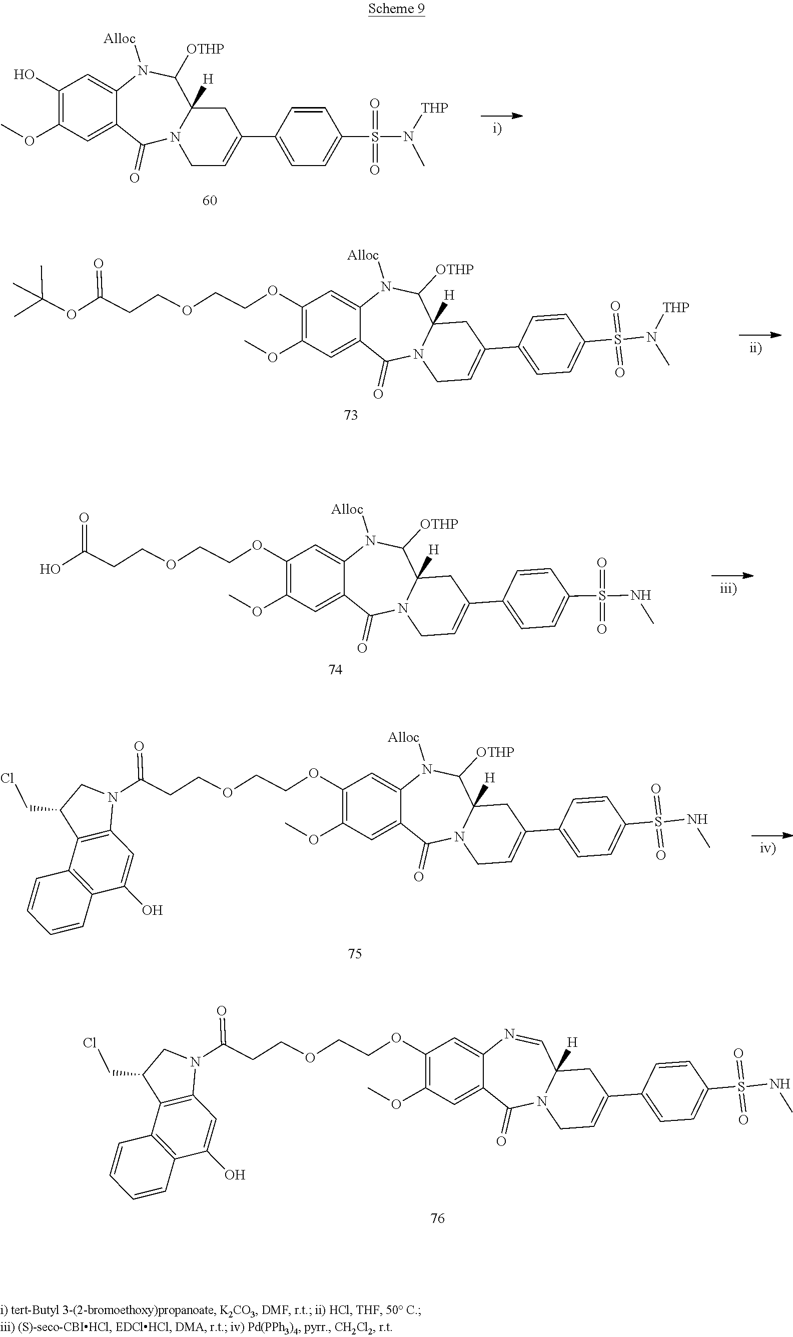

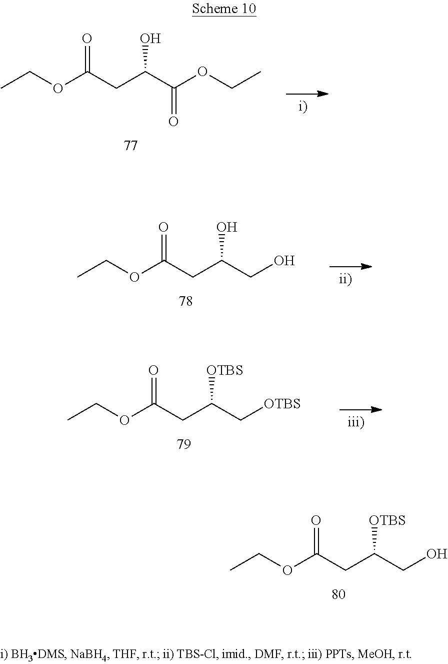

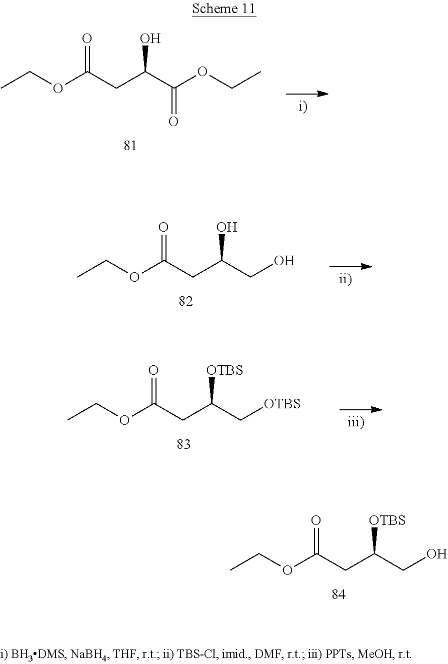

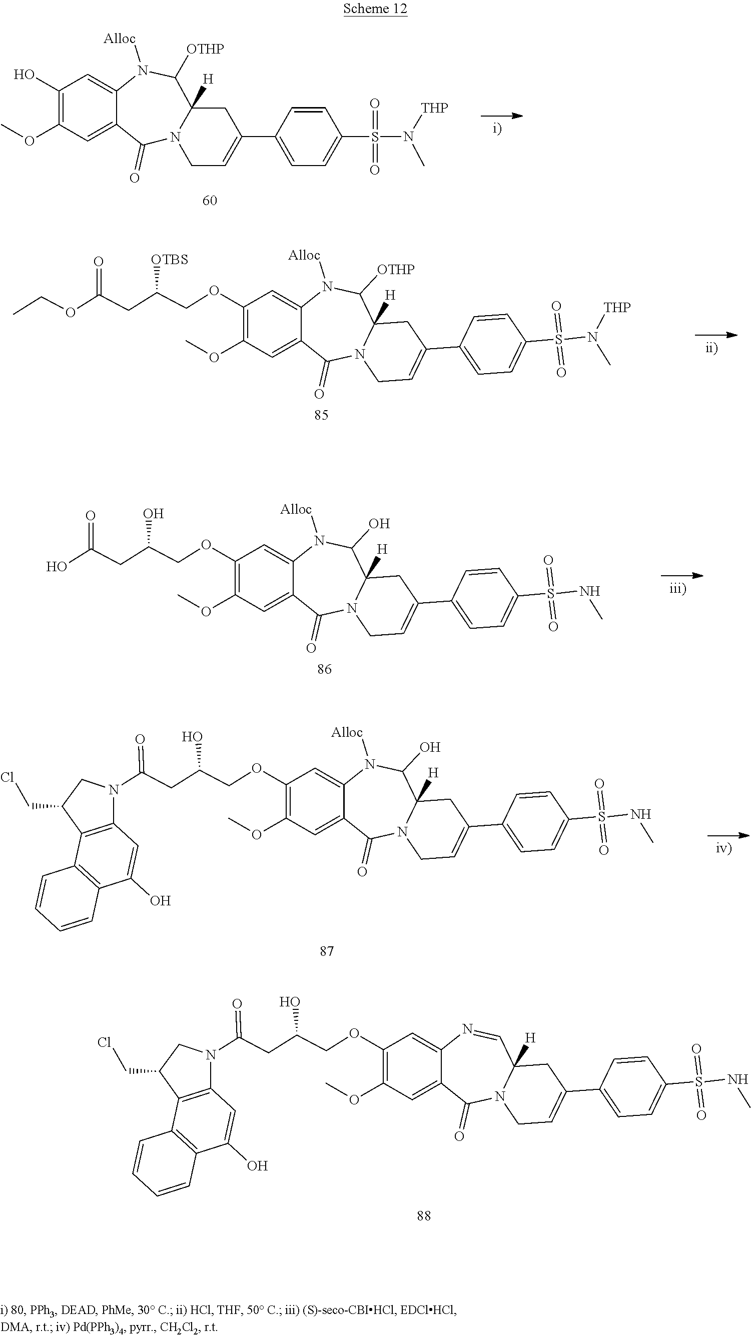

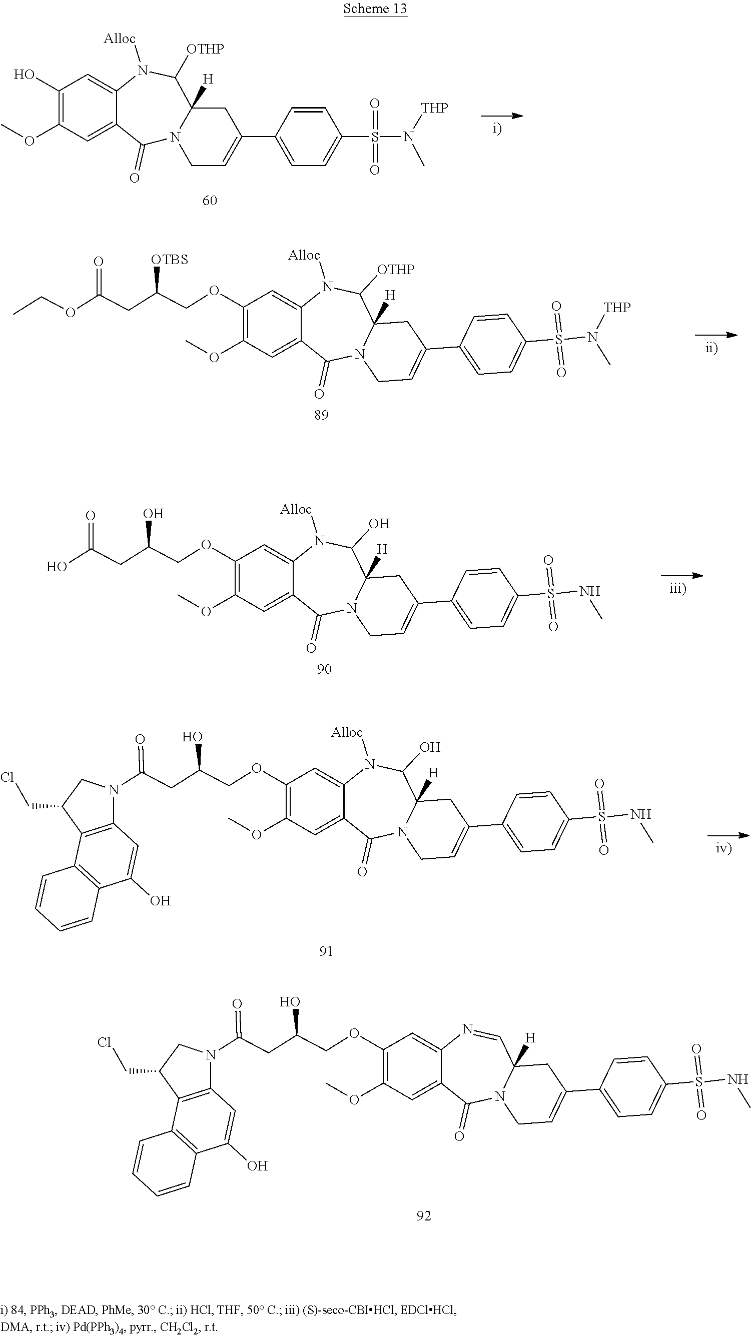

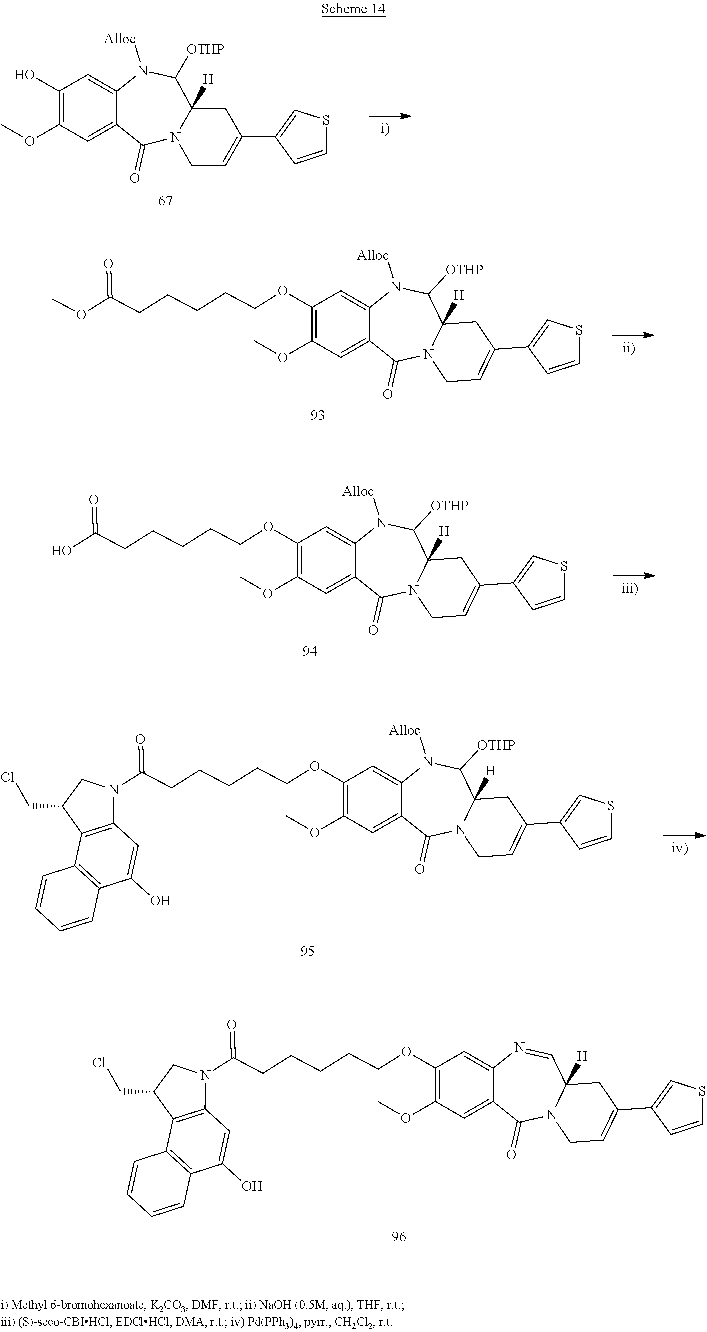

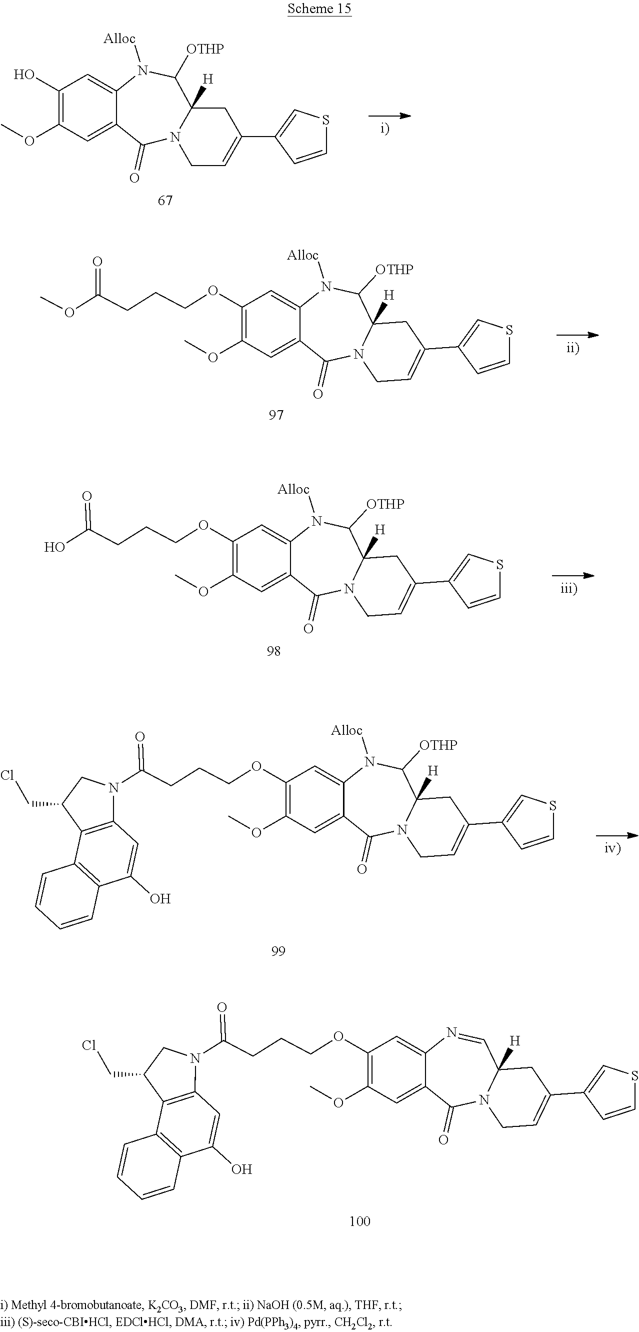

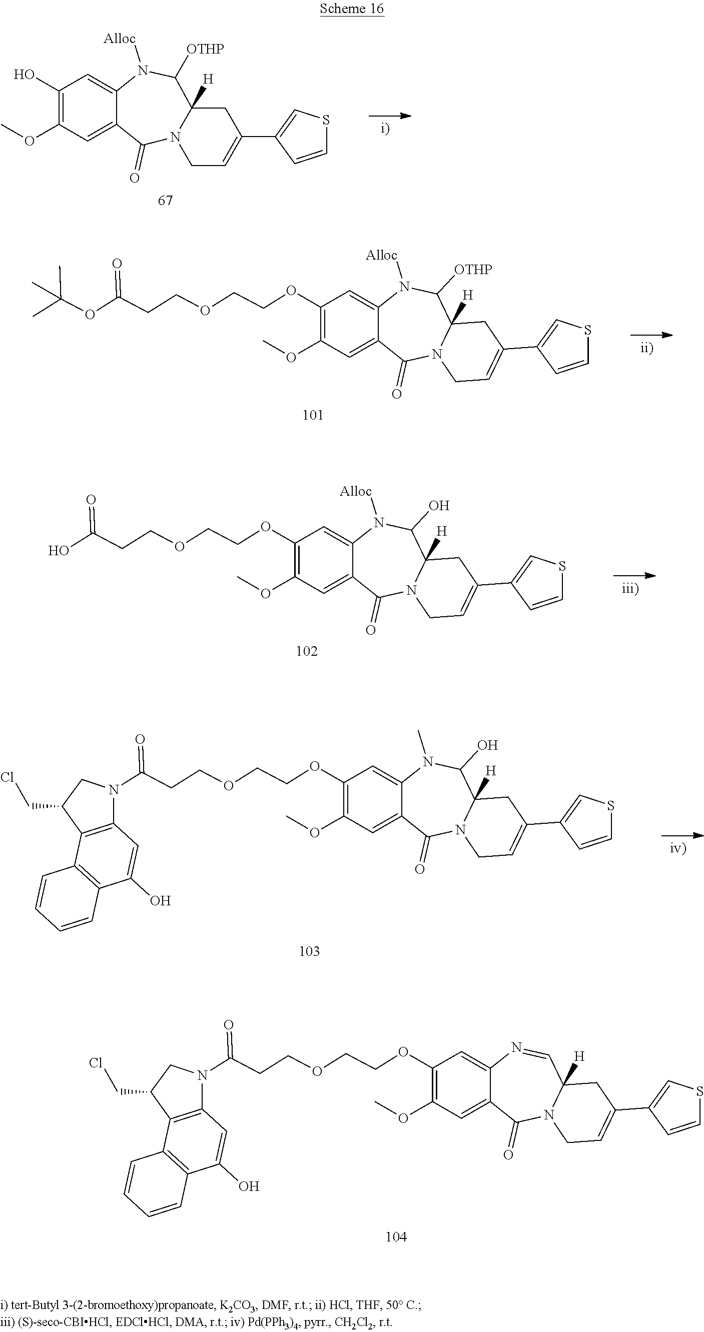

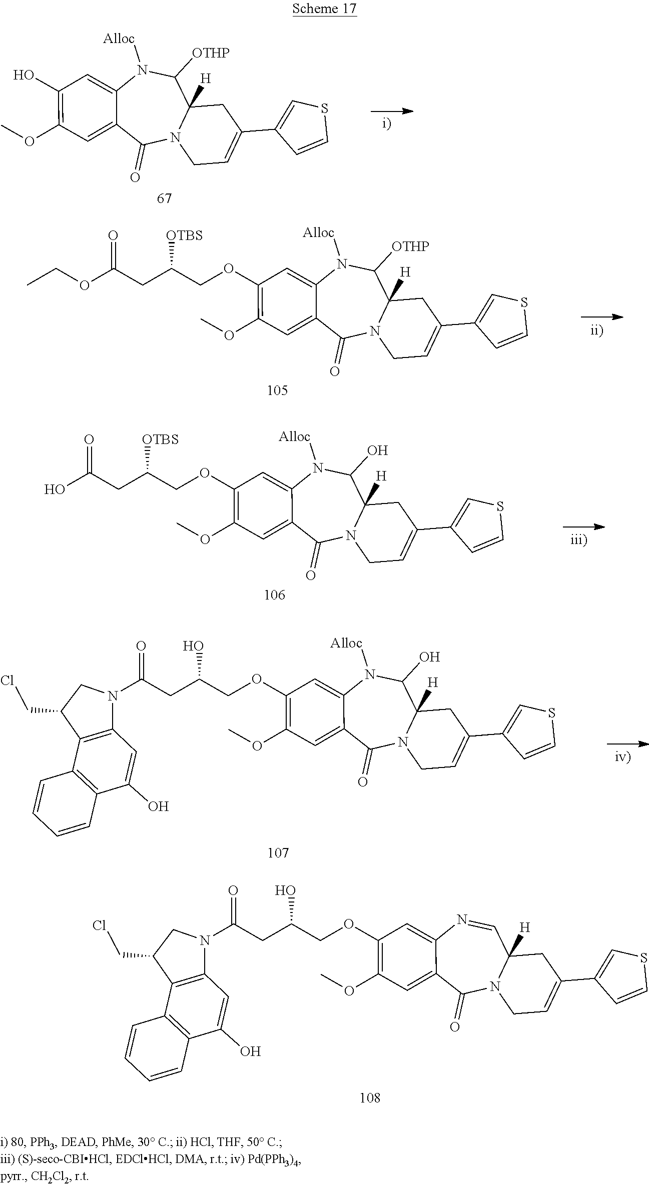

### 3-(Methoxycarbonyl)-4-phenylbut-3-enoic acid (3)

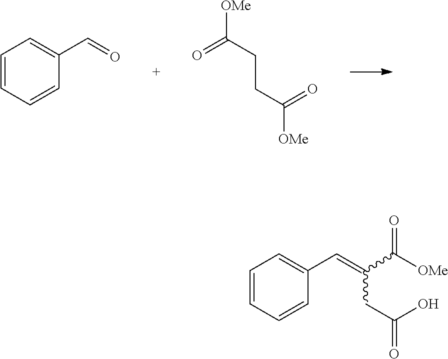

A solution of benzaldehyde (1) (100 g, 942 mmol) and dimethyl succinate (2) (206 g, 1.41 mol) in tert-butanol (500 mL) was added to a refluxing solution of potassium tert-butoxide (158 g, 1.41 mol) in tert-butanol (1.5 L) over 1 h. The mixture was then stirred for a further 30 min before being allowed to cool to room temperature. After concentrating in vacuo, the resulting residue was diluted with water (500 mL) and extracted with ethyl acetate (500 mL). The aqueous phase was then acidified to pH=4-5 with an aqueous solution of hydrochloric acid (6 M), then extracted with ethyl acetate (1 L). The combined organic extracts were dried over sodium sulfate, filtered and concentrated in vacuo to give the title compound (300 g, impure) as a yellow oil which was used in the subsequent step without further purification.

MS (ES+): m/z=221 (M+H)⁺; LCMS (Method F): t_{R}=3.23 min.

### Methyl 4-hydroxy-2-naphthoate (4)

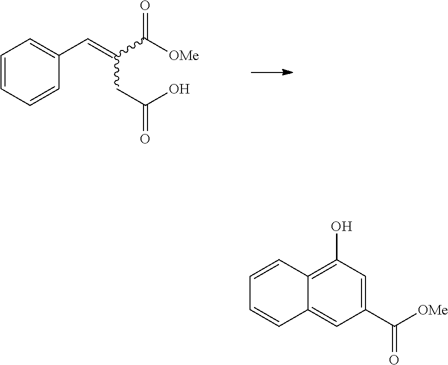

A solution of 3-(methoxycarbonyl)-4-phenylbut-3-enoic acid (3) (300 g) and trifluoroacetic anhydride (99.3 mL, 714 mmol) in tetrahydrofuran (1.5 L) was stirred at 70° C. for 5 h, after which, consumption of starting material was confirmed by TLC. The reaction mixture was then concentrated in vacuo, adjusted to pH=8-9 with an aqueous solution of sodium hydroxide (1 M) and extracted with ethyl acetate (1 L). The organic phase was then dried over sodium sulfate and concentrated in vacuo. Recrystallisation from ethyl acetate/petroleum spirit, 40-60° C. (10%) gave the title compound (100 g, 53%) as a yellow solid.

MS (ES+): m/z=202 (M+H)⁺; LCMS (Method F): t_{R}=3.55 min.

### Methyl 4-(benzyloxy)-2-naphthoate (5)

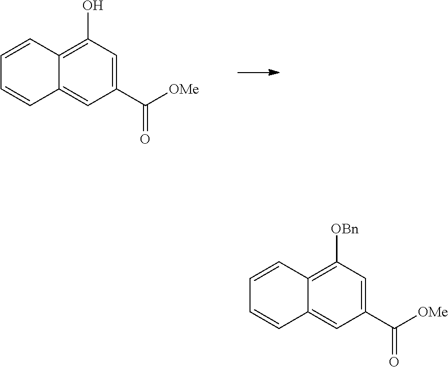 [SMILES: COC(=O)c1cc(O)c2ccccc2c1 ; COC(=O)c1cc(OCc2ccccc2)c2ccccc2c1]

A solution of methyl 4-hydroxy-2-naphthoate (4) (200 g, 990 mmol), benzyl bromide (203 g, 1.19 mol) and caesium carbonate (386 g, 1.19 mol) in N,N-dimethylformamide (800 mL) was stirred at 90° C. for 16 h, after which TLC confirmed consumption of starting material. The mixture was diluted in ethyl acetate (1.5 L), washed with water (1 L×2), then brine (500 mL), dried over sodium sulfate and concentrated in vacuo to give the title compound (250 g, 86%) as a white solid, which was used in the subsequent step without further purification.

### 4-(Benzyloxy)-2-naphthoic acid (6)

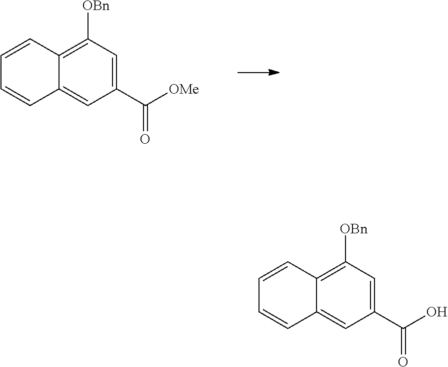 [SMILES: COC(=O)c1cc(OCc2ccccc2)c2ccccc2c1 ; O=C(O)c1cc(OCc2ccccc2)c2ccccc2c1]

A solution of methyl 4-(benzyloxy)-2-naphthoate (5) (250 g, 856 mmol) in toluene (500 mL) was charged with an aqueous solution of sodium hydroxide (12 M, 300 mL) and heated to 100° C. for 16 h, after which TLC confirmed the consumption of starting material. The organic phase was separated and concentrated in vacuo. The residue was then taken up into ethyl acetate (1.5 L) and acidified to pH=2 with an aqueous solution of hydrochloric acid (6 M). The organic phase was separated, dried over sodium sulfate and concentrated in vacuo. Recrystallization from ethyl acetate/petroleum spirit, 40-60° C. (10%) gave the title compound (90 g, 32%) as a white solid.

MS (ES+): m/z=279 (M+H)⁺; LCMS (Method F): t_{R}=4.09 min.

### tert-Butyl (4-(benzyloxy)naphthalen-2-yl)carbamate (7)

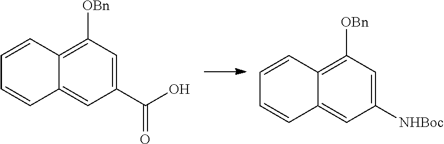 [SMILES: O=C(O)c1cc(OCc2ccccc2)c2ccccc2c1 ; CC(C)(C)OC(=O)Nc1cc(OCc2ccccc2)c2ccccc2c1]

A solution of 4-(benzyloxy)-2-naphthoic acid (6) (50.0 g, 180 mmol), diphenyl phosphoryl azide (41.5 mL, 234 mmol) and triethylamine (28.9 mL, 270 mmol) in toluene (300 mL) was stirred at room temperature for 1 h, after which TLC showed consumption of starting material. tert-Butanol (200 mL) was added and the resulting mixture was stirred at 90° C. for 17 h. This was then diluted with ethyl acetate (1.5 L) and water (500 mL). The organic phase was separated, dried over sodium sulfate, filtered and concentrated in vacuo. Recrystallization from ethyl acetate/petroleum spirit, 40-60° C. (10%) gave the title compound (35 g, 56%) as a pink solid.

MS (ES+): m/z=350 (M+H)⁺; LCMS (Method F): t_{R}=4.67 min.

### tert-Butyl (4-(benzyloxy)-1-iodonaphthalen-2-yl)carbamate (8)

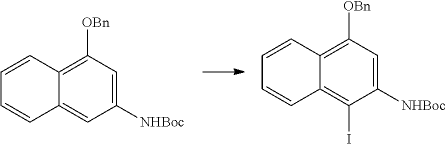 [SMILES: CC(C)(C)OC(=O)Nc1cc(OCc2ccccc2)c2ccccc2c1 ; CC(C)(C)OC(=O)Nc1cc(OCc2ccccc2)c2ccccc2c1I]

A mixture of tert-butyl (4-(benzyloxy)naphthalen-2-yl)carbamate (7) (55.0 g, 157 mmol), iodic acid (5.50 g, 31.5 mmol) and iodine (16.0 g, 63 mmol) in methanol (400 mL) and water (100 mL) was stirred at 80° C. for 5 h, after which TLC showed consumption of starting material. The mixture was diluted with water (1.0 L) and filtered. The resulting cake was washed with methanol (200 mL) and concentrated in vacuo to give the title compound (72 g, 96%) as a brown solid.

MS (ES+): m/z=476 (M+H)⁺; LCMS (Method E): t_{R}=4.91 min.

### tert-Butyl (R)-(4-(benzyloxy)-1-iodonaphthalen-2-yl)(oxiran-2-ylmethyl)carbamate (9)

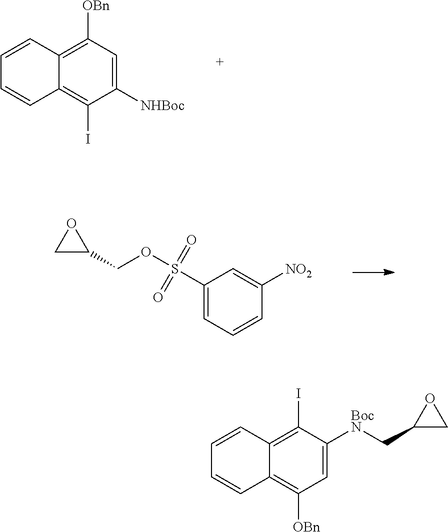

A solution of tert-butyl (4-(benzyloxy)-1-iodonaphthalen-2-yl)carbamate (8) (52 g, 109 mmol) in N,N-dimethylformamide (500 mL) was charged with sodium hydride (60% dispersion in mineral oil, 17 g, 425 mmol) and stirred at room temperature for 30 min, after which (S)-oxiran-2-ylmethyl 3-nitrobenzenesulfonate (51 g, 197 mmol) was added and the resulting mixture stirred for a further 3 h. TLC confirmed consumption of starting material. The reaction mixture was poured cautiously onto ice-water (500 mL) and extracted with ethyl acetate (1.0 L). The organic phase was separated, and washed with water (500 mL) and brine (300 mL), then dried over sodium sulfate and concentrated in vacuo to give the title compound (55 g, 95%) as a white solid.

¹H NMR (400 MHz, CDCl₃) δ 8.33-8.32 (m, 1H), 8.41-8.20 (m, 1H), 7.59-7.48 (m, 4H), 7.45-7.33 (m, 3H), 6.94-6.83 (m, 1H), 5.28 (s, 2H), 4.15-4.09 (m, 1H), 3.50-3.42 (m, 1H), 3.14-3.13 (m, 1H), 2.82-2.60 (m, 1H), 2.41 (ddd, J=12.4, 4.8, 2.8 Hz, 1H), 1.33-1.31 (m, 9H).

### tert-Butyl (S)-5-(benzyloxy)-1-(hydroxymethyl)-1,2-dihydro-3H-benzo[e]indole-3-carboxylate (10)

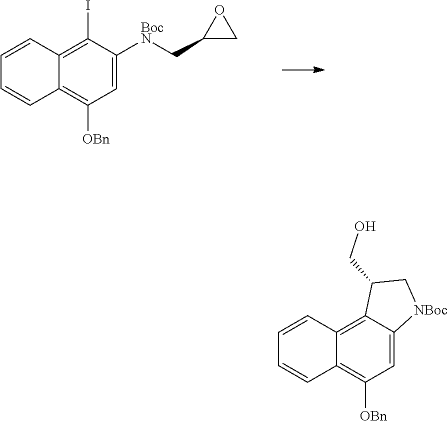 [SMILES: CC(C)(C)OC(=O)N(C[C@@H]1CO1)c1cc(OCc2ccccc2)c2ccccc2c1I ; CC(C)(C)OC(=O)N1C[C@@H](CO)c2c1cc(OCc1ccccc1)c1ccccc21]

Zinc chloride (1 M in tetrahydrofuran, 28 mL) was diluted in anhydrous tetrahydrofuran (40 mL) and cooled to 0° C., under an inert atmosphere of argon. A solution of methyl lithium (1.6 M in diethyl ether, 70.6 mL) was then added to the cooled mixture, dropwise, and stirred for 30 min, before cooling further to −78° C. (Trimethylsilyl)isothiocyanate (4 mL, 28.2 mmol) was added dropwise to the reaction mixture at −78° C., before warming to 0° C. for 30 min and then again cooling to −78° C. A solution of tert-butyl (R)-(4-(benzyloxy)-1-iodonaphthalen-2-yl)(oxiran-2-ylmethyl)carbamate (9) (10 g, 18.8 mmol) in tetrahydrofuran (20 mL) was added dropwise to the reaction mixture at −78° C. for 30 min, then warmed to 0° C. for 1 h, followed by room temperature for 30 min. After quenching with a saturated aqueous solution of ammonium chloride, the mixture was extracted with dichloromethane (500 mL×3) and the combined organics were washed with brine (100 mL), dried over sodium sulfate and concentrated in vacuo to give the title compound (10 g, impure), which was used in the subsequent step without further purification.

¹H NMR (400 MHz, CDCl₃) δ 8.29 (d, J=8.4 Hz, 1H), 7.90 (s, 1H), 7.71 (d, J=8.2 Hz, 1H), 7.55 (d, J=6.8 Hz, 2H), 7.51-7.40 (m, 3H), 7.36-7.32 (m, 2H), 5.27 (s, 2H), 4.22 (d, J=11.4 Hz, 1H), 4.13 (t, J=10.0 Hz, 1H), 4.01-3.95 (m, 1H), 3.85 (bs, 1H), 3.81-3.73 (m, 1H), 1.60 (s, 9H); MS (ES+): m/z=406 (M+H)⁺; LCMS (Method F): t_{R}=4.69 min.

### tert-Butyl (S)-5-(benzyloxy)-1-(chloromethyl)-1,2-dihydro-3H-benzo[e]indole-3-carboxylate (11)

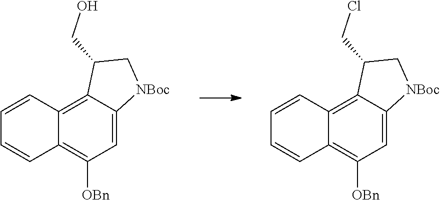 [SMILES: CC(C)(C)OC(=O)N1C[C@@H](CO)c2c1cc(OCc1ccccc1)c1ccccc21 ; CC(C)(C)OC(=O)N1C[C@@H](CCl)c2c1cc(OCc1ccccc1)c1ccccc21]

A solution of tert-butyl (S)-5-(benzyloxy)-1-(hydroxymethyl)-1,2-dihydro-3H-benzo[e]indole-3-carboxylate (10) (10.0 g, 12.4 mmol), carbon tetrachloride (30 mL) and triphenylphosphine (3.90 g, 14.8 mmol) in dichloromethane (50 mL) was stirred at room temperature for 2 h, after which, TLC showed consumption of starting material. The reaction mixture was then concentrated in vacuo. Purification by flash column chromatography (silica), eluting with ethyl acetate/petroleum spirit, 40-60° C. (10%), followed by recrystallisation from dichloromethane/petroleum spirit, 40-60° C. (90%) gave the title compound (1.47 g, 28%) as a white solid.

[α]D²³=−14.5° (c 0.470, CH₂Cl₂); ¹H NMR (400 MHz, CDCl₃) δ 8.29 (d, J=8.4 Hz, 1H), 7.86 (s, 1H), 7.65 (d, J=8.4 Hz, 1H), 7.58-7.30 (m, 7H), 5.27 (s, 2H), 4.27-4.24 (m, 1H), 4.13 (t, J=10.6 Hz, 1H), 4.01-3.87 (m, 2H), 3.44 (t, J=10.4 Hz, 1H), 1.61 (s, 9H); MS (ES+): m/z=424 (M+H)⁺; LCMS (Method D): t_{R}=4.27 min.

### (S)-1-(Chloromethyl)-2,3-dihydro-1H-benzo[e]indol-5-ol hydrochloride (12)

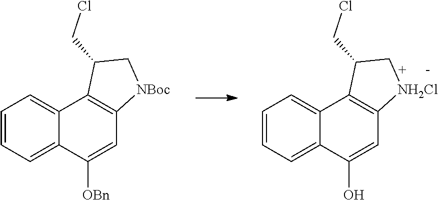

A solution of tert-butyl (S)-5-(benzyloxy)-1-(chloromethyl)-1,2-dihydro-3H-benzo[e]indole-3-carboxylate (11) (100 mg, 0.236 mmol) in anhydrous dichloromethane (3 mL) was charged with boron trichloride (1 M solution in dichloromethane, 708 μL, 0.708 mmol), in a dropwise manner via syringe, at room temperature and stirred under an inert atmosphere of argon. The resulting orange solution was stirred for 5 min before being quenched by cautious addition of methanol (5 mL), then concentrated in vacuo. The residue was charged again with methanol (5 mL) and re-concentrated in vacuo. Diethyl ether (5 mL) was then charged and the residue concentrated in vacuo once again. The residue was then subjected to high vacuum for 30 min to give the title compound (55 mg, impure) as a pale green crystalline solid (unstable), which was used immediately in the subsequent step (amide coupling) without further purification.

MS (ES+): m/z=234 (M+H)⁺; LCMS (Method C): t_{R}=2.62 min.

### 4-(Benzyloxy)-3-methoxybenzaldehyde (14)

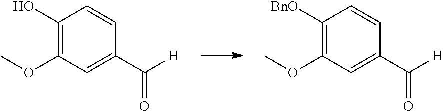 [SMILES: COc1cc(C=O)ccc1O ; COc1cc(C=O)ccc1OCc1ccccc1]

A mixture of compound vanillin (13) (200 g, 1.31 mol), benzyl bromide (236 g, 1.38 mol) and potassium carbonate (545 g, 3.94 mol) in methanol (1.20 L) was refluxed for 5 h. The reaction mixture was filtered, and the filtrate evaporated under reduced pressure to afford the title compound (271 g, 85%) as a pale yellow solid.

¹H NMR (400 MHz, CDCl₃) δ 9.83 (s, 1H), 7.47-7.35 (m, 6H), 7.33 (d, J=70.2 Hz, 1H), 6.98 (d, J=8.2 Hz, 1H), 5.24 (s, 2H), 3.94 (s, 3H); ¹³C NMR (100 MHz, CDCl₃) δ 191.0, 153.6, 150.1, 136.0, 130.3, 128.7, 128.2, 127.2, 126.6, 112.3, 109.3, 70.9, 56.1; MS (ES+): m/z=243 (M+H)⁺; LCMS (Method A): t_{R}=7.53 min.

### 4-(Benzyloxy)-5-methoxy-2-nitrobenzaldehyde (15)

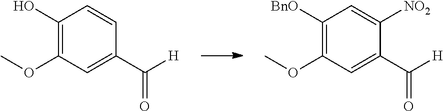 [SMILES: COc1cc(C=O)ccc1O ; COc1cc(C=O)c([N+](=O)[O-])cc1OCc1ccccc1]

A solution of 4-(benzyloxy)-3-methoxybenzaldehyde (14) (130 g, 537 mmol) in trifluoroacetic acid (600 mL) was charged with a solution of potassium nitrate (65 g, 644 mmol), in trifluoroacetic acid (600 mL) dropwise at 0° C. The reaction mixture was stirred for 1 h and then diluted with water (2.40 L). The resulting precipitate was filtered and washed with cold water (500 mL×2) to afford the title compound (125 g, 81%) as a yellow solid.

¹H NMR (400 MHz, CDCl₃) δ 10.43 (s, 1H), 7.67 (s, 1H), 7.46-7.30 (m, 6H), 5.27 (s, 2H), 4.02 (s, 3H); ¹³C NMR (100 MHz, CDCl₃) δ 187.8, 153.7, 151.4, 134.85, 129.0, 128.9, 128.7, 127.6, 125.7, 110.0, 108.9, 71.6, 56.7; MS (ES−): m/z=286 (M−H)⁻; LCMS (Method A): t_{R}=7.87 min.

### 4-(Benzyloxy)-5-methoxy-2-nitrobenzoic acid (16)

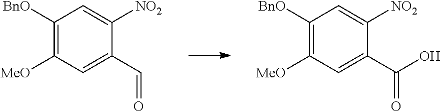 [SMILES: COc1cc(C=O)c([N+](=O)[O-])cc1OCc1ccccc1 ; COc1cc(C(=O)O)c([N+](=O)[O-])cc1OCc1ccccc1]

A solution of 4-(benzyloxy)-5-methoxy-2-nitrobenzaldehyde (15) (8.0 g, 28 mmol) in acetone (300 mL) was quickly charged with a hot (70° C.) solution of potassium permanganate (16.5 g, 104 mmol) in water (150 m1). The mixture was then stirred at 70° C. for 4 h. The reaction mixture was then allowed to cool to room temperature and passed through a pad of celite, which was then washed with hot water (120 mL). A solution of sodium bisulfite in hydrochloric acid (1 M, 120 mL) was added to the filtrate, which was then extracted with dichloromethane (2×200 mL). The combined organic extracts were subsequently dried over sodium sulfate, filtered and concentrated to give the title compound (6.7 g, 79%) as a yellow solid, which was used in the subsequent step without further purification.

¹H NMR (400 MHz, CDCl₃) δ 7-55 (s, 1H), 7.49-7.37 (m, 6H), 5.17 (s, 2H), 4.99 (br s, 1H), 3.93 (s, 3H); ¹³C NMR (100 MHz, MeOD) δ 168.6, 154.1, 151.0, 142.9, 137.3, 129.7, 129.4, 129.0, 123.2, 112.5, 110.0, 72.3, 57.1; MS (ES+): m/z=302 (M+H)+, MS (ES−): m/z=302 (M−1)⁻; LCMS (Method B): t_{R}=3.62 min, LCMS (Method A): t_{R}=7.02 min.

### Methyl (2S,4S)-1-(4-(benzyloxy)-5-methoxy-2-nitrobenzoyl)-4-hydroxypiperidine-2-carboxylate (17)

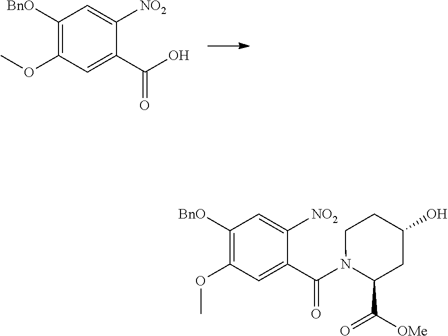 [SMILES: COc1cc(C(=O)O)c([N+](=O)[O-])cc1OCc1ccccc1 ; COC(=O)[C@@H]1C[C@@H](O)CCN1C(=O)c1cc(OC)c(OCc2ccccc2)cc1[N+](=O)[O-]]

A solution of 4-(benzyloxy)-5-methoxy-2-nitrobenzoic acid (16) (20.00 g, 66.0 mmol) in N,N-dimethylformamide (100 mL) was charged with HATU (37.61 g, 98.9 mmol) and N,N-diisopropylethylamine (21-31 g, 164.9 mmol) and stirred for 30 min. The reaction mixture was then cooled to 0° C. and methyl (2S,4S)-4-hydroxypiperidine-2-carboxylate hydrochloride (12.90 g, 66.0 mmol) was added. After the reaction was judged to have completed by TLC, it was diluted with water (300 mL) and extracted with ethyl acetate (500 mL×3). The combined organic extracts were washed with water and dried over solid anhydrous sodium sulfate, filtered and concentrated in vacuo. The residue was purified by flash column chromatography (silica), eluting with acetone/dichloromethane, (from 5% to 40%), to afford the title compound (22.00 g, 75%) as a pale yellow solid.

¹H NMR (400 MHz, DMSO-d₆) mixture of rotamers, δ 7.77 and 7.72 (2×s, 1H), 7.40-7.33 (m, 2H), 7.31-7.20 (m, 3H), 6.94 and 6.73 (2×s, 1H), 5.14 and 5.12 (2×s, 2H), 4.05-3.99 (m, 1H), 3.91 and 3.89 (2×s, 3H), 3.67 and 3.63 (2×s, 3H) 3.60-3.58 (m, 1H), 3.40-3.30 (m, 1H) 2.52 and 2.24 (2×d, J=14.4 and 14.1 Hz, 1H), 1.99-1.90 (m, 1H) 1.89-1.80 (m, 1H) 1.73-1.60 (m, 1H), 1.57-1.48 (m, 1H); MS (ES+): m/z=445 (M+H)⁺; LCMS (Method B): t_{R}=3.25 min.

### (4-(Benzyloxy)-5-methoxy-2-nitrophenyl)((2S,4S)-4-hydroxy-2-(hydroxymethyl)piperidin-1-yl)methanone (18)

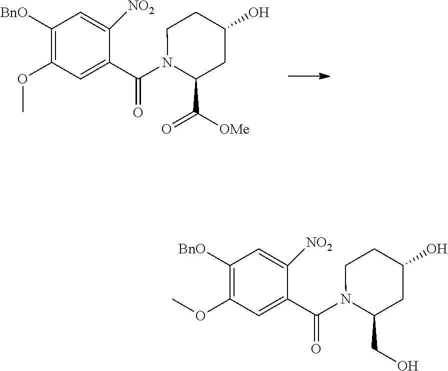 [SMILES: COC(=O)[C@@H]1C[C@@H](O)CCN1C(=O)c1cc(OC)c(OCc2ccccc2)cc1[N+](=O)[O-] ; COc1cc(C(=O)N2CC[C@H](O)C[C@H]2CO)c([N+](=O)[O-])cc1OCc1ccccc1]

A stirred solution of methyl (2S,4S)-1-(4-(benzyloxy)-5-methoxy-2-nitrobenzoyl)-4-hydroxypiperidine-2-carboxylate (17) (4.70 g, 10.5 mmol) in anhydrous tetrahydrofuran (100 mL) was charged with lithium borohydride (2 M in tetrahydrofuran, 7.9 mL, 15.8 mmol) dropwise at 0° C. The reaction was then stirred at room temperature. After 1 h, the reaction was judged to be complete by TLC and was quenched using water (50 mL) and hydrochloric acid solution (1 N, 10 mL). The mixture was then extracted with ethyl acetate (100 mL×3) and the combined organic extracts were washed with sodium hydrogen carbonate (50 mL×2) and brine (50 mL×2), dried over solid anhydrous sodium sulfate, filtered and concentrated in vacuo to afford the title compound (4.0 g, 91%) as an off-white solid.

¹H NMR (400 MHz, MeOD) mixture of rotamers, δ 7.76, 7.74 and 7.71 (3×s, 1H), 7.39-7.22 (m, 5H), 7.01 and 6.91 (2×s, 1H), 5.12 (2×S, 2H), 4.39 (d, J=13.5 Hz, 1H), 4.20 and 4.10 (2×dd, J=90.8, 12.0 and 8.8, 12.0 Hz, 1H), 3.88 and 3.83 (2×s, 3H), 3.65 (dd, J=40.4, 12.0 Hz, 0.5H), 3.57-3.52 (m, 0.8H), 3.50-3.43 (m, 0.6H), 3.34 (dd, J=40.0, 12.0 Hz, 0.6H), 3.26 (dd, J=2.7, 13.3 Hz, 0.5H), 3.12-3.00 (m, 0.5H), 1.90-1.85 (m, 0.5H), 1.85-1.77 (m, 1H), 1.75-1.70 (m, 1H), 1.70-1.55 (m, 2H), 1.52-1.42 (m, 2H); MS (ES+): m/z=417 (M+H)⁺; LCMS (Method B): t_{R}=3.08 min.

### (4-(Benzyloxy)-5-methoxy-2-nitrophenyl)((2S,4S)-2-(((tert-butyldimethylsilyl)oxy)methyl)-4-hydroxypiperidin-1-yl)methanone (19)

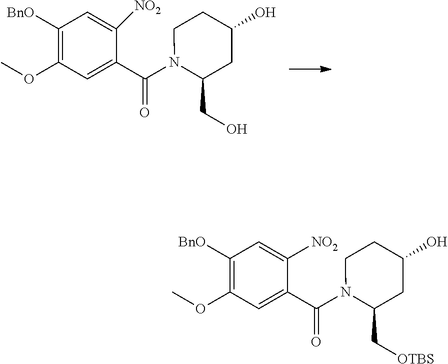 [SMILES: COc1cc(C(=O)N2CC[C@H](O)C[C@H]2CO)c([N+](=O)[O-])cc1OCc1ccccc1 ; COc1cc(C(=O)N2CC[C@H](O)C[C@H]2CO[Si](C)(C)C(C)(C)C)c([N+](=O)[O-])cc1OCc1ccccc1]

A solution of (4-(benzyloxy)-5-methoxy-2-nitrophenyl)((2S,4S)-4-hydroxy-2-(hydroxymethyl)piperidin-1-yl)methanone (18) (7.50 g, 18.0 mmol) in dry N,N-dimethylformamide (15 mL) was charged with imidazole (3.68 g, 54.0 mmol) and tert-butyldimethylsilyl chloride (2.99 g, 19.8 mmol) and then stirred at room temperature. After 2 h, the reaction was found to have partially completed. Additional tert-butyldimethylsilyl chloride (0.54 g, 3.6 mmol) was then added. After stirring for another 30 min, the reaction was judged to have completed by TLC and was diluted with water (100 mL). The mixture was then extracted with ethyl acetate (300 mL×2) and the combined organic extracts washed with citric acid (1 M) (150 mL) and brine (150 mL) and dried over solid anhydrous sodium sulfate, filtered and concentrated in vacuo. The residue was purified by flash column chromatography (silica), eluting with ethyl acetate/petroleum spirit, 40-60° C. (from 25% to 50%) to afford the title compound (6.17 g, 65%) as an orange oil.

¹H NMR (400 MHz, CDCl₃), mixture of rotamers, δ 7.61, 7.60 and 7.57 (3×s, 1H), 7.30-7.15 (m, 5H), 6.61, 6.56 and 6.50 (3×s, 1H), 5.04 and 5.03 (2×S, 2H), 4.86-4.45 (m, 2H), 4.17 (dd, J=30.2, 10.6 Hz, 0.5H), 4.01-3.84 (m, 1-5H), 3.69 (dt, J=50.9, 3.9 Hz, 1H), 3.60 (dd, J=10.6, 2.9 Hz, 1H), 3.47-3.34 (m, 1H), 3.11 and 2.96 (2×t, J=12.2 and 14.5 Hz, 1H), 2.15-1.97 (m, 1H), 1.89 and 1.80 (2×d, J=14.4, 14.9 Hz, 1H), 1.63-1.30 (m, 3H), 0.80-0.67 (m, 9H), 0.15-0.05 (m, 6H); MS (ES+): m/z=531 (M+H)⁺; LCMS (Method B): t_{R}=4.38 min.

### (S)-1-(4-(Benzyloxy)-5-methoxy-2-nitrobenzoyl)-2-(((tert-butyldimethylsilyl)oxy)methyl)piperidin-4-one (20)

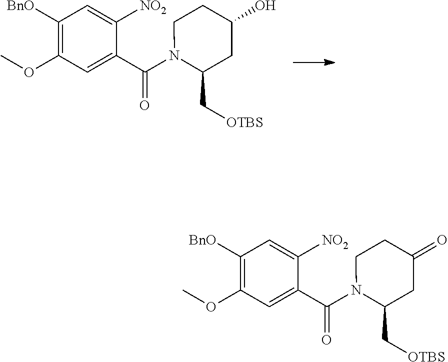 [SMILES: COc1cc(C(=O)N2CC[C@H](O)C[C@H]2CO[Si](C)(C)C(C)(C)C)c([N+](=O)[O-])cc1OCc1ccccc1 ; COc1cc(C(=O)N2CCC(=O)C[C@H]2CO[Si](C)(C)C(C)(C)C)c([N+](=O)[O-])cc1OCc1ccccc1]

A solution of (4-(benzyloxy)-5-methoxy-2-nitrophenyl)((2S,4S)-2-(((tert-butyldimethylsilyl)oxy)methyl)-4-hydroxypiperidin-1-yl)methanone (19) (14.0 g, 26.4 mmol) in dichloromethane (200 mL) was charged with 2,2,6,6-tetramethylpiperidine 1-oxyl (0.41 g, 2.60 mmol) and (diacetoxyiodo)benzene (11.05 g, 34.3 mmol) and stirred at room temperature. After 24 h, the reaction was judged to have completed by TLC. The mixture was then extracted with ethyl acetate (300 mL×2) and the combined organic extracts were washed with a saturated aqueous solution of sodium metabisulfite (150 mL) and brine (150 mL) and dried over anhydrous sodium sulfate, filtered and concentrated in vacuo. The residue was purified by flash column chromatography (silica), eluting with ethyl acetate/petroleum spirit, 40-60° C. (from 25% to 50%) to afford the title compound (13.00 g, 90%) as a yellow solid.

¹H NMR (400 MHz, CDCl₃), mixture of rotamers, δ 7.69, 7.68 and 7.67 (3×s, 1H), 7.40-7.20 (m, 5H), 6.71, 6.70 and 6.65 (3×s, 1H), 5.14 and 5.13 (2×s, 2H), 4.12 and 3.91 (2×dd, J=10.6, 2.6 and 10.4, 2.7 Hz, 2H), 3.89, 3.88 and 3.87 (3×s, 3H), 3.80-3.35 (m, 3H), 2.77-2.15 (m, 4H), 0.85-0.72 (m, 9H), 0.10-0.02 (m, 6H); ¹³C NMR (100 MHz, CDCl₃), mixture of rotamers, δ 205.7, 167.5, 154.9, 148.3, 148.2, 137.6, 137.3, 135.2, 130.0, 128.9, 128.7, 128.6, 127.6, 127.3, 127.1, 127.0, 109.9, 109.2, 108.6, 71.4, 65.6, 65.1, 64.6, 56.8, 56.8, 56.7, 56.5, 51.3, 51.2, 43.3, 41.9, 41.5, 41.3, 39.9, 39.8, 39.6, 37.5, 25.8, 25.8, 25.8, 20.3, 18.3, 18.2, 18.2, 18.1, −5.7, −5.7; MS (ES+): m/z=529 (M+H)⁺; LCMS (Method A): t_{R}=8.28 min.

### (S)-1-(4-(Benzyloxy)-5-methoxy-2-nitrobenzoyl)-2-(((tert-butyldimethylsilyl)-oxy)methyl)-1,2,3,6-tetrahydropyridin-4-yl trifluoromethanesulfonate and (S)-1-(4-(benzyloxy)-5-methoxy-2-nitrobenzoyl)-6-(((tert-butyldimethylsilyl)oxy)methyl)-1,2,3,6-tetrahydropyridin-4-yl trifluoromethanesulfonate (1:1 regioisomers) (21)

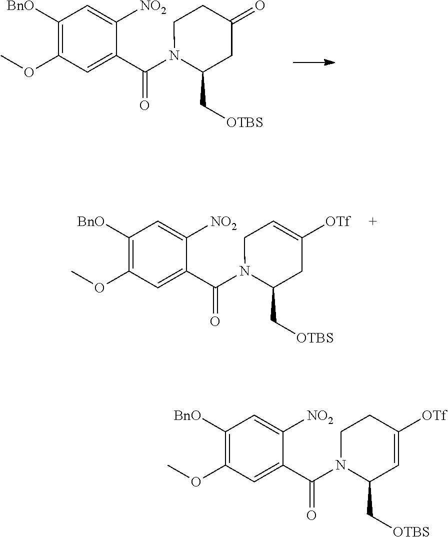 [SMILES: COc1cc(C(=O)N2CCC(=O)C[C@H]2CO[Si](C)(C)C(C)(C)C)c([N+](=O)[O-])cc1OCc1ccccc1 ; COc1cc(C(=O)N2CC=C(OS(=O)(=O)C(F)(F)F)C[C@H]2CO[Si](C)(C)C(C)(C)C)c([N+](=O)[O-])cc1OCc1ccccc1 ; COc1cc(C(=O)N2CCC(OS(=O)(=O)C(F)(F)F)=C[C@H]2CO[Si](C)(C)C(C)(C)C)c([N+](=O)[O-])cc1OCc1ccccc1]

A solution of(S)-1-(4-(benzyloxy)-5-methoxy-2-nitrobenzoyl)-2-(((tert-butyldimethylsilyl)oxy)methyl)piperidin-4-one (20) (13.00 g, 24.6 mmol) in anhydrous tetrahydrofuran (100 mL) was cooled to −78° C., and charged with sodium bis(trimethylsilyl)amide (2 M in tetrahydrofuran, 18.4 mL, 36.9 mmol) dropwise. The reaction mixture was stirred for 1 h, before a solution of N-phenyl-bis(trifluoromethanesulfonimide) (11.42 g, 32.0 mmol) in anhydrous tetrahydrofuran (100 mL) was added dropwise. The resulting mixture was then allowed to warm to room temperature and stirred for 4 h. At this point, the reaction was judged to have completed by TLC. The mixture was then concentrated in vacuo and the residue purified by flash column chromatography (silica), eluting with petroleum spirit, 40-60° C./ethyl acetate (10:1) then acetone/dichloromethane (1%) to afford the title compound (12.8 g, 79%) as a cream solid.

¹H NMR (400 MHz, CDCl₃), mixture of rotamers and regioisomers, δ 7.70-7.60 (m, 1H), 7.39-7.25 (m, 5H), 6.77-6.60 (m, 1H), 5.91-5.45 (m, 1H), 5.15-5.00 (m, 2H), 3.95-3.77 (m, 2H), 3.90-3.85 (m, 3H), 3.75-3.20 (m, 3H), 2.90-2.08 (m, 2H), 0.85-0.6 (m, 9H), 0.22-0.07 (m, 6H); MS (ES+): m/z=661 (M+H)⁺; LCMS (Method A): t_{R}=9.27 min.

### (S)-4-(1-(4-(Benzyloxy)-5-methoxy-2-nitrobenzoyl)-6-(((tert-butyldimethylsilyl)oxy)methyl)-1,2,3,6-tetrahydropyridin-4-yl)-N-methylbenzenesulfonamide and (S)-4-(1-(4-(benzyloxy)-5-methoxy-2-nitrobenzoyl)-2-(((tert-butyldimethylsilyl)oxy)methyl)-1,2,3,6-tetrahydropyridin-4-yl)-N-methylbenzenesulfonamide (1:1 regioisomers) (22)

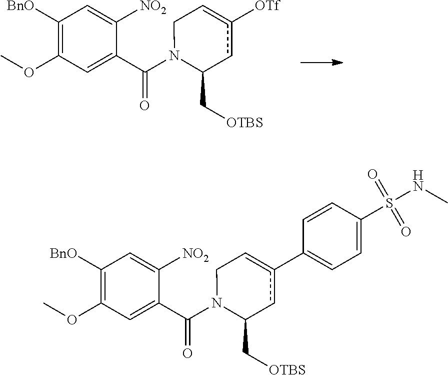

A solution of(S)-1-(4-(benzyloxy)-5-methoxy-2-nitrobenzoyl)-6-(((tert-butyldimethyl-silyl)oxy)methyl)-1,2,3,6-tetrahydropyridin-4-yl trifluoromethanesulfonate and (S)-1-(4-(benzyloxy)-5-methoxy-2-nitrobenzoyl)-2-(((tert-butyldimethylsilyl)oxy)methyl)-1,2,3,6-tetrahydropyridin-4-yl trifluoromethanesulfonate (21) (2.30 g, 3.45 mmol) in acetonitrile (5 mL) was charged with [1,1′-bis(diphenylphosphino)ferrocene]dichloro-palladium(II), complex with dichloromethane (0.284 g, 0-345 mmol), (4-(N-methyl-sulfamoyl)phenyl)boronic acid (0.951 g, 4.40 mmol) and an aqueous solution of potassium carbonate (2 M, 3.45 mL, 6.90 mmol) and irradiated with microwaves at 50° C. for 10 min. The resulting mixture was diluted with ethyl acetate (100 mL) and washed with brine (50 mL), then dried over solid anhydrous magnesium sulfate and concentrated in vacuo. The residual oil was purified by flash column chromatography (silica), eluting with acetone/dichloromethane (10%) to afford the title compound (2.09 g, 88%) as an orange solid.

¹H NMR (400 MHz, CDCl₃), mixture of rotamers and regioisomers, δ 7.88-7.71 (m, 3H), 7.55-7.31 (m, 5H), 6.89-6.73 (m, 1H), 6.44-5.93 (m, 4H), 5.28-4.90 (m, 1H), 5.24 and 5.23 (2×s, 2H), 4.38-4.28 (m, 1H), 4.08-3.41 (m, 3H), 3.97 (2×s, 3H), 3.21-2.70 (m, 1H), 2.70-2.63 (m, 3H), 2.61-2.23 (m, 1H), 0.94-0.71 (m, 9H), 0.17-0.15 (m, 6H); MS (ES+): m/z=682 (M+H)⁺; LCMS (Method B): t_{R}=4.13 min.

### (S)-4-(1-(2-Amino-4-(benzyloxy)-5-methoxybenzoyl)-6-(((tert-butyl-dimethyl-silyl)oxy)methyl)-1,2,3,6-tetrahydropyridin-4-yl)-N-methylbenzenesulfonamide and (S)-4-(1-(2-amino-4-(benzyloxy)-5-methoxybenzoyl)-2-(((tert-butyldimethylsilyl)oxy)methyl)-1,2,3,6-tetra-hydropyridin-4-yl)-N-methylbenzenesulfonamide (1:1 regioisomers) (23)

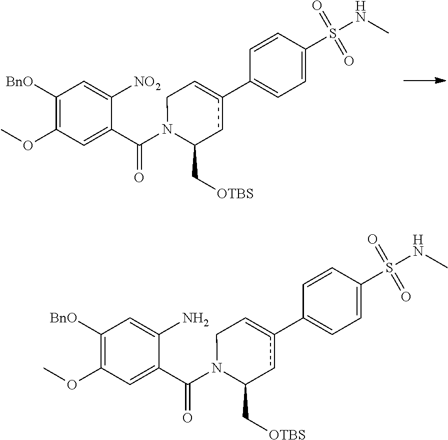

A solution of(S)-4-(1-(4-(Benzyloxy)-5-methoxy-2-nitrobenzoyl)-6-(((tert-butyldimethylsilyl)oxy)methyl)-1,2,3,6-tetrahydropyridin-4-yl)-N-methylbenzenesulfonamide and (S)-4-(1-(4-(benzyloxy)-5-methoxy-2-nitrobenzoyl)-2-(((tert-butyldimethylsilyl)oxy)methyl)-1,2,3,6-tetrahydropyridin-4-yl)-N-methylbenzenesulfonamide (22) (2.40 g, 3.52 mmol) in formic acid (5% v/v in absolute ethanol, 80 mL) was charged with zinc powder (8.45 g, 130.2 mmol) and stirred for 30 min, whilst monitoring by TLC and LCMS. Upon completion, the reaction was quenched by addition of a saturated aqueous solution of sodium hydrogen carbonate (10 mL) and concentrated in vacuo. The residue was then partitioned between ethyl acetate (200 mL) and brine (100 mL) and the organic phase dried over magnesium sulfate, filtered, and concentrated in vacuo. Purification by flash column chromatography (silica), eluting with acetone/dichloromethane (10%) gave the title compound (2.07 g, 90%) as a yellow solid.

¹H NMR (400 MHz, CDCl₃), mixture of rotamers and regioisomers, δ 8.00 (s, 1H), 7.82 (dd, J=8.4, 1.7 Hz, 2H), 7.51 (d, J=8.4 Hz, 2H), 7.45-7.27 (m, 5H), 6.81-6.72 (2×s, 1H), 6.31-6.26 (2×s, 1H), 6.27-6.08 (m, 1H), 5.13 (s, 2H), 4.40 (q, J=50.2 Hz, 1H), 4.05-3.58 (m, 3H), 3.81 (s, 3H), 2.91-2.80 (m, 1H), 2.70-2.62 (m, 3H), 2.53-2.36 (m, 2H), 0.92-0.78 (m, 9H), 0.13-0.04 (m, 6H); MS (ES+): m/z=652 (M+H)⁺; LCMS (Method B): t_{R}=3.97 min.

### Allyl (S)-(5-(benzyloxy)-2-(6-(((tert-butyldimethylsilyl)oxy)methyl)-4-(4-(N-methylsulfamoyl)phenyl)-1,2,3,6-tetrahydropyridine-1-carbonyl)-4-methoxyphenyl)carbamate and allyl (S)-(5-(benzyloxy)-2-(2-(((tert-butyldimethylsilyl)oxy)methyl)-4-(4-(N-methylsulfamoyl)phenyl)-1,2,3,6-tetrahydropyridine-1-carbonyl)-4-methoxyphenyl)carbamate (1:1 regioisomers) (24)

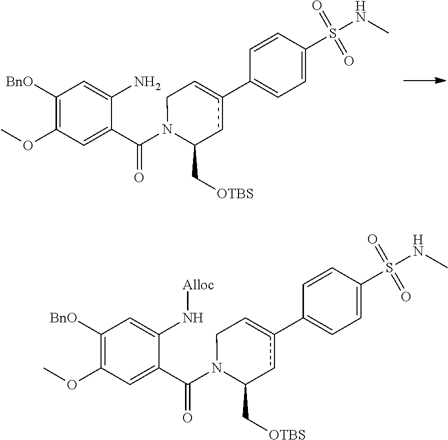

A solution of(S)-4-(1-(2-amino-4-(benzyloxy)-5-methoxybenzoyl)-6-(((tert-butyldimethylsilyl)oxy)methyl)-1,2,3,6-tetrahydropyridin-4-yl)-N-methylbenzenesulfonamide and (S)-4-(1-(2-amino-4-(benzyloxy)-5-methoxybenzoyl)-2-(((tert-butyldimethylsilyl)oxy)methyl)-1,2,3,6-tetrahydropyridin-4-yl)-N-methylbenzenesulfonamide (23) (2.20 g, 3-37 mmol) in dichloromethane (200 mL) was charged with pyridine (0.27 mL, 3.38 mmol) and allyl chloroformate (0.429 mL, 4.25 mmol). After 15 min, the reaction was judged to be complete by TLC and LCMS and was sequentially washed with a saturated aqueous solution of copper sulfate (2×100 mL) and brine (100 mL). The organic extract was then dried over magnesium sulfate and concentrated in vacuo. The resulting oil was then purified by flash column chromatography (silica), eluting with acetone/dichloromethane (5%) to afford the title compound (2.06 g, 83%) as a brown oil.

¹H NMR (400 MHz, CDCl₃), mixture of rotamers and regioisomers, δ 8.43-7.90 (m, 2H), 7.83 (d, J=8.4 Hz, 2H) 7.56-7.28 (m, 7H), 6.97-6.77 (2×s, 1H), 6.23 (s, 1H), 5.92 (ddd, J=22.6, 10.9, 5.7 Hz, 1H), 5.40-5.27 (m, 1H), 5.24-5.12 (m, 3H), 4.08-3.35 (m, 3H), 3.81 (s, 3H), 2.88-2.76 (m, 2H), 2.69-2.64 (m, 3H), 2.62-2.36 (m, 3H), 0.94-0.75 (m, 9H), 0.20-0.06 (m, 6H); MS (ES+): m/z=736 (M+H)⁺; LCMS (Method B): t_{R}=4.22 min.

### Allyl (S)-(5-(benzyloxy)-2-(6-(hydroxymethyl)-4-(4-(N-methylsulfamoyl)-phenyl)-1,2,3,6-tetrahydropyridine-1-carbonyl)-4-methoxyphenyl)-carbamate and allyl (S)-(5-(benzyloxy)-2-(2-(hydroxymethyl)-4-(4-(N-methylsulfamoyl)phenyl)-1,2,3,6-tetrahydropyridine-1-carbonyl)-4-methoxyphenyl)carbamate (1:1 regioisomers) (25)

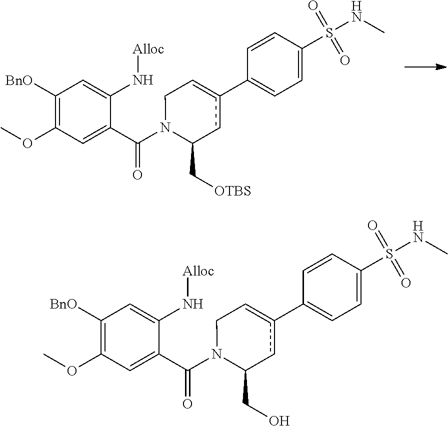

A solution of allyl (S)-(5-(benzyloxy)-2-(6-(((tert-butyldimethylsilyl)oxy)methyl)-4-(4-(N-methylsulfamoyl)phenyl)-1,2,3,6-tetrahydropyridine-1-carbonyl)-4-methoxyphenyl)carbamate and allyl (S)-(5-(benzyloxy)-2-(2-(((tert-butyldimethylsilyl)oxy)methyl)-4-(4-(N-methylsulfamoyl)phenyl)-1,2,3,6-tetrahydropyridine-1-carbonyl)-4-methoxyphenyl)carbamate (24) (1.90 g, 2.58 mmol) in tetrahydrofuran (50 mL) was cooled to 0° C. and charged with tetrabutylammonium fluoride (1 M in tetrahydrofuran, 0.32 mL, 3.23 mmol). The reaction was allowed to warm to room temperature and after 1 h, TLC and LCMS confirmed consumption of starting material. Ethyl acetate (100 mL) was added to the reaction mixture and the resulting solution washed with a saturated aqueous solution of ammonium chloride (50 mL) and brine (50 mL) and dried over magnesium sulfate. Purification by flash column chromatography (silica), eluting with acetone/dichloromethane (20%) gave the title compound (1.52 g, 95%) as a cream solid.

¹H NMR (400 MHz, CDCl₃), mixture of rotamers and regioisomers, δ 8.17-8.06 (br, 1H), 7.83-7.70 (m, 3H), 7.53-7.28 (m, 7H), 6.89-6.77 (s, 1H), 6.23-6.08 (m, 1H), 5.91 (ddd, J=22.8, 10.8, 5.6 Hz, 1H), 5.38-5.18 (m, 2H), 5.15 (s, 2H), 4.66-4.57 (m, 2H), 4.54-4.45 (m, 1H), 4.14-3.89 (m, 1H), 3.85-3.83 (2×s, 3H), 3.81-3.32 (m, 2H), 2.91-2.78 (m, 0.5H), 2.68-2.60 (2×d, J=5.4 Hz, 3H), 2.59-2.31 (m, 1.5H); MS (ES+): m/z=622 (M+H)⁺; LCMS (Method B): t_{R}=3.37 min.

### Allyl (6aS)-3-(benzyloxy)-6-hydroxy-2-methoxy-8-(4-(N-methylsulfamoyl)-phenyl)-12-oxo-6,6a,9,10-tetrahydrobenzo[e]pyrido[1,2-a][1,4]diazepine-5(12H)-carboxylate and allyl (6aS)-3-(benzyloxy)-6-hydroxy-2-methoxy-8-(4-(N-methylsulfamoyl)phenyl)-12-oxo-6,6a,7,10-tetrahydrobenzo[e]-pyrido-[1,2-a][1,4]diazepine-5(12H)-carboxylate (1:1 regioisomers) (26)

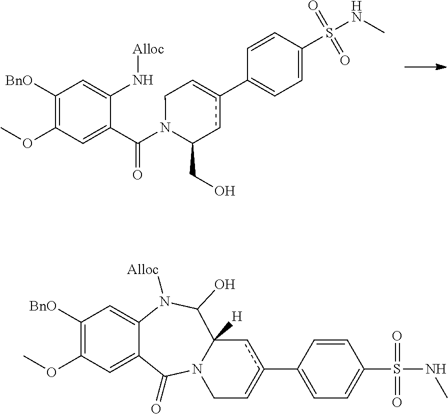

A solution of allyl (S)-(5-(benzyloxy)-2-(6-(hydroxymethyl)-4-(4-(N-methylsulfamoyl)phenyl)-1,2,3,6-tetrahydropyridine-1-carbonyl)-4-methoxyphenyl)carbamate and allyl (S)-(5-(benzyloxy)-2-(2-(hydroxymethyl)-4-(4-(N-methylsulfamoyl)phenyl)-1,2,3,6-tetrahydropyridine-1-carbonyl)-4-methoxyphenyl)carbamate (25) (1.50 g, 2.41 mmol) in dichloromethane (50 mL) was charged with 2,2,6,6-tetramethylpiperidine 1-oxyl (0.037 g, 0.24 mmol) and (diacetoxyiodo)benzene (0.853 g, 2.61 mmol) and stirred at room temperature for 16 h. Dichloromethane (15 mL) was then added to the reaction mixture, which was quenched by addition of a saturated aqueous solution of sodium metabisulfite (50 mL). The organic phase was separated, washed with brine (50 mL), dried over magnesium sulfate, filtered and concentrated in vacuo. Purification by flash column chromatography (silica), eluting with acetone/dichloromethane (10%) gave the title compound (748 mg, 50%) as a yellow solid.

¹H NMR (400 MHz, CDCl₃), mixture of rotamers and regioisomers, δ 7.82 (apparent t, J=70.6 Hz, 2H), 7.54 (apparent t, J=70.8 Hz, 2H), 7.46-7.29 (m, 6H), 7.25-7.22 (2×S, 1H), 6.79-6.68 (m, 1H), 6.39-6.29 (m, 1H), 5.87-5.57 (m, 2H), 5.24-5.03 (m, 4H), 4.89-4.79 (m, 1H), 4.59-4.34 (m, 2H), 4.24-4.01 (m, 1H), 3.95-3.93 (2×s, 3H), 3.81-3.72 (m, 1H), 3.14-3.05 (m, 1H), 2.76-2.73 (m, 3H); MS (ES+): m/z=620 (M+H)⁺; LCMS (Method B): t_{R}=3.32 min.

### Allyl (6aS)-3-hydroxy-2,6-dimethoxy-8-(4-(N-methylsulfamoyl)phenyl)-12-oxo-6,6a,7,10-tetrahydrobenzo[e]pyrido[1,2-a][1,4]diazepine-5(12H)-carboxylate (27)

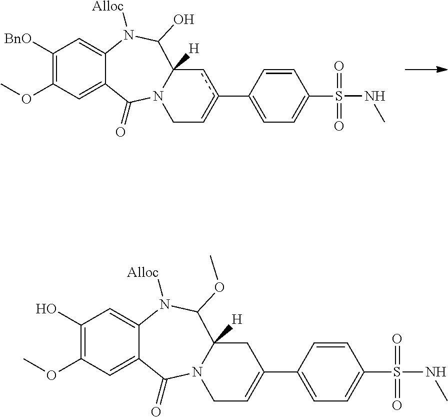

A solution of allyl (6aS)-3-(benzyloxy)-6-hydroxy-2-methoxy-8-(4-(N-methylsulfamoyl)phenyl)-12-oxo-6,6a,9,10-tetrahydrobenzo[e]pyrido[1,2-a][1,4]diazepine-5(12H)-carboxylate and allyl (6aS)-3-(benzyloxy)-6-hydroxy-2-methoxy-8-(4-(N-methylsulfamoyl)phenyl)-12-oxo-6,6a,7,10-tetrahydrobenzo[e]pyrido[1,2-a][1,4]diazepine-5(12H)-carboxylate (26) (700 mg, 1.13 mmol) in anhydrous dichloromethane (20 mL) was charged with boron trichloride (1 M in dichloromethane, 3.39 mL, 3.39 mmol) and stirred under an inert atmosphere of nitrogen for 30 min. Methanol (20 mL) was then charged and the resulting mixture irradiated with microwaves at 55° C. for 1 h. After concentrating the resulting mixture in vacuo, purification was carried out by (multiple, sequential) flash column chromatography (silica), eluting with ethyl acetate/petroleum spirit, 40-60° C. (20%) to afford the (regiopure) title compound (203 mg, 33%) as a white solid.

¹H NMR (400 MHz, CDCl₃) δ 7.84 (d, J=8.5 Hz, 2H), 7.57-7.50 (m, 2H), 7.21 (s, 1H), 6.74 (s, 1H), 6.47-6.39 (m, 1H), 5.81-5.68 (m, 1H), 5.40 (d, J=90.6 Hz, 1H), 5.16-5.01 (m, 2H), 4.56 (dd, J=14.6, 4.4 Hz, 1H), 4.45 (dd, J=10.7, 5.3 Hz, 1H), 4.23-4.15 (m, 2H), 3.91 (s, 3H), 3.73-3.64 (m, 1H), 3.42 (s, 3H), 2.96 (d, J=14.9 Hz, 1H), 2.78-2.59 (m, 5H); MS (ES+): m/z=544 (M+H)⁺; LCMS (Method B): t_{R}=2.97 min.

### Allyl (6aS)-2,6-dimethoxy-3-((3-(2-methoxy-2-oxoethyl)benzyl)oxy)-8-(4-(N-methylsulfamoyl)phenyl)-12-oxo-6,6a,7,10-tetrahydrobenzo[e]pyrido-[1,2-a][1,4]diazepine-5(12H)-carboxylate (28)

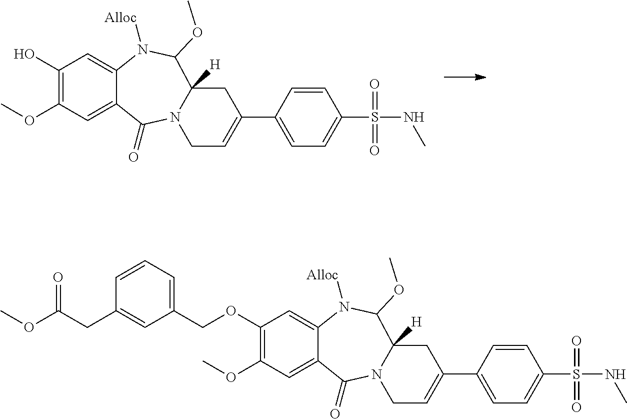

A solution of allyl (6aS)-3-hydroxy-2,6-dimethoxy-8-(4-(N-methylsulfamoyl)phenyl)-12-oxo-6,6a,7,10-tetrahydrobenzo[e]pyrido[1,2-a][1,4]diazepine-5(12H)-carboxylate (27) (100 mg, 0.184 mmol) in N,N-dimethylformamide (1 mL) was charged with potassium carbonate (31 mg, 0.221 mmol) and 3-(bromomethyl)-benzeneacetic acid methyl ester (45 mg, 0.184 mmol) and stirred at room temperature for 16 h. After the reaction was judged to be complete by TLC and LCMS, the mixture was diluted into ethyl acetate (100 mL) and washed with cold brine (2×50 mL), then dried over magnesium sulfate, filtered and concentrated in vacuo. Purification by flash column chromatography (silica), eluting with ethyl acetate/petroleum spirit, 40-60° C. (86%) gave the title compound (65 mg, 50%) as a cream solid.

¹H NMR (400 MHz, CDCl₃) δ 7.85 (d, J=8.5 Hz, 2H), 7.53 (d, J=8.4 Hz, 2H), 7.32 (dd, J=6-5, 3-5 Hz, 3H), 7.28-7.20 (m, 2H), 6.67 (s, 1H), 6.42 (br, 1H), 5.67 (br, 1H), 5.38 (d, J=90.2 Hz, 1H), 5.14-4.99 (m, 4H), 4.88 (q, J=5.3 Hz, 1H), 4.56-4.34 (m, 2H), 4.28-4.20 (m, 1H), 3.91 (s, 3H), 3.66 (s, 3H), 3.62 (s, 2H), 3.38 (s, 3H), 2.95 (d, J=14.3 Hz, 1H), 2.82-2.68 (m, 1H), 2.64 (d, J=5.3 Hz, 3H), 1.88 (br, 1H), 1.18 (br, 1H); ¹³C NMR (100 MHz, CDCl₃) δ 171.8, 169.1, 155.9, 150.3, 149.6, 143.9, 137.9, 136.5, 134.4, 131.9, 129.1, 128.9, 128.3, 128.0, 127.7, 126.2, 125.7, 123.8, 117.3, 114.9, 110.9, 91.2, 71.1, 66.5, 65.8, 56.2, 54.9, 52.1, 41.6, 41.0, 29.3, 26.9; MS (ES+): m/z=706 (M+H)⁺; LCMS (Method A): t_{R}=7.38 min.

### 2-(3-((((6aS)-5-((Allyloxy)carbonyl)-2,6-dimethoxy-8-(4-(N-methyl-sulfamoyl)phenyl)-12-oxo-5,6,6a,7,10,12-hexahydrobenzo[e]pyrido[1,2-a][1,4]diazepin-3-yl)oxy)methyl)phenyl)acetic acid (29)

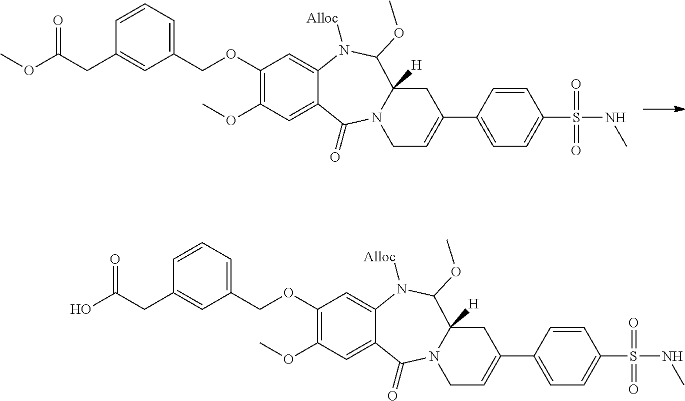

A solution of allyl (6aS)-2,6-dimethoxy-3-((3-(2-methoxy-2-oxoethyl)benzyl)oxy)-8-(4-(N-methylsulfamoyl)phenyl)-12-oxo-6,6a,7,10-tetrahydrobenzo[e]pyrido[1,2-a][1,4]diazepine-5(12H)-carboxylate (28) (65 mg, 0.092 mmol) in tetrahydrofuran (1 mL) was charged with an aqueous solution of sodium hydroxide (0.5 M, 368 μL, 0.184 mmol) and stirred at room temperature for 17 min, whereupon TLC and LCMS confirmed completion of reaction. The mixture was then adjusted to pH=4 by cautious addition of a saturated aqueous solution of citric acid and then extracted with ethyl acetate (2×50 mL). The combined organic extracts were dried over magnesium sulfate, filtered and concentrated in vacuo, to give the title compound as a yellow oil, which was employed in the subsequent step without further purification.

MS (ES+): m/z=692 (M+H)⁺; LCMS (Method A): t_{R}=6.85 min.

### Allyl (6aS)-3-((3-(2-((S)-1-(chloromethyl)-5-hydroxy-1,2-dihydro-3H-benzo[e]indol-3-yl)-2-oxoethyl)benzyl)oxy)-2,6-dimethoxy-8-(4-(N-methylsulfamoyl)phenyl)-12-oxo-6,6a,7,10-tetrahydrobenzo[e]pyrido[1,2-a][1,4]diazepine-5(12H)-carboxylate (30)

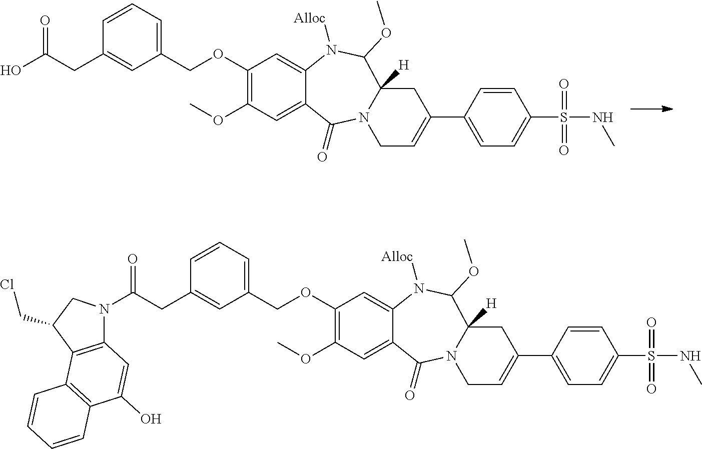

A solution of 2-(3-((((6aS)-5-((Allyloxy)carbonyl)-2,6-dimethoxy-8-(4-(N-methylsulfamoyl)phenyl)-12-oxo-5,6,6a,7,10,12-hexahydrobenzo[e]pyrido[1,2-a][1,4]diazepin-3-yl)oxy)methyl)phenyl)acetic acid (29) (64 mg, 0.092 mmol) in N,N-dimethylacetamide (1 mL) was charged to (S)-1-(chloromethyl)-2,3-dihydro-1H-benzo[e]indol-5-ol hydrochloride (12) (35 mg, 0.129 mmol), followed by N-(3-dimethylaminopropyl)-N′-ethylcarbodiimide hydrochloride (74 mg, 0.387 mmol) and stirred at room temperature for 16 h. The resulting mixture was diluted into ethyl acetate and washed with cold brine (twice), then dried over magnesium sulfate, filtered and concentrated in vacuo. Purification was enacted by flash column chromatography (silica), eluting with ethyl acetate/petroleum spirit, 40-60° C. (from 98%), to give the title compound (24 mg, 29% over two steps) as a grey-green solid.

¹H NMR (400 MHz, CDCl₃) δ 9.85 (br, 1H), 8.34 (s, 1H), 8.22 (d, J=8.3 Hz, 1H), 7.84 (d, J=8.5 Hz, 2H), 7.60 (d, J=8.3 Hz, 1H), 7.51 (d, J=8.5 Hz, 2H), 7.47 (d, J=8.5 Hz, 2H), 7.40-7.31 (m, 4H), 7.22 (s, 1H), 6.67 (s, 1H), 6.43 (br, 1H), 5.60 (br, 1H), 5.36 (d, J=90.1 Hz, 1H), 5.14 (s, 2H), 5.05-4.93 (m, 2H), 4.69 (d, J=50.1 Hz, 1H), 4.50-4.40 (m, 1H), 4.33-4.22 (m, 3H), 4.14-4.03 (m, 2H), 3.95 (s, 2H), 3.90 (br, 1H), 3.82 (s, 3H), 3.70-3.63 (m, 1H), 3.34 (s, 3H), 3.27 (t, J=10.8 Hz, 1H), 2.91 (d, J=15.1 Hz, 1H), 2.65 (d, J=5.3 Hz, 3H), 1.76 (br, 1H);); ¹³C NMR (100 MHz, CDCl₃) δ 169.2, 157.5, 155.2, 150.3, 149.6, 143.9, 141.2, 141.1, 137.8, 137.0, 134.4, 134.0, 131.8, 129.9, 129.3, 128.8, 127.7, 127.6, 126.2, 125.7, 123.9, 123.8, 123.5, 122.9, 122.0, 115.0, 114.8, 112.0, 110.9, 106.5, 100.7, 91.2, 71.0, 66.5, 60.4, 56.3, 56.1, 53.5, 46.1, 43.6, 42.2, 41.6, 30.6, 29.3; MS (ES+): m/z=907 (M+H)⁺; LCMS (Method A): t_{R}=7.90 min.

### 4-((S)-3-((3-(2-((S)-1-(Chloromethyl)-5-hydroxy-1,2-dihydro-3H-benzo[e]indol-3-yl)-2-oxoethyl)benzyl)oxy)-2-methoxy-12-oxo-6a,7,10,12-tetrahydrobenzo[e]pyrido[1,2-a][1,4]diazepin-8-yl)-N-methylbenzenesulfonamide (31)

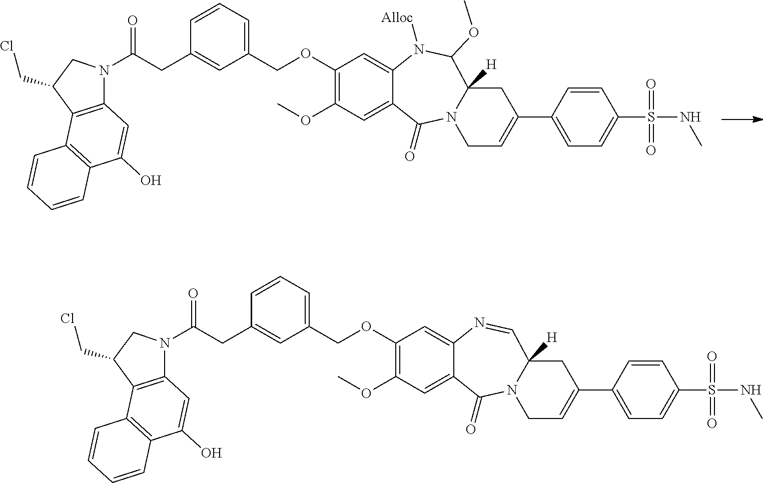

A solution of allyl (6aS)-3-((3-(2-((S)-1-(chloromethyl)-5-hydroxy-1,2-dihydro-3H-benzo[e]indol-3-yl)-2-oxoethyl)benzyl)oxy)-2,6-dimethoxy-8-(4-(N-methylsulfamoyl)phenyl)-12-oxo-6,6a,7,10-tetrahydrobenzo[e]pyrido[1,2-a][1,4]diazepine-5(12H)-carboxylate (30) (24 mg, 0.026 mmol) in dichloromethane (1 mL) was charged with pyrrolidine (2.4 μL, 0.029 mmol), and tetrakis(triphenylphosphine)palladium(0) (3 mg, 0.003 mmol) and stirred at room temperature whilst monitoring by TLC and LCMS. After the reaction was judged to be complete (15 min), the mixture was diluted in dichloromethane and filtered through a pad of celite. The filtrate was concentrated in vacuo, then charged with diethyl ether and concentrated again. Diethyl ether was charged once more, and the residue concentrated in vacuo for a third time. Purification was enacted by flash column chromatography (silica), eluting with ethyl acetate/petroleum spirit, 40-60° C. (from 90% to 100%), to give the title compound (4.8 mg, 23%) as a white solid.

¹H NMR (400 MHz, acetone-d₆) δ 9.54 (br, 1H), 8.27 (d, J=8.6 Hz, 1H), 8.16 (s, 1H), 7.94 (d, J=8.5 Hz, 2H), 7.90 (d, J=40.6 Hz, 1H), 7.86 (d, J=8.5 Hz, 2H), 7.85-7.83 (m, 1H), 7.80-7.56 (m, 2H), 7.54 (d, J=6.7 Hz, 1H), 7.51 (s, 1H), 7.46 (d, J=70.2 Hz, 2H), 7.45-7.34 (m, 1H), 6.95 (s, 1H), 6.79 (br, 1H), 6.51-6.41 (m, 1H), 5.30 (q, J=12.4 Hz, 2H), 4.54-4.33 (m, 3H), 4.20 (d, J=18.7 Hz, 2H), 4.09-3.99 (m, 2H), 3.92 (s, 3H), 3.84-3.79 (m, 1H), 3.75-3.66 (m, 1H), 3.20-3.15 (m, 1H), 2.68-2.62 (m, 4H), 1.35 (s, 1H); MS (ES+): m/z=791 (M+H)⁺; LCMS (Method A): t_{R}=7.33 min.

### Allyl (6aS)-2,6-dimethoxy-3-((6-(2-methoxy-2-oxoethyl)pyridin-2-yl)methoxy)-8-(4-(N-methylsulfamoyl)phenyl)-12-oxo-6,6a,7,10-tetrahydrobenzo[e]pyrido[1,2-a][1,4]diazepine-5(12H)-carboxylate (32)

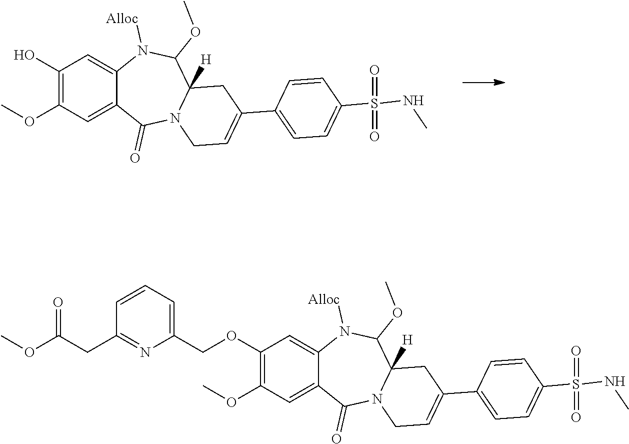

A solution of allyl (6aS)-3-hydroxy-2,6-dimethoxy-8-(4-(N-methylsulfamoyl)phenyl)-12-oxo-6,6a,7,10-tetrahydrobenzo[e]pyrido[1,2-a][1,4]diazepine-5(12H)-carboxylate (27) (114 mg, 0.210 mmol) in N,N-dimethylformamide (0.5 mL) was charged with potassium carbonate (58 mg, 0.420 mmol) and methyl 2-[6-(chloromethyl)-2-pyridyl]acetate hydrochloride (49 mg, 0.210 mmol) and stirred at room temperature for 16 h. After the reaction was judged to be complete by TLC and LCMS, the mixture was diluted into ethyl acetate (100 mL) and washed with cold brine (2×50 mL), then dried over magnesium sulfate, filtered and concentrated in vacuo. Purification by flash column chromatography (silica), eluting with ethyl acetate/petroleum spirit, 40-60° C. (92%) gave the title compound (33 mg, 19%) as a cream solid.

¹H NMR (400 MHz, CDCl₃) □□ 7.78 (d, J=8.3 Hz, 2H), 7.63 (t, J=7.7 Hz, 1H), 7.46 (d, J=8.5 Hz, 2H), 7.42 (d, J=70.8 Hz, 1H), 7.19 (s, 1H), 7.18-7.13 (m, 1H), 6.37 (s, 1H), 5.56 (br, 1H), 5.31 (d, J=90.2 Hz, 1H), 5.18 (s, 2H), 5.01 (d, J=17.2 Hz, 1H), 4.93 (d, J=190.7 Hz, 1H), 4.84 (d, J=5.1 Hz, 1H), 4.45-4.27 (m, 1H), 4.22 (dd, J=190.1, 5.3 Hz, 1H), 4.13 (d, J=18.6 Hz, 1H), 3.89 (s, 3H), 3.77 (s, 2H), 3.63 (m, 3H), 3.43-3.34 (m, 1H), 3.30 (s, 3H), 2.88 (t, J=8.0 Hz, 1H), 2.66 (t, J=8.6 Hz, 1H), 2.57 (s, 3H), 1.57-1.46 (m, 1H), 1.35-1.23 (m, 1H); ¹³C NMR (100 MHz, CDCl₃) D D170.8, 169.2, 156.1, 153.7, 153.6, 149.9, 149.2, 143.8, 137.7, 137.6, 137.5, 134.3, 131.7, 127.6, 126.2, 125.6, 123.7, 122.9, 119.6, 117.4, 114.3, 110.7, 91.0, 73.7, 71.4, 66.4, 64.3, 56.2, 54.8, 52.2, 43.5, 30.5, 29.3, 26.8; MS (ES+): m/z=707 (M+H)⁺; LCMS (Method A): t_{R}=6.85 min.

### 2-(6-((((6aS)-5-((Allyloxy)carbonyl)-2,6-dimethoxy-8-(4-(N-methylsulfamoyl)phenyl)-12-oxo-5,6,6a,7,10,12-hexahydrobenzo[e]pyrido[1,2-a][1,4]diazepin-3-yl)oxy)methyl)pyridin-2-yl)acetic acid (33)

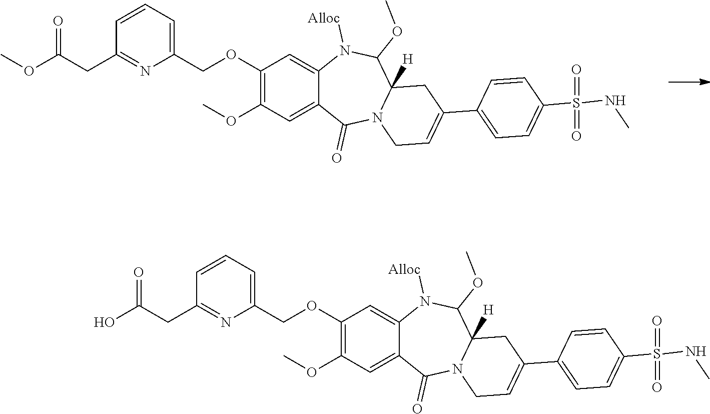

A solution of allyl (6aS)-2,6-dimethoxy-3-((6-(2-methoxy-2-oxoethyl)pyridin-2-yl)methoxy)-8-(4-(N-methylsulfamoyl)phenyl)-12-oxo-6,6a,7,10-tetrahydrobenzo[e]pyrido[1,2-a][1,4]diazepine-5(12H)-carboxylate (32) (57 mg, 0.080 mmol) in tetrahydrofuran (1 mL) was charged with sodium hydroxide (1 M, 200 μL) and stirred at room temperature for 2 h. After the reaction was judged to be complete (by TLC and LCMS), the mixture was adjusted to pH=3 with citric acid (1 M, aqueous), and extracted thrice with ethyl acetate. The combined organic extracts were dried over magnesium sulfate and concentrated in vacuo, to give the title compound as a yellow solid, which was employed in the subsequent step without further purification.

MS (ES+): m/z=693 (M+H)⁺; LCMS (Method A): t_{R}=6.33 min.

### Allyl (6aS)-3-((6-(2-((S)-1-(chloromethyl)-5-hydroxy-1,2-dihydro-3H-benzo[e]indol-3-yl)-2-oxoethyl)pyridin-2-yl)methoxy)-2,6-dimethoxy-8-(4-(N-methylsulfamoyl)phenyl)-12-oxo-6,6a,7,10-tetrahydrobenzo[e]pyrido[1,2-a][1,4]diazepine-5(12H)-carboxylate (34)

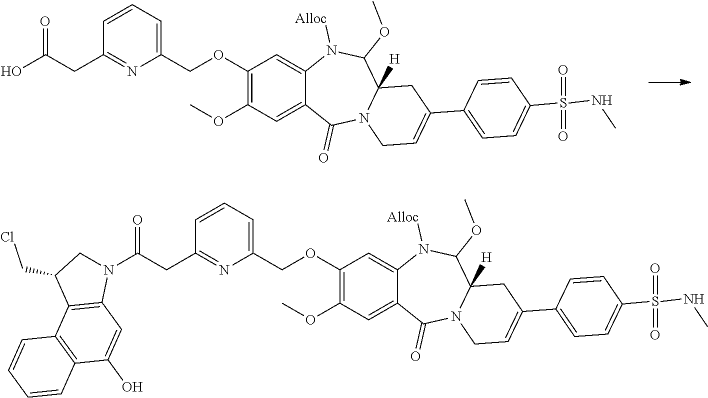

A solution of 2-(6-((((6aS)-5-((allyloxy)carbonyl)-2,6-dimethoxy-8-(4-(N-methylsulfamoyl)phenyl)-12-oxo-5,6,6a,7,10,12-hexahydrobenzo[e]pyrido[1,2-a][1,4]diazepin-3-yl)oxy)methyl)pyridin-2-yl)acetic acid (33) (55 mg, 0.080 mmol) and (S)-1-(chloromethyl)-2,3-dihydro-1H-benzo[e]indol-5-ol hydrochloride (12) (22 mg, 0.080 mmol) in N,N-dimethylacetamide (1 mL) was charged with N-(3-dimethylaminopropyl)-N′-ethylcarbodiimide hydrochloride (61 mg, 0.320 mmol) and stirred at room temperature for 16 h. The resulting mixture was diluted into ethyl acetate and washed with cold brine (twice), then dried over magnesium sulfate, filtered and concentrated in vacuo. Purification was enacted by flash column chromatography (silica), eluting with ethyl acetate/petroleum spirit, 40-60° C. (100%), to give the title compound as a grey-green solid.

MS (ES+): m/z=908 (M+H)⁺; LCMS (Method A): t_{R}=7.53 min.

### 4-((S)-3-((6-(2-((S)-1-(Chloromethyl)-5-hydroxy-1,2-dihydro-3H-benzo[e]indol-3-yl)-2-oxoethyl)pyridin-2-yl)methoxy)-2-methoxy-12-oxo-6a,7,10,12-tetrahydrobenzo[e]pyrido[1,2-a][1,4]diazepin-8-yl)-N-methylbenzenesulfonamide (35)

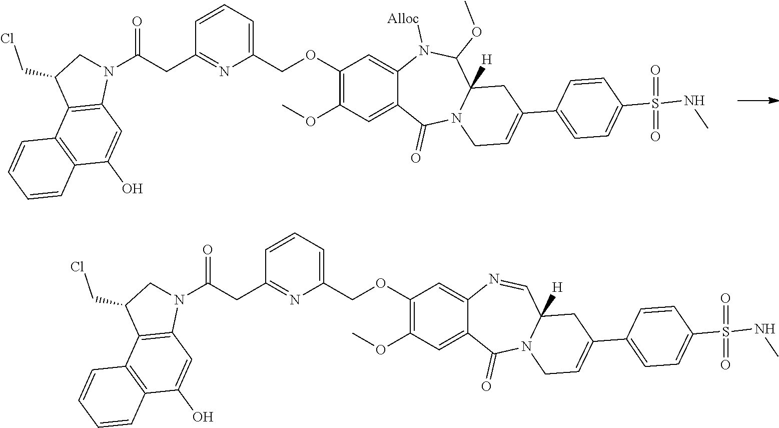

A solution of allyl (6aS)-3-((6-(2-((S)-1-(chloromethyl)-5-hydroxy-1,2-dihydro-3H-benzo[e]indol-3-yl)-2-oxoethyl)pyridin-2-yl)methoxy)-2,6-dimethoxy-8-(4-(N-methylsulfamoyl)phenyl)-12-oxo-6,6a,7,10-tetrahydrobenzo[e]pyrido[1,2-a][1,4]diazepine-5(12H)-carboxylate (34) (18 mg, 0.020) in dichloromethane (1 mL) was charged with tetrakis(triphenylphosphine)palladium(0) (1 mg) and pyrrolidine (100 L) and the resulting mixture stirred at room temperature for 30 min. After concentrating in vacuo, the residue was purified by flash column chromatography (silica), eluting with methanol/ethyl acetate (5%), to give the title compound as a white solid.

¹H NMR (400 MHz, CDCl₃) δ 8.19 (d, J=8.8 Hz, 1H), 8.03 (s, 1H), 7.88 (d, J=8.5 Hz, 2H), 7.85 (d, J=40.2 Hz, 1H), 7.70-7.65 (m, 1H), 7.62-7.58 (m, 1H), 7.56 (d, J=8.4 Hz, 2H), 7.52-7.46 (m, 3H), 7.42-7.38 (m, 1H), 7.36-7.30 (m, 1H), 6.79 (s, 1H), 6.58-6.54 (m, 1H), 6.50-6.44 (m, 1H), 5.30 (s, 2H), 4.61-4.54 (m, 1H), 4.50-4.40 (m, 2H), 4.34-4.22 (m, 3H), 4.17-4.05 (m, 2H), 3.48 (s, 2H), 3.42-3.31 (m, 2H), 3.00-2.86 (m, 2H), 2.17 (s, 3H), 2.40-2.31 (m, 1H), 2.22-2.19 (m, 1H); MS (ES+): m/z=793 (M+H)⁺; LCMS (Method A): t_{R}=6.98 min.

### (S)-(4-(Benzyloxy)-5-methoxy-2-nitrophenyl)(6-(((tert-butyldimethylsilyl)oxy)methyl)-4-(thiophen-3-yl)-3,6-dihydropyridin-1(2H)-yl)methanone and (S)-(4-(benzyloxy)-5-methoxy-2-nitrophenyl)(2-(((tert-butyldimethylsilyl)oxy)methyl)-4-(thiophen-3-yl)-3,6-dihydropyridin-1(2H)-yl)methanone (36) (1:1 regioisomers)

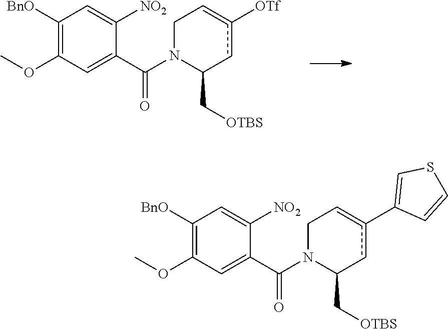

A solution of (S)-1-(4-(benzyloxy)-5-methoxy-2-nitrobenzoyl)-6-(((tert-butyldimethylsilyl)oxy)methyl)-1,2,3,6-tetrahydropyridin-4-yl trifluoromethanesulfonate and (S)-1-(4-(benzyloxy)-5-methoxy-2-nitrobenzoyl)-2-(((tert-butyldimethylsilyl)oxy)methyl)-1,2,3,6-tetrahydropyridin-4-yl trifluoromethanesulfonate (21) (5.00 g, 7.56 mmol) in acetonitrile (10 mL) was charged with [1,1′-bis(diphenylphosphino)ferrocene]dichloropalladium(II), complex with dichloromethane (0.617 g, 0.756 mmol), 3-thienylboronic acid (1.07 g, 8.35 mmol) and an aqueous solution of potassium carbonate (2 M, 7.56 mL, 15.1 mmol) and irradiated with microwaves at 50° C. for 20 min. The mixture was subsequently diluted with ethyl acetate (50 mL) and sequentially washed with water (25 mL) and brine (25 mL), then dried over solid anhydrous magnesium sulfate and concentrated in vacuo. The residue was then purified by flash column chromatography (silica), eluting with ethyl acetate/petroleum spirit, 40-60° C. (from 0% to 100%) to afford the title compound (3.65 g, 81%) as a yellow solid.

¹H NMR (400 MHz, CDCl₃), mixture of rotamers and regioisomers, δ 7.85-7.73 (m, 1H), 7.49-7.34 (m, 5H), 7.33-7.18 (m, 3H), 7.16-7.06 (m, 1H), 6.81-6.72 (m, 1H), 5.28-5.18 (m, 2H), 5.06 (br. s, 1H), 4.01-3.95 (m, 4H), 3.83 (d, J=7.03 Hz, 1H), 3.76 (d, J=90.76 Hz, 1H), 3.71-3.57 (m, 1H), 3.57-3.36 (m, 1H), 2.41 (d, J=190.14 Hz, 1H), 0.98-0.82 (m, 9H), 0.18-0.06 (m, 6H); ¹³C NMR (100 MHz, CDCl₃), mixture of rotamers and regioisomers, δ 166.4, 154.7, 148.1, 142.0, 135.3, 134.6, 130.6, 128.8, 128.5, 127.6, 127.5, 126.0, 124.6, 122.1, 119.6, 119.2, 109.1, 71.4, 60.4, 56.7, 56.7, 52.5, 52.3, 42.9, 27.4, 25.9, 25.8, 21.0, 18.2, −5.4; MS (ES+): m/z=595 (M+H)+.

### (S)-(2-Amino-4-(benzyloxy)-5-methoxyphenyl)(6-(((tert-butyldimethylsilyl)oxy)methyl)-4-(thiophen-3-yl)-3,6-dihydropyridin-1(2H)-yl)methanone and (S)-(2-amino-4-(benzyloxy)-5-methoxyphenyl)(2-(((tert-butyldimethylsilyl)oxy)methyl)-4-(thiophen-3-yl)-3,6-dihydropyridin-1(2H)-yl)methanone (37) (1:1 regioisomers)

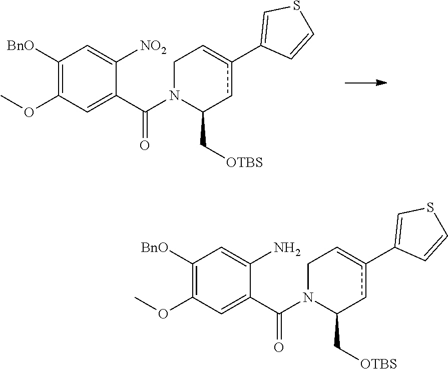

A solution of (S)-(4-(benzyloxy)-5-methoxy-2-nitrophenyl)(6-(((tert-butyldimethylsilyl)oxy)methyl)-4-(thiophen-3-yl)-3,6-dihydropyridin-1(2H)-yl)methanone and (S)-(4-(benzyloxy)-5-methoxy-2-nitrophenyl)(2-(((tert-butyldimethylsilyl)oxy)methyl)-4-(thiophen-3-yl)-3,6-dihydropyridin-1(2H)-yl)methanone (36) (3.62 g, 6.08 mmol) in formic acid (5% v/v in absolute ethanol, 60 mL) was charged with zinc powder (15.9 g, 243 mmol) and stirred for 1 h, whilst monitoring by TLC and LCMS. Upon completion, the mixture was filtered through a pad of celite and the resulting filter cake washed with ethyl acetate. A saturated aqueous solution of sodium hydrogen carbonate (10 mL) was then added to the filtrate and the mixture concentrated in vacuo to remove organics. The residue was then partitioned between ethyl acetate (60 mL) and brine (60 mL) and the organic phase dried over magnesium sulfate, filtered, and concentrated in vacuo. Purification by flash column chromatography (silica), eluting with acetone/dichloromethane (from 0% to 20%) gave the title compound (3.24 g, 80%) as a yellow solid.

¹H NMR (400 MHz, CDCl₃), mixture of rotamers and regioisomers, δ 7.46-7.21 (m, 7H), 7.14 (dd, J=2.7, 1.2 Hz, 1H), 6.82-6.76 (m, 1H), 6.28 (d, J=5.9 Hz, 1H), 6.15 (dd, J=30.9, 2.0 Hz, 1H), 5.13 (s, 2H), 4.10 (d, J=16.8 Hz, 2H), 3.96-3.82 (m, 1H), 3.81 (s, 3H), 3.79-3.69 (m, 1H), 3.69-3.39 (m, 1H), 2.64-2.38 (m, 2H), 0.92-0.85 (m, 9H), 0.07 (s, 3H), 0.02 (br. s, 3H); ¹³C NMR (100 MHz, CDCl₃), mixture of rotamers and regioisomers, δ 150.8, 141.9, 140.1, 136.8, 131.9, 128.6, 127.9, 127.1, 125.9, 124.5, 121.3, 119.4, 119.3, 118.7, 113.1, 112.3, 103.0, 102.9, 70.7, 64.4, 62.1, 57.1, 30.9, 25.9, 18.3, −5.4; MS (ES+): m/z=565 (M+H)+.

### Allyl (S)-(5-(benzyloxy)-2-(2-(((tert-butyldimethylsilyl)oxy)methyl)-4-(thiophen-3-yl)-1,2,3,6-tetrahydropyridine-1-carbonyl)-4-methoxyphenyl)carbamate and allyl (S)-(5-(benzyloxy)-2-(6-(((tert-butyldimethylsilyl)oxy)methyl)-4-(thiophen-3-yl)-1,2,3,6-tetrahydropyridine-1-carbonyl)-4-methoxyphenyl)carbamate (38) (1:1 regioisomers)

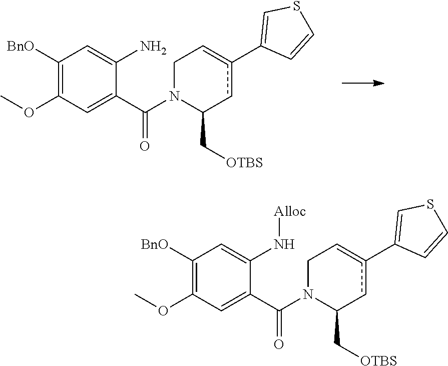

A solution of (S)-(2-amino-4-(benzyloxy)-5-methoxyphenyl)(6-(((tert-butyldimethylsilyl)oxy)methyl)-4-(thiophen-3-yl)-3,6-dihydropyridin-1(2H)-yl)methanone and (S)-(2-amino-4-(benzyloxy)-5-methoxyphenyl)(2-(((tert-butyldimethylsilyl)oxy)methyl)-4-(thiophen-3-yl)-3,6-dihydropyridin-1(2H)-yl)methanone (52) (3.00 g, 5.30 mmol) in dichloromethane (20 mL) was cooled to 0° C. and charged with pyridine (0.429 mL, 5.30 mmol) and allyl chloroformate (0.564 mL, 5.30 mmol). After 10 min, the reaction was judged to be complete by TLC and LCMS and was sequentially washed with a saturated aqueous solution of copper sulfate (20 mL), water (20 mL) and a saturated aqueous solution of sodium hydrogen carbonate (20 mL). The organic extract was then dried over magnesium sulfate and concentrated in vacuo. The resulting oil was then purified by flash column chromatography (silica), eluting with ethyl acetate/petroleum spirit, 40-60° C./(from 0% to 100%) to afford the title compound (3.33 g, 97%) as a white solid.

¹H NMR (400 MHz, CDCl₃), mixture of rotamers and regioisomers, δ 8.57-7.73 (m, 2H), 7.49 (d, J=7.4 Hz, 2H), 7.42-7.22 (m, 6H), 7.15 (br. s, 1H), 6.85 (d, J=12.9 Hz, 1H), 6.12 (br. s, 1H), 5.99-5.82 (m, 1H), 5.33 (d, J=17.2 Hz, 1H), 5.24-5.17 (m, 3H), 4.70-4.53 (m, 2H), 3.85 (s, 4H), 3.75 (br. s, 1H), 3.58 (br. s, 1H), 2.79 (d, J=14.1 Hz, 1H), 2.46 (d, J=16.8 Hz, 1H), 0.92-0.81 (m, 10H), 0.12-0.04 (m, 3H), 0.01 (br. s, 2H); ¹³C NMR (100 MHz, CDCl₃), mixture of rotamers and regioisomers, δ 153.4, 150.2, 144.8, 141.7, 136.4, 132.5, 128.5, 128.0, 127.7, 126.0, 124.5, 124.4, 119.4, 118.0, 111.6, 70.7, 65.7, 64.3, 62.1, 56.7, 25.9, 18.3, −5.4; MS (ES+): m/z=649 (M+H)+.

### Allyl (S)-(5-(benzyloxy)-2-(2-(hydroxymethyl)-4-(thiophen-3-yl)-1,2,3,6-tetrahydropyridine-1-carbonyl)-4-methoxyphenyl)carbamate and allyl (S)-(5-(benzyloxy)-2-(6-(hydroxymethyl)-4-(thiophen-3-yl)-1,2,3,6-tetrahydropyridine-1-carbonyl)-4-methoxyphenyl)carbamate (39) (1:1 regioisomers)

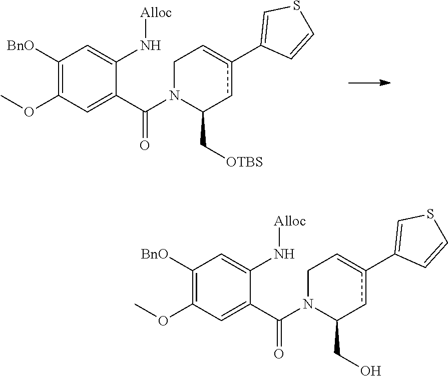

A solution of allyl (S)-(5-(benzyloxy)-2-(2-(((tert-butyldimethylsilyl)oxy)methyl)-4-(thiophen-3-yl)-1,2,3,6-tetrahydropyridine-1-carbonyl)-4-methoxyphenyl)carbamate and allyl (S)-(5-(benzyloxy)-2-(6-(((tert-butyldimethylsilyl)oxy)methyl)-4-(thiophen-3-yl)-1,2,3,6-tetrahydropyridine-1-carbonyl)-4-methoxyphenyl)carbamate (38) (3.27 g, 5.03 mmol) in tetrahydrofuran (20 mL) was cooled to 0° C. and charged with tetrabutylammonium fluoride (1 M in tetrahydrofuran, 6.03 mL, 6.03 mmol). The reaction was allowed to warm to room temperature and after 20 min, TLC and LCMS confirmed consumption of starting material. Ethyl acetate (50 mL) was added to the reaction mixture and the resulting solution was washed with a saturated aqueous solution of ammonium chloride (20 mL) and brine (20 mL) and dried over magnesium sulfate. Purification by flash column chromatography (silica), eluting with acetone/dichloromethane (from 0% to 100%) gave the title compound (2.68 g, 99%) as a pale yellow solid.

¹H NMR (400 MHz, CDCl₃), mixture of rotamers and regioisomers, δ 8.42-8.06 (m, 1H), 7.97-7.64 (m, 1H), 7.47 (d, J=8.2 Hz, 2H), 7.41-7.27 (m, 4H), 7.24-7.10 (m, 2H), 6.84 (br. s, 1H), 6.05 (br. s, 1H), 6.00-5.82 (m, 1H), 5.38-5.26 (m, 1H), 5.24-5.11 (m, 3H), 4.65-4.56 (m, 2H), 3.96-3.85 (m, 1H), 3.83-3.81 (m, 3H), 3.72 (br. s, 1H), 3.64 (d, J=4.7 Hz, 1H), 3.40 (br. s, 1H), 3.22 (br. s, 1H), 2.52-2.40 (m, 2H); ¹³C NMR (100 MHz, CDCl₃), mixture of rotamers and regioisomers, S158.5, 153.9, 150.1, 149.8, 145.3, 141.5, 141.3, 136.4, 136.3, 132.5, 128.5, 127.7, 126.1, 124.5, 120.0, 119.2, 117.9, 110.8, 107.2, 70.8, 65.7, 61.4, 60.4, 56.5, 30.9, 27.9, 25.6, 21.0; MS (ES+): m/z=535 (M+H)⁺; LCMS (Method B): t_{R}=4.07 min.

### Allyl (6S,6aS)-3-(benzyloxy)-6-hydroxy-2-methoxy-12-oxo-8-(thiophen-3-yl)-6,6a,7,10-tetrahydrobenzo[e]pyrido[1,2-a][1,4]diazepine-5(12H)-carboxylate and allyl (6S,6aS)-3-(benzyloxy)-6-hydroxy-2-methoxy-12-oxo-8-(thiophen-3-yl)-6,6a,9,10-tetrahydrobenzo[e]pyrido[1,2-a][1,4]diazepine-5(12H)-carboxylate (40) (1:1 regioisomers)

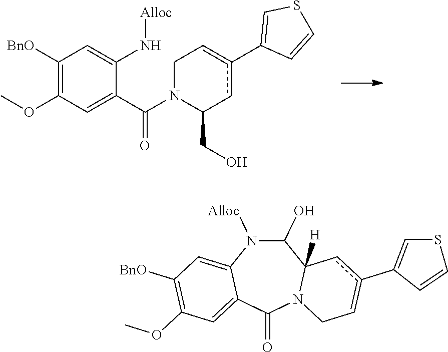

A solution of allyl (S)-(5-(benzyloxy)-2-(2-(hydroxymethyl)-4-(thiophen-3-yl)-1,2,3,6-tetrahydropyridine-1-carbonyl)-4-methoxyphenyl)carbamate and allyl (S)-(5-(benzyloxy)-2-(6-(hydroxymethyl)-4-(thiophen-3-yl)-1,2,3,6-tetrahydropyridine-1-carbonyl)-4-methoxyphenyl)carbamate (39) (2.39 g, 4.48 mmol) in dichloromethane (15 mL) was charged with TEMPO (119 mg, 0.76 mmol) and (diacetoxyiodo)benzene (2.94 g, 9.13 mmol) and stirred at room temperature for 16 h, at which point TLC and LCMS showed consumption of starting material. Dichloromethane (30 mL) was then added to the reaction mixture, which was quenched by addition of a saturated aqueous solution of sodium metabisulfite (20 mL). The organic phase was separated, washed with brine (50 mL), dried over magnesium sulfate, filtered and concentrated in vacuo. Purification by flash column chromatography (silica), eluting with acetone/dichloromethane (from 0% to 100%) gave the title compound (1.70 g, 71%) as a pale yellow solid.

¹H NMR (400 MHz, CDCl₃), mixture of rotamers and regioisomers, 57.47-7.17 (m, 10H), 6.76 (d, J=9.0 Hz, 1H), 6.28 (br. s, 1H), 5.66 (dd, J=10.2, 4.3 Hz, 2H), 5.25-5.03 (m, 2H), 4.46 (br. s, 2H), 4.33-4.15 (m, 1H), 4.04 (br. s, 1H), 3.96-3.88 (m, 3H), 3.80-3.64 (m, 1H), 3.13-2.98 (m, 1H), 2.69-2.55 (m, 2H); ¹³C NMR (100 MHz, CDCl₃), mixture of rotamers and regioisomers, S168.9, 167.6, 149.1, 141.4, 136.2, 133.3, 131.8, 130.5, 128.6, 128.1, 127.3, 126.1, 125.3, 120.2, 119.0, 118.0, 117.6, 114.3, 110.8, 84.5, 84.1, 71.0, 66.7, 60.4, 56.1, 55.9, 54.6, 41.3, 37.1; MS (ES+): m/z=533 (M+H)⁺; LCMS (Method A): t_{R}=7.62 min.

### Allyl (6aS)-3-hydroxy-2,6-dimethoxy-12-oxo-8-(thiophen-3-yl)-6,6a,7,10-tetrahydrobenzo[e]pyrido[1,2-a][1,4]diazepine-5(12H)-carboxylate (41)

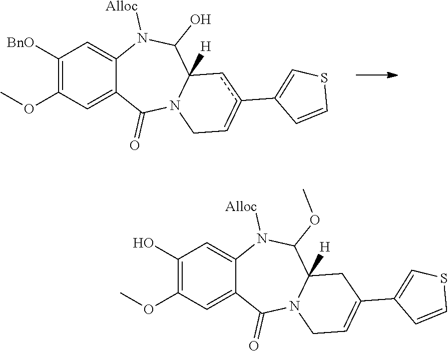

A solution of allyl (6S,6aS)-3-(benzyloxy)-6-hydroxy-2-methoxy-12-oxo-8-(thiophen-3-yl)-6,6a,7,10-tetrahydrobenzo[e]pyrido[1,2-a][1,4]diazepine-5(12H)-carboxylate and allyl (6S,6aS)-3-(benzyloxy)-6-hydroxy-2-methoxy-12-oxo-8-(thiophen-3-yl)-6,6a,9,10-tetrahydrobenzo[e]pyrido[1,2-a][1,4]diazepine-5(12H)-carboxylate (40) (1.59 g, 2.89 mmol) in anhydrous dichloromethane (10 mL) was charged with boron trichloride (1 M in dichloromethane, 8.94 mL, 8.94 mmol) and stirred at room temperature under an inert atmosphere of argon for 15 min. Methanol (30 mL) was then added and the resulting mixture irradiated with microwaves at 55° C. for 1 h. After filtering through a cotton pad, washing with dichloromethane and concentrating in vacuo, purification was carried out by (multiple) flash column chromatography (silica), eluting with ethyl acetate/petroleum spirit, 40-60° C. (from 0% to 100%) to afford the (regiopure) title compound (138 mg, 10%) as a cream solid.

¹H NMR (400 MHz, CDCl₃) δ 7.31 (dd, J=50.1, 2.7 Hz, 1H), 7.25-7.23 (m, 1H), 7.20-7.17 (m, 2H), 6.78-6.69 (m, 2H), 6.28 (br. s, 1H), 5.73 (br. s, 1H), 5.41 (d, J=9.4 Hz, 1H), 5.07 (d, J=10.9 Hz, 2H), 4.55 (dd, J=13.1, 5.7 Hz, 1H), 4.44 (br. s, 1H), 4.23-4.16 (m, 2H), 3.87-3.83 (m, 3H), 3.44-3.41 (m, 3H), 2.98-2.89 (m, 1H), 2.70-2.60 (m, 1H); ¹³C NMR (100 MHz, CDCl₃) δ 169.4, 156.0, 148.4, 146.7, 141.2, 131.9, 130.6, 128.8, 126.4, 124.6, 119.9, 119.2, 117.3, 116.1, 110.3, 91.3, 66.4, 56.1, 54.9, 41.2, 26.9; MS (ES+): m/z=457 (M+H)⁺; LCMS (Method A): t_{R}=7.00 min.

### Allyl (6aS)-2,6-dimethoxy-3-((3-(2-methoxy-2-oxoethyl)benzyl)oxy)-12-oxo-8-(thiophen-3-yl)-6,6a,7,10-tetrahydrobenzo[e]pyrido[1,2-a][1,4]diazepine-5(12H)-carboxylate (42)

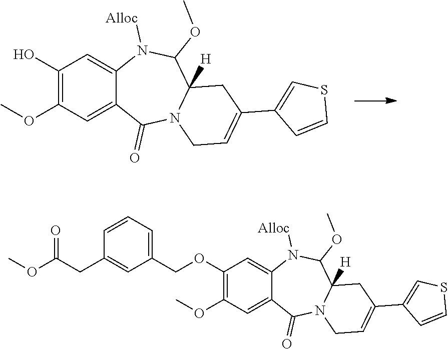

Methyl 2-(3-(bromomethyl)phenyl)acetate (70 mg, 0.29 mmol) and potassium carbonate (58 mg, 0.42 mmol) were added to a solution of allyl (6aS)-3-hydroxy-2,6-dimethoxy-12-oxo-8-(thiophen-3-yl)-6,6a,7,10-tetrahydrobenzo[e]pyrido[1,2-a][1,4]diazepine-5(12H)-carboxylate (41) (129 mg, 0.28 mmol) in N,N-dimethylformamide (1 mL). The resulting mixture was stirred at room temperature for 16 h. After the reaction was judged to be complete (by TLC and LCMS), the mixture was diluted into ethyl acetate (25 mL) and washed with cold brine (2×50 mL). The organic layer was dried over magnesium sulfate, filtered and concentrated in vacuo. Purification by flash column chromatography (silica), eluting with ethyl acetate/petroleum spirit, 40-60° C. (from 0% to 70%), gave the title compound (82 mg, 47%) as a white solid.

MS (ES+): m/z=619 (M+H)⁺; LCMS (Method A): t_{R}=8.10 min.

### 2-(3-((((6aS)-5-((Allyloxy)carbonyl)-2,6-dimethoxy-12-oxo-8-(thiophen-3-yl)-5,6,6a,7,10,12-hexahydrobenzo[e]pyrido[1,2-a][1,4]diazepin-3-yl)oxy)methyl)phenyl)acetic acid (43)

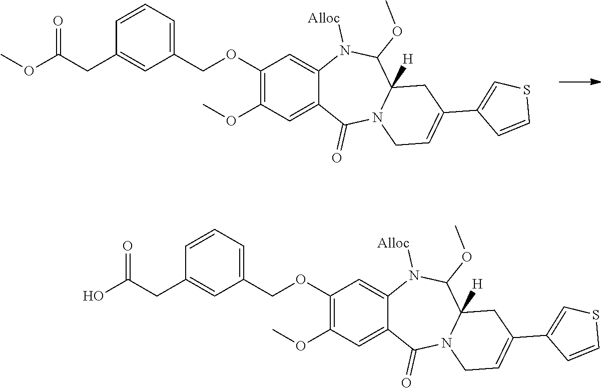

A solution of allyl (6aS)-2,6-dimethoxy-3-((3-(2-methoxy-2-oxoethyl)benzyl)oxy)-12-oxo-8-(thiophen-3-yl)-6,6a,7,10-tetrahydrobenzo[e]pyrido[1,2-a][1,4]diazepine-5(12H)-carboxylate (42) (82 mg, 0.13 mmol) in tetrahydrofuran (1 mL) was charged with an aqueous solution of sodium hydroxide (1 M, 325 μL, 0.325 mmol) and stirred at room temperature for 1 h. After the reaction was judged to be complete (by TLC and LCMS), the reaction mixture was quenched with an aqueous solution of citric acid (1 M, 20 mL) and the pH adjusted to 4. This was then extracted with ethyl acetate (2×25 mL) and the combined organic extracts dried over magnesium sulfate, filtered and concentrated in vacuo. The title compound was collected as a white solid.

MS (ES+): m/z=605 (M+H)⁺; LCMS (Method A): t_{R}=7.38 min.

### Allyl (6aS)-3-((3-(2-((S)-1-(chloromethyl)-5-hydroxy-1,2-dihydro-3H-benzo[e]indol-3-yl)-2-oxoethyl)benzyl)oxy)-2,6-dimethoxy-12-oxo-8-(thiophen-3-yl)-6,6a,7,10-tetrahydrobenzo[e]pyrido[1,2-a][1,4]diazepine-5(12H)-carboxylate (44)

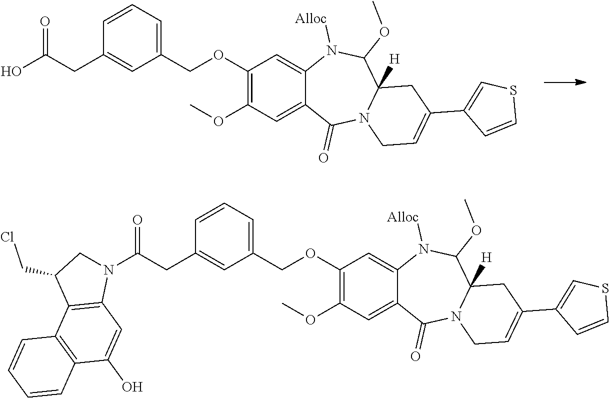

A solution of 2-(3-((((6aS)-5-((Allyloxy)carbonyl)-2,6-dimethoxy-12-oxo-8-(thiophen-3-yl)-5,6,6a,7,10,12-hexahydrobenzo[e]pyrido[1,2-a][1,4]diazepin-3-yl)oxy)methyl)phenyl)acetic acid (43) (50 mg, 0.08 mmol) and (S)-1-(chloromethyl)-2,3-dihydro-1H-benzo[e]indol-5-ol hydrochloride (12) (22 mg, 0.08 mmol) in N,N-dimethylacetamide (1 mL) was charged with N-(3-dimethylaminopropyl)-N′-ethylcarbodiimide hydrochloride (61 mg. 0.32 mmol) and stirred at room temperature for 16 h. The reaction mixture was then diluted into ethyl acetate (25 mL) and washed with cold brine (2×50 mL). The organic layer was then dried over magnesium sulfate, filtered and concentrated in vacuo. Purification by flash column chromatography (silica), eluting with ethyl acetate/petroleum spirit, 40-60° C. (from 30% to 100%), gave the title compound (30 mg, 46%) as a grey solid.

MS (ES+): m/z=821 (M+H)⁺; LCMS (Method A): t_{R}=7.08 min.

### (S)-3-((3-(2-((S)-1-(Chloromethyl)-5-hydroxy-1,2-dihydro-3H-benzo[e]indol-3-yl)-2-oxoethyl)benzyl)oxy)-2-methoxy-8-(thiophen-3-yl)-7,10-dihydrobenzo[e]pyrido[1,2-a][1,4]diazepin-12(6aH)-one (45)

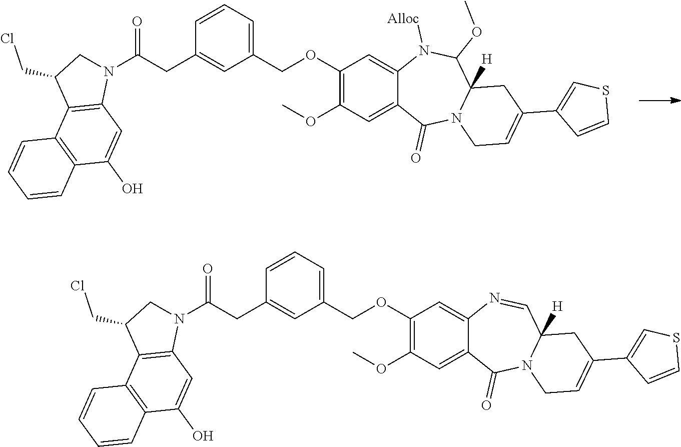

A solution of allyl (6aS)-3-((3-(2-((S)-1-(chloromethyl)-5-hydroxy-1,2-dihydro-3H-benzo[e]indol-3-yl)-2-oxoethyl)benzyl)oxy)-2,6-dimethoxy-12-oxo-8-(thiophen-3-yl)-6,6a,7,10-tetrahydrobenzo[e]pyrido[1,2-a][1,4]diazepine-5(12H)-carboxylate (44) (50 mg, 0.06 mmol) in dichloromethane (3 mL) was charged with pyrrolidine (5 μL, 0.072 mmol) and tetrakis(triphenylphosphine)palladium(0) (7 mg, 0.006 mmol). The resulting mixture was stirred for 15 min and then concentrated in vacuo. Diethyl ether (10 mL) was charged and the residue concentrated in vacuo once again and this process was repeated twice more. Purification was carried out by flash column chromatography (silica), eluting with ethyl acetate/petroleum spirit, 40-60° C. (from 60% to 90%), to give the title compound (13 mg, 30%) as a cream solid.

MS (ES+): m/z=705 (M+H)⁺; LCMS (Method A): t_{R}=7.60 min.

### 4-Hydroxy-5-methoxy-2-nitrobenzaldehyde (46)

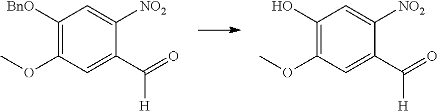 [SMILES: COc1cc(C=O)c([N+](=O)[O-])cc1OCc1ccccc1 ; COc1cc(C=O)c([N+](=O)[O-])cc1O]

A solution of 4-(benzyloxy)-5-methoxy-2-nitrobenzaldehyde (15) (100 g, 348 mmol) in glacial acetic acid (800 mL) was charged with an aqueous solution of hydrobromic acid (48% v/v, 88.0 mL, 522 mmol) and heated to 85° C., with stirring for 1 h, after which the reaction was judged to be complete by TLC. After allowing the resulting mixture to cool to room temperature, it was then diluted in water (1.60 L), and the resulting precipitate filtered, and washed with cold water (100 mL×3) to give the title compound (50.0 g, 73%) as a yellow solid, which was used immediately in the subsequent step without further purification.

¹H NMR (400 MHz, DMSO-d₆) δ 11.11 (br s, 1H), 10.15 (br s, 1H), 7.50 (s, 1H), 7.35 (s, 1H), 3.94 (s, 3H); MS (ES−): m/z=196 (M−H)⁻; LCMS (Method B): t_{R}=2.55 min.

### 5-Methoxy-2-nitro-4-((triisopropylsilyl)oxy)benzaldehyde (47)

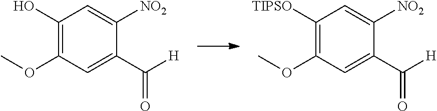

A mixture of 4-hydroxy-5-methoxy-2-nitrobenzaldehyde (46) (50.0 g, 254 mmol), triisopropylsilyl chloride (59.7 mL, 279 mmol) and imidazole (51.8 g, 761 mmol) was heated and stirred at 100° C. for 30 min. The reaction mixture was poured onto ice-water and extracted with ethyl acetate (500 mL×3). The organic extract was dried over sodium sulfate, filtered and concentrated under reduced pressure. The resulting residue was purified by flash column chromatography (silica), eluting with ethyl acetate/petroleum spirit, 40-60° C. (5%) to give the title compound (57.5 g, 64%) as a yellow solid.

¹H NMR (400 MHz, CDCl₃) δ 10.42 (s, 1H), 7.59 (s, 1H), 7.40 (s, 1H), 3.95 (s, 3H), 1.33-1.24 (m, 3H), 1.07 (s, 18H).

### 5-Methoxy-2-nitro-4-((triisopropylsilyl)oxy)benzoic acid (48)

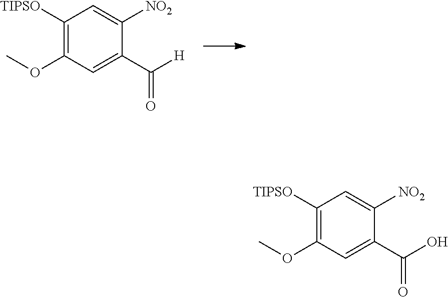

A solution of sodium chlorite (80%, 46.0 g, 407 mmol) and sodium phosphate monobasic dihydrate (35-5 g, 228 mmol) in water (200 mL) was added to a solution of 5-methoxy-2-nitro-4-((triisopropylsilyl)oxy)benzaldehyde (47) (575 g, 163 mmol) in tetrahydrofuran (800 mL) at room temperature. Hydrogen peroxide (30% w/w, 235 mL, 2.28 mol) was immediately added to the vigorously stirred biphasic mixture. The starting material dissolved, and the temperature of the reaction mixture rose to 45° C. After 30 min, the reaction was judged to have completed by TLC. The mixture was subsequently acidified to pH=3-4 with citric acid and extracted with ethyl acetate (500 mL×3). The combined organic extracts were washed with water (150 mL) and brine (150 mL), dried over anhydrous sodium sulfate, filtered and concentrated in vacuo. The residue was then purified by flash column chromatography (silica), eluting with ethyl acetate/petroleum spirit, 40-60° C. (10%) then methanol/dichloromethane (10%) to afford the title compound (38.0 g, 63%) as a yellow oil.

¹H NMR (400 MHz, CDCl₃) δ 9.81 (s, 1H), 7.35 (s, 1H), 7.25 (s, 1H), 3.91 (s, 3H), 1.26 (q, J=7.4 Hz, 3H), 1.09 (d, J=7.4 Hz, 18H); MS (ES−): m/z=368 (M−H)⁻; LCMS (Method D): t_{R}=4.75 min.

### Methyl (2S,4R)-4-hydroxy-1-(5-methoxy-2-nitro-4-((triisopropylsilyl)oxy)benzoyl)piperidine-2-carboxylate (49)

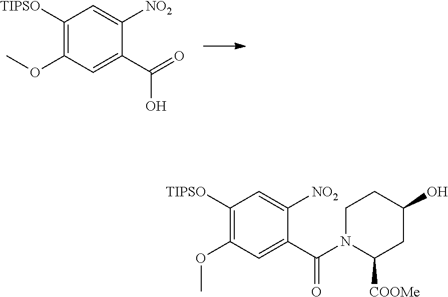

A solution of 5-methoxy-2-nitro-4-((triisopropylsilyl)oxy)benzoic acid (48) (110 g, 0.30 mol) in dichloromethane (1.5 L) was charged with N,N-diisopropylethylamine (154 g, 1.19 mol), HATU (170 g, 0-45 mol) and (2S,4R)-methyl 4-hydroxypiperidine-2-carboxylate hydrochloride (55.4 g, 0.28 mol) and the resulting mixture stirred at room temperature for 1 h. After diluting with water (2 L), the mixture was extracted with dichloromethane (2×1 L) and the combined organic extracts dried over sodium sulfate, filtered and concentrated in vacuo. The residue was purified by flash column chromatography (silica), eluting with ethyl acetate/petroleum spirit, 40-60° C. (50%) to give the title compound (98.0 g, 64%) as a yellow solid.

¹H NMR (400 MHz, CDCl₃) δ 7.69-7.65 (m, 1H), 6.83-6.77 (m, 1H), 5.56 (d, J=6.6 Hz, 1H), 4.25-4.21 (m, 1H), 4.04-3.98 (m, 1H), 3.92-3.83 (m, 4H), 3.80-3.72 (m, 3H), 3.70-3.63 (m, 1H), 3.15-3.07 (m, 1H), 2.62-2.46 (m, 1H), 2.20-1.69 (m, 3H), 1.32-1.27 (m, 3H), 1.12-1.08 (m, 18H); MS (ES+): m/z=511 (M+H)⁺; LCMS (Method E): t_{R}=3.70 min.

### ((2S,4R)-4-Hydroxy-2-(hydroxymethyl)piperidin-1-yl)(5-methoxy-2-nitro-4-((triisopropylsilyl)oxy)phenyl)methanone (50)

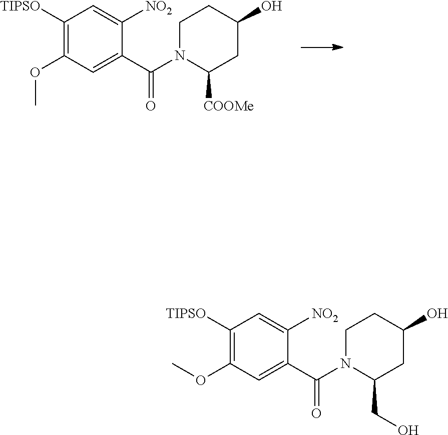

A solution of methyl (2S,4R)-4-hydroxy-1-(5-methoxy-2-nitro-4-((triisopropylsilyl)oxy)benzoyl)piperidine-2-carboxylate (49) (98.0 g, 0.19 mol) in tetrahydrofuran (1 L) was charged with lithium borohydride (2 M in THF, 0.38 L) at 0° C. The resulting mixture was stirred for 2 h, before quenching with water (2 L) and extracting with ethyl acetate (2×1 L). The combined organic extracts were then dried over anhydrous sodium sulfate, filtered and concentrated in vacuo. The residue was washed with diethyl ether and filtered to afford the title compound (84.5 g, 91%) as a white solid.

¹H NMR (400 MHz, CDCl₃) δ 7.71-7.65 (m, 1H), 6.88-6.72 (m, 1H), 5.01-4.62 (m, 1H), 4.35-4.16 (m, 1H), 4.14-3.80 (m, 5H), 3.71-3.54 (m, 1H), 3.43-3.07 (m, 1H), 2.21-1.88 (m, 2H), 1.79-1.51 (m, 2H), 1.32-1.25 (m, 3H), 1.11-1.08 (m, 18H); MS (ES+): m/z=483 (M+H)⁺; LCMS (Method E): t_{R}=3.30 min.

### ((2S,4R)-2-(((tert-Butyldimethylsilyl)oxy)methyl)-4-hydroxypiperidin-1-yl)(5-methoxy-2-nitro-4-((triisopropylsilyl)oxy)phenyl)methanone (51)

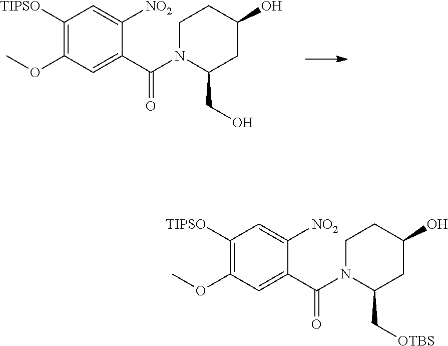

A solution of ((2S,4R)-4-hydroxy-2-(hydroxymethyl)piperidin-1-yl)(5-methoxy-2-nitro-4-((triisopropylsilyl)oxy)phenyl)methanone (50) (84.5 g, 0.18 mol) in N,N-dimethylformamide (1 L) was charged with imidazole (29.8 g, 0.44 mol) and tert-butyldimethylsilyl chloride (39.6 g, 0.26 mol). The reaction mixture was stirred at room temperature for 3 h and then diluted with water (2 L) and extracted with dichloromethane (2×1.5 L). The combined organic extracts were then dried over anhydrous sodium sulfate, filtered and concentrated in vacuo. The residue was purified by flash column chromatography (silica), eluting with ethyl acetate/petroleum spirit, 40-60° C. (50%) to give the title compound (81.0 g, 77%) as a pale yellow oil.

¹H NMR (400 MHz, CDCl₃) δ 7.70-7.67 (m, 1H), 6.73-6.63 (m, 1H), 5.04-4.64 (m, 1H), 4.37-4.02 (m, 2H), 3.89 (d, J=7.0 Hz, 3H), 3.80-3.76 (m, 1H), 3.64-3.51 (m, 1H), 3.32-3.05 (m, 1H), 2.30-2.13 (m, 1H), 2.05-1.91 (m, 1H), 1.81-1.65 (m, 1H), 1.62-1.50 (m, 1H), 1.32-1.24 (m, 3H), 1.11-1.09 (m, 18H), 0.97-0.87 (m, 9H), 0.24-0.02 (m, 6H); MS (ES+): m/z=597 (M+H)⁺; LCMS (Method D): t_{R}=1.73 min.

### (S)-2-(((tert-Butyldimethylsilyl)oxy)methyl)-1-(5-methoxy-2-nitro-4-((triisopropylsilyl)oxy)benzoyl)piperidin-4-one (52)

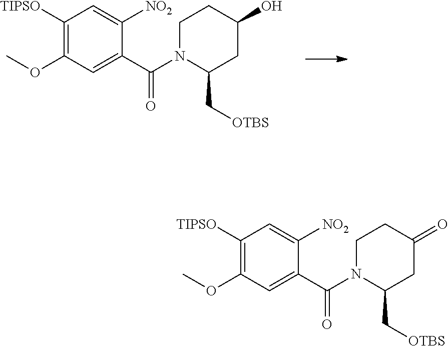

A solution of ((2S,4R)-2-(((tert-butyldimethylsilyl)oxy)methyl)-4-hydroxypiperidin-1-yl)(5-methoxy-2-nitro-4-((triisopropylsilyl)oxy)phenyl)methanone (51) (41.0 g, 68.7 mmol) in dichloromethane (400 mL) was charged with TEMPO (1.07 g, 6.90 mmol) and (diacetoxyiodo)benzene (28.8 g, 89.4 mmol). The reaction mixture was stirred at room temperature for 16 h and then diluted with water (1 L) and extracted with DCM (2×1 L). The combined organic extracts were then dried over anhydrous sodium sulfate, filtered and concentrated in vacuo. The residue was then purified by flash column chromatography (silica), eluting with ethyl acetate/petroleum spirit, 40-60° C. (25%) to give the title compound (37.0 g, 90%) as a colourless oil.

¹H NMR (400 MHz, CDCl₃) δ 7.72-7.67 (m, 1H), 6.77-6.68 (m, 1H), 5.24-4.99 (m, 1H), 4.02-3.74 (m, 5H), 3.72-3.29 (m, 2H), 2.86-2.52 (m, 2H), 2.49-2.24 (m, 2H), 1.32-1.26 (m, 3H), 1.11-1.08 (m, 18H), 0.90-0.83 (m, 9H), 0.08-0.01 (m, 6H); MS (ES+): m/z=617 (M+Na)+; LCMS (Method D): t_{R}=3.57 min.

### (S)-2-(((tert-Butyldimethylsilyl)oxy)methyl)-1-(5-methoxy-2-nitro-4-((triisopropylsilyl)oxy)benzoyl)-1,2,3,6-tetrahydropyridin-4-yl trifluoromethanesulfonate (53)

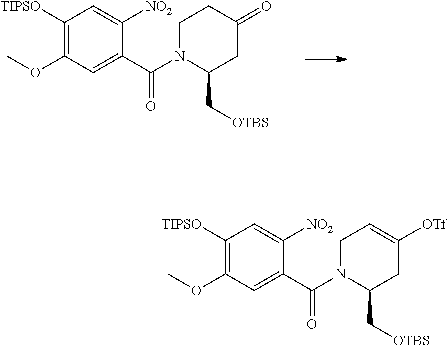

A solution of(S)-2-(((tert-butyldimethylsilyl)oxy)methyl)-1-(5-methoxy-2-nitro-4-((triisopropylsilyl)oxy)benzoyl)piperidin-4-one (52) (33.0 g, 55.5 mmol) in tetrahydrofuran (300 mL) was charged with sodium bis(trimethylsilyl)amide (2 M in tetrahydrofuran, 41 mL) at −78° C., under an inert atmosphere of nitrogen. The resulting mixture was stirred at this temperature for 1 h before N-phenyl-bis(trifluoromethanesulfonimide) (25.8 g, 72.2 mmol) in tetrahydrofuran (100 mL) was added and stirred at room temperature for 3 h. After quenching with water (1 L), the mixture was extracted with ethyl acetate (2×1 L). The combined organic extracts were then dried over anhydrous sodium sulfate, filtered and concentrated in vacuo. The residue was then purified by flash column chromatography (silica), eluting with ethyl acetate/petroleum spirit, 40-60° C. (10%) to give the title compound (13.0 g, 32%) as a brown oil.

¹H NMR (400 MHz, CDCl₃) δ 7.74-7.68 (m, 1H), 6.79-6.63 (m, 1H), 5.88-5.60 (m, 1H), 5.38-5.00 (m, 1H), 3.92-3.89 (m, 3H), 3.83-3.73 (m, 2H), 3.73-3.56 (m, 2H), 3.02-2.71 (m, 1H), 2.66-2.49 (m, 1H), 2.38-1.98 (m, 1H), 1.32-1.26 (m, 3H), 1.11-1.09 (m, 18H), 0.93-0.77 (m, 9H), 0.10-0.00 (m, 6H); MS (ES+): m/z=727 (M+H)⁺; LCMS (Method D): t_{R}=1.80 min.

### (S)-4-(2-(((tert-Butyldimethylsilyl)oxy)methyl)-1-(5-methoxy-2-nitro-4-((triisopropylsilyl)oxy)benzoyl)-1,2,3,6-tetrahydropyridin-4-yl)-N-methylbenzenesulfonamide (54)

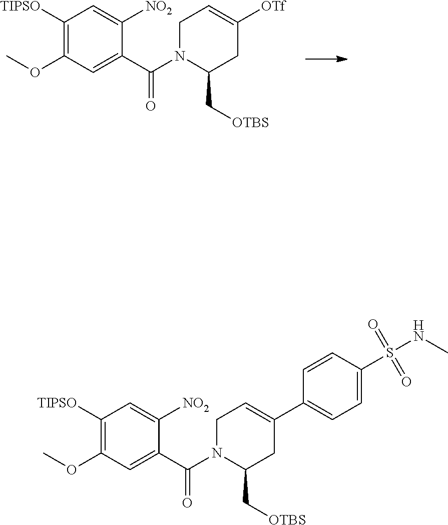

A solution of(S)-2-(((tert-butyldimethylsilyl)oxy)methyl)-1-(5-methoxy-2-nitro-4-((triisopropylsilyl)oxy)benzoyl)-1,2,3,6-tetrahydropyridin-4-yl trifluoromethanesulfonate (53) (9.70 g, 13.4 mmol) in acetonitrile (150 mL) was charged with (4-(N-methylsulfamoyl)phenyl)boronic acid (2.90 g, 13.4 mmol), [1,1′-bis(diphenylphosphino)ferrocene]dichloropalladium(II), complex with dichloromethane (1.00 g, 1.30 mmol), and potassium carbonate (5.54 g, 40.0 mmol), and the resulting mixture heated to 50° C. for 3 h, under an inert atmosphere of nitrogen. After cooling to room temperature, the mixture was diluted with water (300 mL) and extracted with ethyl acetate (3×200 mL). The combined organic extracts were washed with water (200 mL) and then dried over anhydrous sodium sulfate, filtered, and concentrated in vacuo. The residue was then purified by flash column chromatography (silica), eluting with ethyl acetate/petroleum spirit, 40-60° C. (33%) to give the title compound (5.6 g, 56%) as a yellow solid.

¹H NMR (400 MHz, DMSO-d₆) δ 7.75-7.73 (m, 2H), 7.67-7.59 (m, 3H), 7.45-7.41 (m, 1H), 7.21-7.02 (m, 1H), 6.46-6.22 (m, 1H), 5.01-4.78 (m, 1H), 3.99-3.50 (m, 7H), 2.90-2.54 (m, 2H), 2.40-2.38 (m, 3H), 1.31-1.26 (m, 3H), 1.08-1.06 (m, 18H), 0.86-0.80 (m, 9H), 0.05-0.22 (m, 6H); MS (ES+): m/z=748 (M+H)⁺; LCMS (Method D): t_{R}=3.60 min.

### (S)-4-(1-(2-Amino-5-methoxy-4-((triisopropylsilyl)oxy)benzoyl)-2-(((tert-butyldimethylsilyl)oxy)methyl)-1,2,3,6-tetrahydropyridin-4-yl)-N-methylbenzenesulfonamide (55)

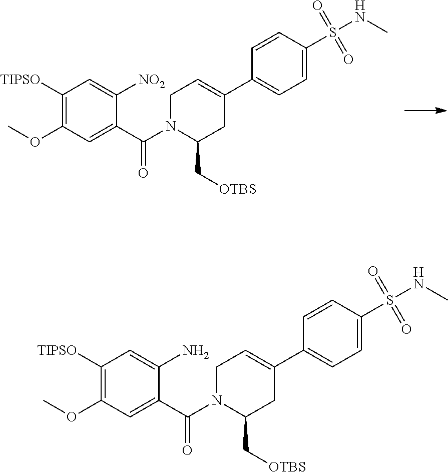

A mixture of(S)-4-(2-(((tert-butyldimethylsilyl)oxy)methyl)-1-(5-methoxy-2-nitro-4-((triisopropylsilyl)oxy)benzoyl)-1,2,3,6-tetrahydropyridin-4-yl)-N-methylbenzenesulfonamide (54) (5.60 g, 7.50 mmol) in ethanol (60 mL) and water (15 mL) was charged with iron (2.10 g, 37.4 mmol) and ammonium chloride (2.00 g, 37.4 mmol) and the resulting mixture stirred at 80° C. for 3 h. after cooling to room temperature, the mixture was diluted into water (200 mL) and extracted with ethyl acetate (3×150 mL). The combined organic extracts were then dried over sodium sulfate, filtered and concentrated in vacuo. Purification by flash column chromatography (silica), eluting with ethyl acetate/petroleum spirit, 40-60° C. (33%) gave the title compound (4.40 g, 82%) as a yellow solid.

¹H NMR (400 MHz, DMSO-d₆) δ 7.74-7.72 (m, 2H), 7.67-7.65 (m, 2H), 7.44-7.40 (m, 1H), 6.67 (s, 1H), 6.38-6.34 (m, 2H), 4.89 (s, 2H), 4.51-4.21 (m, 1H), 3.64-3.59 (m, 5H), 2.82 (s, 1H), 2.55 (s, 1H), 2.39 (d, J=5.0 Hz, 3H), 1.27-1.22 (m, 3H), 1.07-1.05 (m, 18H), 0.79 (s, 9H), 0.06 (s, 6H); MS (ES+): m/z=718 (M+H)⁺; LCMS (Method D): t_{R}=3.20 min.

### Allyl (S)-(2-(2-(((tert-Butyldimethylsilyl)oxy)methyl)-4-(4-(N-methylsulfamoyl)phenyl)-1,2,3,6-tetrahydropyridine-1-carbonyl)-4-methoxy-5-((triisopropylsilyl)oxy)phenyl)carbamate (56)

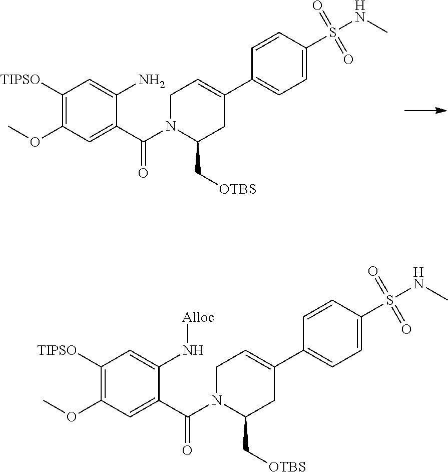

A solution of(S)-4-(1-(2-amino-5-methoxy-4-((triisopropylsilyl)oxy)benzoyl)-2-(((tert-butyldimethylsilyl)oxy)methyl)-1,2,3,6-tetrahydropyridin-4-yl)-N-methylbenzenesulfonamide (55) (4.40 g, 6.13 mmol) in dichloromethane (50 mL) was charged with pyridine (0.97 g, 12.3 mmol) and allyl chloroformate (0.77 g, 6.44 mmol) at 0° C., and the resulting mixture stirred for 2 h, before being concentrated in vacuo, and then purified by flash column chromatography (silica), eluting with ethyl acetate/petroleum spirit, 40-60° C. (33%), to give the title compound (4.70 g, 95%) as a pale yellow solid.

¹H NMR (400 MHz, DMSO-d₆) δ 9.01 (s, 1H), 7.75-7.73 (m, 2H), 7.66-7.64 (m, 2H), 7.44-7.40 (m, 1H), 6.92-6.82 (m, 1H), 6.44-6.24 (m, 1H), 5.84 (s, 1H), 5.25 (d, J=17.2 Hz, 1H), 5.11 (d, J=10.4 Hz, 1H), 4.61-4.50 (m, 3H), 4.09 (s, 1H), 3.75-3.50 (m, 6H), 3.37-3.36 (m, 1H), 2.94-2.72 (m, 1H), 2.39 (d, J=5.0 Hz, 3H), 1.27-1.22 (m, 3H), 1.06-1.04 (m, 18H), 0.81-0.74 (m, 9H), 0.09-0.03 (m, 6H); MS (ES+): m/z=802 (M+H)⁺; LCMS (Method D): t_{R}=3.70 min.

### Allyl (S)-(2-(2-(hydroxymethyl)-4-(4-(N-methylsulfamoyl)phenyl)-1,2,3,6-tetrahydropyridine-1-carbonyl)-4-methoxy-5-((triisopropylsilyl)oxy)phenyl)carbamate (57)

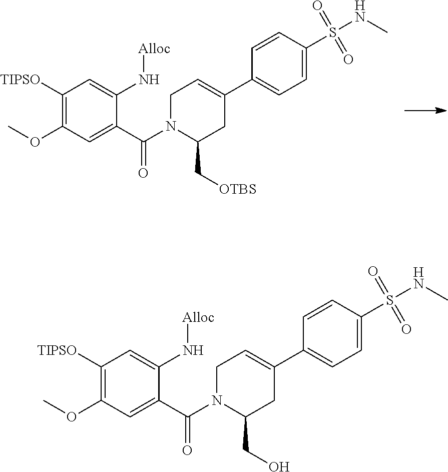

Allyl (S)-(2-(2-(((tert-butyldimethylsilyl)oxy)methyl)-4-(4-(N-methylsulfamoyl)phenyl)-1,2,3,6-tetrahydropyridine-1-carbonyl)-4-methoxy-5-((triisopropylsilyl)oxy)phenyl)carbamate (56) (4.70 g, 5.86 mmol) in acetic acid (70 mL), methanol (10 mL), tetrahydrofuran (10 mL) and water (20 mL) was stirred at room temperature for 3 h. The mixture was then charged with a saturated aqueous solution of sodium hydrogen carbonate (400 mL) and extracted with ethyl acetate (2×200 mL). The combined organic extracts were washed with a saturated aqueous solution of sodium chloride, dried over sodium sulfate, filtered and concentrated in vacuo. Purification by flash column chromatography (silica), eluting with ethyl acetate/petroleum spirit, 40-60° C. (50%), gave the title compound (3.50 g, 87%) as an off-white solid.

¹H NMR (400 MHz, DMSO-d₆) δ 8.87-8.71 (m, 1H), 7.75-7.73 (m, 2H), 7.67-7.65 (m, 2H), 7.44-7.40 (m, 1H), 7.23 (s, 1H), 6.93 (s, 1H), 6.41-6.23 (m, 1H), 5.88 (s, 1H), 5.36-4.65 (m, 4H), 4.53-4.48 (m, 2H), 3.98-3.94 (m, 1H), 3.80-3.73 (m, 4H), 3.50 (s, 1H), 2.82-2.69 (m, 1H), 2.40 (d, J=5.0 Hz, 4H), 1.28-1.21 (m, 3H), 1.07-1.05 (m, 18H); MS (ES+): m/z=688 (M+H)⁺; LCMS (Method E): t_{R}=3.93 min.

### Allyl (6aS)-6-hydroxy-2-methoxy-8-(4-(N-methylsulfamoyl)phenyl)-12-oxo-3-((triisopropylsilyl)oxy)-6,6a,7,10-tetrahydrobenzo[e]pyrido[1,2-a][1,4]diazepine-5(12H)-carboxylate (58)

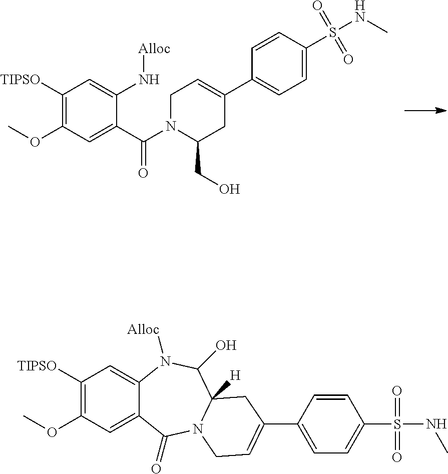

A solution of allyl (S)-(2-(2-(hydroxymethyl)-4-(4-(N-methylsulfamoyl)phenyl)-1,2,3,6-tetrahydropyridine-1-carbonyl)-4-methoxy-5-((triisopropylsilyl)oxy)phenyl)carbamate (57) (3.20 g, 4.65 mmol) in dichloromethane (80 mL) was charged with TEMPO (73.0 mg, 0.50 mmol) and (diacetoxyiodo)benzene (1.95 g, 6.05 mmol) and the resulting mixture was stirred at room temperature for 16 h. The mixture was then diluted into water (100 mL) and extracted with ethyl acetate (2×200 mL). The combined organic extracts were washed with a saturated aqueous solution of sodium chloride, dried over sodium sulfate, filtered and concentrated in vacuo. Purification by flash column chromatography (silica), eluting with ethyl acetate/petroleum spirit, 40-60° C. (50%), gave the title compound (2.40 g, 75%) as a white solid.

¹H NMR (400 MHz, DMSO-d₆) δ 7.80-7.77 (m, 2H), 7.71-7.69 (m, 2H), 7.48-7.44 (m, 1H), 7.13 (s, 1H), 6.71-6.60 (m, 3H), 5.76 (s, 1H), 5.44 (dd, J=90.8, 4.8 Hz, 1H), 5.11-5.05 (m, 2H), 4.54-4.49 (m, 1H), 4.40-4.35 (m, 1H), 4.19-4.04 (m, 2H), 3.83 (s, 3H), 3.55-3.51 (m, 1H), 2.93-2.89 (m, 1H), 2.80-2.76 (m, 1H), 2.42 (d, J=5.0 Hz, 3H), 1.24-1.19 (m, 3H), 1.06-1.03 (m, 18H); MS (ES+): m/z=686 (M+H)⁺; LCMS (Method D): t_{R}=1.53 min.

### Allyl (6aS)-2-methoxy-8-(4-(N-methyl-N-(tetrahydro-2H-pyran-2-yl)sulfamoyl)phenyl)-12-oxo-6-((tetrahydro-2H-pyran-2-yl)oxy)-3-((triisopropylsilyl)oxy)-6,6a,7,10-tetrahydrobenzo[e]pyrido[1,2-a][1,4]diazepine-5(12H)-carboxylate (59)

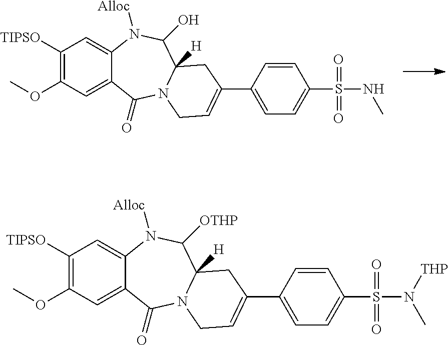

A mixture of allyl (6aS)-6-hydroxy-2-methoxy-8-(4-(N-methylsulfamoyl)phenyl)-12-oxo-3-((triisopropylsilyl)oxy)-6,6a,7,10-tetrahydrobenzo[e]pyrido[1,2-a][1,4]diazepine-5(12H)-carboxylate (58) (2.40 g, 3.50 mmol) in tetrahydrofuran (50 mL) was charged with 3,4-dihydro-2H-pyran (11.8 g, 140 mmol) and p-toluenesulfonic acid monohydrate (240 mg) and the resulting mixture stirred at room temperature for 16 h. The mixture was then charged with water (200 mL) and extracted with ethyl acetate (2×200 mL). The combined organic extracts were then washed with a saturated aqueous solution of sodium chloride, dried over sodium sulfate, filtered and concentrated in vacuo. Purification by flash column chromatography (silica), eluting with ethyl acetate/petroleum spirit, 40-60° C. (50%), gave the title compound (3.00 g, 99%) as a yellow solid.

¹H NMR (400 MHz, CDCl₃) δ 7.88-7.79 (m, 2H), 7.62-7.47 (m, 2H), 7.19-7.16 (m, 1H), 7.06-6.94 (m, 1H), 6.68-6.57 (m, 1H), 6.48-6.36 (m, 1H), 5.87-5.64 (m, 2H), 5.20-4.83 (m, 4H), 4.64-4.15 (m, 4H), 3.89-3.83 (m, 4H), 3.79-3.68 (m, 1H), 3.52-3.35 (m, 2H), 3.25-3.07 (m, 1H), 2.76-2.73 (m, 3H), 2.34-2.18 (m, 1H), 1.92 (s, 1H), 1.71-1.59 (m, 6H), 1.52-1.37 (m, 6H), 1.24-1.18 (m, 3H), 1.09-1.06 (m, 18H); MS (ES+): m/z=854 (M+H)⁺; LCMS (Method D): t_{R}=3.60 min.

### Allyl (6aS)-3-hydroxy-2-methoxy-8-(4-(N-methyl-N-(tetrahydro-2H-pyran-2-yl)sulfamoyl)phenyl)-12-oxo-6-((tetrahydro-2H-pyran-2-yl)oxy)-6,6a,7,10-tetrahydrobenzo[e]pyrido[1,2-a][1,4]diazepine-5(12H)-carboxylate (60)

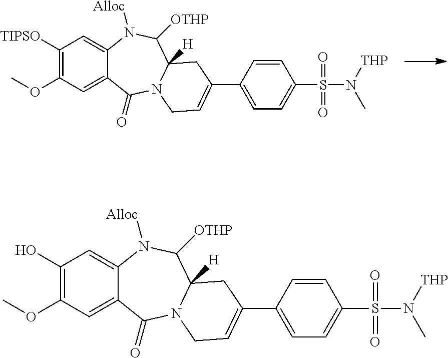

A solution of allyl (6aS)-2-methoxy-8-(4-(N-methyl-N-(tetrahydro-2H-pyran-2-yl)sulfamoyl)phenyl)-12-oxo-6-((tetrahydro-2H-pyran-2-yl)oxy)-3-((triisopropylsilyl)oxy)-6,6a,7,10-tetrahydrobenzo[e]pyrido[1,2-a][1,4]diazepine-5(12H)-carboxylate (59) (3.00 g, 3.51 mmol) in tetrahydrofuran (50 mL) was charged with tetrabutylammonium fluoride (1 M in tetrahydrofuran, 4.6 mL) and the resulting mixture stirred at room temperature for 1.5 h. Water (200 mL) was then added, and the mixture extracted with ethyl acetate (2×200 mL). The combined organic extracts were washed with a saturated aqueous solution of sodium chloride, dried over sodium sulfate, filtered and concentrated in vacuo. Purification by flash column chromatography (silica), eluting with ethyl acetate/petroleum spirit, 40-60° C. (from 70% to 90%), gave the title compound (1.50 g, 61%) as a pale yellow solid.

¹H NMR (400 MHz, DMSO-d₆) δ 9.84-9.80 (m, 1H), 7.81-7.67 (m, 4H), 7.10-7.08 (m, 1H), 6.80-6.52 (m, 2H), 5.76 (s, 1H), 5.70-5.47 (m, 1H), 5.09-5.00 (m, 3H), 4.82 (s, 1H), 4.49 (s, 2H), 4.08 (s, 2H), 3.84 (s, 3H), 3.76-3.70 (m, 1H), 3.67-3.67 (m, 1H), 3.51-3.41 (m, 2H), 3.11-2.78 (m, 3H), 2.67 (s, 3H), 1.82 (s, 1H), 1.63-1.36 (m, 11H); MS (ES+): m/z=698 (M+H)⁺; LCMS (Method E): t_{R}=2.53 min.

### (S)-(2-(((tert-Butyldimethylsilyl)oxy)methyl)-4-(thiophen-3-yl)-3,6-dihydropyridin-1(2H)-yl)(5-methoxy-2-nitro-4-((triisopropylsilyl)oxy)phenyl)methanone (61)

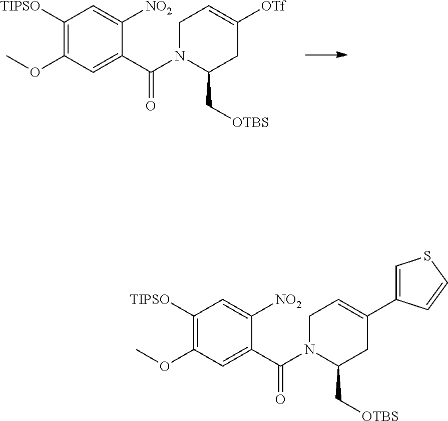

A solution of(S)-2-(((tert-butyldimethylsilyl)oxy)methyl)-1-(5-methoxy-2-nitro-4-((triisopropylsilyl)oxy)benzoyl)-1,2,3,6-tetrahydropyridin-4-yl trifluoromethanesulfonate (53) (22.9 g, 31.5 mmol) in acetonitrile (230 mL) was charged with 3-thienylboronic acid (6.05 g, 47.3 mmol), [1,1′-bis(diphenylphosphino)ferrocene]dichloropalladium(II), complex with dichloromethane (691 mg, 0.95 mmol), and potassium carbonate (13.0 g, 94.5 mmol), and the resulting mixture was heated to 80° C., under nitrogen, for 16 h. After cooling to room temperature, and diluting into water (600 mL), the mixture was extracted with ethyl acetate (3×200 mL) and the combined organic extracts were washed with brine (200 mL), dried over sodium sulfate and concentrated in vacuo. The residue was then purified by flash column chromatography (silica), eluting with ethyl acetate/petroleum spirit, 40-60° C. (10%), to give the title compound (11.9 g, 57%) as a yellow oil.

¹H NMR (400 MHz, DMSO-d₆) δ 7.56-7.15 (m, 6H), 6.25-6.01 (m, 2H), 4.92-4.77 (m, 2H), 3.89-3.62 (m, 7H), 2.62-2.57 (m, 2H), 1.26-1.07 (m, 26H), 0.15-0.01 (m, 6H); MS (ES+): m/z=683 (M+Na)+; LCMS (Method D): t_{R}=4.44 min.

### (S)-(2-Amino-5-methoxy-4-((triisopropylsilyl)oxy)phenyl)(2-(((tert-butyldimethylsilyl)oxy)methyl)-4-(thiophen-3-yl)-3,6-dihydropyridin-1(2H)-yl)methanone (62)

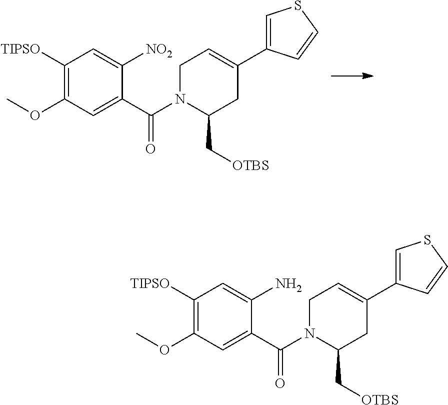

A solution of(S)-(2-(((tert-butyldimethylsilyl)oxy)methyl)-4-(thiophen-3-yl)-3,6-dihydropyridin-1(2H)-yl)(5-methoxy-2-nitro-4-((triisopropylsilyl)oxy)phenyl)methanone (61) (10.9 g, 16.4 mmol) in ethanol (80 mL) and water (20 mL) was charged with iron (4.60 g, 82.2 mmol) and ammonium chloride (4.40 g, 82.2 mmol) and the resulting mixture was stirred at 80° C. for 2 h. After cooling to room temperature, the mixture was diluted into water (600 mL) and extracted with ethyl acetate (3×200 mL). The combined organic extracts were dried over sodium sulfate, filtered and concentrated in vacuo. Purification by flash column chromatography (silica), eluting with ethyl acetate/petroleum spirit, 40-60° C. (10%), gave the title compound (8.15 g, 79%) as a yellow solid.

¹H NMR (400 MHz, DMSO-d₆) δ 7.55-7.48 (m, 1H), 7.42-7.32 (m, 2H), 6.65 (s, 1H), 6.34 (s, 1H), 6.21 (s, 1H), 4.87 (s, 2H), 3.66-3.54 (m, 5H), 2.67 (s, 1H), 2.54 (s, 1H), 1.28-1.18 (m, 4H), 1.09-1.01 (m, 20H), 0.81 (s, 9H), −0.04 (s, 6H); MS (ES+): m/z=631 (M+H)⁺; LCMS (Method D): t_{R}=2.73 min.

### Allyl (S)-(2-(2-(((tert-butyldimethylsilyl)oxy)methyl)-4-(thiophen-3-yl)-1,2,3,6-tetrahydropyridine-1-carbonyl)-4-methoxy-5-((triisopropylsilyl)oxy)phenyl)carbamate (63)

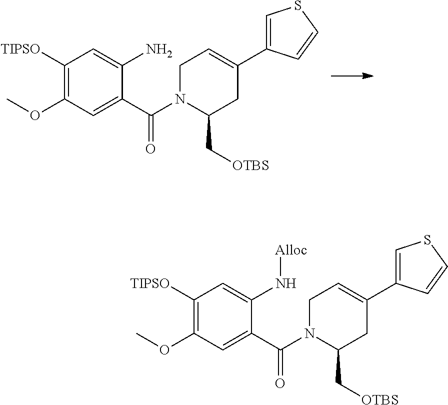

A solution of (S)-(2-amino-5-methoxy-4-((triisopropylsilyl)oxy)phenyl)(2-(((tert-butyldimethylsilyl)oxy)methyl)-4-(thiophen-3-yl)-3,6-dihydropyridin-1(2H)-yl)methanone (62) (8.15 g, 12.9 mmol) in dichloromethane (80 mL) was charged with allyl chloroformate (1.71 g, 14.2 mmol) and pyridine (1.53 g, 19.4 mmol) and the resulting mixture stirred at 0° C. for 1 h, before water (200 mL) was added. After extracting with ethyl acetate (3×70 mL), the combined organic extracts were dried over sodium sulfate, filtered and concentrate in vacuo. Purification by flash column chromatography (silica), eluting with ethyl acetate/petroleum spirit, 40-60° C. (10%), gave the title compound (7.90 g, 85%) as a pale yellow solid.

¹H NMR (400 MHz, DMSO-d₆) δ 9.07-8.83 (m, 1H), 7.56-7.47 (m, 1H), 7.46-7.25 (m, 2H), 6.95-6.75 (m, 1H), 6.33-6.01 (m, 1H), 5.96-5.79 (m, 1H), 5.33-5.09 (m, 2H), 4.61-4.28 (m, 3H), 4.15-3.85 (m, 1H), 3.81-3.42 (m, 6H), 2.85-2.56 (m, 1H), 2.48-2.27 (m, 1H), 2.20-1.73 (m, 1H), 1.51-0.94 (m, 21H), 0.91-0.57 (m, 9H), 0.22-0.17 (m, 6H); MS (ES+): m/z=715 (M+H)⁺; LCMS (Method D): t_{R}=2.32 min.

### Allyl (S)-(2-(2-(hydroxymethyl)-4-(thiophen-3-yl)-1,2,3,6-tetrahydropyridine-1-carbonyl)-4-methoxy-5-((triisopropylsilyl)oxy)phenyl)carbamate (64)

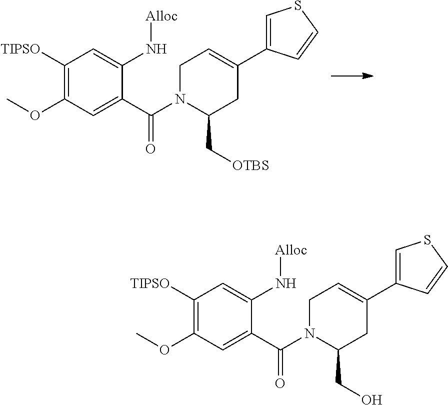

A solution of allyl (S)-(2-(2-(((tert-butyldimethylsilyl)oxy)methyl)-4-(thiophen-3-yl)-1,2,3,6-tetrahydropyridine-1-carbonyl)-4-methoxy-5-((triisopropylsilyl)oxy)phenyl)carbamate (63) (7.90 g, 11.1 mmol) in acetic acid (51 mL), methanol (7 mL), tetrahydrofuran (7 mL) and water (14 mL) was stirred at room temperature for 16 h. Water (600 mL) was then added, and the resulting mixture was extracted with ethyl acetate (3×200 mL). The combined organic extracts were then dried over sodium sulfate, filtered and concentrated in vacuo. Purification by flash column chromatography (silica), eluting with ethyl acetate/petroleum spirit, 40-60° C. (10%), gave the title compound (5.70 g, 86%) as a white solid.

¹H NMR (400 MHz, DMSO-d₆) δ 8.93-8.54 (m, 1H), 7.54-7.50 (m, 1H), 7.47-7.12 (m, 3H), 6.90 (s, 1H), 6.32-5.99 (m, 1H), 5.98-5.65 (m, 1H), 5.27 (d, J=17.2 Hz, 1H), 5.14 (d, J=10.8 Hz, 1H), 4.73-4.36 (m, 3H), 3.92 (s, 1H), 3.79-3.65 (m, 5H), 3.53-3.40 (m, 2H), 2.74-2.55 (m, 1H), 2.45-2.30 (m, 1H), 1.26-1.22 (m, 3H), 1.07 (d, J=70.2 Hz, 18H); MS (ES+): m/z=601 (M+H)⁺; LCMS (Method D): t_{R}=2.22 min.

### Allyl (6aS)-6-hydroxy-2-methoxy-12-oxo-8-(thiophen-3-yl)-3-((triisopropylsilyl)oxy)-6,6a,7,10-tetrahydrobenzo[e]pyrido[1,2-a][1,4]diazepine-5(12H)-carboxylate (65)

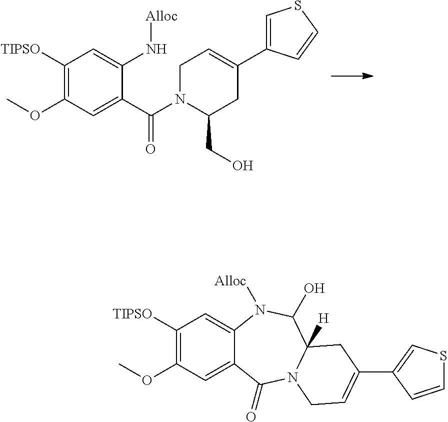

A solution of allyl (S)-(2-(2-(hydroxymethyl)-4-(thiophen-3-yl)-1,2,3,6-tetrahydropyridine-1-carbonyl)-4-methoxy-5-((triisopropylsilyl)oxy)phenyl)carbamate (64) (5.70 g, 9.49 mmol) in dichloromethane (60 mL) was charged with TEMPO (148 mg, 0.95 mmol) and (diacetoxyiodo)benzene (3.67 g, 11.4 mmol). The resulting mixture was stirred at room temperature for 16 h and then diluted into water (600 mL). After extracting with ethyl acetate (3×200 mL), the combined organic extracts were washed with brine (200 mL), dried over sodium sulfate and concentrated in vacuo. Purification by flash column chromatography (silica), eluting with ethyl acetate/petroleum spirit, 40-60° C. (10%), gave the title compound (3.79 g, 67%) as a pale yellow solid.

¹H NMR (400 MHz, DMSO-d₆) δ 7.58 (dd, J=50.0, 2.8 Hz, 1H), 7.53-7.47 (m, 1H), 7.45-7.39 (m, 1H), 7.11 (s, 1H), 6.68 (s, 1H), 6.62 (d, J=5.0 Hz, 1H), 6.48-6.42 (m, 1H), 5.76 (s, 1H), 5.43 (dd, J=90.6, 4.8 Hz, 1H), 5.17-5.02 (m, 2H), 4.57-4.33 (m, 2H), 4.11-3.98 (m, 2H), 3.82 (s, 3H), 3.52-3.43 (m, 1H), 2.91 (dd, J=16.4, 2.0 Hz, 1H), 2.71-2.62 (m, 1H), 1.26-1.19 (m, 3H), 1.05 (dd, J=70.4, 3.0 Hz, 18H); MS (ES+): m/z=599 (M+H)⁺; LCMS (Method D): t_{R}=1.64 min.

### Allyl (6aS)-2-methoxy-12-oxo-6-((tetrahydro-2H-pyran-2-yl)oxy)-8-(thiophen-3-yl)-3-((triisopropylsilyl)oxy)-6,6a,7,10-tetrahydrobenzo[e]pyrido[1,2-a][1,4]diazepine-5(12H)-carboxylate (66)

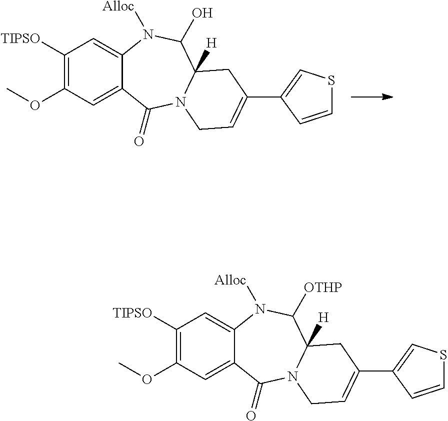

A mixture of allyl (6aS)-6-hydroxy-2-methoxy-12-oxo-8-(thiophen-3-yl)-3-((triisopropylsilyl)oxy)-6,6a,7,10-tetrahydrobenzo[e]pyrido[1,2-a][1,4]diazepine-5(12H)-carboxylate (65) (3.79 g, 6.33 mmol) in tetrahydrofuran (40 mL) was charged with 3,4-dihydro-2H-pyran (5.32 g, 63.3 mmol) and p-toluenesulfonic acid monohydrate (38 mg) and the resulting mixture was stirred at room temperature for 18 h. Water (300 mL) was then added and the mixture extracted with ethyl acetate (3×100 mL). The combined organic extracts were washed with brine (100 mL), dried over sodium sulfate, filtered and concentrated in vacuo. Purification by flash column chromatography (silica), eluting with ethyl acetate/petroleum spirit, 40-60° C. (10%), gave the title compound (3.98 g, 92%) as a pale yellow solid.

¹H NMR (400 MHz, DMSO-d₆) δ 7.62-7.54 (m, 1H), 7.52 (s, 1H), 7.46-7.39 (m, 1H), 7.17-7.06 (m, 1H), 6.93-6.60 (m, 1H), 6.55-6.40 (m, 1H), 5.84-5.67 (m, 1H), 5.67-5.47 (m, 1H), 5.15-4.98 (m, 2H), 4.89-4.69 (m, 1H), 4.58-4.27 (m, 2H), 4.15-3.95 (m, 2H), 3.86-3.50 (m, 5H), 3.44-3.33 (m, 1H), 2.92 (br, 1H), 2.77-2.64 (m, 1H), 1.73-1.59 (m, 2H), 1.53-1.33 (m, 4H), 1.27-1.20 (m, 3H), 1.11-0.97 (m, 18H); MS (ES+): m/z=683 (M+H)⁺; LCMS (Method E): t_{R}=2.27 min.

### Allyl (6aS)-3-hydroxy-2-methoxy-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (67)

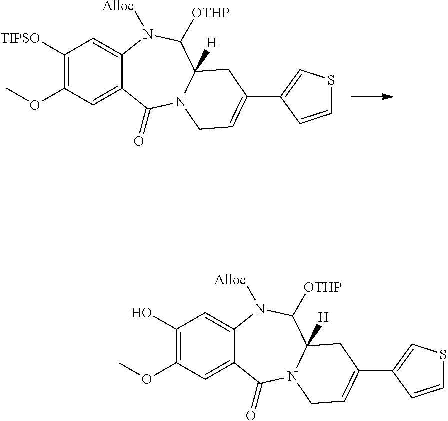

A solution of allyl (6aS)-2-methoxy-12-oxo-6-((tetrahydro-2H-pyran-2-yl)oxy)-8-(thiophen-3-yl)-3-((triisopropylsilyl)oxy)-6,6a,7,10-tetrahydrobenzo[e]pyrido[1,2-a][1,4]diazepine-5(12H)-carboxylate (66) (3.98 g, 5.83 mmol) in tetrahydrofuran (40 mL) was charged with tetrabutylammonium fluoride (1 M in tetrahydrofuran, 7.6 mL) and stirred at room temperature for 1 h. Water (300 mL) was then added and the mixture extracted with ethyl acetate (3×100 mL). The combined organics were then washed with brine (100 mL), dried over sodium sulfate, filtered and concentrated in vacuo. Purification by flash column chromatography (silica), eluting with ethyl acetate/petroleum spirit, 40-60° C. (from 5% to 20%), gave the title compound (2.30 g, 75%) as a pale yellow solid.

[α]_{D}²⁵=+142.8° (c 0.5, CH₂Cl₂); ¹H NMR (400 MHz, DMSO-d₆) δ 9.82-9.78 (m, 1H), 7.60-7.41 (m, 3H), 7.09-7.07 (m, 1H), 6.77-6.61 (m, 1H), 6.44-6.43 (m, 1H), 5.65-5.48 (m, 2H), 5.09-4.85 (m, 3H), 4.59-4.47 (m, 2H), 4.01-3.97 (m, 1H), 3.83 (s, 3H), 3.67-3.57 (m, 2H), 3.25-3.22 (m, 1H), 2.96-2.71 (m, 1H), 2.69-2.67 (m, 1H), 1.68-1.63 (m, 2H), 1.50-1.44 (m, 4H); MS (ES+): m/z=527 (M+H)⁺; LCMS (Method E): t_{R}=1.87 min.

### Allyl (6aS)-2-methoxy-3-((6-methoxy-6-oxohexyl)oxy)-8-(4-(N-methyl-N-(tetrahydro-2H-pyran-2-yl)sulfamoyl)phenyl)-12-oxo-6-((tetrahydro-2H-pyran-2-yl)oxy)-6,6a,7,10-tetrahydrobenzo[e]pyrido[1,2-a][1,4]diazepine-5(12H)-carboxylate (68)

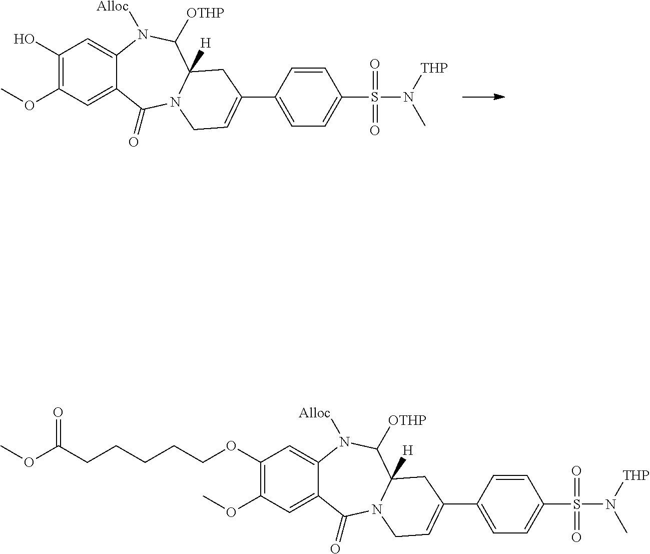

A solution of allyl (6aS)-3-hydroxy-2-methoxy-8-(4-(N-methyl-N-(tetrahydro-2H-pyran-2-yl)sulfamoyl)phenyl)-12-oxo-6-((tetrahydro-2H-pyran-2-yl)oxy)-6,6a,7,10-tetrahydrobenzo[e]pyrido[1,2-a][1,4]diazepine-5(12H)-carboxylate (60) (300 mg, 0.43 mmol) in N,N-dimethylformamide (10 mL) was charged with methyl 6-bromohexanoate (135 mg, 0.66 mmol) and potassium carbonate (178 mg, 1.29 mmol). The reaction mixture was stirred at room temperature for 3 h and then diluted with water (50 mL) and extracted with dichloromethane (2×50 mL). The combined organic extracts were then dried over anhydrous sodium sulfate, filtered and concentrated in vacuo. Purification by flash column chromatography (silica), eluting with ethyl acetate/petroleum spirit, 40-60° C. (50%), gave the title compound (277 mg, 78%) as a white solid.

¹H NMR (400 MHz, CDCl₃) δ 7.84-7.81 (m, 2H), 7.61-7.58 (m, 2H), 7.22-7.20 (m, 2H), 6.54-6.40 (m, 2H), 5.92-5.70 (m, 2H), 5.21-5.06 (m, 3H), 4.93-4.84 (m, 1H), 4.57-4.44 (m, 2H), 4.28-4.19 (m, 1H), 4.03-3.83 (m, 7H), 3.78-3.72 (m, 1H), 3.67 (s, 3H), 3.58-3.30 (m, 3H), 3.22-3.12 (m, 2H), 2.77-2.69 (m, 4H), 2.36-2.33 (m, 2H), 1.99-1.79 (m, 4H), 1.74-1.65 (m, 6H), 1.56-1.34 (m, 10H); MS (ES+): m/z=826 (M+H)⁺; LCMS (Method D): t_{R}=1.23 min.

### 6-(((6aS)-5-((Allyloxy)carbonyl)-2-methoxy-8-(4-(N-methyl-N-(tetrahydro-2H-pyran-2-yl)sulfamoyl)phenyl)-12-oxo-6-((tetrahydro-2H-pyran-2-yl)oxy)-5,6,6a,7,10,12-hexahydrobenzo[e]pyrido[1,2-a][1,4]diazepin-3-yl)oxy)hexanoic acid (69)

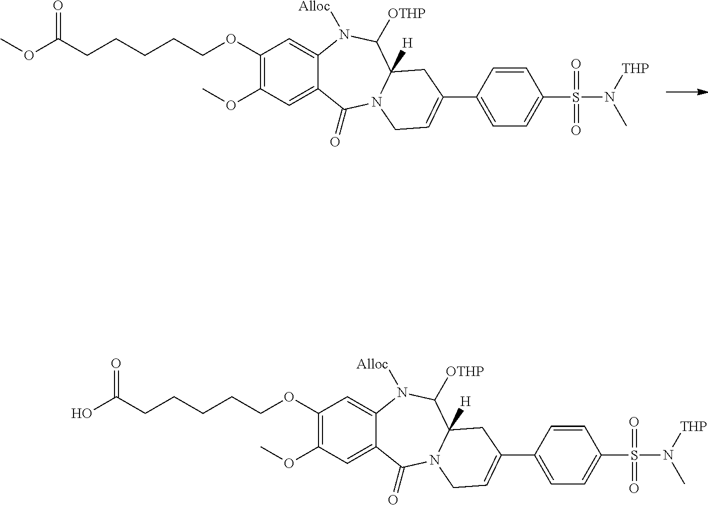

A solution of allyl (6aS)-2-methoxy-3-((6-methoxy-6-oxohexyl)oxy)-8-(4-(N-methyl-N-(tetrahydro-2H-pyran-2-yl)sulfamoyl)phenyl)-12-oxo-6-((tetrahydro-2H-pyran-2-yl)oxy)-6,6a,7,10-tetrahydrobenzo[e]pyrido[1,2-a][1,4]diazepine-5(12H)-carboxylate (68) (277 mg, 0.33 mmol) in tetrahydrofuran (6 mL) was charged with an aqueous solution of sodium hydroxide (0.5 M, 2.7 mL). The reaction mixture was stirred at room temperature for 3 h, and then diluted into water (50 mL), before extracting with ethyl acetate (2×50 mL). The combined organic extracts were then dried over anhydrous sodium sulfate, filtered and concentrated in vacuo. Purification by flash column chromatography (silica), eluting with ethyl acetate/petroleum spirit, 40-60° C. (67%), gave the title compound (240 mg, 88%) as a white solid.

¹H NMR (400 MHz, DMSO-d₆) δ 11.99 (s, 1H), 7.83-7.78 (m, 2H), 7.71-7.65 (m, 2H), 7.11 (s, 1H), 6.86-6.82 (m, 1H), 6.69-6.60 (m, 1H), 5.69-5.66 (m, 1H), 5.12-4.87 (m, 4H), 4.48 (s, 2H), 4.15-3.91 (m, 4H), 3.83 (s, 3H), 3.77-3.62 (m, 2H), 3.51-3.40 (m, 2H), 3.11-2.91 (m, 2H), 2.82-2.78 (m, 1H), 2.67-2.66 (m, 3H), 2.24-2.20 (m, 3H), 1.82 (s, 1H), 1.76-1.48 (m, 9H), 1.44-1.29 (m, 8H); MS (ES+): m/z=812 (M+H)⁺; LCMS (Method F): t_{R}=3.87 min.

### Allyl (6aS)-3-((6-((S)-1-(chloromethyl)-5-hydroxy-1,2-dihydro-3H-benzo[e]indol-3-yl)-6-oxohexyl)oxy)-2-methoxy-8-(4-(N-methyl-N-(tetrahydro-2H-pyran-2-yl)sulfamoyl)phenyl)-12-oxo-6-((tetrahydro-2H-pyran-2-yl)oxy)-6,6a,7,10-tetrahydrobenzo[e]pyrido[1,2-a][1,4]diazepine-5(12H)-carboxylate (70)

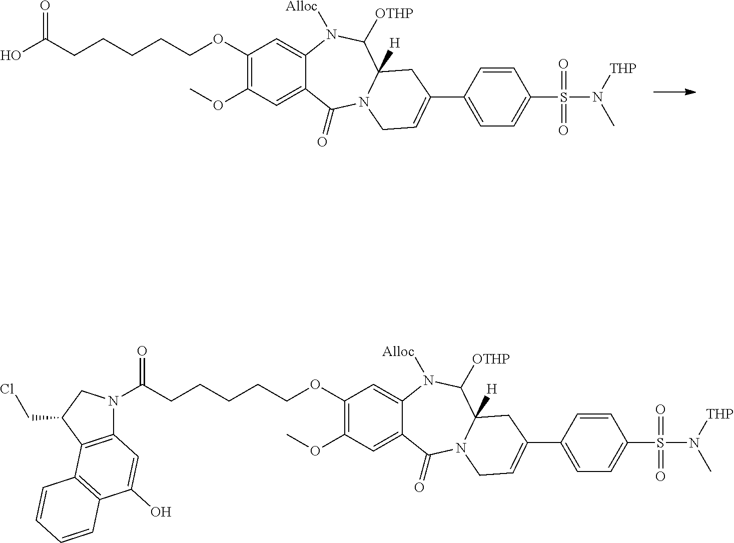

A solution of 6-(((6aS)-5-((allyloxy)carbonyl)-2-methoxy-8-(4-(N-methyl-N-(tetrahydro-2H-pyran-2-yl)sulfamoyl)phenyl)-12-oxo-6-((tetrahydro-2H-pyran-2-yl)oxy)-5,6,6a,7,10,12-hexahydrobenzo[e]pyrido[1,2-a][1,4]diazepin-3-yl)oxy)hexanoic acid (69) (240 mg, 0.30 mmol) in N,N-dimethylacetamide (8 mL) was charged with (S)-1-(chloromethyl)-2,3-dihydro-1H-benzo[e]indol-5-ol hydrochloride (12) (112 mg, 0.41 mmol) and N-(3-dimethylaminopropyl)-N′-ethylcarbodiimide hydrochloride (227 mg, 1.18 mmol). The reaction mixture was stirred at room temperature for 16 h and then diluted into water (50 mL) before extracting with dichloromethane (2×50 mL). The combined organic extracts were dried over anhydrous sodium sulfate, filtered and concentrated in vacuo. Purification by flash column chromatography (silica), eluting with methanol/dichloromethane (5%), gave the title compound (114 mg, 38%) as a white solid.

¹H NMR (400 MHz, DMSO-d₆) δ 10.33 (s, 1H), 8.11-8.05 (m, 1H), 7.98 (s, 1H), 7.84-7.75 (m, 3H), 7.71-7.65 (m, 2H), 7.50-7.46 (m, 1H), 7.33-7.27 (m, 1H), 7.11 (s, 1H), 6.88-6.84 (m, 1H), 6.69-6.60 (m, 1H), 5.87-5.63 (m, 2H), 5.15-4.96 (m, 3H), 4.91-4.86 (m, 1H), 4.48 (s, 2H), 4.35-4.30 (m, 1H), 4.20-3.93 (m, 7H), 3.86-3.81 (m, 3H), 3.80-3.70 (m, 2H), 3.67-3.63 (m, 1H), 3.50-3.40 (m, 2H), 3.11-3.02 (m, 1H), 2.97-2.93 (m, 2H), 2.81-2.78 (m, 1H), 2.67-2.66 (m, 3H), 1.80-1.70 (m, 3H), 1.67-1.60 (m, 6H), 1.55-1.27 (m, 11H); MS (ES+): m/z=1027 (M+H)⁺; LCMS (Method E): t_{R}=3.93 min.

### Allyl (6aS)-3-((6-((S)-1-(chloromethyl)-5-hydroxy-1,2-dihydro-3H-benzo[e]indol-3-yl)-6-oxohexyl)oxy)-6-hydroxy-2-methoxy-8-(4-(N-methylsulfamoyl)phenyl)-12-oxo-6,6a,7,10-tetrahydrobenzo[e]pyrido[1,2-a][1,4]diazepine-5(12H)-carboxylate (71)

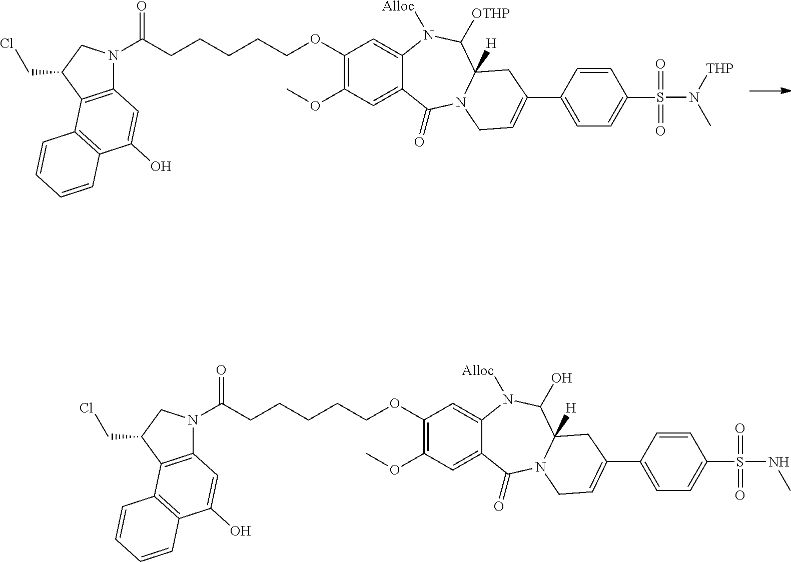

A solution of allyl (6aS)-3-((6-((S)-1-(chloromethyl)-5-hydroxy-1,2-dihydro-3H-benzo[e]indol-3-yl)-6-oxohexyl)oxy)-2-methoxy-8-(4-(N-methyl-N-(tetrahydro-2H-pyran-2-yl)sulfamoyl)phenyl)-12-oxo-6-((tetrahydro-2H-pyran-2-yl)oxy)-6,6a,7,10-tetrahydrobenzo[e]pyrido[1,2-a][1,4]diazepine-5(12H)-carboxylate (70) (110 mg, 0.11 mmol) in methanol (10 mL) was charged with p-toluenesulfonic acid monohydrate (400 mg, 2.14 mmol) and the reaction mixture stirred at room temperature for 3 h before diluting with water (80 mL) and then extracting with dichloromethane (2×50 mL). The combined organic extracts were dried over anhydrous sodium sulfate, filtered and concentrated in vacuo. Purification by flash column chromatography (silica), eluting with ethyl acetate/petroleum spirit, 40-60° C. (67%), gave the title compound (54 mg, 59%) as a white solid.

¹H NMR (400 MHz, DMSO-d₆) δ 10-33 (s, 1H), 8.07 (d, J=8.4 Hz, 1H), 7.98 (s, 1H), 7.81-7.76 (m, 3H), 7.71-7.69 (m, 2H), 7.52-7.43 (m, 2H), 7.33-7.27 (m, 1H), 7.11 (s, 1H), 6.89 (d, J=20.0 Hz, 1H), 6.65 (s, 1H), 5-75 (s, 1H), 5.37-5.21 (m, 2H), 5.10-4.97 (m, 2H), 4.55-4.44 (m, 2H), 4.36-4.29 (m, 1H), 4.17-4.10 (m, 3H), 4.05-3.95 (m, 3H), 3.82-3.75 (m, 4H), 3.66-3.60 (m, 1H), 3.53-3.46 (m, 1H), 2.87-2.80 (m, 2H), 2.43-2.41 (m, 3H), 2.02-1.96 (m, 1H), 1.80-1.69 (m, 5H), 1.54-1.49 (m, 2H); MS (ES+): m/z=859 (M+H)⁺; LCMS (Method E): t_{R}=2.73 min.

### 4-((S)-3-((6-((S)-1-(Chloromethyl)-5-hydroxy-1,2-dihydro-3H-benzo[e]indol-3-yl)-6-oxohexyl)oxy)-2-methoxy-12-oxo-6a,7,10,12-tetrahydrobenzo[e]pyrido[1,2-a][1,4]diazepin-8-yl)-N-methylbenzenesulfonamide (72)

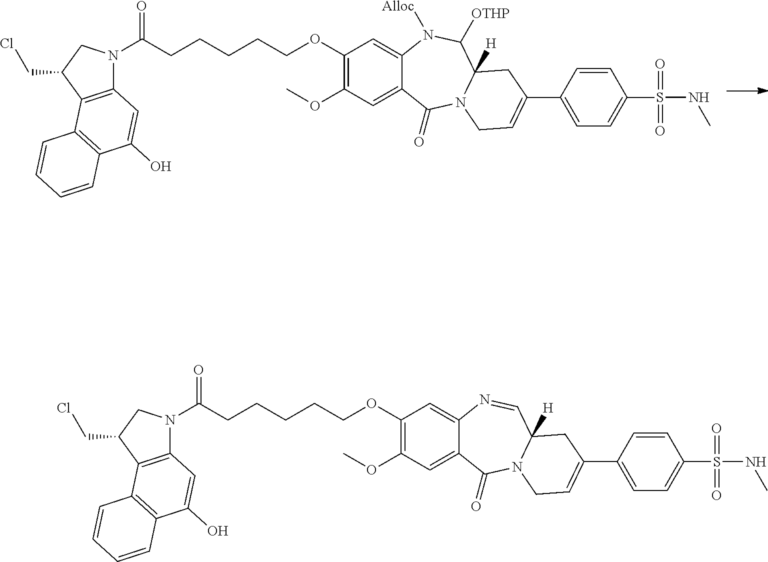

A solution of allyl (6aS)-3-((6-((S)-1-(chloromethyl)-5-hydroxy-1,2-dihydro-3H-benzo[e]indol-3-yl)-6-oxohexyl)oxy)-6-hydroxy-2-methoxy-8-(4-(N-methylsulfamoyl)phenyl)-12-oxo-6,6a,7,10-tetrahydrobenzo[e]pyrido[1,2-a][1,4]diazepine-5(12H)-carboxylate (71) (46 mg, 0.053 mmol) in dichloromethane (3 mL) was charged with tetrakis(triphenylphosphine)palladium(0) (6.2 mg, 0.005 mmol) and pyrrolidine (4.6 mg, 0.064 mmol). The reaction mixture was stirred at room temperature for 1 hour and then concentrated in vacuo. The residue was purified by preparative HPLC to afford the title compound (7.3 mg, 18%) as a pale yellow solid.

¹H NMR (400 MHz, CD₂Cl₂) δ 9.46 (s, 1H), 8.24 (d, J=8.0 Hz, 1H), 8.19 (s, 1H), 7.87-7.85 (m, 2H), 7.68-7.66 (m, 1H), 7.65-7.57 (m, 3H), 7.52-7.48 (m, 1H), 7.46 (s, 1H), 7.36-7.32 (m, 1H), 6.83 (s, 1H), 6.62-6.57 (m, 1H), 4.57-4.53 (m, 1H), 4.48-4.42 (m, 1H), 4.29-4.24 (m, 2H), 4.17-4.12 (m, 2H), 4.10-4.04 (m, 2H), 3.97-3.95 (m, 2H), 3.86 (s, 3H), 3.52-3.46 (m, 1H), 3.02-2.91 (m, 2H), 2.65 (d, J=8.0, 3H), 2.05-1.82 (m, 6H), 1.66-1.62 (m, 2H); MS (ES+): m/z=757 (M+H)⁺; LCMS (Method E): t_{R}=1.54 min.

### Allyl (6aS)-3-(2-(3-(tert-butoxy)-3-oxopropoxy)ethoxy)-2-methoxy-8-(4-(N-methyl-N-(tetrahydro-2H-pyran-2-yl)sulfamoyl)phenyl)-12-oxo-6-((tetrahydro-2H-pyran-2-yl)oxy)-6,6a,7,10-tetrahydrobenzo[e]pyrido[1,2-a][1,4]diazepine-5(12H)-carboxylate (73)

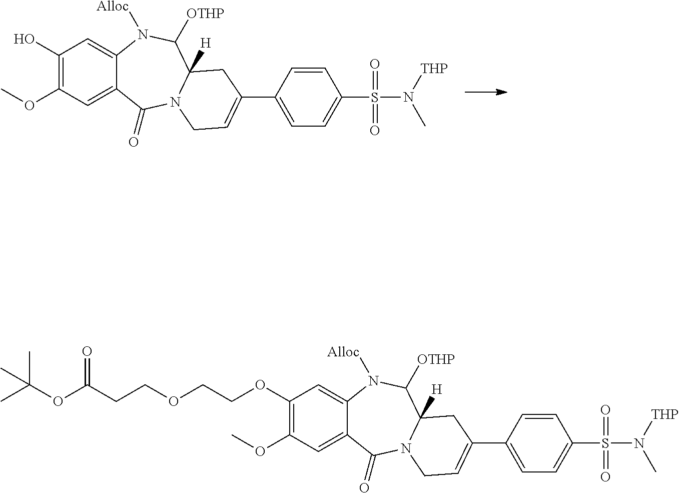

A solution of allyl (6aS)-3-hydroxy-2-methoxy-8-(4-(N-methyl-N-(tetrahydro-2H-pyran-2-yl)sulfamoyl)phenyl)-12-oxo-6-((tetrahydro-2H-pyran-2-yl)oxy)-6,6a,7,10-tetrahydrobenzo[e]pyrido[1,2-a][1,4]diazepine-5(12H)-carboxylate (60) (300 mg, 0.35 mmol) in N,N-dimethylformamide (10 mL) was charged with tert-butyl 3-(2-bromoethoxy)propanoate (134 mg, 0.53 mmol) and potassium carbonate (144 mg, 1.05 mmol) and the resulting mixture was stirred at room temperature for 16 h, before diluting with water (50 mL) and extracting with dichloromethane (2×50 mL). The combined organic extracts were dried over anhydrous sodium sulfate, filtered and concentrated in vacuo. Purification by flash column chromatography (silica), eluting with ethyl acetate/petroleum spirit, 40-60° C. (50%), gave the title compound (220 mg, 59%) as a white solid.

¹H NMR (400 MHz, CDCl₃) δ 7.83-7.81 (m, 2H), 7.61-7.58 (m, 2H), 7.23 (s, 1H), 6.59 (s, 1H), 6.39 (s, 1H), 5.90 (d, J=90.8 Hz, 1H), 5.79-5.71 (m, 2H), 5.19-5.05 (m, 4H), 4.86 (s, 1H), 4.57-4.53 (m, 1H), 4.49-4.43 (m, 1H), 4.24-4.22 (m, 1H), 4.14-4.11 (m, 2H), 3.93-3.89 (m, 3H), 3.86-3.84 (m, 3H), 3.80-3.75 (m, 3H), 3.55-3.48 (m, 1H), 2.74 (s, 3H), 2.54-2.50 (m, 2H), 1.93 (s, 2H), 1.72-1.63 (m, 9H), 1.44-1.42 (m, 13H); MS (ES+): m/z=870 (M+H)⁺; LCMS (Method E): t_{R}=3.53 min.

### 3-(2-(((6aS)-5-((allyloxy)carbonyl)-6-hydroxy-2-methoxy-8-(4-(N-methylsulfamoyl)phenyl)-12-oxo-5,6,6a,7,10,12-hexahydrobenzo[e]pyrido[1,2-a][1,4]diazepin-3-yl)oxy)ethoxy)propanoic acid (74)

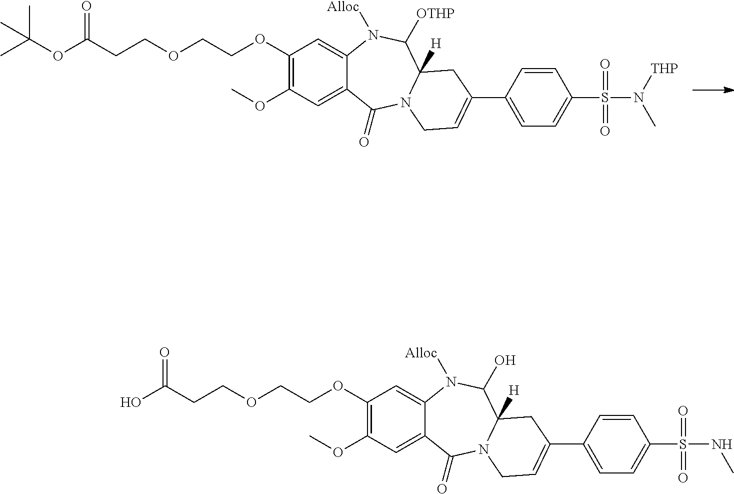

A solution of allyl (6aS)-3-(2-(3-(tert-butoxy)-3-oxopropoxy)ethoxy)-2-methoxy-8-(4-(N-methyl-N-(tetrahydro-2H-pyran-2-yl)sulfamoyl)phenyl)-12-oxo-6-((tetrahydro-2H-pyran-2-yl)oxy)-6,6a,7,10-tetrahydrobenzo[e]pyrido[1,2-a][1,4]diazepine-5(12H)-carboxylate (73) (200 mg, 0.23 mmol) in tetrahydrofuran (5 mL) was charged with hydrochloric acid (4M, 5 mL) and the resulting mixture heated at 50° C. for 2 h, before diluting into water (50 mL) and then extracting with dichloromethane (2×50 mL). The combined organic extracts were dried over anhydrous sodium sulfate, filtered and concentrated in vacuo, to give the title compound (120 mg, 81%) as a white solid.

¹H NMR (400 MHz, DMSO-d₆) δ 12.16 (s, 1H), 7.80-7.78 (m, 2H), 7.72-7.70 (m, 2H), 7.48-7.44 (m, 1H), 7.11 (s, 1H), 6.81 (s, 1H), 6.66-6.61 (m, 2H), 5.82 (s, 1H), 5.47-5.41 (m, 1H), 5.10-5.03 (m, 2H), 4.57-4.54 (m, 1H), 4.14-4.05 (m, 4H), 3.83 (s, 3H), 3.74-3.71 (m, 3H), 3.69-3.65 (m, 3H), 3.59-3.55 (m, 1H), 2.93-2.89 (m, 1H), 2.80-2.76 (m, 1H), 2.48-2.46 (m, 2H), 2.43 (d, J=40.8 Hz, 3H); MS (ES+): m/z=646 (M+H)⁺; LCMS (Method F): t_{R}=2.80 min.

### Allyl (6aS)-3-(2-(3-((S)-1-(chloromethyl)-5-hydroxy-1,2-dihydro-3H-benzo[e]indol-3-yl)-3-oxopropoxy)ethoxy)-6-hydroxy-2-methoxy-8-(4-(N-methylsulfamoyl)phenyl)-12-oxo-6,6a,7,10-tetrahydrobenzo[e]pyrido[1,2-a][1,4]diazepine-5(12H)-carboxylate (75)

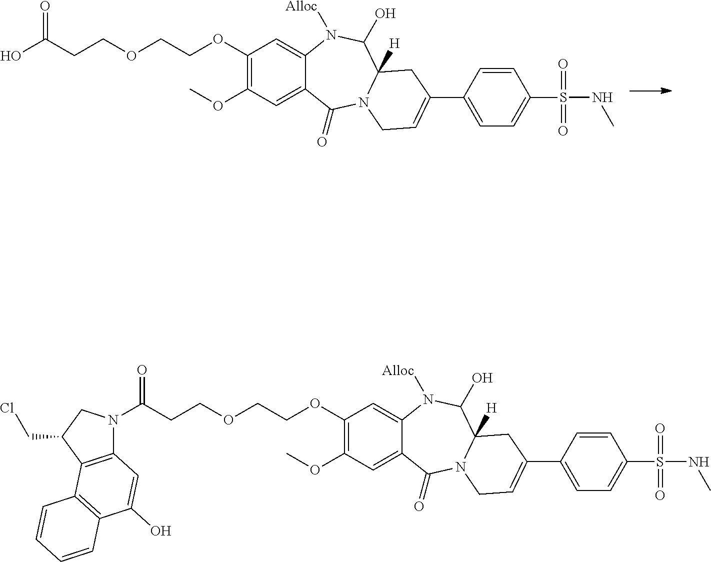

A solution of 3-(2-(((6aS)-5-((allyloxy)carbonyl)-6-hydroxy-2-methoxy-8-(4-(N-methylsulfamoyl)phenyl)-12-oxo-5,6,6a,7,10,12-hexahydrobenzo[e]pyrido[1,2-a][1,4]diazepin-3-yl)oxy)ethoxy)propanoic acid (74) (130 mg, 0.20 mmol) in N,N-dimethylacetamide (10 mL) was charged with (S)-1-(chloromethyl)-2,3-dihydro-1H-benzo[e]indol-5-ol hydrochloride (12) (76 mg, 0.28 mmol) and N-(3-dimethylaminopropyl)-N′-ethylcarbodiimide hydrochloride (154 mg, 0.80 mmol) and the resulting mixture stirred at room temperature for 16 h, before diluting into water (50 mL) and then extracting with dichloromethane (2×50 mL). The combined organic extracts were dried over sodium sulfate and concentrated in vacuo. Purification by flash column chromatography (silica), eluting with methanol/dichloromethane (5%), gave the title compound (54 mg, 31%) as a white solid.

¹H NMR (400 MHz, CDCl₃) δ 8.84 (s, 1H), 8.24 (d, J=8.4 Hz, 1H), 8.03 (s, 1H), 7.79 (d, J=8.4 Hz, 2H), 7.64-7.62 (m, 1H), 7.56-7.54 (m, 2H), 7.53-7.49 (m, 1H), 7.40-7.35 (m, 1H), 7.25 (s, 1H), 7.11 (s, 1H), 6.41 (s, 1H), 5.83 (d, J=9.6 Hz, 1H), 5.67-5.61 (m, 1H), 5.47 (s, 1H), 5.05-4.96 (m, 2H), 4.54-4.48 (m, 3H), 4.44-4.40 (m, 1H), 4.32-4.17 (m, 5H), 4.04-3.95 (m, 3H), 3.91 (s, 3H), 3.83-3.66 (m, 5H), 3.08-3.04 (m, 1H), 2.83-2.73 (m, 3H), 2.62 (d, J=5.4 Hz, 3H); MS (ES+): m/z=861 (M+H)⁺; LCMS (Method F): t_{R}=3.50 min.

### 4-((S)-3-(2-(3-((S)-1-(Chloromethyl)-5-hydroxy-1,2-dihydro-3H-benzo[e]indol-3-yl)-3-oxopropoxy)ethoxy)-2-methoxy-12-oxo-6a,7,10,12-tetrahydrobenzo[e]pyrido[1,2-a][1,4]diazepin-8-yl)-N-methylbenzenesulfonamide (76)

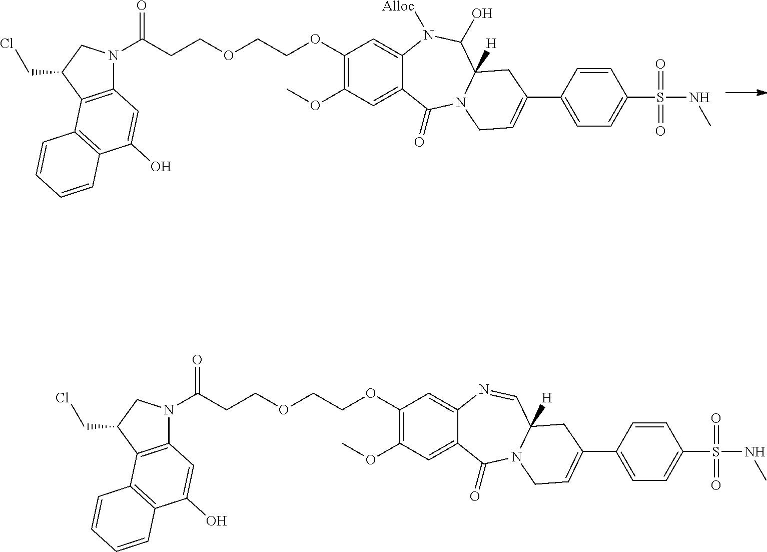

A solution of allyl (6aS)-3-(2-(3-((S)-1-(chloromethyl)-5-hydroxy-1,2-dihydro-3H-benzo[e]indol-3-yl)-3-oxopropoxy)ethoxy)-6-hydroxy-2-methoxy-8-(4-(N-methylsulfamoyl)phenyl)-12-oxo-6,6a,7,10-tetrahydrobenzo[e]pyrido[1,2-a][1,4]diazepine-5(12H)-carboxylate (75) (54 mg, 0.063 mmol) in dichloromethane (4 mL) was charged with tetrakis(triphenylphosphine)palladium(0) (7.2 mg, 0.006 mmol) and pyrrolidine (5.4 mg, 0.075 mmol) and the resulting mixture stirred at room temperature for 30 min, before concentrating in vacuo. The residue was purified by preparative HPLC to give the title compound (15 mg, 31%) as a pale yellow solid.

¹H NMR (400 MHz, CD₂Cl₂) δ 9.65 (s, 1H), 8.23 (d, J=8.4 Hz, 1H), 8.15 (s, 1H), 7.85-7.83 (m, 2H), 7.66-7.64 (m, 1H), 7.63-7.56 (m, 3H), 7.52-7.47 (m, 1H), 7.41 (s, 1H), 7.38-7.33 (m, 1H), 6.83 (s, 1H), 6.60-6.55 (m, 1H), 4.65-4.60 (m, 1H), 4.46-4.40 (m, 1H), 4.37-4.33 (m, 1H), 4.29-4.26 (m, 1H), 4.24-4.20 (m, 1H), 4.17-4.09 (m, 2H), 4.03-3.98 (m, 2H), 3.98-3.93 (m, 2H), 3.92-3.87 (m, 3H), 3.75 (s, 3H), 3.47 (dd, J=11.2, 10.0 Hz, 1H), 3.03-2.93 (m, 2H), 2.86-2.82 (m, 2H), 2.64 (d, J=5.4 Hz, 3H); MS (ES+): m/z=759 (M+H)⁺; LCMS (Method F): t_{R}=2.00 min.

### Ethyl (S)-3,4-dihydroxybutanoate (78)

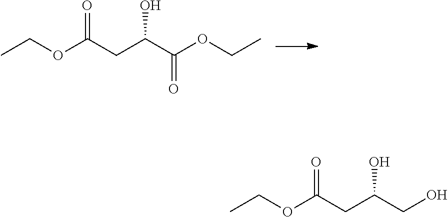

A solution of diethyl (S)-2-hydroxysuccinate (77) (10.0 g, 52.6 mmol) in tetrahydrofuran (80 mL) was charged with a solution of borane dimethyl sulfide complex (2 M in tetrahydrofuran, 30 mL, 57.8 mmol), dropwise at room temperature, and stirred for 30 min. After cooling to 0° C., sodium borohydride (99 mg, 2.63 mmol) was charged in one portion, after which the mixture was allowed to stand at room temperature for 30 min. Ethanol (25 mL) and p-toluenesulfonic acid monohydrate (500 mg, 2.63 mmol) were then added and the resulting solution stirred for 30 min, before concentrating in vacuo. Purification by flash column chromatography (silica), eluting with ethyl acetate/petroleum spirit, 40-60° C. (50%), gave the title compound (6.30 g, 81%) as a colourless oil.

¹H NMR (400 MHz, DMSO-d₆) δ 4.75 (s, 1H), 4.64-4.58 (m, 1H), 4.06-4.02 (m, 2H), 3.88-3.80 (m, 1H), 3.36-3.32 (m, 1H), 3.25-3.21 (m, 1H), 2.51-2.47 (m, 1H), 2.23-2.17 (m, 1H), 1.19-1.17 (m, 3H).

### Ethyl (S)-3,4-bis((tert-butyldimethylsilyl)oxy)butanoate (79)

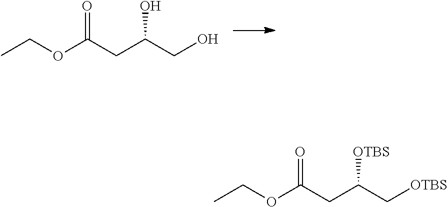

A solution of ethyl (S)-3,4-dihydroxybutanoate (78) (6.00 g, 40.5 mmol) in N,N-dimethylformamide (80 mL) was charged with tert-butyldimethylsilyl chloride (18.3 g, 122 mmol) and imidazole (16.6 g, 243 mmol) and the resulting mixture stirred at room temperature for 16 h. After diluting into water (500 mL) and extracting with ethyl acetate (500 mL), the organic phase was washed with brine (500 mL), then dried over sodium sulfate and concentrated in vacuo. Purification by flash column chromatography (silica), eluting with petroleum spirit, 40-60° C., gave the title compound (11.4 g, 75%) as a colourless oil.

¹H NMR (400 MHz, CDCl₃) s4.03-3.99 (m, 2H), 3.56-3.43 (m, 2H), 2.57-2.51 (m, 1H), 2.33-2.27 (m, 1H), 1.19-1.16 (m, 4H), 0.87-0.81 (m, 18H), 0.05-0.04 (m, 12H).

### Ethyl (S)-3-((tert-butyldimethylsilyl)oxy)-4-hydroxybutanoate (80)

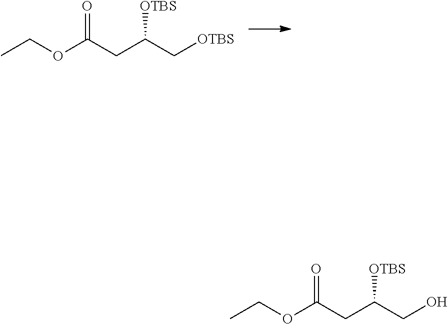 [SMILES: CCOC(=O)C[C@@H](CO[Si](C)(C)C(C)(C)C)O[Si](C)(C)C(C)(C)C ; CCOC(=O)C[C@@H](CO)O[Si](C)(C)C(C)(C)C]

A solution of ethyl (S)-3,4-bis((tert-butyldimethylsilyl)oxy)butanoate (79) (11.0 g, 29.2 mmol) in methanol (150 mL) was charged with pyridinium p-toluenesulfonate (9.00 g, 35.0 mmol) and the resulting mixture stirred at room temperature for 16 h, before diluting into water (500 mL), then extracting with ethyl acetate (500 mL). The organic phase was then washed with brine (500 mL), dried over magnesium sulfate and concentrated in vacuo. Purification by flash column chromatography (silica), eluting with ethyl acetate/petroleum spirit, 40-60° C. (from 10% to 20%), gave the title compound (3.30 g, 43%) as a colourless oil.

¹H NMR (400 MHz, DMSO-d₆) δ 4.72-4.69 (m, 1H), 4.00-3.92 (m, 2H), 3.43-3.27 (m, 1H), 3.21-3.13 (m, 1H), 2.56-2.14 (m, 2H), 1.16-1.12 (m, 3H), 0.81-0.77 (m, 9H), 0.02 (d, J=16.0 Hz, 6H).

### Ethyl (R)-3,4-dihydroxybutanoate (82)

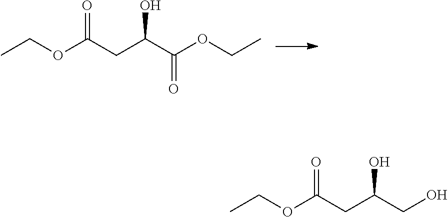

A solution of diethyl (R)-2-hydroxysuccinate (81) (10.0 g, 52.6 mmol) in tetrahydrofuran (80 mL) was charged with a solution of borane dimethyl sulfide complex (2 M in tetrahydrofuran, 30 mL, 57.8 mmol), dropwise at room temperature, and stirred for 30 min. After cooling to 0° C., sodium borohydride (99 mg, 2.63 mmol) was charged in one portion, after which the mixture was allowed to stand at room temperature for 30 min. Ethanol (25 mL) and p-toluenesulfonic acid monohydrate (500 mg, 2.63 mmol) were then added and the resulting solution stirred for 30 min, before concentrating in vacuo. Purification by flash column chromatography (silica), eluting with ethyl acetate/petroleum spirit, 40-60° C. (50%), gave the title compound (5.60 g, 72%) as a colourless oil.

¹H NMR (400 MHz, DMSO-d₆) δ 4.78 (d, J=8.0 Hz, 1H), 4.66-4.61 (m, 1H), 4.08-4.04 (m, 2H), 3.89-3.83 (m, 1H), 3.39-3.20 (m, 2H), 2.49-2.45 (m, 1H), 2.25-2.20 (m, 1H), 1.21-1.17 (m, 3H).

### Ethyl (R)-3,4-bis((tert-butyldimethylsilyl)oxy)butanoate (83)

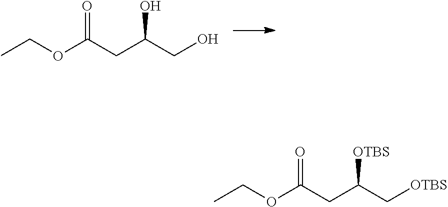

A solution of ethyl (R)-3,4-dihydroxybutanoate (82) (5.60 g, 37.8 mmol) in N,N-dimethylformamide (100 mL) was charged with tert-butyldimethylsilyl chloride (17.1 g, 122 mmol) and imidazole (15.4 g, 243 mmol) and the resulting mixture stirred at room temperature for 16 h. After diluting into water (500 mL) and extracting with ethyl acetate (500 mL), the organic phase was washed with brine (500 mL), then dried over sodium sulfate and concentrated in vacuo. Purification by flash column chromatography (silica), eluting with petroleum spirit, 40-60° C., gave the title compound (12.3 g, 87%) as a colourless oil.

¹H NMR (400 MHz, DMSO-d₆) δ 4.04-4.02 (m, 2H), 3.56-3.43 (m, 2H), 2.56-2.51 (m, 1H), 2.33-2.27 (m, 1H), 1.19-1.17 (m, 3H), 0.88-0.81 (m, 18H), 0.05-0.02 (m, 12H).

### Ethyl (R)-3-((tert-butyldimethylsilyl)oxy)-4-hydroxybutanoate (84)

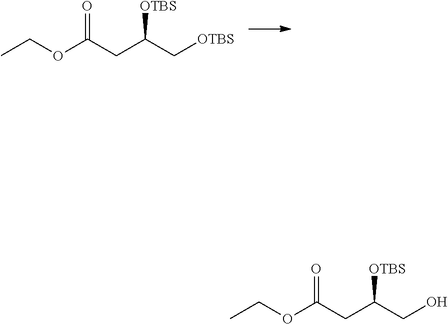 [SMILES: CCOC(=O)C[C@H](CO[Si](C)(C)C(C)(C)C)O[Si](C)(C)C(C)(C)C ; CCOC(=O)C[C@H](CO)O[Si](C)(C)C(C)(C)C]

A solution of ethyl (R)-3,4-bis((tert-butyldimethylsilyl)oxy)butanoate (83) (12.3 g, 32.7 mmol) in methanol (150 mL) was charged with pyridinium p-toluenesulfonate (9.85 g, 39.2 mmol) and the resulting mixture stirred at room temperature for 16 h, before diluting into water (500 mL), then extracting with ethyl acetate (500 mL). The organic phase was then washed with brine (500 mL), dried over sodium sulfate and concentrated in vacuo. Purification by flash column chromatography (silica), eluting with ethyl acetate/petroleum spirit, 40-60° C. (from 10% to 20%), gave the title compound (4.10 g, 48%) as a colourless oil.

¹H NMR (400 MHz, DMSO-d₆) δ 4.73-4.70 (m, 1H), 4.01-3.93 (m, 3H), 3.35-3.31 (m, 1H), 3.20-3.13 (m, 1H), 2.55-2.49 (m, 1H), 2.20-2.14 (m, 1H), 1.15-1.11 (m, 3H), 0.76 (s, 9H), 0.05-0.01 (m, 6H).

### Allyl (6aS)-3-((S)-2-((tert-butyldimethylsilyl)oxy)-4-ethoxy-4-oxobutoxy)-2-methoxy-8-(4-(N-methyl-N-(tetrahydro-2H-pyran-2-yl)sulfamoyl)phenyl)-12-oxo-6-((tetrahydro-2H-pyran-2-yl)oxy)-6,6a,7,10-tetrahydrobenzo[e]pyrido[1,2-a][1,4]diazepine-5(12H)-carboxylate (85)

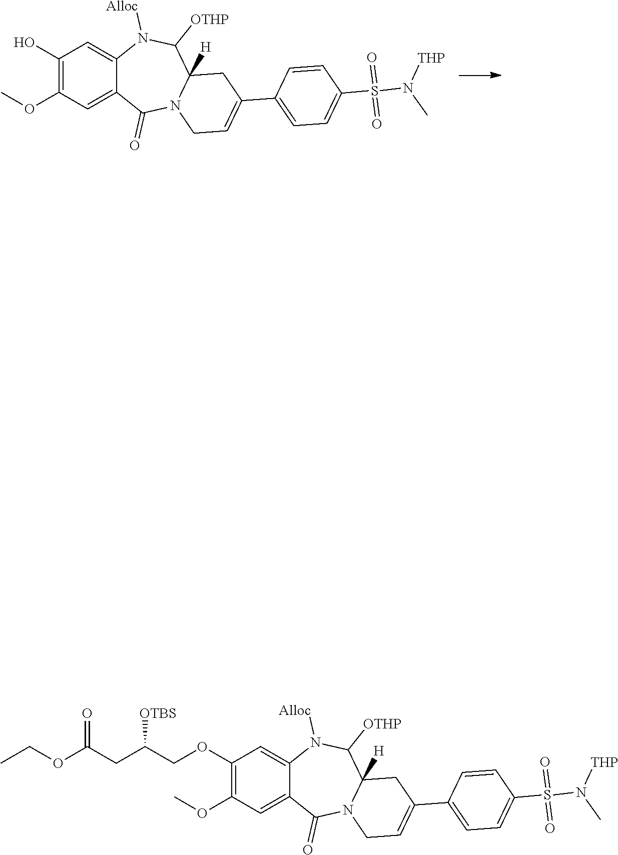

A solution of triphenylphosphine (129 mg, 0.49 mmol) in toluene (2 mL) at 0° C. was charged with diethyl azodicarboxylate (78 mg, 0-45 mmol), and stirred for 30 min (under an inert atmosphere of nitrogen). Allyl (6aS)-3-hydroxy-2-methoxy-8-(4-(N-methyl-N-(tetrahydro-2H-pyran-2-yl)sulfamoyl)phenyl)-12-oxo-6-((tetrahydro-2H-pyran-2-yl)oxy)-6,6a,7,10-tetrahydrobenzo[e]pyrido[1,2-a][1,4]diazepine-5(12H)-carboxylate (60) (100 mg, 0.14 mmol) and ethyl (S)-3-((tert-butyldimethylsilyl)oxy)-4-hydroxybutanoate (80) (55 mg, 0.21 mmol) in toluene (2 mL) were added to the mixture at 0° C., which was then stirred at 30° C. for 16 h. After diluting into water (50 mL) and extracting with ethyl acetate (50 mL), the organic phase was washed with brine (50 mL), dried over sodium sulfate, filtered and concentrated in vacuo. Purification by flash column chromatography (silica), eluting with ethyl acetate/petroleum spirit, 40-60° C. (from 33% to 50%), gave the title compound (120 mg, 91%) as a white solid.

¹H NMR (400 MHz, CDCl₃) δ 7.83 (d, J=8.0 Hz, 1H), 7.61-7.60 (m, 1H), 7.57-7.52 (m, 6H), 7.25-7.20 (m, 1H), 6.62-6.40 (m, 1H), 5.94-5.68 (m, 1H), 5.35-5.30 (m, 1H), 5.18-4.86 (m, 3H), 4.54-4.52 (m, 2H), 4.31-4.08 (m, 3H), 4.01-3.70 (m, 6H), 3.62-3.32 (m, 2H), 3.27-3.10 (m, 1H), 2.78-2.50 (m, 5H), 2.24-2.18 (m, 1H), 2.08-1.86 (m, 2H), 1.75-1.65 (m, 3H), 1.52-1.42 (m, 5H), 1.35-1.27 (m, 7H), 0.86 (s, 9H), 0.13-0.03 (m, 6H); MS (ES+): m/z=942 (M+H)⁺; LCMS (Method D): t_{R}=2.70 min.

### (3S)-4-(((6aS)-5-((Allyloxy)carbonyl)-6-hydroxy-2-methoxy-8-(4-(N-methylsulfamoyl)phenyl)-12-oxo-5,6,6a,7,10,12-hexahydrobenzo[e]pyrido[1,2-a][1,4]diazepin-3-yl)oxy)-3-hydroxybutanoic acid (86)

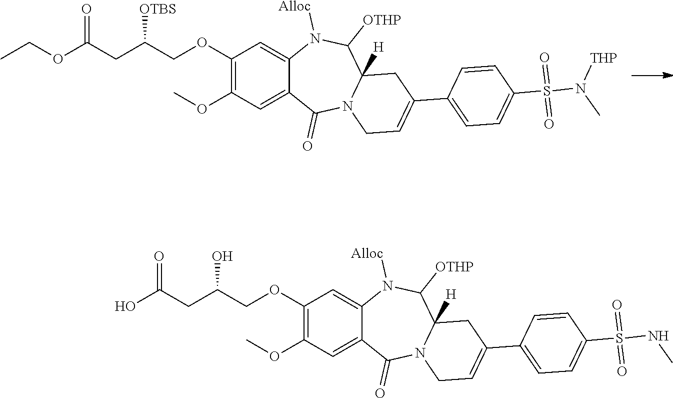

A solution of allyl (6aS)-3-((S)-2-((tert-butyldimethylsilyl)oxy)-4-ethoxy-4-oxobutoxy)-2-methoxy-8-(4-(N-methyl-N-(tetrahydro-2H-pyran-2-yl)sulfamoyl)phenyl)-12-oxo-6-((tetrahydro-2H-pyran-2-yl)oxy)-6,6a,7,10-tetrahydrobenzo[e]pyrido[1,2-a][1,4]diazepine-5(12H)-carboxylate (85) (106 mg, 0.14 mmol) in tetrahydrofuran (2 mL) was charged with an aqueous solution of hydrochloric acid (4 M, 2 mL) and stirred for 2 h at 50° C. After diluting into water (50 mL) and extracting with ethyl acetate (2×50 mL), the combined organic extracts were washed with brine (50 mL), dried over sodium sulfate, filtered and concentrated in vacuo. Purification by preparative TLC, eluting with methanol/dichloromethane (5%; plus one drop of acetic acid), gave the title compound (55 mg, 74%) as a white solid.

¹H NMR (400 MHz, DMSO-d₆) δ 12.13 (s, 1H), 7.80-7.78 (m, 2H), 7.72-7.70 (m, 2H), 7.49-7.45 (m, 1H), 7.12 (s, 1H), 6.81 (s, 1H), 6.70-6.60 (m, 2H), 5.80 (s, 1H), 5.46-5.42 (m, 1H), 5.11-5.04 (m, 3H), 4.57-4.53 (m, 1H), 4.46-4.38 (m, 1H), 4.22-4.08 (m, 3H), 3.96-3.88 (m, 2H), 3.84 (s, 3H), 3.60-3.56 (m, 1H), 2.94-2.89 (m, 1H), 2.80-2.76 (m, 1H), 2.59-2.52 (m, 1H), 2.42 (d, J=16.0 Hz, 3H), 2.40-2.34 (m, 1H); MS (ES+): m/z=632 (M+H)⁺; LCMS (Method F): t_{R}=0.47 min.

### Allyl (6aS)-3-((S)-4-((S)-1-(chloromethyl)-5-hydroxy-1,2-dihydro-3H-benzo[e]indol-3-yl)-2-hydroxy-4-oxobutoxy)-6-hydroxy-2-methoxy-8-(4-(N-methylsulfamoyl)phenyl)-12-oxo-6,6a,7,10-tetrahydrobenzo[e]pyrido[1,2-a][1,4]diazepine-5(12H)-carboxylate (87)

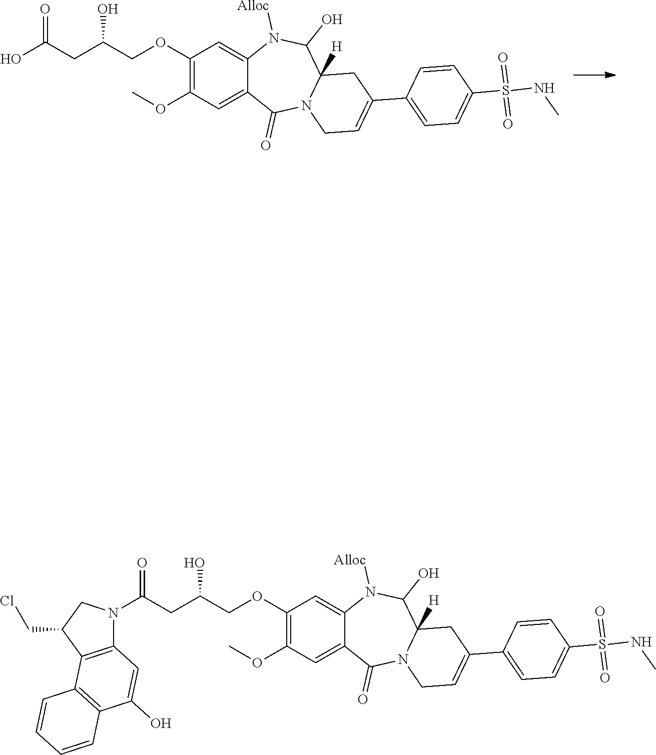

A solution of (3S)-4-(((6aS)-5-((Allyloxy)carbonyl)-6-hydroxy-2-methoxy-8-(4-(N-methylsulfamoyl)phenyl)-12-oxo-5,6,6a,7,10,12-hexahydrobenzo[e]pyrido[1,2-a][1,4]diazepin-3-yl)oxy)-3-hydroxybutanoic acid (86) (50 mg, 0.079 mmol) in N,N-dimethylacetamide (2 mL) was charged with (S)-1-(chloromethyl)-2,3-dihydro-1H-benzo[e]indol-5-ol hydrochloride (12) (30 mg, 0.11 mmol) and N-(3-dimethylaminopropyl)-N′-ethylcarbodiimide hydrochloride (61 mg, 0.32 mmol) and the resulting mixture stirred at room temperature for 18 h, before diluting into water (50 mL) and then extracting with ethyl acetate (2×25 mL). The combined organic extracts were washed with brine (25 mL), dried over sodium sulfate, filtered and concentrated in vacuo. Purification by preparative TLC, eluting with methanol/dichloromethane (10%), gave the title compound (35 mg, 52%) as a grey solid.

MS (ES+): m/z=869 (M+Na)+; LCMS (Method F): t_{R}=3.40 min. 4-((S)-3-((S)-4-((S)-1-(Chloromethyl)-5-hydroxy-1,2-dihydro-3H-benzo[e]indol-3-yl)-2-hydroxy-4-oxobutoxy)-2-methoxy-12-oxo-6a,7,10,12-tetrahydrobenzo[e]pyrido[1,2-a][1,4]diazepin-8-yl)-N-methylbenzenesulfonamide (88)

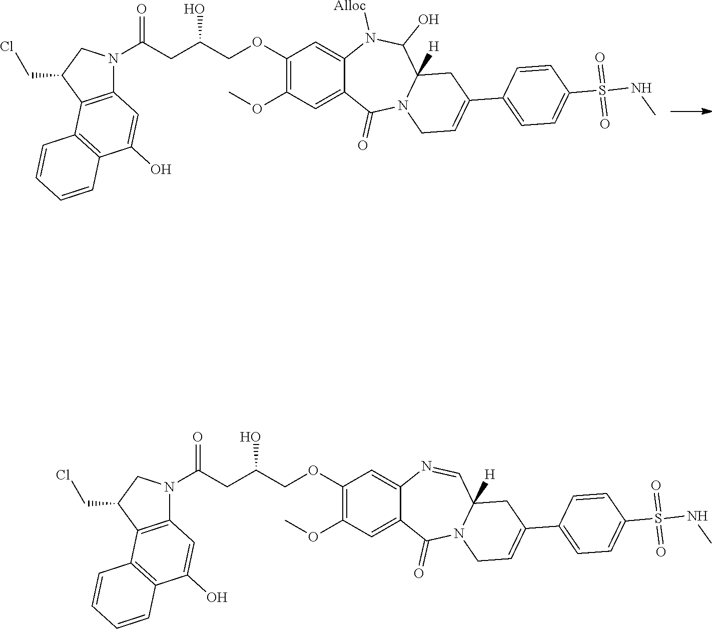

A solution of allyl (6aS)-3-((S)-4-((S)-1-(chloromethyl)-5-hydroxy-1,2-dihydro-3H-benzo[e]indol-3-yl)-2-hydroxy-4-oxobutoxy)-6-hydroxy-2-methoxy-8-(4-(N-methylsulfamoyl)phenyl)-12-oxo-6,6a,7,10-tetrahydrobenzo[e]pyrido[1,2-a][1,4]diazepine-5(12H)-carboxylate (87) (35 mg, 0.041 mmol) in dichloromethane (4 mL) was charged with tetrakis(triphenylphosphine)palladium(0) (5 mg, 0.004 mmol) and pyrrolidine (4 mg, 0.050 mmol) and the resulting mixture stirred at room temperature for 30 min, before concentrating in vacuo. Purification by preparative HPLC gave the title compound (6.4 mg, 21%) as an off-white solid.

¹H NMR (400 MHz, DMSO-d₆) δ 10.39 (s, 1H), 8.09-8.07 (d, J=8.0 Hz, 1H), 7.99 (s, 1H), 7.79 (s, 3H), 7.77 (s, 1H), 7.73-7.62 (m, 2H), 7.49-7.47 (m, 2H), 7.38-7.29 (m, 2H), 6.92 (s, 1H), 6.71-6.60 (m, 2H), 5.34-5.20 (m, 1H), 4.41-4.36 (m, 2H), 4.21-4.18 (m, 2H), 4.15-4.10 (m, 2H), 3.98-3.95 (m, 3H), 3.85 (s, 3H), 3.78-3.73 (m, 1H), 2.85-2.82 (m, 1H), 2.77-2.72 (m, 1H), 2.46-2.40 (m, 3H), 2.02-1.97 (m, 2H); MS (ES+): m/z=745 (M+H)⁺; LCMS (Method F): t_{R}=1.90 min.

### Allyl (6aS)-3-((R)-2-((tert-butyldimethylsilyl)oxy)-4-ethoxy-4-oxobutoxy)-2-methoxy-8-(4-(N-methyl-N-(tetrahydro-2H-pyran-2-yl)sulfamoyl)phenyl)-12-oxo-6-((tetrahydro-2H-pyran-2-yl)oxy)-6,6a,7,10-tetrahydrobenzo[e]pyrido[1,2-a][1,4]diazepine-5(12H)-carboxylate (89)

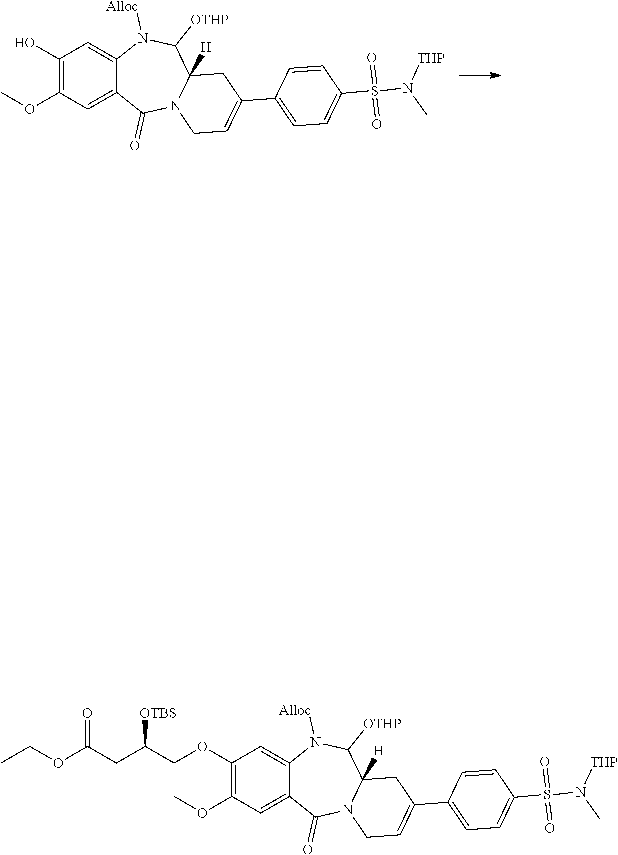

A solution of triphenylphosphine (212 mg, 0.81 mmol) in toluene (2 mL) at 0° C. was charged with diethyl azodicarboxylate (165 mg, 0.95 mmol), and stirred for 30 min (under an inert atmosphere of nitrogen). Allyl (6aS)-3-hydroxy-2-methoxy-8-(4-(N-methyl-N-(tetrahydro-2H-pyran-2-yl)sulfamoyl)phenyl)-12-oxo-6-((tetrahydro-2H-pyran-2-yl)oxy)-6,6a,7,10-tetrahydrobenzo[e]pyrido[1,2-a][1,4]diazepine-5(12H)-carboxylate (60) (190 mg, 0.27 mmol) and ethyl (R)-3-((tert-butyldimethylsilyl)oxy)-4-hydroxybutanoate (84) (212 mg, 0.81 mmol) in toluene (4 mL) were added to the mixture at 0° C., which was then stirred at 30° C. for 16 h. After diluting into water (100 mL) and extracting with ethyl acetate (100 mL), the organic phase was washed with brine (100 mL), dried over sodium sulfate, filtered and concentrated in vacuo. Purification by flash column chromatography (silica), eluting with ethyl acetate/petroleum spirit, 40-60° C. (from 33% to 50%), gave the title compound (220 mg, 87%) as an off-white solid.

¹H NMR (400 MHz, CDCl₃) δ 7.87-7.81 (m, 2H), 7.62-7.58 (m, 1H), 7.54-7.50 (m, 6H), 7.22-7.18 (m, 1H), 6.62-6.37 (m, 1H), 5.93-5.70 (m, 1H), 5.20-5.05 (m, 3H), 4.93-4.51 (m, 2H), 4.30-4.09 (m, 3H), 3.94-3.47 (m, 6H), 2.82-2.67 (m, 4H), 2.58-2.15 (m, 1H), 2.07-1.89 (m, 2H), 1.52-1.42 (m, 5H), 1.26-1.22 (m, 8H), 0.90-0.82 (m, 9H), 0.20-0.05 (m, 6H); MS (ES+): m/z=942 (M+H)⁺; LCMS (Method D): t_{R}=2.73 min.

### (3R)-4-(((6aS)-5-((Allyloxy)carbonyl)-6-hydroxy-2-methoxy-8-(4-(N-methylsulfamoyl)phenyl)-12-oxo-5,6,6a,7,10,12-hexahydrobenzo[e]pyrido[1,2-a][1,4]diazepin-3-yl)oxy)-3-hydroxybutanoic acid (90)

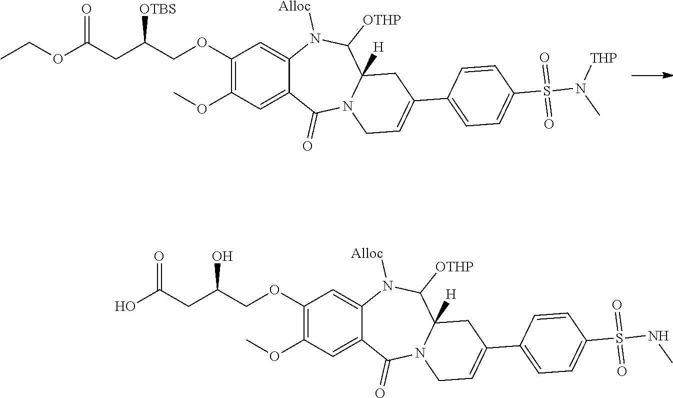

A solution of allyl (6aS)-3-((R)-2-((tert-butyldimethylsilyl)oxy)-4-ethoxy-4-oxobutoxy)-2-methoxy-8-(4-(N-methyl-N-(tetrahydro-2H-pyran-2-yl)sulfamoyl)phenyl)-12-oxo-6-((tetrahydro-2H-pyran-2-yl)oxy)-6,6a,7,10-tetrahydrobenzo[e]pyrido[1,2-a][1,4]diazepine-5(12H)-carboxylate (89) (220 mg, 0.23 mmol) in tetrahydrofuran (4 mL) was charged with an aqueous solution of hydrochloric acid (4 M, 4 mL) and stirred for 2 h at 50° C. After diluting into water (100 mL) and extracting with ethyl acetate (2×100 mL), the combined organic extracts were washed with brine (100 mL), dried over sodium sulfate, filtered and concentrated in vacuo. Purification by preparative TLC, eluting with methanol/dichloromethane (5%; plus one drop of acetic acid), gave the title compound (50 mg, 35%) as a white solid.

¹H NMR (400 MHz, DMSO-d₆) δ 7.81-7.79 (m, 2H), 7.73-7.71 (m, 2H), 7.52-7.48 (m, 1H), 7.12 (s, 1H), 6.82 (s, 1H), 6.74-6.62 (m, 2H), 5.46-5.41 (m, 1H), 5.36-5.03 (m, 3H), 4.57-4.44 (m, 1H), 4.49-4.38 (m, 1H), 4.23-4.08 (m, 3H), 3.91-3.89 (m, 2H), 3.85 (s, 3H), 3.58 (s, 1H), 2.96-2.88 (m, 1H), 2.85-2.74 (m, 1H), 2.70-2.53 (m, 1H), 2.42 (d, J=12.0 Hz, 3H), 2.37-2.32 (m, 1H); MS (ES+): m/z=632 (M+H)⁺; LCMS (Method E): t_{R}=0.77 min.

### Allyl (6aS)-3-((R)-4-((S)-1-(chloromethyl)-5-hydroxy-1,2-dihydro-3H-benzo[e]indol-3-yl)-2-hydroxy-4-oxobutoxy)-6-hydroxy-2-methoxy-8-(4-(N-methylsulfamoyl)phenyl)-12-oxo-6,6a,7,10-tetrahydrobenzo[e]pyrido[1,2-a][1,4]diazepine-5(12H)-carboxylate (91)

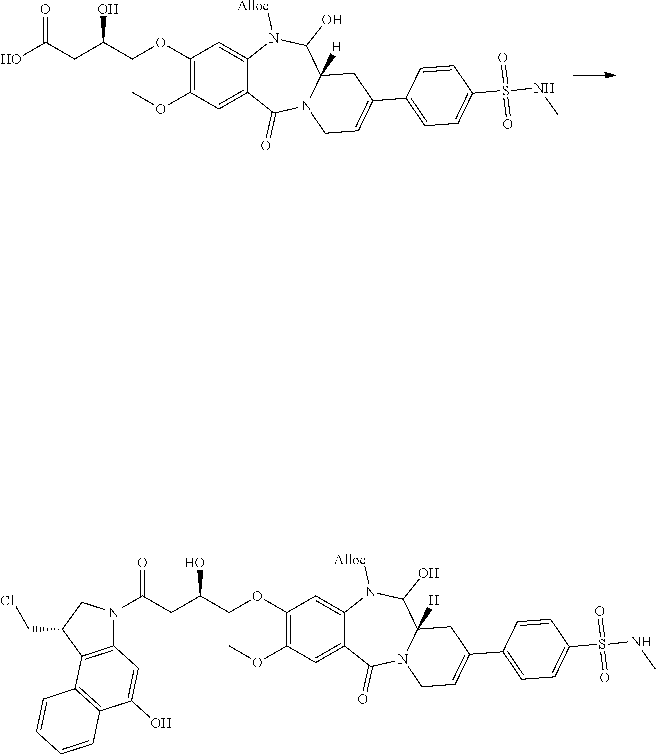

A solution of (3R)-4-(((6aS)-5-((allyloxy)carbonyl)-6-hydroxy-2-methoxy-8-(4-(N-methylsulfamoyl)phenyl)-12-oxo-5,6,6a,7,10,12-hexahydrobenzo[e]pyrido[1,2-a][1,4]diazepin-3-yl)oxy)-3-hydroxybutanoic acid (90) (50 mg, 0.079 mmol) in N,N-dimethylacetamide (2 mL) was charged with (S)-1-(chloromethyl)-2,3-dihydro-1H-benzo[e]indol-5-ol hydrochloride (12) (30 mg, 0.11 mmol) and N-(3-dimethylaminopropyl)-N′-ethylcarbodiimide hydrochloride (61 mg, 0.32 mmol) and the resulting mixture stirred at room temperature for 18 h, before diluting into water (50 mL) and then extracting with ethyl acetate (2×25 mL). The combined organic extracts were washed with brine (25 mL), dried over sodium sulfate, filtered and concentrated in vacuo. Purification by preparative TLC, eluting with methanol/dichloromethane (10%), gave the title compound (40 mg, 60%) as a grey solid.

MS (ES−): m/z=845 (M−H)⁻; LCMS (Method F): t_{R}=2.05 min. 4-((S)-3-((R)-4-((S)-1-(Chloromethyl)-5-hydroxy-1,2-dihydro-3H-benzo[e]indol-3-yl)-2-hydroxy-4-oxobutoxy)-2-methoxy-12-oxo-6a,7,10,12-tetrahydrobenzo[e]pyrido[1,2-a][1,4]diazepin-8-yl)-N-methylbenzenesulfonamide (92)

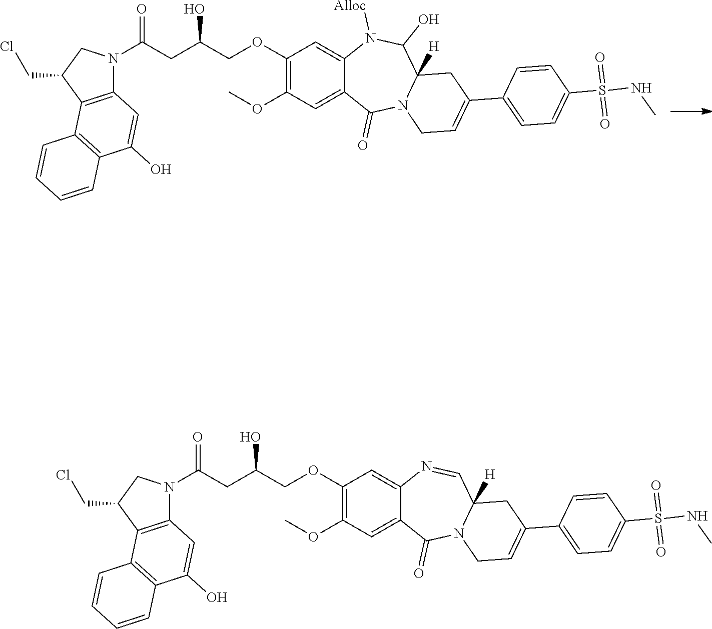

A solution of allyl (6aS)-3-((R)-4-((S)-1-(chloromethyl)-5-hydroxy-1,2-dihydro-3H-benzo[e]indol-3-yl)-2-hydroxy-4-oxobutoxy)-6-hydroxy-2-methoxy-8-(4-(N-methylsulfamoyl)phenyl)-12-oxo-6,6a,7,10-tetrahydrobenzo[e]pyrido[1,2-a][1,4]diazepine-5(12H)-carboxylate (91) (35 mg, 0.041 mmol) in dichloromethane (4 mL) was charged with tetrakis(triphenylphosphine)palladium(0) (5 mg, 0.004 mmol) and pyrrolidine (4 mg, 0.050 mmol) and the resulting mixture stirred at room temperature for 30 min, before concentrating in vacuo. Purification by preparative HPLC gave the title compound (5.4 mg, 17%) as a white solid.

¹H NMR (400 MHz, DMSO-d₆) δ 10.40 (s, 1H), 8.08 (d, J=8.0 Hz, 1H), 8.00 (s, 1H), 7.82-7.77 (m, 4H), 7.73-7.68 (m, 1H), 7.65-7.62 (m, 1H), 7.52-7.46 (m, 2H), 7.36-7.30 (m, 2H), 6.92 (s, 1H), 6.70 (s, 1H), 5.34-5.28 (m, 1H), 4.45-4.25 (m, 4H), 4.16-4.08 (m, 3H), 4.02-3.88 (m, 3H), 3.82 (s, 3H), 3.74-3.68 (m, 1H), 3.02-2.94 (m, 1H), 2.81-2.70 (m, 2H), 2.45-2.38 (m, 3H), 2.04-1.96 (m, 1H); MS (ES+): m/z=745 (M+H)⁺; LCMS (Method E): t_{R}=1.27 min.

### Allyl (6aS)-2-methoxy-3-((6-methoxy-6-oxohexyl)oxy)-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (93)

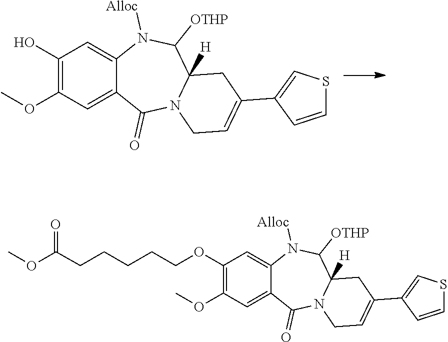

A solution of allyl (6aS)-3-hydroxy-2-methoxy-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (67) (220 mg, 0.42 mmol) in N,N-dimethylformamide (10 mL) was charged with methyl 6-bromohexanoate (132 mg, 0.63 mmol) and potassium carbonate (173 mg, 1.25 mmol) and the resulting mixture stirred at room temperature for 6 h, before diluting into water (100 mL) and extracting with ethyl acetate (100 mL). The organic phase was dried over sodium sulfate and then concentrated in vacuo. Purification by flash column chromatography (silica), eluting with ethyl acetate/petroleum spirit, 40-60° C. (33%), gave the title compound (250 mg, 92%) as a pale yellow solid.

¹H NMR (400 MHz, CDCl₃) δ 7.31-7.29 (m, 2H), 7.23-7.15 (m, 1H), 6.53 (s, 1H), 6.38-6.23 (m, 1H), 5.91-5.73 (m, 2H), 5.17-4.90 (m, 3H), 4.68-4.19 (m, 4H), 4.00 (m, 2H), 3.93-3.89 (m, 3H), 3.67 (s, 3H), 3.63-3.36 (m 2H), 3.14-2.96 (m, 1H), 2.70-2.65 (m, 1H), 2.37-2.33 (m, 2H), 1.89-1.68 (m, 6H), 1.51-1.50 (m, 8H); MS (ES+): m/z=655 (M+H)⁺; LCMS (Method D): t_{R}=1.24 min.

### 6-(((6aS)-5-((Allyloxy)carbonyl)-2-methoxy-12-oxo-6-((tetrahydro-2H-pyran-2-yl)oxy)-8-(thiophen-3-yl)-5,6,6a,7,10,12-hexahydrobenzo[e]pyrido[1,2-a][1,4]diazepin-3-yl)oxy)hexanoic acid (94)

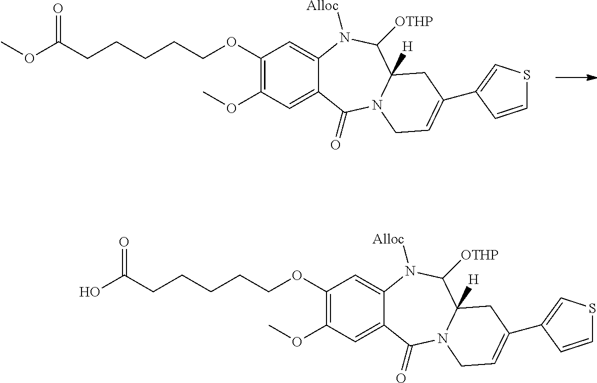

A solution of allyl (6aS)-2-methoxy-3-((6-methoxy-6-oxohexyl)oxy)-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (93) (240 mg, 0.37 mmol) in tetrahydrofuran (10 mL) was charged with an aqueous solution of sodium hydroxide (0.5 M, 3 mL, 1.47 mmol) and the resulting mixture stirred at room temperature for 2 h, before diluting into water (50 mL) and extracting with ethyl acetate (50 mL). The organic phase was then dried over sodium sulfate and concentrated in vacuo. Purification by flash column chromatography (silica), eluting with ethyl acetate/petroleum spirit, 40-60° C. (33%), gave the title compound (190 mg, 80%) as a white solid.

¹H NMR (400 MHz, CDCl₃) δ 7.31-7.29 (m, 2H), 7.23-7.14 (m, 1H), 6.55 (s, 1H), 6.38-6.23 (m, 1H), 5.92-5.70 (m, 2H), 5.15-4.90 (m, 3H), 4.69-4.19 (m, 4H), 4.02-3.97 (m, 2H), 3.93-3.88 (m, 3H), 3.76-3.58 (m, 2H), 3.35 (s, 1H), 3.14-2.96 (m, 1H), 2.72-2.58 (m, 1H), 2.40-2.37 (m, 2H), 1.91-1.64 (m, 8H), 1.55-1.53 (m, 6H); MS (ES+): m/z=641 (M+H)⁺; LCMS (Method E): t_{R}=2.50 min.

### Allyl (6aS)-3-((6-((S)-1-(chloromethyl)-5-hydroxy-1,2-dihydro-3H-benzo[e]indol-3-yl)-6-oxohexyl)oxy)-2-methoxy-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (95)

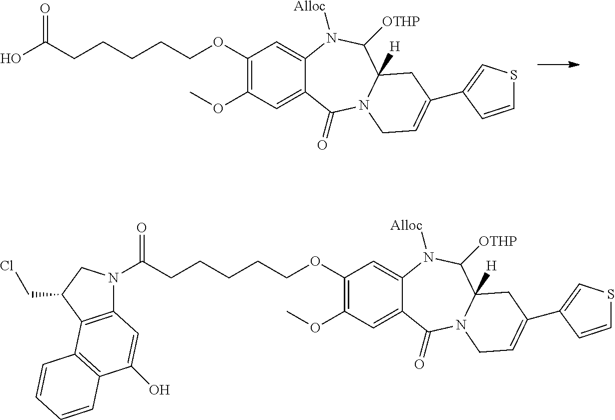

A solution of 6-(((6aS)-5-((allyloxy)carbonyl)-2-methoxy-12-oxo-6-((tetrahydro-2H-pyran-2-yl)oxy)-8-(thiophen-3-yl)-5,6,6a,7,10,12-hexahydrobenzo[e]pyrido[1,2-a][1,4]diazepin-3-yl)oxy)hexanoic acid (94) (190 mg, 0.30 mmol) in N,N-dimethylacetamide (8 mL) was charged with (S)-1-(chloromethyl)-2,3-dihydro-1H-benzo[e]indol-5-ol hydrochloride (12) (113 mg, 0.42 mmol) and N-(3-dimethylaminopropyl)-N′-ethylcarbodiimide hydrochloride (230 mg, 1.20 mmol), and the resulting mixture was stirred at room temperature for 16 h, before diluting into water (100 mL), and then extracting with ethyl acetate (2×100 mL). The combined organic extracts were concentrated in vacuo and purification by flash column chromatography (silica), eluting with ethyl acetate/petroleum spirit, 40-60° C. (from 20% to 33%), gave the title compound (84 mg, 33%) as a pale yellow solid.

¹H NMR (400 MHz, CDCl₃) δ 9.13 (s, 1H), 8.29-8.22 (m, 2H), 7.66-7.64 (m, 1H), 7.54-7.48 (m, 1H), 7.39-7.28 (m, 3H), 7.25-7.13 (m, 2H), 6.66 (s, 1H), 6.35-6.30 (m, 1H), 5.92-5.73 (m, 2H), 5.18-4.93 (m, 3H), 4.35-4.19 (m, 3H), 4.14-4.02 (m, 3H), 3.90-3.87 (m, 3H), 3.75-3.60 (m, 2H), 3.44-3.39 (m, 1H), 3.36-3.06 (m, 2H), 3.01 (s, 2H), 2.94 (s, 2H), 2.72-2.52 (m, 3H), 2.09 (s, 2H), 2.02-1.86 (m, 4H), 1.79-1.71 (m, 4H), 1.54-1.38 (m, 4H); MS (ES+): m/z=856 (M+H)⁺; LCMS (Method D): t_{R}=1.50 min.

### (S)-3-((6-((S)-1-(Chloromethyl)-5-hydroxy-1,2-dihydro-3H-benzo[e]indol-3-yl)-6-oxohexyl)oxy)-2-methoxy-8-(thiophen-3-yl)-7,10-dihydrobenzo[e]pyrido[1,2-a][1,4]diazepin-12(6aH)-one (96)

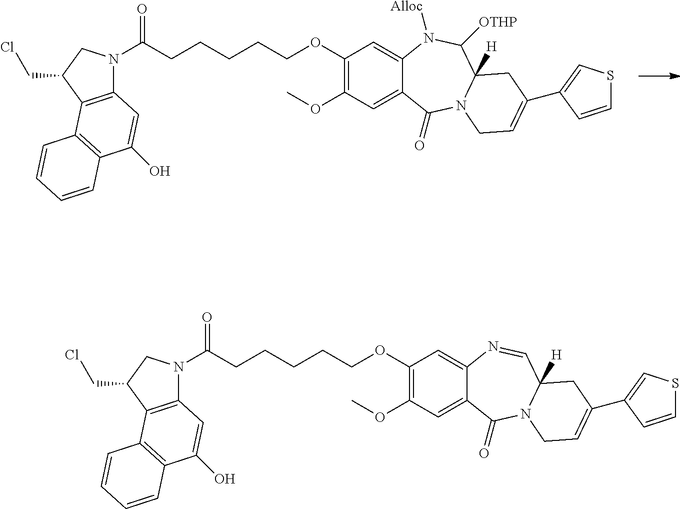

A solution of allyl (6aS)-3-((6-((S)-1-(chloromethyl)-5-hydroxy-1,2-dihydro-3H-benzo[e]indol-3-yl)-6-oxohexyl)oxy)-2-methoxy-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (95) (84 mg, 0.098 mmol) in dichloromethane (4 mL) was charged with tetrakis(triphenylphosphine)palladium(0) (11 mg, 0.01 mmol) and pyrrolidine (8 mg, 0.112 mmol) and the resulting mixture stirred at room temperature for 1 h, before concentrating in vacuo. Purification by preparative HPLC gave the title compound (4.1 mg, 6%) as a yellow solid.

¹H NMR (400 MHz, CD₂Cl₂) δ 8.25-8.23 (d, J=8.0 Hz, 1H), 8.19 (s, 1H), 7.69-7.67 (m, 1H), 7.60-7.59 (m, 1H), 7.53-7.44 (m, 1H), 7.41-7.36 (m, 2H), 7.35-7.29 (m, 1H), 6.83 (s, 1H), 6.50-6.46 (m, 1H), 4.43-4.36 (m, 1H), 4.29-4.27 (m, 2H), 4.17-4.03 (m, 6H), 3.97-3.92 (m, 3H), 3.86 (s, 3H), 2.95-2.93 (m, 2H), 2.67-2.52 (m, 3H), 2.03-1.87 (m, 6H); MS (ES+): m/z=670 (M+H)⁺; LCMS (Method F): t_{R}=3.80 min.

### Allyl (6aS)-2-methoxy-3-(4-methoxy-4-oxobutoxy)-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (97)

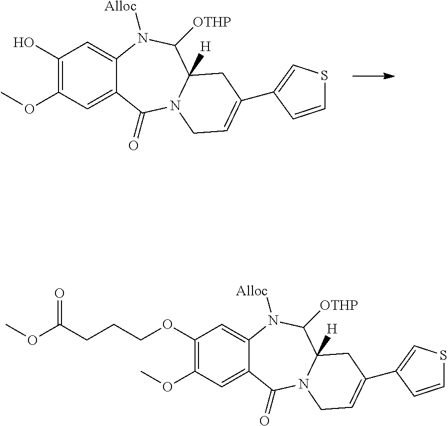

A solution of allyl (6aS)-3-hydroxy-2-methoxy-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (67) (200 mg, 0.38 mmol) in N,N-dimethylformamide (2 mL) was charged with methyl 4-bromobutanoate (73 mg, 0.40 mmol) and potassium carbonate (79 mg, 0.57 mmol) and the resulting mixture stirred at room temperature for 18 h, after which it was diluted into water (100 mL) and extracted with ethyl acetate (100 mL). The organic phase was then dried over sodium sulfate, filtered and concentrated in vacuo to give the title compound (224 mg, 94%) as a yellow solid.

¹H NMR (400 MHz, DMSO-d₆) δ 7.60-7.55 (m, 1H), 7.51-7.50 (m, 1H), 7.44-7.41 (m, 1H), 7.11 (s, 1H), 6.88 (s, 1H), 6.45-6.43 (m, 1H), 6.82-5.70 (m, 1H), 5.66 (d, J=9.7 Hz, 1H), 5.16-5.00 (m, 2H), 4.95 (m, 1H), 4.57-4.36 (m, 3H), 4.12-4.08 (m, 1H), 4.01-3.95 (m, 2H), 3.85-3.82 (m, 3H), 3.64-3.58 (m, 5H), 3.26-3.22 (m, 1H), 2.97-2.92 (m, 1H), 2.73-2.66 (m, 1H), 2.07-2.03 (m, 1H), 1.95-1.93 (m, 1H), 1.69-1.66 (m, 2H), 1.48-1.45 (m, 4H), 1.23 (s, 2H); MS (ES+): m/z=627 (M+H)⁺; LCMS (Method E): t_{R}=2.61 min.

### 4-(((6aS)-5-((Allyloxy)carbonyl)-2-methoxy-12-oxo-6-((tetrahydro-2H-pyran-2-yl)oxy)-8-(thiophen-3-yl)-5,6,6a,7,10,12-hexahydrobenzo[e]pyrido[1,2-a][1,4]diazepin-3-yl)oxy)butanoic acid (98)

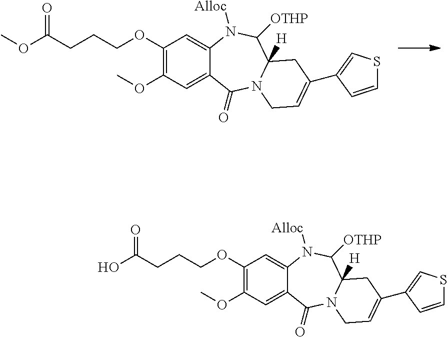

A solution of allyl (6aS)-2-methoxy-3-(4-methoxy-4-oxobutoxy)-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (97) (220 mg, 0.37 mmol) in 1,4-dioxane (1 mL) was charged with an aqueous solution of sodium hydroxide (0.5 M, 2.8 mL) and the resulting mixture stirred at room temperature for 2 h, before diluting into water (50 mL) and extracting with ethyl acetate (50 mL). The organic phase was dried over sodium sulfate, filtered and concentrated in vacuo to give the title compound (186 mg, 85%) as a white solid.

¹H NMR (400 MHz, DMSO-d₆) δ 12.15 (s, 1H), 7.60-7.55 (m, 1H), 7.51-7.50 (m, 1H), 7.44-7.41 (m, 1H), 7.11 (s, 1H), 6.90-6.81 (m, 1H), 6.51-6.44 (m, 1H), 5.83-5.71 (m, 1H), 5.67-5.50 (m, 1H), 5.15-5.01 (m, 2H), 5.00-4.95 (m, 1H), 4.65-4.36 (m, 3H), 4.15-4.04 (m, 1H), 4.01-3.94 (m, 2H), 3.86-3.81 (m, 3H), 3.67-3.57 (m, 1H), 3.52-3.43 (m, 1H), 3.27-3.20 (m, 1H), 2.92 (t, J=16.3 Hz, 1H), 2.75-2.65 (m, 1H), 2.38 (t, J=7.4 Hz, 2H), 1.99-1.89 (m, 2H), 1.71-1.60 (m, 2H), 1.52-1.36 (m, 4H); MS (ES+): m/z=613 (M+H)⁺; LCMS (Method E): t_{R}=2.23 min.

### Allyl (6aS)-3-(4-((S)-1-(chloromethyl)-5-hydroxy-1,2-dihydro-3H-benzo[e]indol-3-yl)-4-oxobutoxy)-2-methoxy-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate_(99)

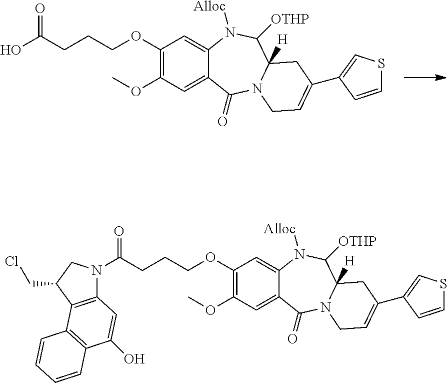

A solution of 4-(((6aS)-5-((allyloxy)carbonyl)-2-methoxy-12-oxo-6-((tetrahydro-2H-pyran-2-yl)oxy)-8-(thiophen-3-yl)-5,6,6a,7,10,12-hexahydrobenzo[e]pyrido[1,2-a][1,4]diazepin-3-yl)oxy)butanoic acid (98) (100 mg, 0.16 mmol) in N,N-dimethylacetamide (1 mL) was charged with (S)-1-(chloromethyl)-2,3-dihydro-1H-benzo[e]indol-5-ol hydrochloride (12) (54 mg, 0.23 mmol) and N-(3-dimethylaminopropyl)-N′-ethylcarbodiimide hydrochloride (125 mg, 0.65 mmol) and the resulting mixture was stirred at room temperature for 18 h, before diluting into water (50 mL) and extracting with ethyl acetate (50 mL). The organic phase was then dried over sodium sulfate, filtered and concentrated in vacuo. Purification by flash column chromatography (silica), eluting with methanol/dichloromethane (from 2% to 3%), gave the title compound (45 mg, 33%) as a green solid.

¹H NMR (400 MHz, CDCl₃) δ 9.50-8.95 (m, 1H), 8.38-8.01 (m, 2H), 7.69-7.60 (m, 1H), 7.51 (t, J=70.6 Hz, 1H), 7.43-7.11 (m, 5H), 6.39-6.26 (m, 1H), 5.95-5.61 (m, 2H), 5.30-4.86 (m, 3H), 4.59-4.46 (m, 1H), 4.40-4.19 (m, 5H), 4.08-4.01 (d, J=6.7 Hz, 1H), 3.99-3.84 (m, 4H), 3.80-3.50 (m, 2H), 3.39-3.32 (m, 1H), 3.15-2.85 (m, 2H), 2.79-2.62 (m, 2H), 2.44-2.25 (m, 2H), 1.83-1.59 (m, 6H), 1.54-1.35 (m, 4H); MS (ES+): m/z=828 (M+H)⁺; LCMS (Method E): t_{R}=2.77 min.

### (S)-3-(4-((S)-1-(Chloromethyl)-5-hydroxy-1,2-dihydro-3H-benzo[e]indol-3-yl)-4-oxobutoxy)-2-methoxy-8-(thiophen-3-yl)-7,10-dihydrobenzo[e]pyrido[1,2-a][1,4]diazepin-12(6aH)-one (100)

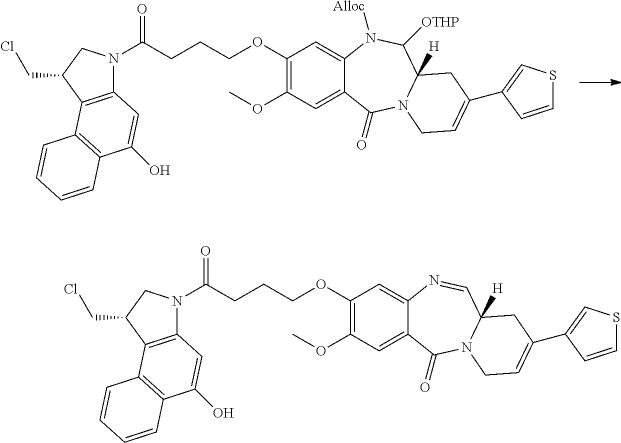

A solution of allyl (6aS)-3-(4-((S)-1-(chloromethyl)-5-hydroxy-1,2-dihydro-3H-benzo[e]indol-3-yl)-4-oxobutoxy)-2-methoxy-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (99) (40 mg, 0.048 mmol) in dichloromethane (1 mL) was charged with tetrakis(triphenylphosphine)palladium(0) (6 mg, 0.005 mmol) and pyrrolidine (4 mg, 0.058 mmol) and the resulting mixture stirred at room temperature for 0.5 h, before concentrating in vacuo. Purification by preparative HPLC gave the title compound (8 mg, 56%) as a grey solid.

¹H NMR (400 MHz, CD₂Cl₂) δ 9.71 (br, 1H), 8.37-8.09 (m, 2H), 7.66 (d, J=8.4 Hz, 1H), 7.61-7.47 (m, 2H), 7.47-7.33 (m, 3H), 7.33-7.21 (m, 2H), 6.87 (s, 1H), 6.50-6.30 (m, 1H), 4.35-4.17 (m, 4H), 4.14-3.88 (m, 4H), 3.87-3.59 (m, 4H), 3.46 (t, J=10.6 Hz, 1H), 3.27 (s, 1H), 2.98-2.82 (s, 2H), 2.26-2.10 (m, 1H), 1.99-1.90 (m, 1H), 1.59 (s, 1H); MS (ES+): m/z=642 (M+H)⁺; LCMS (Method E): t_{R}=1.73 min.

### Allyl (6aS)-3-(2-(3-(tert-butoxy)-3-oxopropoxy)ethoxy)-2-methoxy-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (101)

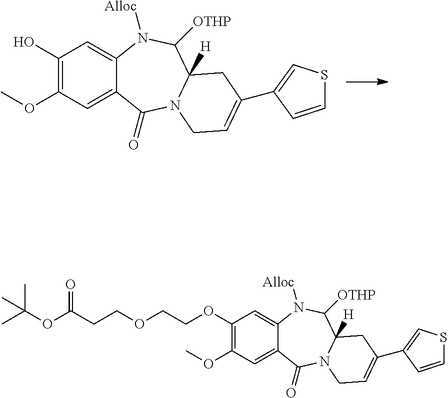

A solution of allyl (6aS)-3-hydroxy-2-methoxy-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (67) (150 mg, 0.29 mmol) in N,N-dimethylformamide (8 mL) was charged with tert-butyl 3-(2-bromoethoxy)propanoate (108 mg, 0.43 mmol) and potassium carbonate (118 mg, 0.85 mmol) and the resulting mixture stirred for 16 h, at room temperature, before diluting into water (50 mL) and then extracting with dichloromethane (2×50 mL). The combined organic extracts were then dried over sodium sulfate, filtered and concentrated in vacuo. Purification by flash column chromatography (silica), eluting with ethyl acetate/petroleum spirit, 40-60° C. (50%), gave the title compound (150 mg, 75%) as a white solid.

¹H NMR (400 MHz, CDCl₃) δ 7.35-7.31 (m, 1H), 7.29-7.28 (m, 1H), 7.23-7.14 (m, 1H), 6.88-6.58 (m, 1H), 6.35-6.29 (m, 1H), 5.90-5.72 (m, 2H), 5.15-4.89 (m, 3H), 4.65-4.06 (m, 6H), 3.91-3.90 (m, 3H), 3.87-3.84 (m, 2H), 3.79 (t, J=6.4 Hz, 2H), 3.74-3.32 (m, 3H), 3.14-2.96 (m, 1H), 2.72-2.59 (m, 1H), 2.54-2.51 (m, 2H), 1.80-1.69 (m, 2H), 1.58-1.46 (m, 4H), 1.44 (s, 9H); MS (ES+): m/z=699 (M+H)⁺; LCMS (Method D): t_{R}=1.53 min.

### 3-(2-(((6aS)-5-((Allyloxy)carbonyl)-6-hydroxy-2-methoxy-12-oxo-8-(thiophen-3-yl)-5,6,6a,7,10,12-hexahydrobenzo[e]pyrido[1,2-a][1,4]diazepin-3-yl)oxy)ethoxy)propanoic acid (102)

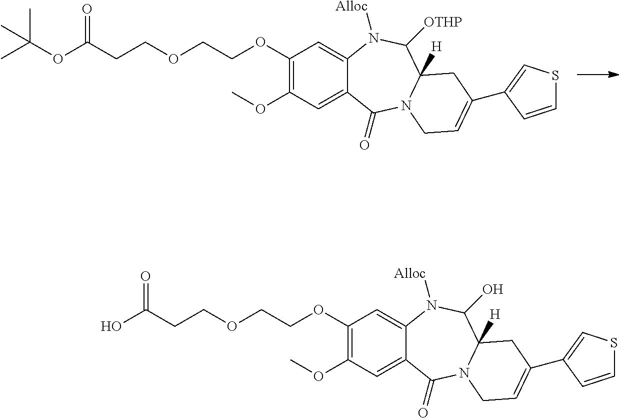

A solution of allyl (6aS)-3-(2-(3-(tert-butoxy)-3-oxopropoxy)ethoxy)-2-methoxy-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (101) (140 mg, 0.20 mmol) in hydrochloric acid (4 M in 1,4-dioxane, 4 mL) was stirred for 2 h at room temperature, before concentrating in vacuo. Purification by flash column chromatography (silica), eluting with methanol/dichloromethane (5%), gave the title compound (70 mg, 62%) as a white solid.

¹H NMR (400 MHz, DMSO-d₆) δ 7.59-7.57 (m, 1H), 7.50-7.49 (m, 1H), 7.43-7.41 (m, 1H), 7.10 (s, 1H), 6.80 (s, 1H), 6.47-6.43 (m, 1H), 5.43 (d, J=10.0 Hz, 1H), 5.10-5.02 (m, 2H), 4.53-4.39 (m, 3H), 4.09-4.04 (m, 4H), 3.83 (s, 3H), 3.76-3.62 (m, 5H), 3.52-3.49 (m, 1H), 2.95-2.88 (m, 1H), 2.70-2.62 (m, 1H), 2.44 (t, J=6.4 Hz, 2H); MS (ES+): m/z=559 (M+H)⁺; LCMS (Method F): t_{R}=3.10 min.

### Allyl (6aS)-3-(2-(3-((S)-1-(chloromethyl)-5-hydroxy-1,2-dihydro-3H-benzo[e]indol-3-yl)-3-oxopropoxy)ethoxy)-6-hydroxy-2-methoxy-12-oxo-8-(thiophen-3-yl)-6,6a,7,10-tetrahydrobenzo[e]pyrido[1,2-a][1,4]diazepine-5(12H)-carboxylate (103)

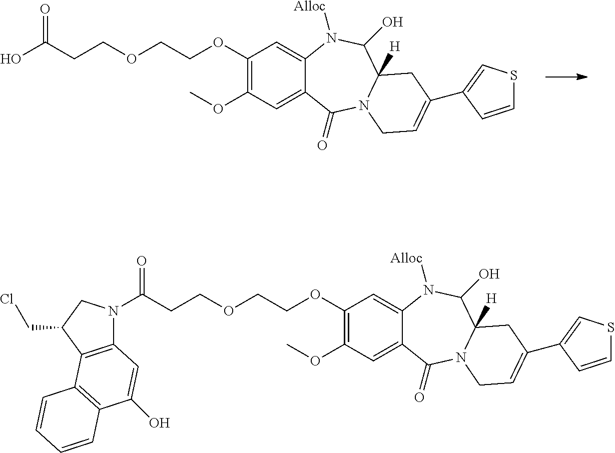

A solution of 3-(2-(((6aS)-5-((Allyloxy)carbonyl)-6-hydroxy-2-methoxy-12-oxo-8-(thiophen-3-yl)-5,6,6a,7,10,12-hexahydrobenzo[e]pyrido[1,2-a][1,4]diazepin-3-yl)oxy)ethoxy)propanoic acid (102) (70 mg, 0.13 mmol) in N,N-dimethylacetamide (6 mL) was charged with (S)-1-(chloromethyl)-2,3-dihydro-1H-benzo[e]indol-5-ol hydrochloride (12) (48 mg, 0.18 mmol) and N-(3-dimethylaminopropyl)-N′-ethylcarbodiimide hydrochloride (96 mg, 0.50 mmol) and the resulting mixture was stirred at room temperature for 16 h, before diluting into water (50 mL) and extracting with dichloromethane (2×50 mL). The combined organic extracts were then dried over sodium sulfate, filtered and concentrated in vacuo. Purification by flash column chromatography (silica), eluting with methanol/dichloromethane (5%), gave the title compound (40 mg, 42%) as a pale yellow solid.

¹H NMR (400 MHz, DMSO-d₆) δ 10.36 (s, 1H), 8.08 (d, J=8.4 Hz, 1H), 7.97 (s, 1H), 7.78 (d, J=8.4 Hz, 1H), 7.60-7.56 (m, 1H), 7.50-7.46 (m, 2H), 7.42-7.40 (m, 1H), 7.33-7.29 (m, 1H), 7.09 (s, 1H), 6.81 (s, 1H), 6.66-6.64 (m, 1H), 6.46-6.43 (m, 1H), 5.82-5.75 (m, 1H), 5.43 (dd, J=90.8, 5.8 Hz, 1H), 5.08-5.00 (m, 2H), 4.56-4.39 (m, 3H), 4.35-4.30 (m, 1H), 4.22-4.18 (m, 1H), 4.12-4.04 (m, 5H), 3.97-3.93 (m, 1H), 3.85-3.75 (m, 8H), 3.52-3.48 (m, 1H), 2.92-2.82 (m, 2H), 2.72-2.62 (d, 2H); MS (ES+): m/z=774 (M+H)⁺; LCMS (Method F): t_{R}=2.37 min.

### (S)-3-(2-(3-((S)-1-(Chloromethyl)-5-hydroxy-1,2-dihydro-3H-benzo[e]indol-3-yl)-3-oxopropoxy)ethoxy)-2-methoxy-8-(thiophen-3-yl)-7,10-dihydrobenzo[e]pyrido[1,2-a][1,4]diazepin-12(6aH)-one (104)

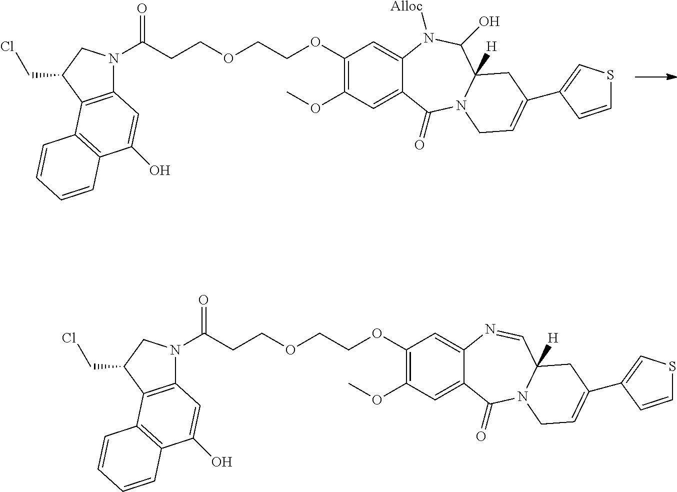

A solution of allyl (6aS)-3-(2-(3-((S)-1-(chloromethyl)-5-hydroxy-1,2-dihydro-3H-benzo[e]indol-3-yl)-3-oxopropoxy)ethoxy)-6-hydroxy-2-methoxy-12-oxo-8-(thiophen-3-yl)-6,6a,7,10-tetrahydrobenzo[e]pyrido[1,2-a][1,4]diazepine-5(12H)-carboxylate (103) (57 mg, 0.073 mmol) in dichloromethane (4 mL) was charged with tetrakis(triphenylphosphine)palladium(0) (8.5 mg, 0.007 mmol) and pyrrolidine (6.3 mg, 0.088 mmol) and the resulting mixture was stirred at room temperature for 0.5 h, before concentrating in vacuo. Purification by preparative HPLC gave the title compound (11 mg, 22%) as a pale yellow solid.

¹H NMR (400 MHz, CD₂Cl₂) δ 8.15-8.11 (m, 1H), 7.69-7.55 (m, 2H), 7.52-7.43 (m, 2H), 7.42-7.37 (m, 2H), 7.36-7.29 (m, 3H), 6.91-6.79 (m, 2H), 6.48-6.45 (m, 1H), 4.43-4.27 (m, 2H), 4.26-4.09 (m, 4H), 4.08-3.99 (m, 2H), 3.97-3.85 (m, 8H), 3.76 (s, 1H), 2.94-2.92 (m, 1H), 2.87-2.75 (m, 3H); MS (ES+): m/z=672 (M+H)⁺; LCMS (Method F): t_{R}=2.27 min.

### Allyl (6aS)-3-((S)-2-((tert-butyldimethylsilyl)oxy)-4-ethoxy-4-oxobutoxy)-2-methoxy-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (105)

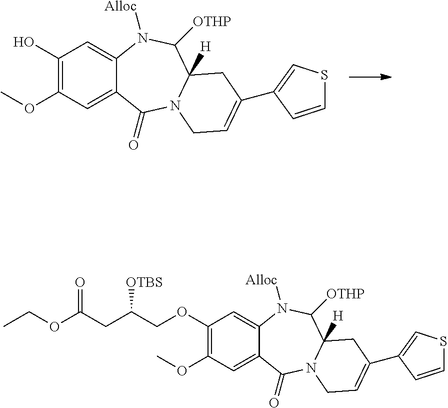

A solution of triphenylphosphine (610 mg, 2.33 mmol) in toluene (2 mL) at 0° C. was charged with diethyl azodicarboxylate (347 mg, 1.99 mmol), and stirred for 30 min (under an inert atmosphere of nitrogen). Allyl (6aS)-3-hydroxy-2-methoxy-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (67) (350 mg, 0.66 mmol) and ethyl (S)-3-((tert-butyldimethylsilyl)oxy)-4-hydroxybutanoate (80) (262 mg, 1.00 mmol) in toluene (2 mL) were added to the mixture at 0° C., which was then stirred at room temperature for 18 h. After diluting into ethyl acetate (100 mL) and extracting with water (2×30 mL), the organic phase was washed with brine (30 mL), dried over sodium sulfate, filtered and concentrated in vacuo. Purification by flash column chromatography (silica), eluting with ethyl acetate/petroleum spirit, 40-60° C. (from 20% to 50%), and then by preparative TLC, eluting with ethyl acetate/petroleum spirit, 40-60° C. (33%), gave the title compound (120 mg, 29%) as a white solid.

¹H NMR (400 MHz, CDCl₃) δ 7.36-7.27 (m, 2H), 7.24-6.82 (m, 2H), 6.52-6.25 (m, 3H), 6.94-5.69 (m, 1H), 5.18-4.60 (m, 3H), 4.60-4.41 (m, 3H), 4.41-4.25 (m, 2H), 4.16-4.09 (m, 2H), 4.00-3.90 (m, 2H), 3.90-3.51 (m, 6H), 3.05 (br, 1H), 2.78-2.61 (m, 2H), 2.59-2.46 (m, 1H), 1.83-1.66 (m, 2H), 1.62 (s, 4H), 0.86 (s, 9H), 0.12-0.06 (m, 6H), 0.00 (s, 3H); MS (ES+): m/z=771 (M+H)⁺; LCMS (Method D): t_{R}=2.26 min.

### (3S)-4-(((6aS)-5-((Allyloxy)carbonyl)-6-hydroxy-2-methoxy-12-oxo-8-(thiophen-3-yl)-5,6,6a,7,10,12-hexahydrobenzo[e]pyrido[1,2-a][1,4]diazepin-3-yl)oxy)-3-hydroxybutanoic acid (106)

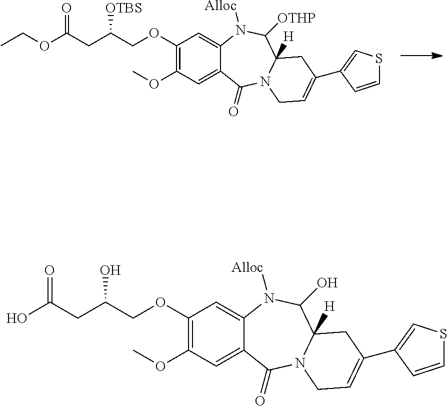

A solution of allyl (6aS)-3-((S)-2-((tert-butyldimethylsilyl)oxy)-4-ethoxy-4-oxobutoxy)-2-methoxy-12-oxo-6-((tetrahydro-2H-pyran-2-yl)oxy)-8-(thiophen-3-yl)-6,6a,7,10-tetrahydrobenzo[e]pyrido[1,2-a][1,4]diazepine-5(12H)-carboxylate (105) (106 mg, 0.14 mmol) in tetrahydrofuran (1 mL) was charged with an aqueous solution of hydrochloric acid (4 M, 2 mL) and stirred at 50° C. for 4 h, before diluting into water (100 mL) and extracting with ethyl acetate (3×30 mL). The combined organic extracts were then washed with brine (30 mL), dried over sodium sulfate, filtered and concentrated in vacuo. Purification by preparative TLC, eluting with methanol/dichloromethane (5%; plus one drop of acetic acid) gave the title compound (43 mg, 58%) as a yellow solid.

¹H NMR (400 MHz, CDCl₃) s7.33-7.28 (m, 1H), 7.21 (d, J=90.6 Hz, 2H), 6.90-6.70 (m, 1H), 6.32-6.16 (m, 1H), 5.86-5.60 (m, 2H), 5.17-5.05 (m, 2H), 4.82-4.53 (m, 2H), 4.43 (s, 2H), 4.29 (dd, J=18.8, 5.6 Hz, 1H), 4.21-4.11 (m, 1H), 4.03 (s, 3H), 3.92-3.79 (m, 4H), 3.74-3.67 (m, 1H), 3.05 (d, J=16.4 Hz, 1H), 2.70-2.57 (m, 3H); MS (ES+): m/z=545 (M+H)⁺; LCMS (Method F): t_{R}=1.63 min.

### Allyl (6aS)-3-((S)-4-((S)-1-(chloromethyl)-5-hydroxy-1,2-dihydro-3H-benzo[e]indol-3-yl)-2-hydroxy-4-oxobutoxy)-6-hydroxy-2-methoxy-12-oxo-8-(thiophen-3-yl)-6,6a,7,10-tetrahydrobenzo[e]pyrido[1,2-a][1,4]diazepine-5(12H)-carboxylate (107)

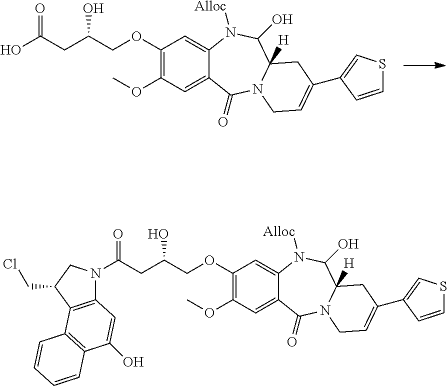

A solution of (3S)-4-(((6aS)-5-((allyloxy)carbonyl)-6-hydroxy-2-methoxy-12-oxo-8-(thiophen-3-yl)-5,6,6a,7,10,12-hexahydrobenzo[e]pyrido[1,2-a][1,4]diazepin-3-yl)oxy)-3-hydroxybutanoic acid (106) (43 mg, 0.079 mmol) in N,N-dimethylacetamide (1 mL) was charged with (S)-1-(chloromethyl)-2,3-dihydro-1H-benzo[e]indol-5-ol hydrochloride (12) (30 mg, 0.11 mmol) and N-(3-dimethylaminopropyl)-N′-ethylcarbodiimide hydrochloride (61 mg, 0.32 mmol) and the resulting mixture was stirred at room temperature for 18 h, before diluting into water (60 mL) and extracting with ethyl acetate (2×20 mL). The combined organic extracts were then washed with brine (20 mL), dried over sodium sulfate, filtered and concentrated in vacuo to give the title compound (34 mg, 57%) as a grey solid.

¹H NMR (400 MHz, CDCl₃) δ 8.21 (d, J=8.4 Hz, 1H), 7.92 (s, 1H), 7.43-7.40 (m, 1H), 7.39-7.33 (m, 2H), 7.32-7.27 (m, 2H), 7.24-7.14 (m, 4H), 6.90 (s, 1H), 6.35-6.11 (m, 1H), 5.82-5.62 (m, 2H), 5.16-5.07 (m, 2H), 4.91 (s, 1H), 4.53-4.01 (m, 2H), 4.35-4.15 (m, 4H), 4.07-3.56 (m, 10H), 2.91-2.77 (m, 2H), 2.72-2.52 (m, 2H); MS (ES+): m/z=759 (M+H)⁺; LCMS (Method F): t_{R}=2.25 min.

### (S)-3-((S)-4-((S)-1-(Chloromethyl)-5-hydroxy-1,2-dihydro-3H-benzo[e]indol-3-yl)-2-hydroxy-4-oxobutoxy)-2-methoxy-8-(thiophen-3-yl)-7,10-dihydrobenzo[e]pyrido[1,2-a][1,4]diazepin-12(6aH)-one (108)

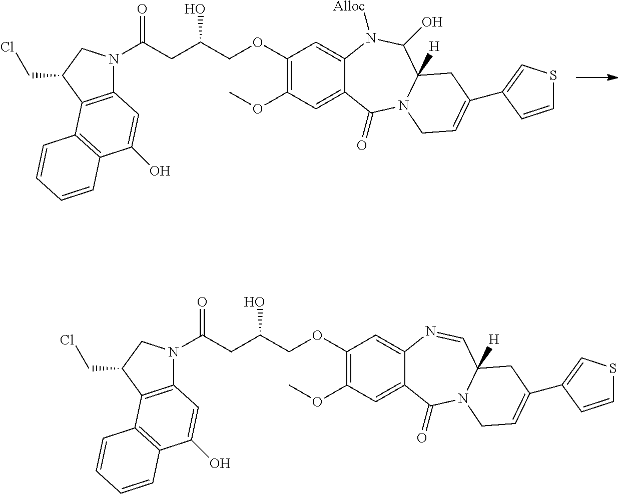

A solution of allyl (6aS)-3-((S)-4-((S)-1-(chloromethyl)-5-hydroxy-1,2-dihydro-3H-benzo[e]indol-3-yl)-2-hydroxy-4-oxobutoxy)-6-hydroxy-2-methoxy-12-oxo-8-(thiophen-3-yl)-6,6a,7,10-tetrahydrobenzo[e]pyrido[1,2-a][1,4]diazepine-5(12H)-carboxylate (107) (34 mg, 0.045 mmol) in dichloromethane (1 mL) was charged with tetrakis(triphenylphosphine)palladium(0) (5 mg) and pyrrolidine (4 mg) and the resulting mixture at room temperature for 1 h, before concentrating in vacuo. Purification by preparative HPLC gave the title compound (4.3 mg, 15%) as a yellow solid.

¹H NMR (400 MHz, DMSO-d₆) δ 10.42 (s, 1H), 8.10 (d, J=8.4 Hz, 1H), 8.02 (s, 1H), 7.80 (d, J=8.4 Hz, 1H), 7.74-7.66 (m, 1H), 7.66-7.58 (m, 2H), 7.54-7.47 (m, 2H), 7.40-7.31 (m, 2H), 7.11-6.91 (m, 1H), 6.66-6.38 (m, 1H), 5.27 (d, J=50.2 Hz, 1H), 4.46-4.37 (m, 2H), 4.26-4.22 (m, 1H), 4.21-4.15 (m, 2H), 4.11-3.95 (m, 4H), 3.91 (s, 1H), 3.87 (s, 2H), 3.82-3.73 (m, 2H), 3.12 (d, J=16.0 Hz, 1H), 2.94-2.81 (m, 2H), 2.79-2.72 (m, 1H); MS (ES+): m/z=658 (M+H)⁺; LCMS (Method F): t_{R}=2.14 min.

### Biological Characterisation

### Biological and Biophysical Characterisation of Free Payloads

### In Vitro Cytotoxicity

The in vitro cytotoxicity of a selection of compounds was determined in a panel of cell lines using the standard MTT assay for a 72 hour incubation period (Table 1).

TABLE 1
In vitro cytotoxicity of a selection of compounds against six cell-lines.
Values in brackets for compound 31 are Std Dev after 3 runs.

| | Cytotoxicity (nM) | | | | | |
|---|---|---|---|---|---|---|
| Compound | SW48 | LIM1215 | SW620 | RAJI | JVM2 | Jurkat |
| Number | (Gastric) | (Gastric) | (Gastric) | (Lymphoma) | (Lymphoma) | (Leukaemia) |
| 31 | 1.3 | 2.7 | 0.35 | | | |
| | (0.19) | (0.68) | (0.07) | | | |
| 35 | 3.15 | 4.38 | 2.12 | | | |
| 72 | 0.29 | 0.32 | 0.21 | 0.0019 | 0.0016 | 0.0015 |
| 76 | | | | 0.0172 | 0.0138 | 0.0095 |
| 88 | | | | 0.954 | 0.49 | 0.97 |
| 92 | | | | 0.41 | 0.12 | 0.55 |
| 96 | | | | 0.0014 | 0.0008 | 0.0005 |
| 100 | | | | 0.0009 | 0.001 | 0.0009 |
| 104 | | | | 0.0025 | 0.0018 | 0.0095 |
| 108 | | | | 0.01 | 0.0047 | 0.014 |

### Methodology

### MTT Cytotoxicity Methodology

Tumor cell lines were maintained in RPMI1640 medium supplemented with 10% heat-inactivated fetal bovine serum, 2 mM L-glutamine and 1 mM sodium pyruvate. 1800 cells per well were seeded in a volume of 180 μl in a 96-well flat bottom polystyrene plate. The cells were allowed to adhere overnight at 37° C. in a CO₂ incubator. Ligands were initially formulated in DMSO, and stocks stored at −80° C. They were then further formulated at lox concentration in RPMI1640 medium. 20 μl of diluted samples were added into each treatment well. On each plate, blank wells with no cells, and untreated wells containing cells, were included. Plates were then cultured at 37° C. in a CO₂ incubator for 72 hrs. Cytotoxicity was evaluated using a tetrazolium salt-based assay, the MTT assay. After 72 hours, the supernatant was removed from each well and 200 μl of a sterile filtered 500 μg/ml MTT solution in water added to each well. The plates were then incubated at 37° C. in a CO₂ incubator for 4 hrs. The supernatant was then removed and the formazan crystals formed solubilized by adding 150 μl of DMSO to each well. The plate was then read on a plate reader at 540 nm, and percentage cell survival calculated as follows: ((mean absorbance treated wells at concentration×−mean absorbance blank wells)÷(mean absorbance untreated wells at concentration×−mean absorbance blank wells))×100. Data were plotted as concentration in nM vs. % cell survival in Microsoft Excel, and IC₅₀ values (concentration where cell survival is reduced by a half) were determined from the graph.

### Hydrophobicity Investigation of Free Payloads

### Method

Both mobile phases were filtered through 0.22 μm filters and degassed by sonication under argon. Caffeine (Merck, UPLC grade) was employed a standard. Blank solvent samples (i.e., DMSO) were used during sample runs to confirm lack of carryover between injections and to monitor column performance.

UPLC-MS analysis was performed on a Waters Acquity H-Class Ultra-Performance Liquid Chromatography (UPLC) System equipped with an Acquity UPLC BEH, 1.7 m 50×2.10 mm (Waters, UK) column and a photodiode array detector (502.93 n), using Waters MassLynx software. The gradient system used for LC analysis consisted of 0.1% trifluoroacetic acid (TFA) in water as Solvent A, and 0.1% trifluoroacetic acid (TFA) in acetonitrile as Solvent B. TFA was purchased from Merck (UK), and diluted to the required concentration with UPLC grade water and acetonitrile for Solvent A and Solvent B, respectively. TFA was added to ensure acidic conditions throughout the analysis. A flow rate of 0.4 mL/min was used, and the detection wavelength was 254 nm. Mass spectrometry data were collected using a Waters SQ Detector 2 coupled to a Waters Acquity H Class UPLC and a Photo Diode Array Detector (ACQ-PDA). The Waters SQ Detector 2 parameters were set to: Capillary (kV), 3.00; Cone (V), 30; De-solvation Temperature (° C.), 600; Cone flow rate (L/h), 50; De-solvation flow rate (L/h), 600.

Gradient method used: from 95% A/5% B to 20% B over 5 min. Then from 20% B to 60% B over 35 min, followed by from 60% B to 85% B over 10 min. It was further changed from 85% B to 90% B over 5 min and kept for a further 5 min. Finally, from 90% B to 95% B over 2.5 min, and then to 20% B over 2.5 min. 10 μL was split via a zero-dead volume T-piece which passed into the mass spectrometer.

Compounds were provided in powder form and were dissolved in DMSO to achieve 5 mM stock solution. This solution was then diluted to 500 μM with 50% acetonitrile/50% water.

### Analysis

The retention times achieved are presented in Table 2. A number of commercially available ADC payloads and internal reference compounds were also incorporated into the analysis, and their structures are presented in Table 3.

TABLE 2
Retention times obtained from the UPLC analysis of
compounds using hydrophobicity model. All
compounds were analysed in triplicate.

| Compound | | Retention Time | Retention Time |
|---|---|---|---|
| number | Run | (min) seco form | (min) spiro form |
| 96 | 1 | 28.08 | 21.02 |
| | 2 | 28.06 | 21.02 |
| | 3 | 28.04 | 21.00 |
| 104 | 1 | 24.10 | 16.90 |
| | 2 | 24.09 | 16.93 |
| | 3 | 24.09 | 16.91 |
| 72 | 1 | 24.02 | 17.11 |
| | 2 | 24.04 | 17.11 |
| | 3 | 24.04 | 17.15 |
| 76 | 1 | 20.14 | 13.29 |
| | 2 | 20.14 | 13.29 |
| | 3 | 20.14 | 13.28 |
| 100 | 1 | 25.24 | 17.94 |
| | 2 | 25.23 | 17.89 |
| | 3 | 25.24 | 17.93 |
| 88 | 1 | 18.73 | 12.47 |
| | 2 | 18.70 | 12.45 |
| | 3 | 18.70 | 12.45 |
| 92 | 1 | 18.82 | 12.16 |
| | 2 | 18.84 | 12.18 |
| | 3 | 18.84 | 12.18 |
| 108 | 1 | 22.54 | 15.71 |
| | 2 | 22.53 | 15.69 |
| | 3 | 22.53 | 15.70 |
| A | 1 | 22.55 | 14.85 |
| | 2 | 22.55 | 14.83 |
| | 3 | 22.56 | 14.84 |
| B | 1 | 23.64 | 16.10 |
| | 2 | 23.65 | 16.12 |
| | 3 | 23.64 | 16.09 |
| C | 1 | 20.16 | 12.49 |
| | 2 | 20.15 | 12.48 |
| | 3 | 20.15 | 12.45 |
| D | Average | 17.03 | N/A |
| SN38 | Average | 7.13 | N/A |
| SG3199 | Average | 12.02 | N/A |
| MMAE | Average | 12.04 | N/A |
| SGD1882 | Average | 12.55 | N/A |
| DGN549 | Average | 16.60 | N/A |
| DM1 | Average | 21.24 | N/A |

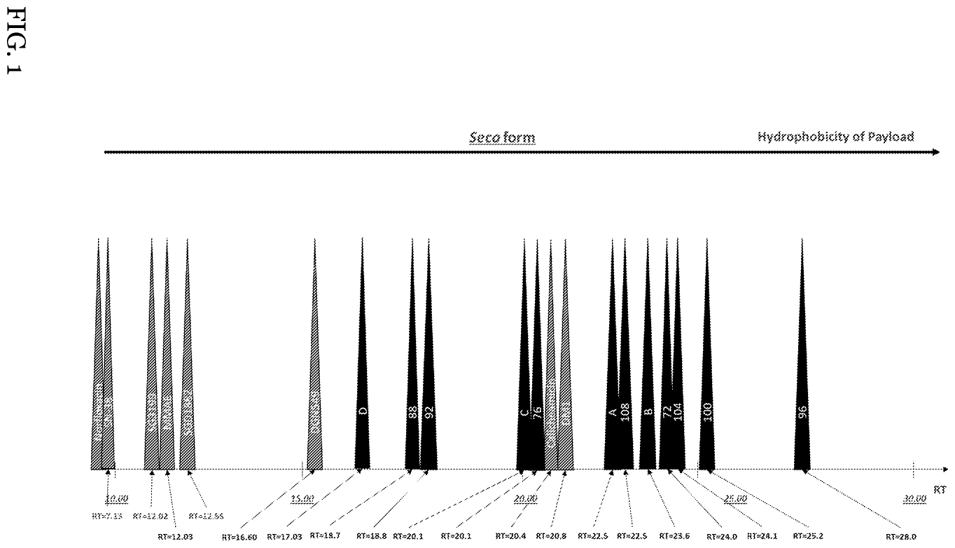

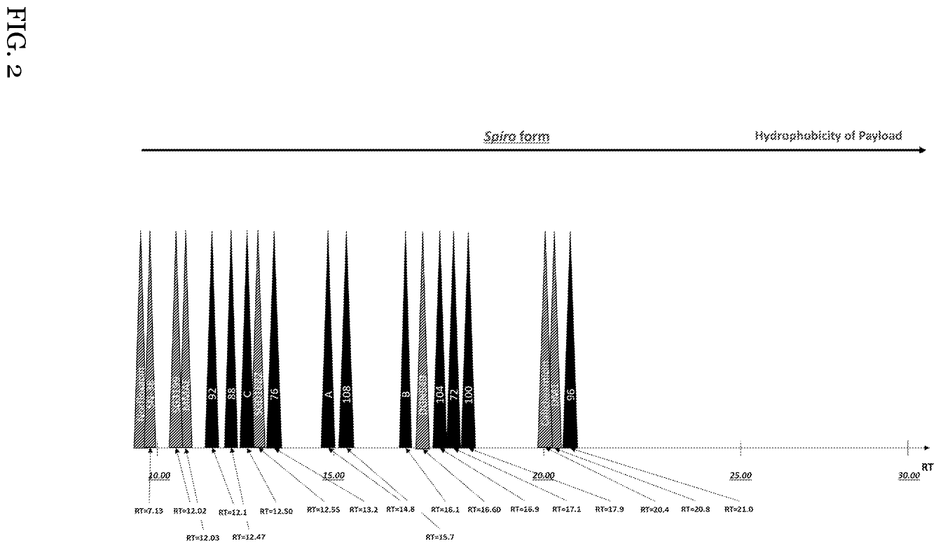

A visual representation of data presented in Table 2 (UPLC data showing the retention times of the compounds analysed) is shown in FIGS. 1 and 2.

Presence of seco- and spiro-forms were observed for each unsymmetrical dimer, aside from reference compound D. Reference compound D contains a carbamate pro-drug, so is only observable in its seco-form. SN38, SG3199, MMAE, DGN549 and DM1 do not contain a CXI moiety, so are also only observable as single species.

The retention time difference was consistent between the unsymmetrical dimer compounds, showing approximately 7 min retention time shift between seco- and spiro-forms; spiro-form being more hydrophilic. Cumulative conversion to spiro form was observed with each run, corresponding to length of time the compounds were exposed to water. Furthermore, the addition of a carbamate pro-drug moiety enhanced hydrophilicity of a reference compound (D) by approximately 10 mins compared to the unsubstituted compound (B), suggesting a similar approach could be adopted in unsymmetrical dimers disclosed in this application.

TABLE 3
Structures of reference compounds A-D, and commercially relevent ADC payloads SN38, SG3199, MMAE, DGN549, SGD1882 and DM1.

| Reference | |
|---|---|
| Compound | Structure |
| A | 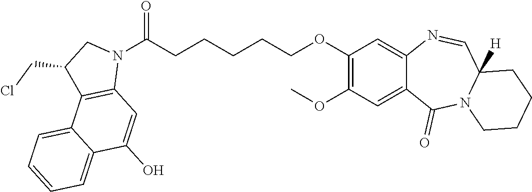 [SMILES: COc1cc2c(cc1OCCCCCC(=O)N1C[C@@H](CCl)c3c1cc(O)c1ccccc31)N=C[C@@H]1CCCCN1C2=O] |
| B | 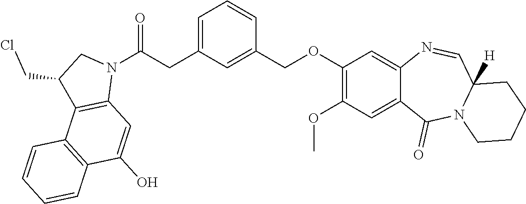 [SMILES: COc1cc2c(cc1OCc1cccc(CC(=O)N3C[C@@H](CCl)c4c3cc(O)c3ccccc43)c1)N=C[C@@H]1CCCCN1C2=O] |
| C | 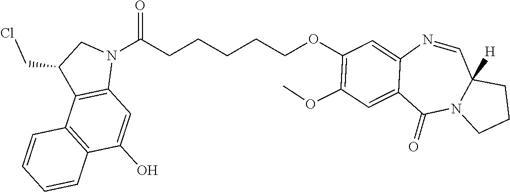 [SMILES: COc1cc2c(cc1OCCCCCC(=O)N1C[C@@H](CCl)c3c1cc(O)c1ccccc31)N=C[C@@H]1CCCN1C2=O] |
| D | 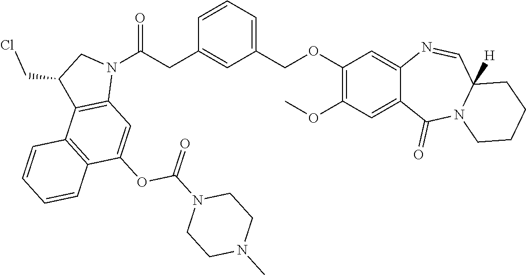 [SMILES: COc1cc2c(cc1OCc1cccc(CC(=O)N3C[C@@H](CCl)c4c3cc(OC(=O)N3CCN(C)CC3)c3ccccc43)c1)N=C[C@@H]1CCCCN1C2=O] |
| SN38 | 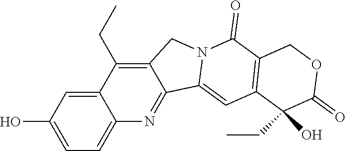 [SMILES: CCc1c2c(nc3ccc(O)cc13)-c1cc3c(c(=O)n1C2)COC(=O)[C@]3(O)CC] |
| SG3199 | 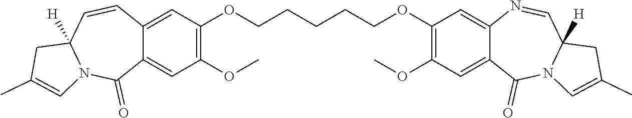 |
| MMAE | 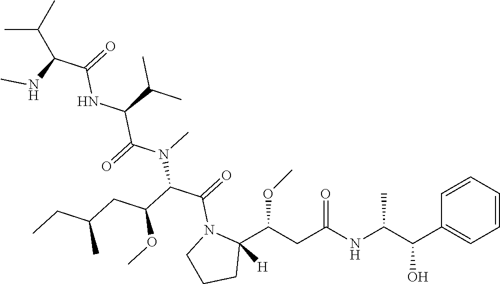 [SMILES: CC[C@H](C)C[C@H](OC)[C@@H](C(=O)N1CCC[C@H]1[C@@H](CC(=O)N[C@H](C)[C@@H](O)c1ccccc1)OC)N(C)C(=O)[C@@H](NC(=O)[C@@H](NC)C(C)C)C(C)C] |
| DGN549 | 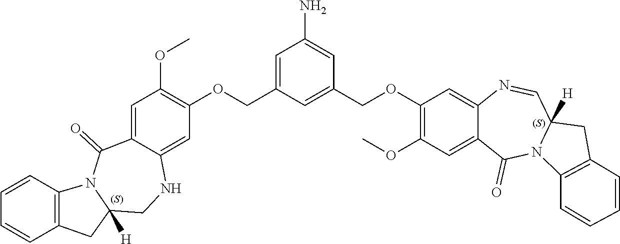 |
| SGD1882 | 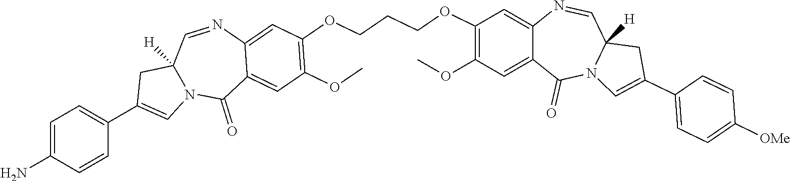 [SMILES: COc1ccc(C2=CN3C(=O)c4cc(OC)c(OCCCOc5cc6c(cc5OC)C(=O)N5C=C(c7ccc(N)cc7)C[C@H]5C=N6)cc4N=C[C@@H]3C2)cc1] |
| DM1 | 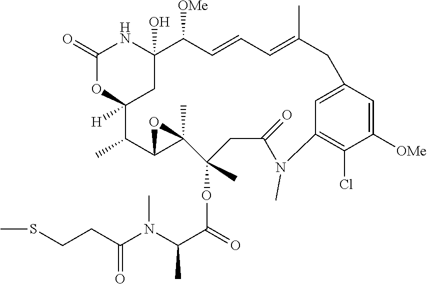 [SMILES: COc1cc2cc(c1Cl)N(C)C(=O)C[C@](C)(OC(=O)[C@@H](C)N(C)C(=O)CCSC)[C@]1(C)O[C@H]1[C@H](C)[C@@H]1C[C@@](O)(NC(=O)O1)[C@H](OC)/C=C/C=C(\C)C2] |

### Compound Numbers

Table 4 describes compound numbers for compounds in the application.

TABLE 4
Compound Numbers

| Structure | Cpd # |
|---|---|
| 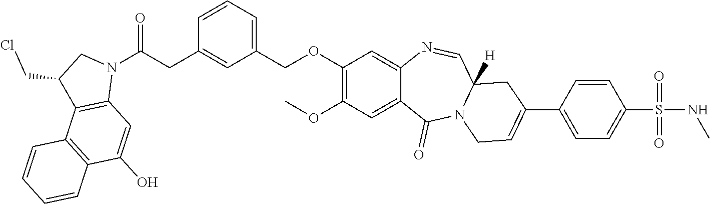 [SMILES: CNS(=O)(=O)c1ccc(C2=CCN3C(=O)c4cc(OC)c(OCc5cccc(CC(=O)N6C[C@@H](CCl)c7c6cc(O)c6ccccc76)c5)cc4N=C[C@@H]3C2)cc1] | 31 |
| 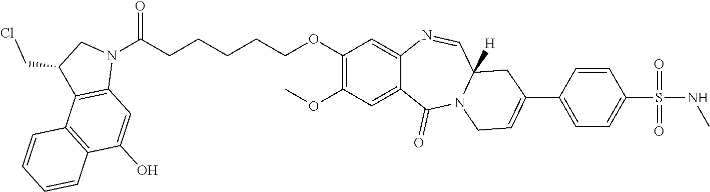 [SMILES: CNS(=O)(=O)c1ccc(C2=CCN3C(=O)c4cc(OC)c(OCCCCCC(=O)N5C[C@@H](CCl)c6c5cc(O)c5ccccc65)cc4N=C[C@@H]3C2)cc1] | 72 |
| 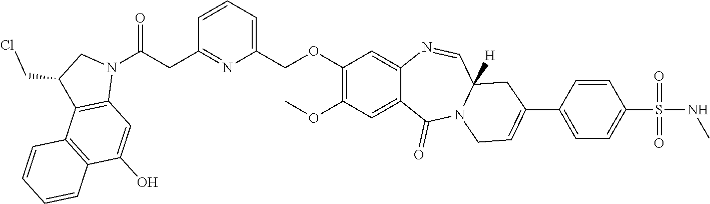 [SMILES: CNS(=O)(=O)c1ccc(C2=CCN3C(=O)c4cc(OC)c(OCc5cccc(CC(=O)N6C[C@@H](CCl)c7c6cc(O)c6ccccc76)n5)cc4N=C[C@@H]3C2)cc1] | 35 |
| 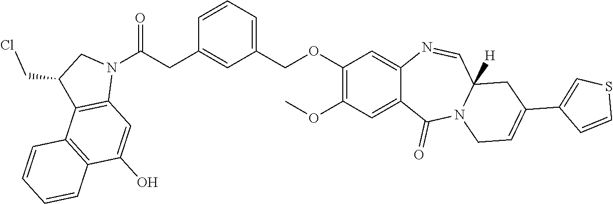 [SMILES: COc1cc2c(cc1OCc1cccc(CC(=O)N3C[C@@H](CCl)c4c3cc(O)c3ccccc43)c1)N=C[C@@H]1CC(c3ccsc3)=CCN1C2=O] | 45 |
| 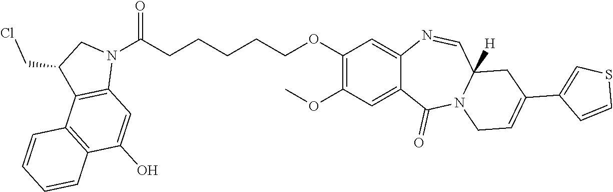 [SMILES: COc1cc2c(cc1OCCCCCC(=O)N1C[C@@H](CCl)c3c1cc(O)c1ccccc31)N=C[C@@H]1CC(c3ccsc3)=CCN1C2=O] | 96 |
| 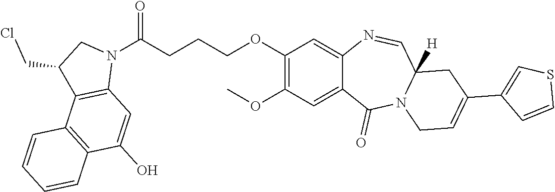 [SMILES: COc1cc2c(cc1OCCCC(=O)N1C[C@@H](CCl)c3c1cc(O)c1ccccc31)N=C[C@@H]1CC(c3ccsc3)=CCN1C2=O] | 100 |
| 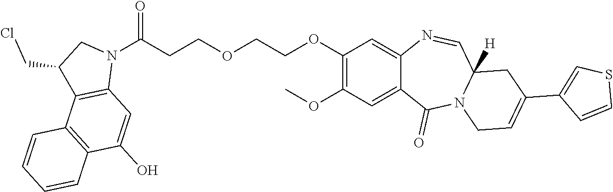 [SMILES: COc1cc2c(cc1OCCOCCC(=O)N1C[C@@H](CCl)c3c1cc(O)c1ccccc31)N=C[C@@H]1CC(c3ccsc3)=CCN1C2=O] | 104 |
| 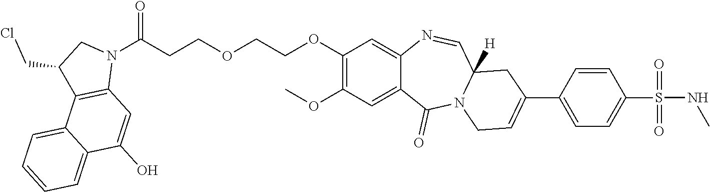 [SMILES: CNS(=O)(=O)c1ccc(C2=CCN3C(=O)c4cc(OC)c(OCCOCCC(=O)N5C[C@@H](CCl)c6c5cc(O)c5ccccc65)cc4N=C[C@@H]3C2)cc1] | 76 |
| 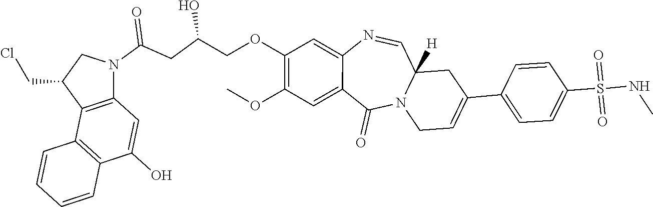 [SMILES: CNS(=O)(=O)c1ccc(C2=CCN3C(=O)c4cc(OC)c(OC[C@@H](O)CC(=O)N5C[C@@H](CCl)c6c5cc(O)c5ccccc65)cc4N=C[C@@H]3C2)cc1] | 88 |
| 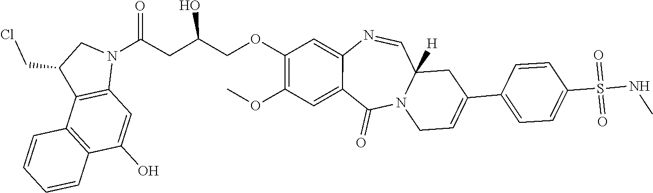 [SMILES: CNS(=O)(=O)c1ccc(C2=CCN3C(=O)c4cc(OC)c(OC[C@H](O)CC(=O)N5C[C@@H](CCl)c6c5cc(O)c5ccccc65)cc4N=C[C@@H]3C2)cc1] | 92 |
| 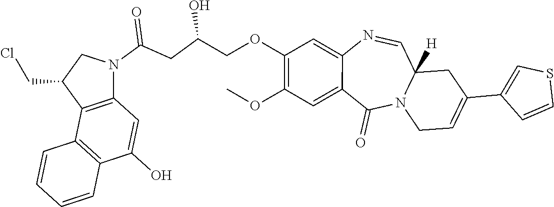 [SMILES: COc1cc2c(cc1OC[C@@H](O)CC(=O)N1C[C@@H](CCl)c3c1cc(O)c1ccccc31)N=C[C@@H]1CC(c3ccsc3)=CCN1C2=O] | 108 |

### REFERENCES

[1] aD. Antonow, T. C. Jenkins, P. W. Howard, D. E. Thurston, Bioorganic & Medicinal Chemistry 2007, 15, 3041-3053; bD. Antonow, D. E. Thurston, Chem Rev 2011, 111, 2815-2864.
[2] aL. Cipolla, A. C. Araujo, C. Airoldi, D. Bini, Anticancer Agents Med Chem 2009, 9, 1-31; bB. Gerratana, Med Res Rev 2012, 32, 254-293; cJ. A. Hartley, Expert Opin Investig Drugs 2011, 20, 733-744; dA. Kamal, K. L. Reddy, V. Devaiah, N. Shankaraiah, D. R. Reddy, Mini Rev Med Chem 2006, 6, 53-69.
[3] D. S. Bose, G. B. Jones, D. E. Thurston, Tetrahedron 1992, 48, 751-758.
[4] L. H. Hurley, T. Reck, D. E. Thurston, D. R. Langley, K. G. Holden, R. P. Hertzberg, J. R. Hoover, G. Gallagher, Jr., L. F. Faucette, S. M. Mong, et al., Chem Res Toxicol 1988, 1, 258-268.
[5] G. Wells, C. R. Martin, P. W. Howard, Z. A. Sands, C. A. Laughton, A. Tiberghien, C. K. Woo, L. A. Masterson, M. J. Stephenson, J. A. Hartley, T. C. Jenkins, S. D. Shnyder, P. M. Loadman, M. J. Waring, D. E. Thurston, Journal of medicinal chemistry 2006, 49, 5442-5461.
[6] aF. Brucoli, R. M. Hawkins, C. H. James, P. J. Jackson, G. Wells, T. C. Jenkins, T. Ellis, M. Kotecha, D. Hochhauser, J. A. Hartley, P. W. Howard, D. E. Thurston, Journal of medicinal chemistry 2013, 56, 6339-6351; bM. Kotecha, J. Kluza, G. Wells, C. C. O'Hare, C. Forni, R. Mantovani, P. W. Howard, P. Morris, D. E. Thurston, J. A. Hartley, D. Hochhauser, Mol Cancer Ther 2008, 7, 1319-1328.
[7] aM. S. Puvvada, J. A. Hartley, T. C. Jenkins, D. E. Thurston, Nucleic Acids Res 1993, 21, 3671-3675; bP. H. Clingen, I. U. De Silva, P. J. McHugh, F. J. Ghadessy, M. J. Tilby, D. E. Thurston, J. A. Hartley, Nucleic Acids Res 2005, 33, 3283-3291.
[8] M. S. Puvvada, S. A. Forrow, J. A. Hartley, P. Stephenson, I. Gibson, T. C. Jenkins, D. E. Thurston, Biochemistry 1997, 36, 2478-2484.
[9] aM. D. Barkley, S. Cheatham, D. E. Thurston, L. H. Hurley, Biochemistry 1986, 25, 3021-3031; bJ. Seifert, S. Pezeshki, A. Kamal, K. Weisz, Organic & Biomolecular Chemistry 2012, 10, 6850-6860.
[10] M. Smellie, D. S. Bose, A. S. Thompson, T. C. Jenkins, J. A. Hartley, D. E. Thurston, Biochemistry 2003, 42, 8232-8239.
[11] M. L. Kopka, D. S. Goodsell, I. Baikalov, K. Grzeskowiak, D. Cascio, R. E. Dickerson, Biochemistry 1994, 33, 13593-13610.
[12] R. Kizu, P. H. Draves, L. H. Hurley, Biochemistry 1993, 32, 8712-8722.
[13] Q. Zhou, W. Duan, D. Simmons, Y. Shayo, M. A. Raymond, R. T. Dorr, L. H. Hurley, J Am Chem Soc 2001, 123, 4865-4866.
[14] M. Tercel, S. M. Stribbling, H. Sheppard, B. G. Siim, K. Wu, S. M. Pullen, K. J. Botting, W. R. Wilson, W. A. Denny, Journal of medicinal chemistry 2003, 46, 2132-2151.
[15] K. K. Kothakonda, D. S. Bose, Bioorganic Medicinal Chemistry Letters 2004, 14, 4371-4373.

All publications mentioned in the above specification are herein incorporated by reference. Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

1. A compound of formula (I):
A-X₁—SP—X₂-B (I)
or salts, solvates, isomers or tautomers thereof,
wherein;
A is a group selected from the group consisting of: 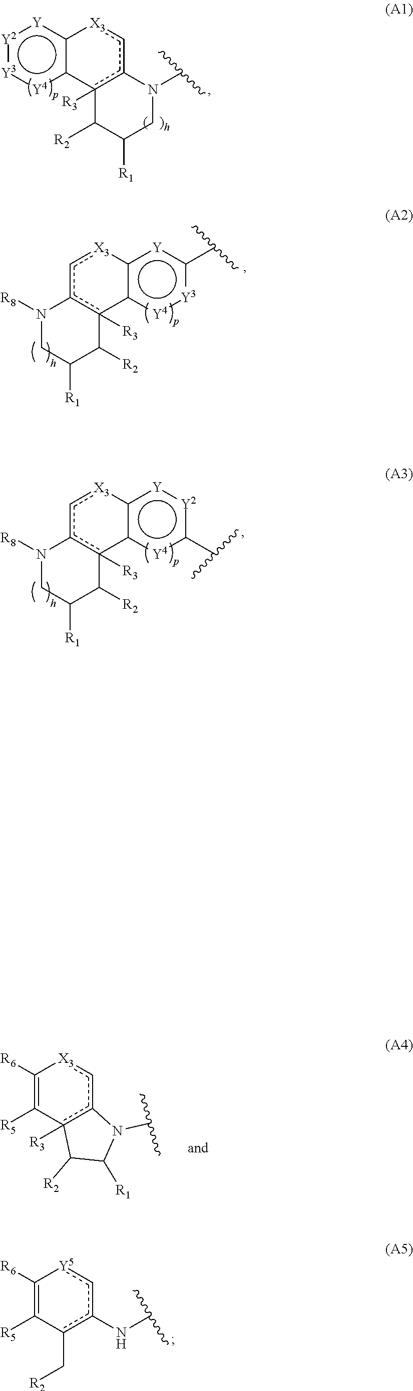
h is 0 or 1;
R₁ is selected from the group consisting of H and halogen;
either R₂ is selected from the group consisting of —CH₂-halogen, C₁₋₆ alkyl and H, and R₃ is H or is absent;
or R₂ and R₃ together with the carbon atoms to which they are attached form a cyclopropyl ring;
p is 0 or 1; and when p is 1 then Y is C—R₇, Y² is C—R₆, Y³ is C—R₅ and Y⁴ is C—R₄;
and for (A1) and (A2) when p is 0 either (a) Y is selected from the group consisting of N—R₁₉, O and S; Y² is selected from the group consisting of C—R₆ and N; and Y³ is C—R₅; or (b) Y³ is selected from the group consisting of N—R₁₉, O and S; Y² is selected from the group consisting of C—R₆ and N; and Y is C—R₇;
and for (A3) when p is 0, Y is selected from the group consisting of N—R₁₉, O and S; and Y² is selected from the group consisting of C—R₆ and N;
R₄, R₅, R₆ and R₇ are each independently selected from the group consisting of H, C₁₋₆ alkyl, OC₁₋₆ alkyl, CO₂H, CO₂C₁₋₆ alkyl, OCH₂Ph and R₂₀;
or one of R₄ and R₅, or R₅ and R₆, or R₆ and R₇ together with the carbon atoms to which they are attached form a 6-membered aryl, or a 5- or 6-membered cyclic, heterocyclic, or heteroaryl ring optionally substituted with 1, 2 or 3 optional groups independently selected from C₁₋₆ alkyl, OC₁₋₆ alkyl, OCH₂Ph and R₂₀;
R₈ is selected from the group consisting of H, C₁₋₆ alkyl, OC₁₋₆ alkyl, OCH₂Ph, nitrogen protecting groups and R₂₀;
X₃ is selected from the group consisting of C═O, C—OH and C—R₁₈; or Y⁵ is selected from the group consisting of C═O, C—OH, C—NH₂ and C—R₁₈; with the carbon forming part of the ring; and
when X₃ or Y⁵ is C═O then [symbol] represents an α,β-unsaturated double bond conjugated with the C═O; and when X₃ is C—OH or C—R₁₈ or Y⁵ is C—OH, C—NH₂ or C—R₁₈ then [symbol] represents the double bonds of an aromatic 6-membered ring and R₃ is absent;
wherein R¹⁸ is a prodrug moiety containing carbonyl, carbamoyl, glycosyl, O-amino, O-acylamino, para-aminobenzyl ether, peptidyl or phosphate groups;
X₁ is selected from the group consisting of O, S, NR₂₁, CR₂₁R₂₂, CR₂₁R₂₂O, C(O), C(O)NR₂₁, NR₂₁C(O), O—C(O), C(O)—O or is absent;
SP is selected from the group consisting of an amino acid, a peptide chain having from 2 to 12 amino acids, a paraformaldehyde chain —(CH₂O)₁₋₂₄—, a polyethylene glycol chain —(CH₂CH₂O)₁₋₁₂— and —(CH₂)ₘ—Y⁶—(CH₂)ₙ— wherein
m is 0, 1, 2, 3, 4, 5, 6, 7, 8, 9, 10, 11 or 12;
n is 0, 1, 2, 3, 4, 5, 6, 7, 8, 9, 10, 11 or 12; and
Y⁶ is selected from the group consisting of —(CH₂)_{z}—, arylene, heteroarylene, cycloalkylene, cycloalkenylene and heterocyclylene and the Y6 group is optionally substituted with 1, 2 or 3 independently selected optional C₁₋₆ alkyl, OC₁₋₆ alkyl, OCH₂Ph and R₂₀ groups;
z is 1, 2, 3, 4 or 5;
X₂ is selected from the group consisting of O, S, NR₂₃, CR₂₃R₂₄, CR₂₃R₂₄O, C(O), C(O)NR₂₃, NR₂₄C(O), O—C(O), C(O)—O or is absent;
B is a polycyclic group: 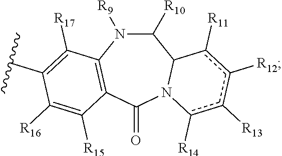
wherein the dotted lines indicate the optional presence of one or more double bonds;
and R₉ and R₁₀ are selected such that either:
(i) R₉ and R₁₀ together form a double bond;
(ii) R₉ is H and R₁₀ is OH;
(iii) R₉ is H and R₁₀ is OC₁₋₆ alkyl;
(iv) R₉ is SO₃H, a nitrogen protecting group or R₂₀; and R₁₀ is H; or
(v) R₉ is H or C₁₋₆ alkyl, and R₁₀ is oxo or H;
R₁₁, R₁₂, R₁₃ and R₁₄ are selected such that either:
(aa) one of R₁₁, R₁₂, R₁₃ and R₁₄ is E and another of R₁₁, R₁₂, R₁₃ and R₁₄ is an Ar¹ group optionally substituted with 1, 2 or 3 optional groups independently selected from the group consisting of C₁₋₆ alkyl, OC₁₋₆ alkyl, OCH₂Ph, E, R₂₀ and R₂₅; or
(ab) one of R₁₁, R₁₂, R₁₃ and R₁₄ is R^{X}, wherein R^{X} is —Ar¹—Z¹-E or -E¹-Z¹—Ar¹ and the Ar¹ is optionally further substituted with 1 or 2 optional groups independently selected from the group consisting of C₁₋₆ alkyl, OC₁₋₆ alkyl, OCH₂Ph, E, R₂₀ and R₂₅; or
(ac) one of R₁₁, R₁₂, R₁₃ and R₁₄ is R^{Y}, wherein R^{Y} is a 5- or 6-membered cyclic, heterocyclic, or heteroaryl ring optionally substituted with up to two groups independently selected from the group consisting of C₁₋₆ alkyl, OC₁₋₆ alkyl, OCH₂Ph, E, R₂₀ and R₂₅;
and for any of (aa) or (ab) or (ac) the remaining of R₁₁, R₁₂, R₁₃ and R₁₄ are independently selected from the group consisting of H, C₁₋₆ alkyl, OC₁₋₆ alkyl, OCH₂Ph, (CH₂)ⱼ—S(O)₂—NR₂₆R₂₇, R₂₀, R₂₅, ═CH₂, ═CH—(CH₂)ₛ—CH₃, ═CH—(CH₂)ₛ—R₂₅, ═O, (CH₂)ₛ—OR₂₅, (CH₂)ₛ—CO₂R₂₅, (CH₂)ₛ—NR₂₅R₂₆, O—(CH₂)ₜ—NR₂₅R₂₆, NH—C(O)—R₂₅, O—(CH₂)ₜ—NH—C(O)—R₂₅, O—(CH₂)ₜ—C(O)—NH—R₂₅, (CH₂)ₛ—SO₂R₂₅, O—SO₂R₂₅, (CH₂)ₛ—C(O)R₂₅ and (CH₂)ₛ—C(O)NR₂₅R₂₆;
wherein Ar¹ is an optionally substituted C₅₋₂₀ aryl or C₅₋₁₀ heteroaryl group; and
each E is independently selected from the group consisting of (CH₂)ⱼ—S(O)₂—NR₂₅R₂₆, (CH₂)ⱼ—S(O)₂—OH, CH₂CH₂[OCH₂CH₂]_{w}R₂₅ and E¹;
each E¹ is independently selected from the group consisting of pentose; hexose; C₅₋₆ heterocyclyl; C₅₋₁₀ heteroaryl group; C₅₋₁₀ heteroaryl group substituted with a C₅₋₆ heteroaryl or a C₅₋₆ heterocyclyl group; and phenyl substituted with a C₅₋₆ heteroaryl or a C₅₋₆ heterocyclyl group; wherein each E¹ group may be independently optionally substituted with 1, 2 or 3 optional groups independently selected from the group consisting of OC₁₋₆ alkyl, OCH₂Ph, R₂₀ and R₂₇; with the proviso that the E¹ group comprises at least two heteroatoms;
Z¹ is NR₂₆, C(═O)—O, O or is absent;
each w is independently 1, 2, 3, 4, 5, 6, 7, 8, 9 or 10;
each s is independently 0, 1, 2, 3, 4, 5 or 6;
each t is independently 1, 2, 3, 4, 5 or 6;
R₁₅, R₁₆ and R₁₇ are independently selected from the group consisting of H, C₁₋₆ alkyl, OC₁₋₆ alkyl, OCH₂Ph, R₂₀ and R₂₅;
each R₂₀ is independently selected from the group consisting of (CH₂)ⱼ—OH, (CH₂)ⱼ—CO₂R₂₇, C(O)R₂₇, O—(CH₂)ₖ—NR₂₇R₂₈, (CH₂)ⱼ—NR₂₇R₂₈, Ki-R*, NR₂₇NR₂₇R₂₈, C(O)—O—(CH₂)ₖ-NR₂₇R₂₈, C(O)NR₂₇R₂₈, NR₂₇—CO₂R₂₇, C(O)—NH—(CH₂)ₖ-NR₂₇R₂₈, C(O)—NH—C₆H₄—(CH₂)ⱼ—R₂₇ and C(O)—NH—(CH₂)ₖ—C(═NH)NR₂₇R₂₈;
each j is independently 0, 1, 2, 3, 4, 5 or 6;
each k is independently 1, 2, 3, 4, 5 or 6;
each R₁₉, R₂₁, R₂₂, R₂₃, R₂₄, R₂₆ and R₂₈ is independently selected from the group consisting of H and C₁₋₆ alkyl;
each R₂₅ is independently selected from the group consisting of H, C₁₋₁₂ alkyl, C₅₋₉ heteroaryl, C₆₋₁₆ heteroarylalkyl, phenyl and C₆₋₂₆ aralkyl groups; wherein the heteroaryl, heteroarylalkyl, phenyl and aralkyl groups are optionally substituted with 1, 2 or 3 independently selected optional C₁₋₆ alkyl, OC₁₋₆ alkyl and R₂₀ groups;
each R₂₇ is independently selected from the group consisting of H, C₁₋₆ alkyl, C₅₋₂₀ aryl and C₆₋₂₆ aralkyl; and
K₁ is a bond or a linker moiety having 1-200 non-hydrogen atoms selected from the group consisting of C, N, O, S or halogen, and optionally incorporates alkyl, ether, oxo, carboxyl, carboxamide, carboxamidyl, ester, urethanyl, branched, cyclic, unsaturated, heterocyclyl, aryl or heteroaryl moieties; and
R* is an azide, alkyne, bisulfone, carbohydrazide, hydrazine, hydroxylamine, iodoacetamide, isothiocyanate, maleimide, phosphine, pyrridopyridazine, semihydrazide, succinimidyl ester, sulfodichlorophenol ester, sulfonyl halide, sulfosuccinimidyl ester, 4-sulfotetrafluorophenyl ester, tetrafluorophenyl ester, thiazole, R₂₀, O—(CH₂)ₖ—NR₂₆R₂₆, NHNH₂, or is a targeting agent wherein the targeting agent is selected from the group consisting of a protein, a portion of a protein, a polypeptide, a nucleic acid, a hormone, an antibody or an antibody fragment;
with the proviso that when R₂ is C₁₋₆ alkyl or H, that R₉ and R₁₀ are selected from the group consisting of options (i), (ii), (iii) or (iv); and
with the proviso that when (v) R₉ is H or C₁₋₆ alkyl, and R₁₀ is oxo or H; then either R₂ is —CH₂-halogen and R₃ is H;
or R₂ and R₃ together with the carbon atoms to which they are attached form a cyclopropyl ring.

2. The compound of formula (I) or salts, solvates, isomers or tautomers thereof according to claim 1, wherein A is (A1).

3. The compound of formula (I) according to claim 1, wherein the compound is a compound of formula (VII): 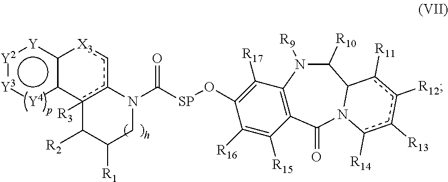
or salts, solvates, isomers or tautomers thereof.

4. The compound of formula (I) or salts, solvates, isomers or tautomers thereof according to claim 1, wherein A is:

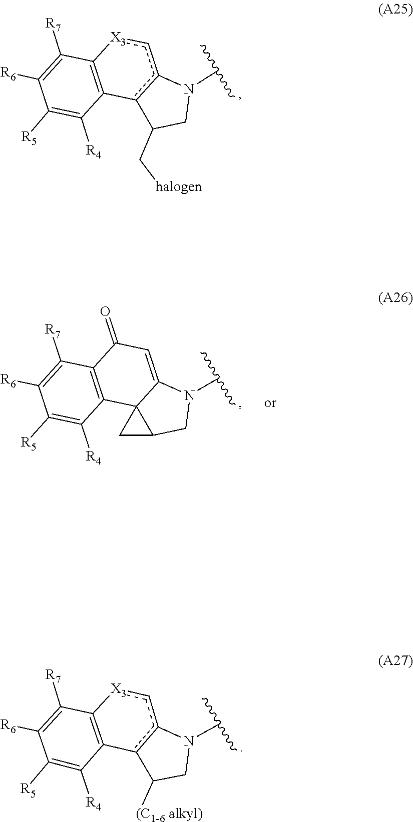

5. The compound of formula (I) or salts, solvates, isomers or tautomers thereof according to claim 1, wherein A is:

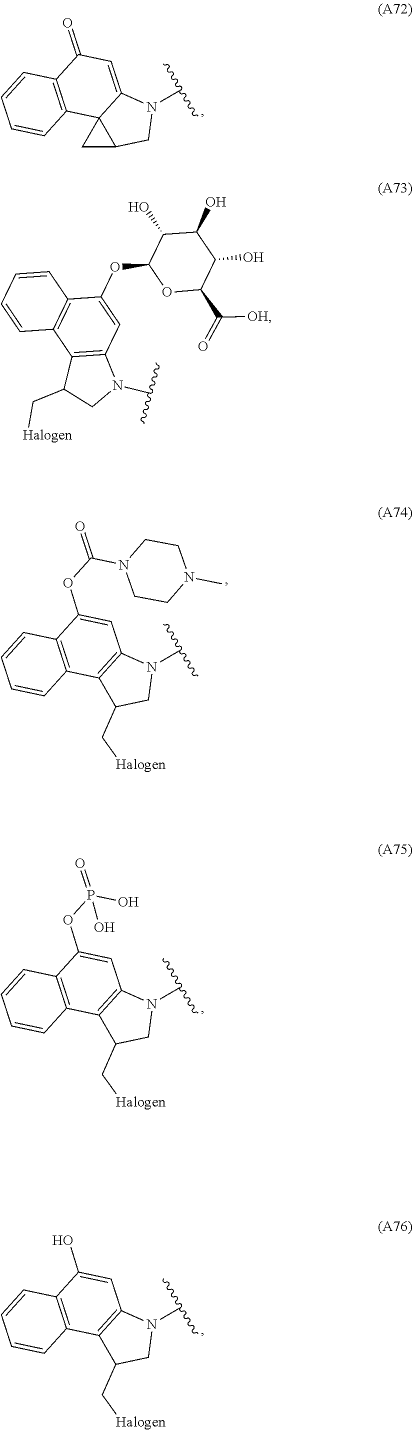

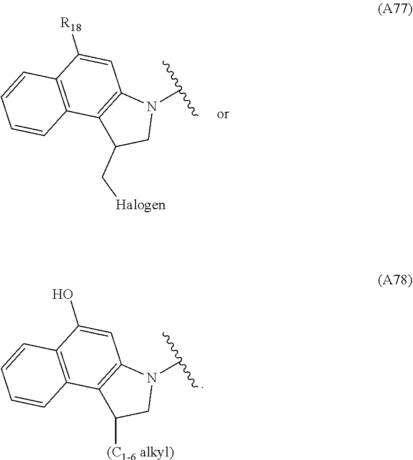

6. The compound of formula (I) or salts, solvates, isomers or tautomers thereof according to claim 1, wherein SP is selected from —CH₂O—, —CH₂O—CH₂O—, —CH₂O—CH₂O—CH₂O—, —CH₂CH₂O—, —CH₂CH₂O—CH₂CH₂O—, —CH₂CH₂O—CH₂CH₂O—CH₂CH₂O—, —(CH₂)ₘ(CH₂)_{z}—(CH₂)ₙ—, 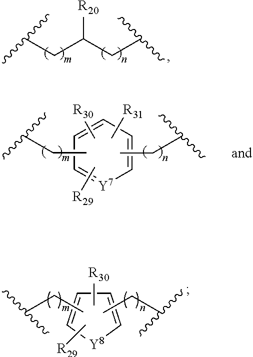
wherein R₂₉, R₃₀, R₃₁ and R₃₂ are each independently selected from H, C₁₋₆ alkyl, OC₁₋₆ alkyl, OCH₂Ph and R₂₀.

7. The compound of formula (I) or salts, solvates, isomers or tautomers thereof according to claim 6, wherein SP is:

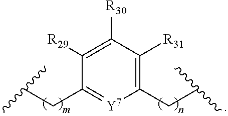

8. The compound of formula (I) or salts, solvates, isomers or tautomers thereof according to a claim 1, wherein B is:

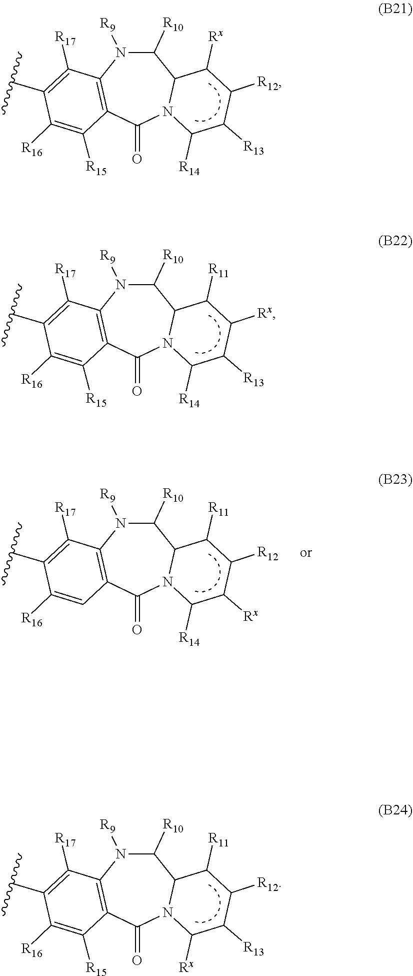

9. The compound of formula (I) or salts, solvates, isomers or tautomers thereof according to claim 1, wherein B is: 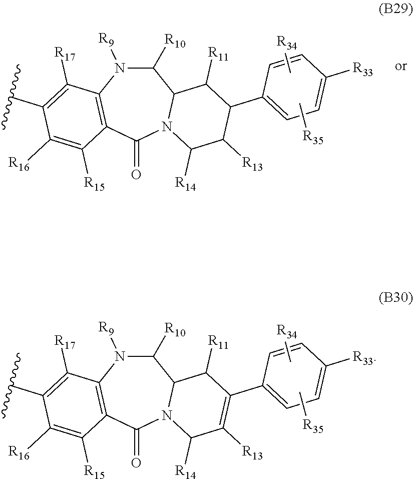
wherein R₃₃, R₃₄ and R₃₅ are each independently selected from the group consisting of H, E, C₁₋₆ alkyl, OC₁₋₆ alkyl, OCH₂Ph, and R₂₀.

10. The compound of formula (I) or salts, solvates, isomers or tautomers thereof according to claim 1, wherein the compound comprises a Ki-R* group.

11. The compound of formula (I) according to claim 1, wherein the compound is: 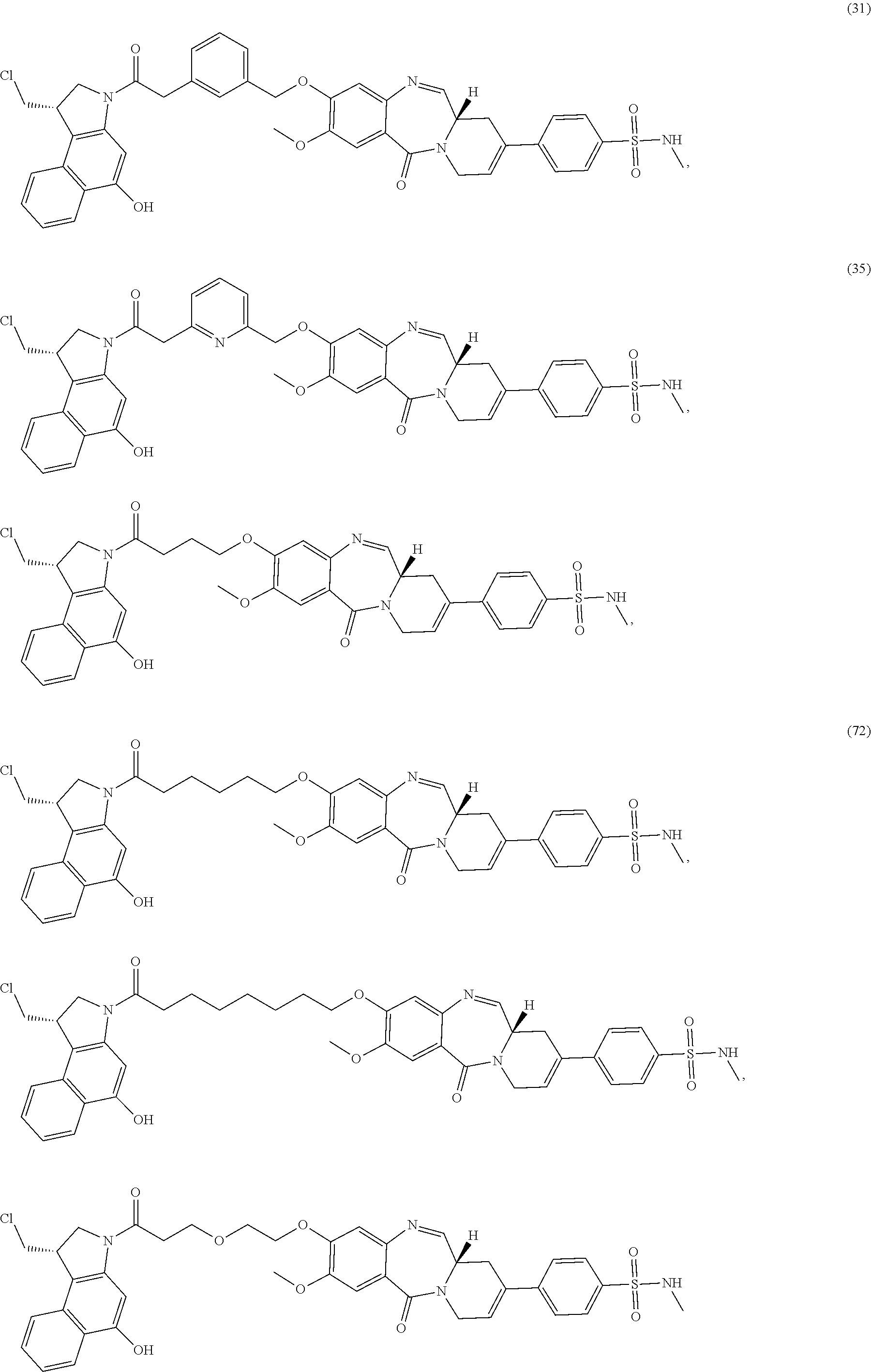 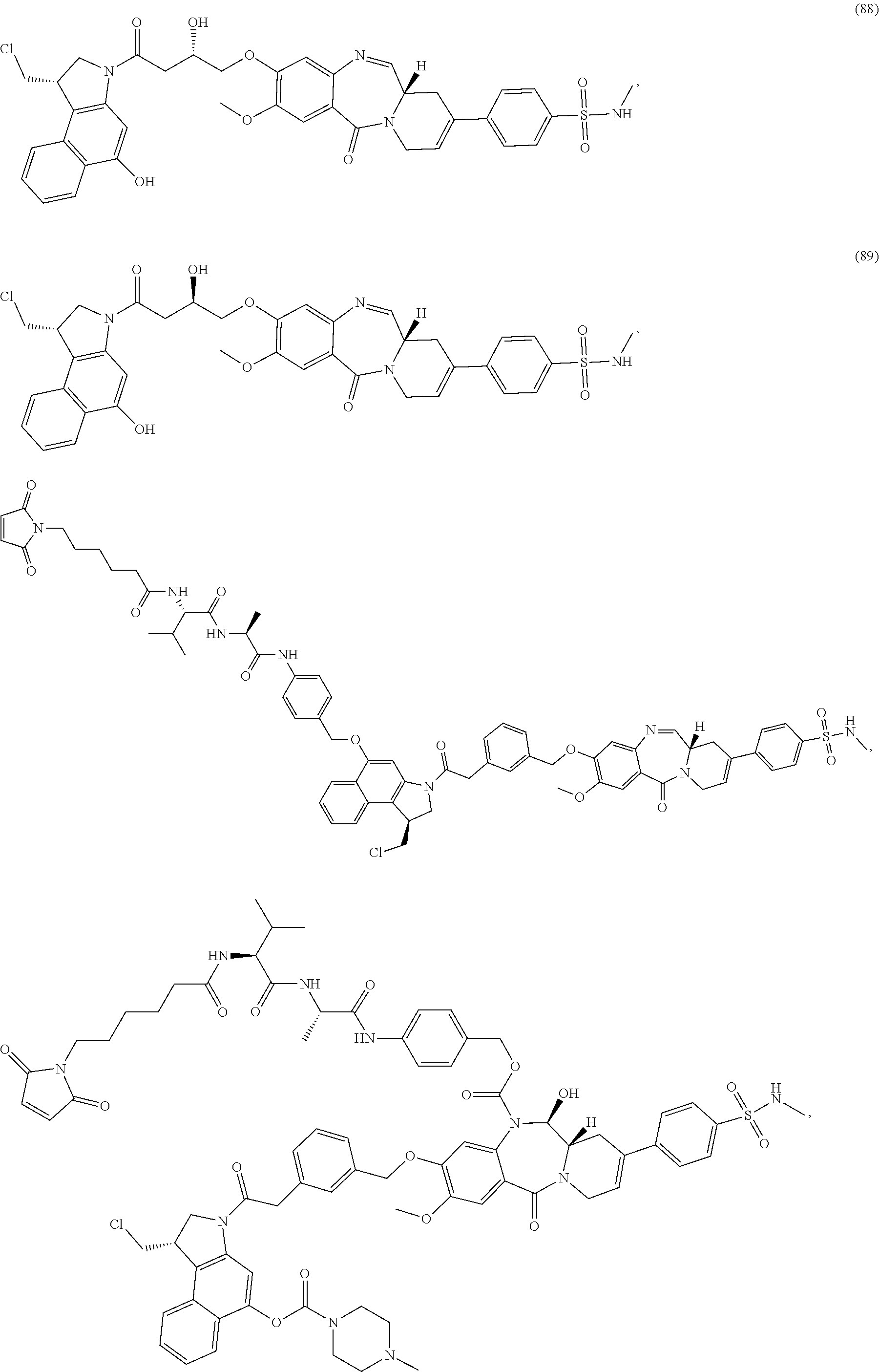 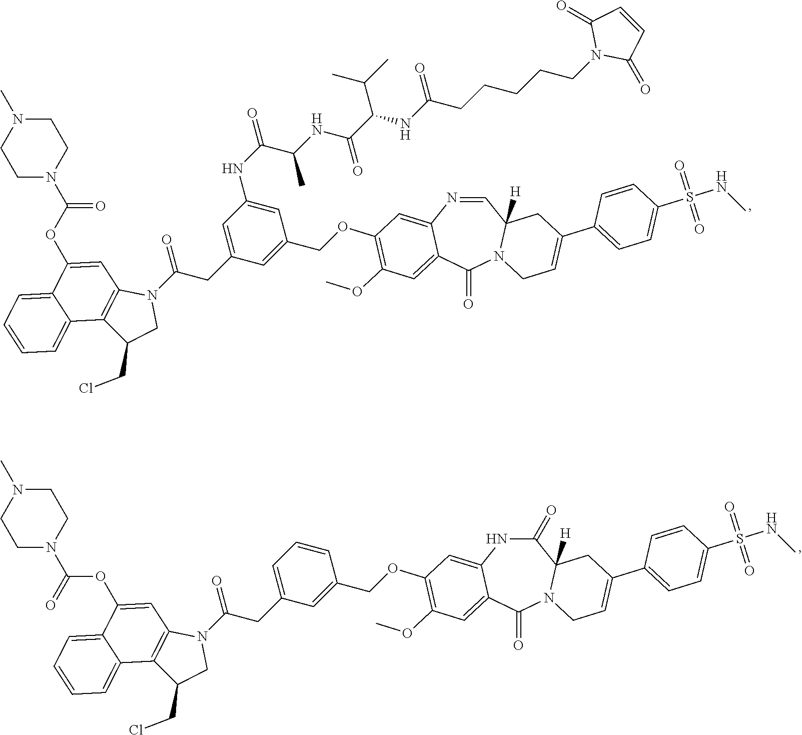 [SMILES: CNS(=O)(=O)c1ccc(C2=CCN3C(=O)c4cc(OC)c(OCc5cc(CC(=O)N6C[C@@H](CCl)c7c6cc(OC(=O)N6CCN(C)CC6)c6ccccc76)cc(NC(=O)[C@H](C)NC(=O)[C@@H](NC(=O)CCCCCN6C(=O)C=CC6=O)C(C)C)c5)cc4N=C[C@@H]3C2)cc1 ; CNS(=O)(=O)c1ccc(C2=CCN3C(=O)c4cc(OC)c(OCc5cccc(CC(=O)N6C[C@@H](CCl)c7c6cc(OC(=O)N6CCN(C)CC6)c6ccccc76)c5)cc4NC(=O)[C@@H]3C2)cc1] 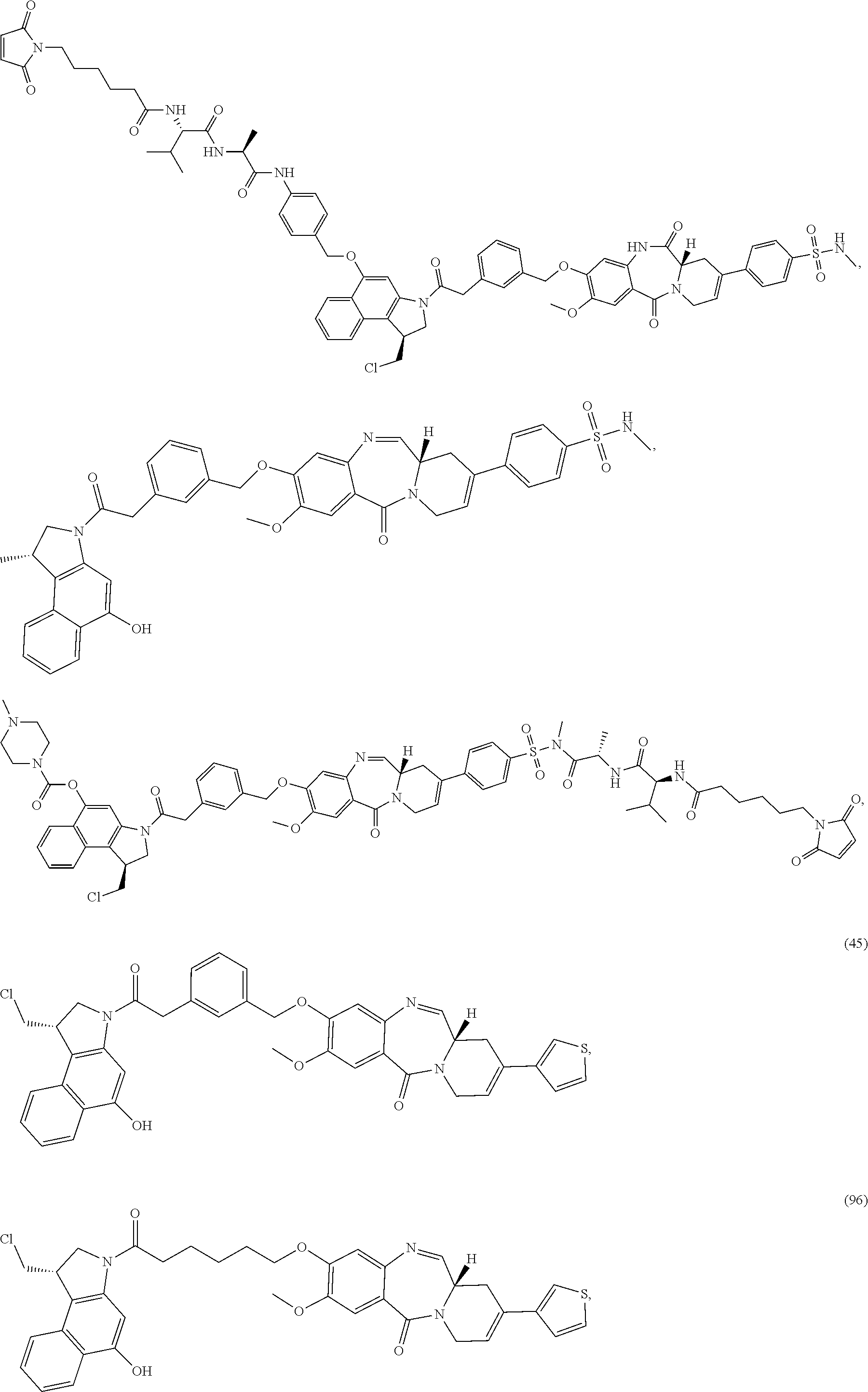 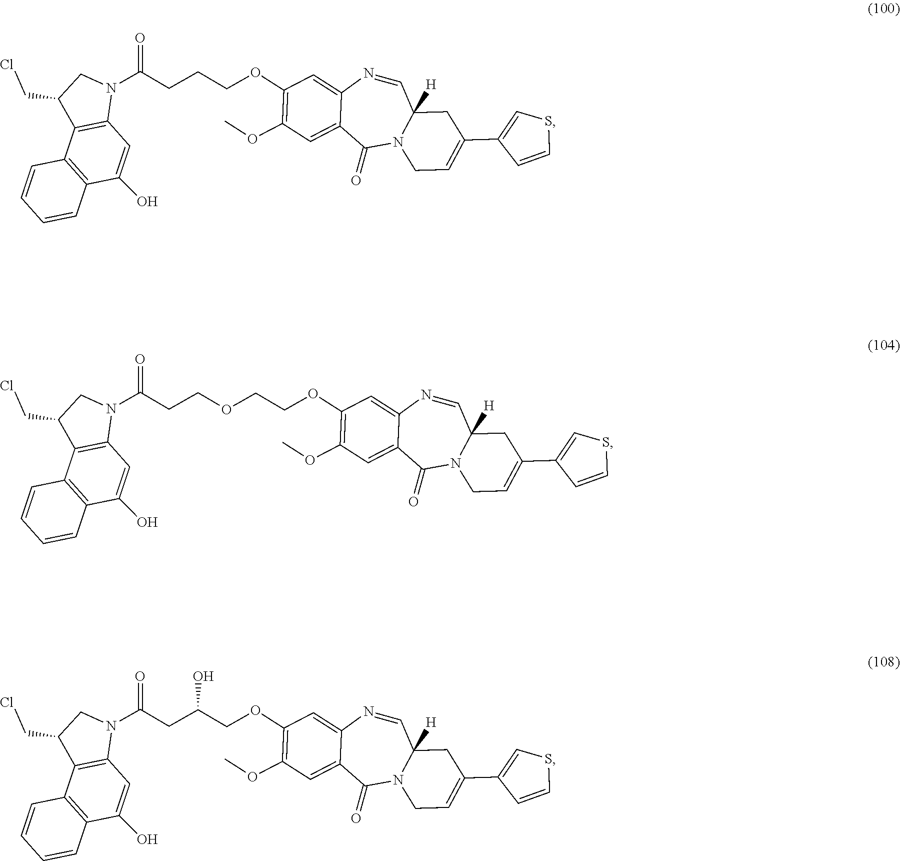 [SMILES: COc1cc2c(cc1OCCCC(=O)N1C[C@@H](CCl)c3c1cc(O)c1ccccc31)N=C[C@@H]1CC(c3ccsc3)=CCN1C2=O ; COc1cc2c(cc1OCCOCCC(=O)N1C[C@@H](CCl)c3c1cc(O)c1ccccc31)N=C[C@@H]1CC(c3ccsc3)=CCN1C2=O ; COc1cc2c(cc1OC[C@@H](O)CC(=O)N1C[C@@H](CCl)c3c1cc(O)c1ccccc31)N=C[C@@H]1CC(c3ccsc3)=CCN1C2=O]
or salts, solvates, isomers or tautomers thereof.

12. A pharmaceutical composition comprising the compound of formula (I) or salts, solvates, tautomers, isomers or mixtures thereof according to claim 1, and a pharmaceutically acceptable carrier, diluent, or excipient.

13. The compound of formula (I) or salts, solvates, tautomers, isomers or mixtures thereof according to claim 1 for use as a medicament.

14. The compound of formula (I) or salts, solvates, tautomers, isomers or mixtures thereof according to claim 1, for use as a drug in an antibody-drug conjugate.

15. The compound of formula (I) or salts, solvates, tautomers, isomers or mixtures thereof according to claim 1, for use in the treatment of a proliferative disease, a bacterial infection, a malarial infection and inflammation.

16. The compound of formula (I) or salts, solvates, tautomers, isomers or mixtures thereof, wherein the proliferative disease is selected from bladder cancer, bone cancer, bowel cancer, brain cancer, breast cancer, cervical cancer, colon cancer, head and neck cancer, leukemia, liver cancer, lung cancer, lymphoma, melanoma, oesophageal cancer, oral cancer, ovarian cancer, pancreatic cancer, prostate cancer, rectal cancer, renal cancer, retinoblastoma, sarcoma, skin cancer, stomach cancer, testicular cancer, thyroid cancer and uterine cancer.

17. The pharmaceutical composition according to claim 12, for use as a medicament.

18. The pharmaceutical composition according to claim 12 for use in the treatment of a proliferative disease, a bacterial infection, a malarial infection and inflammation.

19. The pharmaceutical composition for use in the treatment of a proliferative disease according to claim 18, wherein the proliferative disease is selected from bladder cancer, bone cancer, bowel cancer, brain cancer, breast cancer, cervical cancer, colon cancer, head and neck cancer, leukemia, liver cancer, lung cancer, lymphoma, melanoma, oesophageal cancer, oral cancer, ovarian cancer, pancreatic cancer, prostate cancer, rectal cancer, renal cancer, retinoblastoma, sarcoma, skin cancer, stomach cancer, testicular cancer, thyroid cancer and uterine cancer.
